(19) United States
(12) Patent Application Publication
(10) Pub. No.: US 2022/0143012 A1
(43) Pub. Date: May 12, 2022

(54) Macromolecule-Supported TLR Agonists

(71) Applicants: Bolt Biotherapeutics, Inc., Redwood City, CA (US); The Board of Trustees of The Leland Stanford Junior University, Stanford, CA (US)

(72) Inventors: Shelley Erin Ackerman, Redwood City, CA (US); Michael N. Alonso, Redwood City, CA (US); David Dornan, Redwood City, CA (US); Justin Kenkel, Redwood City, CA (US); Romas Kudirka, Redwood City, CA (US); Arthur Lee, Redwood City, CA (US); Brian Safina, Redwood City, CA (US); Matthew Zhou, Redwood City, CA (US); Edgar George Engleman, Atherton, CA (US)

(73) Assignees: Bolt Biotherapeutics, Inc., Redwood City, CA (US); The Board of Trustees of The Leland Stanford Junior University, Stanford, CA (US)

(21) Appl. No.: 17/439,136

(22) PCT Filed: Mar. 13, 2020
(86) PCT No.: PCT/US2020/022740

§ 371 Date: Sep. 14, 2021

Related U.S. Application Data
(60) Provisional application No. 62/819,365, filed on Mar. 15, 2019.

(51) Int. Cl. A61K 31/496; A61K 31/4745; A61K 47/64; A61K 47/68; A61K 47/54; A61K 47/69; A61K 47/60

(52) U.S. Cl. CPC A61K 31/496; A61K 31/4745; A61K 47/64; A61K 47/60; A61K 47/549; A61K 47/6931; A61K 47/6803

(57) ABSTRACT

The invention provides a macromolecule-supported compound of formula (I) or (II). Macromolecule-supported compounds of the invention, comprising macromolecular support linked to one or more TLR agonists, are recognized by TLRs (e.g., TLR7 and/or TLR8) with high affinity providing utility in therapeutics, diagnostics, and chemical assays. The invention further provides compositions comprising and methods of treating cancer with the macromolecule-supported compounds.

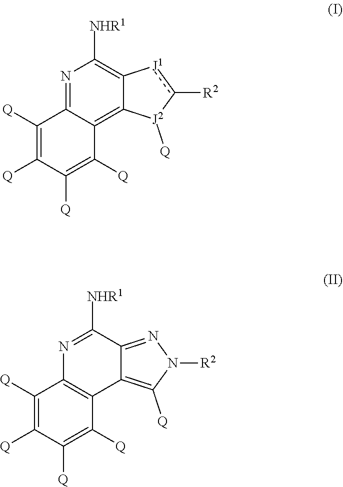

### CROSS-REFERENCE TO RELATED APPLICATIONS

This patent application claims the benefit of U.S. Provisional Patent Application No. 62/819,365, filed Mar. 15, 2019, which is incorporated by reference in its entirety herein.

### BACKGROUND OF THE INVENTION

It is now well appreciated that tumor growth necessitates the acquisition of mutations that facilitate immune evasion. Even so, tumorigenesis results in the accumulation of mutated antigens, or neoantigens, that are readily recognized by the host immune system following ex vivo stimulation. Why and how the immune system fails to recognize neoantigens are beginning to be elucidated. Groundbreaking studies by Carmi et al. (Nature, 521: 99-104 (2015)) have indicated that immune ignorance can be overcome by delivering neoantigens to activated dendritic cells via antibody-tumor immune complexes. In these studies, simultaneous delivery of tumor binding antibodies and dendritic cell adjuvants via intratumoral injections resulted in robust anti-tumor immunity. New dendritic cell adjuvants (i.e., toll-like receptor adjuvants) and methods for the delivery of dendritic cell adjuvants are needed in order to reach inaccessible tumors and/or to expand treatment options for cancer patients and other subjects. The invention provides such dendritic cell adjuvants, compositions, and methods.

### BRIEF SUMMARY OF THE INVENTION

The invention provides macromolecule-supported compound of formula (I):

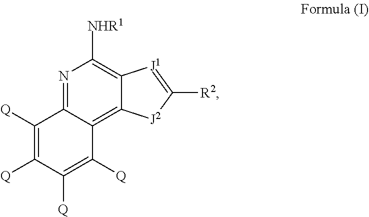

a pharmaceutically acceptable salt thereof, or a quaternary ammonium salt thereof, wherein

R¹ and R² independently are hydrogen or of formula:

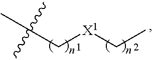

J¹ is CH or N,

J² is CHQ, NQ, O, or S,

each Q independently is Y or Z, wherein exactly one Q is Y,

Y is of formula:

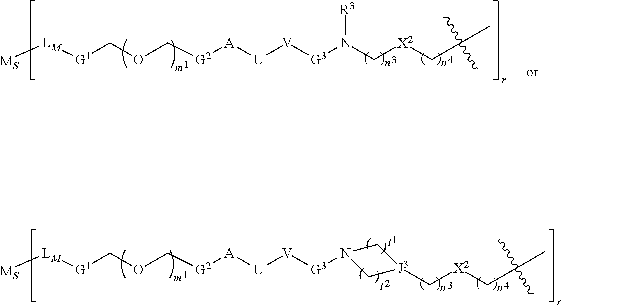

each Z independently is hydrogen or of formula:

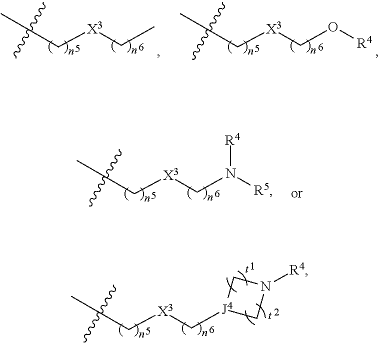

A is optionally present and is NR⁶ or of formula:

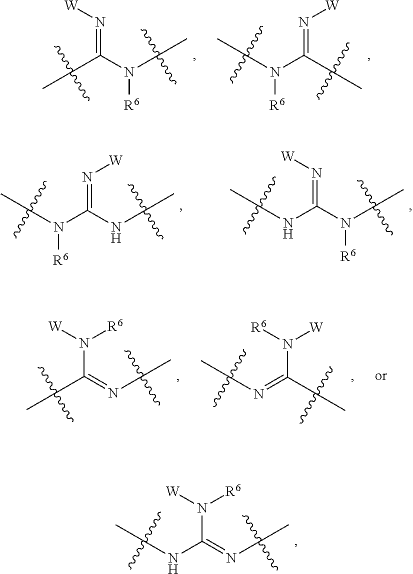

U is optionally present and is CH₂, C(O), CH₂C(O), or C(O)CH₂,

R⁶ and W independently are hydrogen, Ar¹, or of formula:

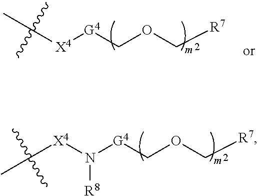

V is optionally present and is of formula:

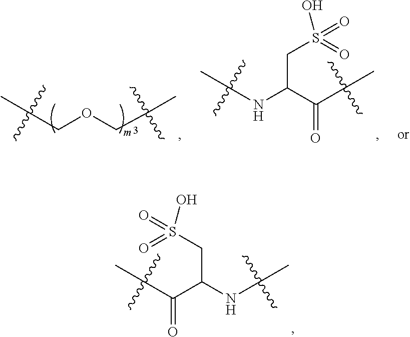

J³ and J⁴ independently are CH or N,

m¹, m², and m³ independently are an integer from 0 to 25, except that at least one of m¹, m², and m³ is a non-zero integer,

n¹, n², n³, n⁴, n⁵, and n⁶ independently are an integer from 0 to 10,

t¹ and t² independently are an integer from 1 to 3,

G¹, G², G³, and G⁴ independently are CH₂, C(O), CH₂C(O), C(O)CH₂, or a bond, X¹, X², X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

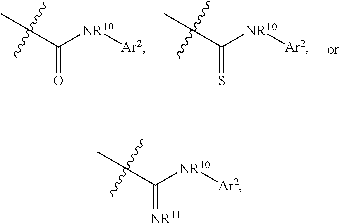

R³, R⁵, R⁷, R⁸, R⁹, R¹⁰, and R¹¹ independently are hydrogen or C₁-C₄ alkyl,
Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

The invention provides a macromolecule-supported compound of formula (II):

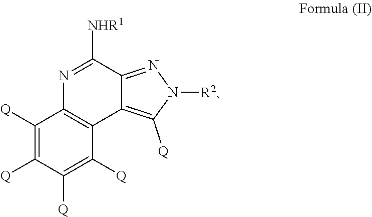

a pharmaceutically acceptable salt thereof, or a quaternary ammonium salt thereof, wherein

R¹ and R² independently are hydrogen or of formula:

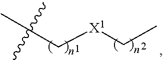

each Q independently is Y or Z, wherein exactly one Q is Y,

Y is of formula:

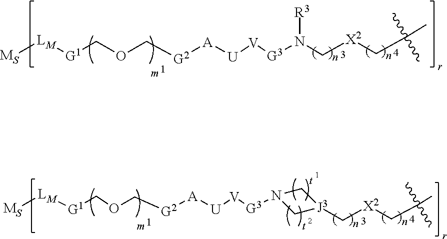

each Z independently is hydrogen or of formula:

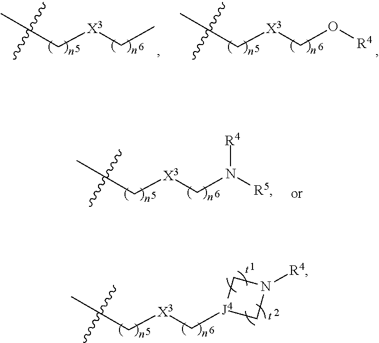

A is optionally present and is NR⁶ or of formula:

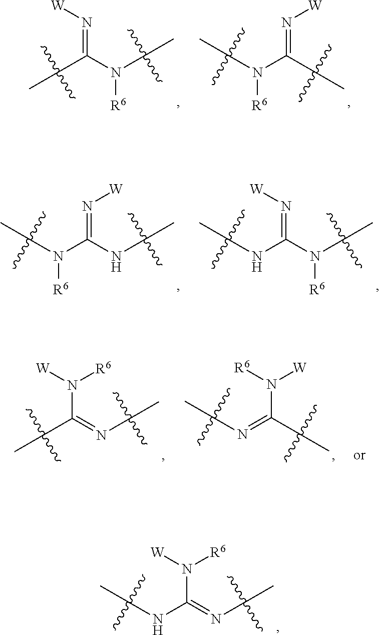

U is optionally present and is CH₂, C(O), CH₂C(O), or C(O)CH₂,

R⁶ and W independently are hydrogen, Ar¹, or of formula:

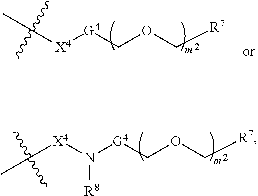

V is optionally present and is of formula:

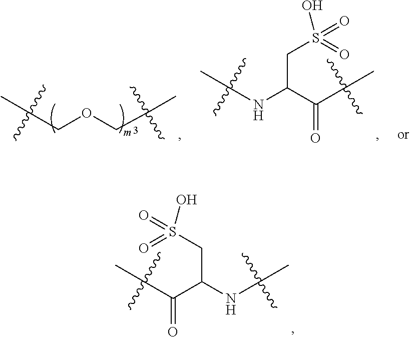

J³ and J⁴ independently are CH or N,

m¹, m², and m³ independently are an integer from 0 to 25, except that at least one of m¹, m², and m³ is a non-zero integer,

n¹, n², n³, n⁴, n⁵, and n⁶ independently are an integer from 0 to 10,

t¹ and t² independently are an integer from 1 to 3,

G¹, G², G³, and G⁴ independently are CH₂, C(O), CH₂C(O), C(O)CH₂, or a bond,

X¹, X², X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

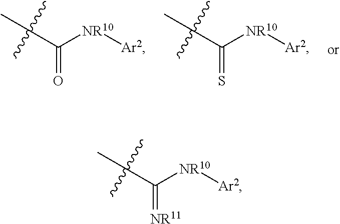

R³, R⁵, R⁷, R⁸, R⁹, R¹⁰, and R¹¹ independently are hydrogen or C₁-C₄ alkyl, Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

The invention provides a composition comprising a plurality of macromolecule-supported compounds described herein.

The invention provides a method of recognizing TLR (e.g., TLR7 and/or TLR8) for use in therapeutics, diagnostics, or chemical assays.

The invention provides a method for treating cancer in a subject comprising administering a therapeutically effective amount of a macromolecule-supported compound or a composition described herein to a subject in need thereof.

The invention provides a use of a macromolecule-supported compound or a composition of macromolecule-supported compounds for a chemical assay for TLR engagement and/or activity (e.g., TLR7 and/or TLR8 engagement and/or activity).

### DETAILED DESCRIPTION OF THE INVENTION

The invention provides a macromolecule-supported compound of formula (I) or (II). Macromolecule-supported compounds of the invention, comprising a macromolecular support linked to one or more toll-like receptor (“TLR”) agonists, maintain elevated function of the one or more TLR agonists, facilitating their use in therapeutic applications, diagnostic applications, and chemical assays. Additional embodiments and benefits of the inventive macromolecule-supported compounds will be apparent from description herein.

### Definitions

As used herein, the phrase “macromolecule-supported compound” refers to a macromolecular support that is covalently bonded to a TLR agonist via a linking moiety.

As used herein, the terms “macromolecule support,” “macromolecular support,” or “macromolecule” can be used interchangeably to refer to an organic or inorganic structure having a chemical moiety on a surface of the structure that can be modified. In some embodiments, the macromolecular support is a resin, bead, probe, tag, well, plate, or any other surface that can be used for therapeutics, diagnostics, or chemical assays. The resin, bead, probe, tag, well, plate, or any other surface can be made of any suitable material so long as the material can be surface modified. In some embodiments, the macromolecular support is a chemical structure (e.g., a biological structure or an inorganic framework) having a molecular weight of at least about 200 Da (e.g., at least about 500 Da, at least about 1,000 Da, at least about 2,000 Da, at least about 5,000 Da, or at least about 10,000 Da). As a singular entity, the macromolecular support can be biologically active or biologically inactive relative to the TLR agonist described herein. However, when used in combination with the TLR agonist, the biological activity of the TLR agonist desirably is enhanced, for example, by providing a targeted effect (i.e., TLR activity), beneficial off-target effects (i.e., biological activity other than TLR activity), improved pharmacokinetic properties (e.g., half-life extension), enhanced biological delivery (e.g., tumor penetration), or additional biological stimulation, differentiation, up-regulation, and/or down-regulation. In certain embodiments, the biological effect of the macromolecular support and the TLR agonist is synergistic, i.e., greater than the sum of the biological activity of each of the macromolecular support and TLR agonist as singular entities. For example, the macromolecular support can be a biopolymer (e.g., a glycopolymer, a cellulosic polymer, etc.), a nanoparticle (e.g., a carbon nanotube, a quantum dot, a metal nanoparticle (e.g., silver, gold, titanium dioxide, silicon dioxide, zirconium dioxide, aluminum oxide, or ytterbium trifluoride), etc.), a lipid (e.g., lipid vesicles, micelles, liposomes, etc.), a carbohydrate (e.g., sugar, starch, cellulose, glycogen, etc.), a peptide (e.g., a polypeptide, a protein, a peptide mimetic, a glycopeptide, etc.), an alternative protein scaffold, an antibody construct (e.g., antibody, an antibody-derivative (including Fc fusions, Fab fragments and scFvs), etc.), a nucleotide (e.g., RNA, DNA, antisense, siRNA, an aptamer, etc.), or any combination thereof. In some embodiments, the macromolecular support is a peptide, a nucleotide, a sugar, a lipid, or an antibody. In certain embodiments, the macromolecular support is an immune checkpoint inhibitor.

As used herein, the term “biopolymer” refers to any polymer produced by a living organism. For example, biopolymer can include peptides, polypeptides, proteins, oligonucleotides, nucleic acids (e.g., RNA and DNA) antibodies, polysaccharides, carbohydrates, sugars, peptide hormones, glycoproteins, glycogen, etc. Alternatively, a subunit of a biopolymer, such as a fatty acid, glucose, an amino acid, a succinate, a ribonucleotide, a ribonucleoside, a deoxyribonucleotide, and a deoxyribonucleoside can be used. Illustrative examples include antibodies or fragments thereof; extracellular matrix proteins such as laminin, fibronectin, growth factors, peptide hormones, and other polypeptides. In some embodiments, the biopolymer comprises suberin, melanin, lignin, or cellulose, or the biopolymer is glycosidic.

As used herein, the term “nanoparticle” refers to a support structure having a diameter of about 1 nm to about 100 nm. Exemplary structure types include nanopowders, nanoparticles, nanoclusters, nanorods, nanotubes, nanocrystals, nanospheres, nanochains, nanoreefs, nanoboxes, and quantum dots. The nanoparticles can contain an inorganic material (e.g., silver, gold, hydroxyapatite, clay, titanium dioxide, silicon dioxide, zirconium dioxide, carbon (graphite), diamond, aluminum oxide, ytterbium trifluoride, etc.) or an organic material (e.g., micelles, dendrimers, vesicles, liposomes, etc.). Alternatively, the nanoparticle can have a mixture of organic and inorganic material.

As used herein the term “lipid” refers to a hydrophobic or amphiphilic biomolecule. Exemplary lipids include fatty acids, waxes, sterols, fat-soluble vitamins, monoglycerides, diglycerides, triglycerides, phospholipids, sphingolipids, saccharolipids, polyketides, sterol lipids, glycerophospholipids, prenol lipids, etc. The lipid can exist in any suitable macromolecular structure, for example, a vesicle, a micelle, a liposome, etc.

As used herein, the term “carbohydrate” refers to any chemical entity comprising a monosaccharide, disaccharide, oligosaccharide, or polysaccharide. For example, the chemical entity can comprise a sugar (e.g., fructose, glucose, sucrose, lactose, galactose, etc.), starch, glycogen, or cellulose.

The terms “polypeptide,” “peptide,” and “protein” are used interchangeably herein to refer to a polymer of amino acid residues. The terms also apply to amino acid polymers in which one or more amino acid residues is an artificial chemical mimetic of a corresponding naturally occurring amino acid, as well as to naturally occurring amino acid polymers and non-naturally occurring amino acid polymers. The peptide can have any suitable posttranslational modification (e.g., phosphorylation, hydroxylation, sulfonation, palmitoylation, glycosylation, disulfide formation, galactosylation, fucosylation, etc.).

As used herein, the phrase “alternative protein scaffold” refers to a non-immunoglobulin derived protein or peptide. Such proteins and peptides are generally amenable to engineering and can be designed to confer monospecificity against a given antigen, bispecificity, or multispecificity. Engineering of an alternative protein scaffold can be conducted using several approaches. A loop grafting approach can be used where sequences of known specificity are grafted onto a variable loop of a scaffold. Sequence randomization and mutagenesis can be used to develop a library of mutants, which can be screened using various display platforms (e.g., phage display) to identify a novel binder. Site-specific mutagenesis can also be used as part of a similar approach. Alternative protein scaffolds exist in a variety of sizes, ranging from small peptides with minimal secondary structure to large proteins of similar size to a full-sized antibody. Examples of scaffolds include, but are not limited to, cystine knotted miniproteins (also known as knottins), cyclic cystine knotted miniproteins (also known as cyclotides), avimers, affibodies, the tenth type III domain of human fibronectin, DARPins (designed ankyrin repeats), and anticalins (also known as lipocalins). Naturally occurring ligands with known specificity can also be engineered to confer novel specificity against a given target. Examples of naturally occurring ligands that may be engineered include the EGF ligand and VEGF ligand. Engineered proteins can either be produced as monomeric proteins or as multimers, depending on the desired binding strategy and specificities. Protein engineering strategies can be used to fuse alternative protein scaffolds to Fc domains.

As used herein, the term “nucleotide” refers to any chemical entity comprising deoxyribonucleic acid (“DNA”), ribonucleic acid (“RNA”), a deoxyribonucleic acid derivative, or a ribonucleic acid derivative. Exemplary nucleotide-based structures include RNA, DNA, antisense oligonucleotides, siRNA, aptamers, etc. As used herein, the terms “deoxyribonucleic acid derivative” and “ribonucleic acid derivative” refer to DNA and RNA, respectively, that have been modified, such as, for example, by removing the phosphate backbone, methylating a hydroxyl group, or replacing a hydroxyl group with a thiol group.

As used herein, the phrase “antibody construct” refers to polypeptide comprising an antigen binding domain and an Fc domain. An antibody construct can comprise or be an antibody.

As used herein, the phrase “antigen binding domain” refers to a protein, or a portion of a protein, that specifically binds a specified antigen (e.g., a paratope), for example, that portion of an antigen-binding protein that contains the amino acid residues that interact with an antigen and confer on the antigen-binding protein its specificity and affinity for the antigen.

As used herein, the phrase “Fc domain” refers to the fragment crystallizable region, or the tail region of an antibody. The Fc domain interacts with Fc receptors on cell surfaces.

As used herein, the phrase “targeting binding domain” refers to a protein, or a portion of a protein, that specifically binds a second antigen that is distinct from the antigen bound by the antigen binding domain of an antibody construct. The targeting binding domain can be conjugated to the antibody construct at a C-terminal end of the Fc domain.

As used herein, the term “antibody” refers to a polypeptide comprising an antigen binding region (including the complementarity determining region (CDRs)) from an immunoglobulin gene or fragments thereof that specifically binds and recognizes an antigen. The recognized immunoglobulin genes include the kappa, lambda, alpha, gamma, delta, epsilon, and mu constant region genes, as well as numerous immunoglobulin variable region genes.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains, respectively. Light chains are classified as either kappa or lambda. Heavy chains are classified as gamma, mu, alpha, delta, or epsilon, which in turn define the immunoglobulin classes IgG, IgM, IgA, IgD, and IgE, respectively.

IgG antibodies are large molecules of about 150 kDa composed of four peptide chains. IgG antibodies contain two identical class γ heavy chains of about 50 kDa and two identical light chains of about 25 kDa, forming a tetrameric quaternary structure. The two heavy chains are linked to each other and to a light chain each by disulfide bonds. The resulting tetramer has two identical halves, which together form the Y-like shape. Each end of the fork contains an identical antigen binding site. There are four IgG subclasses (IgG1, 2, 3, and 4) in humans, named in order of their abundance in serum (IgG1 being the most abundant). Typically, the antigen-binding region of an antibody will be most critical in specificity and affinity of binding.

Dimeric IgA antibodies are about 320 kDa. IgA has two subclasses (IgA1 and IgA2) and can be produced as a monomeric as well as a dimeric form. The IgA dimeric form (secretory or sIgA) is the most abundant.

Antibodies can exist, for examples, as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into a Fab′ monomer. The Fab′ monomer is essentially Fab with part of the hinge region (see, e.g., Fundamental Immunology (Paul, editor, 7th edition, 2012)). While various antibody fragments are defined in terms of the digestion of an intact antibody, such fragments may be synthesized de novo either chemically or by using recombinant DNA methodology. Thus, the term antibody, as used herein, also includes antibody fragments produced by the modification of whole antibodies, synthesized de novo using recombinant DNA methodologies (e.g., single chain Fv), or identified using phage display libraries (see, e.g., McCafferty et al., Nature, 348: 552-554 (1990)).

The term “antibody” is used in the broadest sense and specifically encompasses monoclonal antibodies (including full length monoclonal antibodies), polyclonal antibodies, multispecific antibodies (e.g., bispecific antibodies), and antibody fragments so long as they exhibit the desired biological activity. “Antibody fragment” and all grammatical variants thereof as used herein are defined as a portion of an intact antibody comprising the antigen binding site or variable region of the intact antibody, wherein the portion is free of the constant heavy chain domains (i.e., CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules; (2) single chain polypeptides containing only one light chain variable domain, or a fragment thereof that contains the three CDRs of the light chain variable domain, without an associated heavy chain moiety; (3) single chain polypeptides containing only one heavy chain variable region, or a fragment thereof containing the three CDRs of the heavy chain variable region, without an associated light chain moiety; (4) nanobodies comprising single Ig domains from non-human species or other specific single-domain binding modules; and (5) multispecific or multivalent structures formed from antibody fragments. In an antibody fragment comprising one or more heavy chains, the heavy chain(s) can contain any constant domain sequence (e.g., CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

As used herein, the term “biosimilar” in reference to a biological product means that the biological product is highly similar to the reference product notwithstanding minor differences in clinically inactive components, and there are no clinically meaningful differences between the biological product and the reference product in terms of the safety, purity, and potency of the product.

As used herein, the term “epitope” means any antigenic determinant on an antigen to which binds the antigen-binding site, also referred to as the paratope, of an antibody. Epitopic determinants usually consist of chemically active surface groupings of molecules such as amino acids or sugar side chains and usually have specific three-dimensional structural characteristics, as well as specific charge characteristics.

The terms “polypeptide,” “peptide,” and “protein” are used interchangeably herein to refer to a polymer of amino acid residues. The terms also apply to amino acid polymers in which one or more amino acid residues are artificial chemical mimetics of a corresponding naturally occurring amino acids, as well as to naturally occurring amino acid polymers and non-naturally occurring amino acid polymer.

As used herein, the term “immune checkpoint inhibitors” refers to any modulator that inhibits the activity of the immune checkpoint molecule. Immune checkpoint inhibitors can include, but are not limited to, immune checkpoint molecule binding proteins, antibodies, antibody-derivatives (including Fc fusions, Fab fragments and scFvs), antisense oligonucleotides, siRNA, aptamers, peptides and peptide mimetics.

As used herein, the terms “Toll-like receptor” and “TLR” refer to any member of a family of highly-conserved mammalian proteins which recognizes pathogen-associated molecular patterns and acts as key signaling elements in innate immunity. TLR polypeptides share a characteristic structure that includes an extracellular domain that has leucine-rich repeats, a transmembrane domain, and an intracellular domain that is involved in TLR signaling.

The terms “Toll-like receptor 7” and “TLR7” refer to nucleic acids or polypeptides sharing at least about 70%, about 80%, about 90%, about 95%, about 96%, about 97%, about 98%, about 99%, or more sequence identity to a publicly-available TLR7 sequence, e.g., GenBank accession number AAZ99026 for human TLR7 polypeptide, or GenBank accession number AAK62676 for murine TLR7 polypeptide.

The terms “Toll-like receptor 8” and “TLR8” refer to nucleic acids or polypeptides sharing at least about 70%, about 80%, about 90%, about 95%, about 96%, about 97%, about 98%, about 99%, or more sequence identity to a publicly-available TLR7 sequence, e.g., GenBank accession number AAZ95441 for human TLR8 polypeptide, or GenBank accession number AAK62677 for murine TLR8 polypeptide.

A “TLR agonist” is a substance that binds, directly or indirectly, to a TLR (e.g., TLR7 and/or TLR8) to induce TLR signaling. Any detectable difference in TLR signaling can indicate that an agonist stimulates or activates a TLR. Signaling differences can be manifested, for example, as changes in the expression of target genes, in the phosphorylation of signal transduction components, in the intracellular localization of downstream elements such as nuclear factor-κB (NF-κB), in the association of certain components (such as IL-1 receptor associated kinase (IRAK)) with other proteins or intracellular structures, or in the biochemical activity of components such as kinases (such as mitogen-activated protein kinase (MAPK)).

As used herein, the term “amino acid” refers to any monomeric unit that can be incorporated into a peptide, polypeptide, or protein. Amino acids include naturally-occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid). The amino acids can be glycosylated (e.g., N-linked glycans, O-linked glycans, phosphoglycans, C-linked glycans, or glypiation) or deglycosylated.

Naturally-occurring amino acids are those encoded by the genetic code, as well as those amino acids that are later modified, e.g., hydroxyproline, γ-carboxyglutamate, and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, and methionine methyl sulfonium. “Amino acid mimetics” refer to chemical compounds that have a structure that is different from the general chemical structure of an amino acid, but that functions in a manner similar to a naturally-occurring amino acid.

Amino acids may be referred to herein by either the commonly known three letter symbols or by the one-letter symbols recommended by the IUPAC-IUB Biochemical Nomenclature Commission.

As used herein, the term “linker” refers to a functional group that covalently bonds two or more moieties in a compound or material. For example, the linking moiety can serve to covalently bond a TLR agonist to a macromolecular support in a macromolecule-supported compound.

As used herein, the term “linking moiety” refers to a functional group that covalently bonds two or more moieties in a compound or material. For example, the linking moiety can serve to covalently bond a TLR agonist to a macromolecular support in a macromolecule-supported compound. Useful bonds for connecting linking moieties to proteins and other materials include, but are not limited to, amides, amines, esters, carbamates, ureas, thioethers, thiocarbamates, thiocarbonates, and thioureas.

As used herein, the term “divalent” refers to a chemical moiety that contains two points of attachment for linking two functional groups; polyvalent linking moieties can have additional points of attachment for linking further functional groups. For example, divalent linking moieties include divalent polymer moieties such as divalent poly(ethylene glycol), divalent cycloalkyl, divalent heterocycloalkyl, divalent aryl, and divalent heteroaryl group. A “divalent cycloalkyl, heterocycloalkyl, aryl, or heteroaryl group” refers to a cycloalkyl, heterocycloalkyl, aryl, or heteroaryl group having two points of attachment for covalently linking two moieties in a molecule or material. Cycloalkyl, heterocycloalkyl, aryl, or heteroaryl groups can be substituted or unsubstituted. Cycloalkyl, heterocycloalkyl, aryl, or heteroaryl groups can be substituted with one or more groups selected from halo, hydroxy, amino, alkylamino, amido, acyl, nitro, cyano, and alkoxy.

As used herein, when the term “optionally present” is used to refer to a chemical structure (e.g., “A”, “U”, “V”, “X¹”, “X²”, and “X³”), if that chemical structure is not present, the bond originally made to the chemical structure is made directly to the adjacent atom.

As used herein, the wavy line (“ [symbol] ”) represents a point of attachment of the specified chemical moiety. If the specified chemical moiety has two wavy lines (“ [symbol] ”) present, it will be understood that the chemical moiety can be used bilaterally, i.e., as read from left to right or from right to left. In some embodiments, a specified moiety having two wavy lines (“ [symbol] ”) present is considered to be used as read from left to right.

As used herein, the term “linker” refers to a functional group that covalently bonds two or more moieties in a compound or material. For example, the linker can serve to covalently bond a TLR agonist to a macromolecular support in a macromolecule-supported compound.

As used herein, the term “alkyl” refers to a straight or branched, saturated, aliphatic radical having the number of carbon atoms indicated. Alkyl can include any number of carbons, such as C₁-C₂, C₁-C₃, C₁-C₄, C₁-C₅, C₁-C₆, C₁-C₇, C₁-C₈, C₁-C₉, C₁-C₁₀, C₂-C₃, C₂-C₄, C₂-C₅, C₂-C₆, C₃-C₄, C₃-C₅, C₃-C₆, C₄-C₅, C₄-C₆ and C₅-C₆. For example, C₁-C₄ alkyl includes, but is not limited to, methyl, ethyl, propyl, isopropyl, butyl, isobutyl, sec-butyl, and tert-butyl. Alkyl can also refer to alkyl groups having up to 30 carbons atoms, such as, but not limited to heptyl, octyl, nonyl, decyl, etc. Alkyl groups can be substituted or unsubstituted. “Substituted alkyl” groups can be substituted with one or more groups selected from halo, hydroxy, amino, oxo (═O), alkylamino, amido, acyl, nitro, cyano, and alkoxy. The term “alkylene” refers to a divalent alkyl radical.

As used herein, the term “heteroalkyl” refers to an alkyl group as described herein, wherein one or more carbon atoms are optionally and independently replaced with heteroatom selected from N, O, and S. The term “heteroalkylene” refers to a divalent heteroalkyl radical.

As used herein, the term “cycloalkyl” refers to a saturated or partially unsaturated, monocyclic, fused bicyclic, or bridged polycyclic ring assembly containing from 3 to 12 ring atoms, or the number of atoms indicated. Carbocycles can include any number of carbons, such as C₃-C₆, C₄-C₆, C₅-C₆, C₃-C₈, C₄-C₈, C₅-C₈, C₆-C₈, C₃-C₉, C₃-C₁₀, C₃-C₁₁, and C₃-C₁₂. Saturated monocyclic carbocyclic rings include, for example, cyclopropyl, cyclobutyl, cyclopentyl, cyclohexyl, and cyclooctyl. Saturated bicyclic and polycyclic carbocyclic rings include, for example, norbornane, [2.2.2] bicyclooctane, decahydronaphthalene and adamantane. Carbocyclic groups can also be partially unsaturated, having one or more double or triple bonds in the ring. Representative carbocyclic groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene.

As used herein, the term “aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl.

As used herein, the terms “heterocycloalkyl” and “heteroaryl” refer to a “cycloalkyl” or “aryl” group as described herein, wherein one or more carbon atoms are optionally and independently replaced with heteroatom selected from N, O, and S. “Heteroaryl,” by itself or as part of another substituent, refers to a monocyclic or fused bicyclic or tricyclic aromatic ring assembly containing 5 to 16 ring atoms, where from 1 to 5 of the ring atoms are a heteroatom such as N, O or S. Additional heteroatoms can also be useful, including, but not limited to, B, Al, Si and P. The heteroatoms can be oxidized to form moieties such as, but not limited to, —S(O)— and —S(O)₂—. Heteroaryl groups can include any number of ring atoms, such as 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted. “Substituted heteroaryl” groups can be substituted with one or more groups selected from halo, hydroxy, amino, oxo (═O), alkylamino, amido, acyl, nitro, cyano, and alkoxy.

Heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinoazoline, cinnoline includes 3- and 4-cinnoline, benzothiophene includes 2- and 3-benzothiophene, and benzofuran includes 2- and 3-benzofuran.

“Heterocycloalkyl,” by itself or as part of another substituent, refers to a saturated ring system having from 3 to 12 ring members and from 1 to 4 heteroatoms of N, O and S. Additional heteroatoms can also be useful, including, but not limited to, B, Al, Si and P. The heteroatoms can be oxidized to form moieties such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline. Heterocycloalkyl groups can be unsubstituted or substituted.

Heterocycloalkyl groups can be linked via any position on the ring. For example, aziridine can be 1- or 2-aziridine, azetidine can be 1- or 2-azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-isothiazolidine, and morpholine can be 2-, 3- or 4-morpholine.

As used herein, the terms “halo” and “halogen,” by themselves or as part of another substituent, refer to a fluorine, chlorine, bromine, or iodine atom.

As used herein, the term “carbonyl,” by itself or as part of another substituent, refers to C(O) or —C(O)—, i.e., a carbon atom double-bonded to oxygen and bound to two other groups in the moiety having the carbonyl.

As used herein, the term “amino” refers to a moiety —NR3, wherein each R group is H or alkyl. An amino moiety can be ionized to form the corresponding ammonium cation.

As used herein, the phrase “quaternary ammonium salt” refers to a tertiary amine that has been quaternized with an alkyl substituent (e.g., a C₁-C₄ alkyl such as methyl, ethyl, propyl, or butyl).

As used herein, the term “hydroxyl” refers to the moiety —OH.

As used herein, the term “cyano” refers to a carbon atom triple-bonded to a nitrogen atom (i.e., the moiety —C≡N).

As used herein, the terms “treat,” “treatment,” and “treating” refer to any indicia of success in the treatment or amelioration of an injury, pathology, condition (e.g., cancer), or symptom (e.g., cognitive impairment), including any objective or subjective parameter such as abatement; remission; diminishing of symptoms or making the symptom, injury, pathology, or condition more tolerable to the patient; reduction in the rate of symptom progression; decreasing the frequency or duration of the symptom or condition; or, in some situations, preventing the onset of the symptom. The treatment or amelioration of symptoms can be based on any objective or subjective parameter, including, for example, the result of a physical examination.

The terms “cancer,” “neoplasm,” and “tumor” are used herein to refer to cells which exhibit autonomous, unregulated growth, such that the cells exhibit an aberrant growth phenotype characterized by a significant loss of control over cell proliferation. Cells of interest for detection, analysis, and/or treatment in the context of the invention include cancer cells (e.g., cancer cells from an individual with cancer), malignant cancer cells, pre-metastatic cancer cells, metastatic cancer cells, and non-metastatic cancer cells. Cancers of virtually every tissue are known. The phrase “cancer burden” refers to the quantum of cancer cells or cancer volume in a subject. Reducing cancer burden accordingly refers to reducing the number of cancer cells or the cancer cell volume in a subject. The term “cancer cell” as used herein refers to any cell that is a cancer cell (e.g., from any of the cancers for which an individual can be treated, e.g., isolated from an individual having cancer) or is derived from a cancer cell, e.g., clone of a cancer cell. For example, a cancer cell can be from an established cancer cell line, can be a primary cell isolated from an individual with cancer, can be a progeny cell from a primary cell isolated from an individual with cancer, and the like. In some embodiments, the term can also refer to a portion of a cancer cell, such as a sub-cellular portion, a cell membrane portion, or a cell lysate of a cancer cell. Many types of cancers are known to those of skill in the art, including solid tumors such as carcinomas, sarcomas, glioblastomas, melanomas, lymphomas, and myelomas, and circulating cancers such as leukemias.

Examples of different types of cancer include, but are not limited to, lung cancer (e.g., non-small cell lung cancer or NSCLC), ovarian cancer, prostate cancer, colorectal cancer, liver cancer (i.e., hepatocarcinoma), renal cancer (i.e., renal cell carcinoma), bladder cancer, breast cancer, thyroid cancer, pleural cancer, pancreatic cancer, uterine cancer, cervical cancer, testicular cancer, anal cancer, bile duct cancer, gastrointestinal carcinoid tumors, esophageal cancer, gall bladder cancer, appendix cancer, small intestine cancer, stomach (gastric) cancer, cancer of the central nervous system, skin cancer (e.g., melanoma), choriocarcinoma, head and neck cancer, blood cancer, osteogenic sarcoma, fibrosarcoma, neuroblastoma, glioma, melanoma, B-cell lymphoma, non-Hodgkin's lymphoma, Burkitt's lymphoma, Small Cell lymphoma, Large Cell lymphoma, monocytic leukemia, myelogenous leukemia, acute lymphocytic leukemia, acute myelocytic leukemia, and multiple myeloma.

Carcinomas are malignancies that originate in the epithelial tissues. Epithelial cells cover the external surface of the body, line the internal cavities, and form the lining of glandular tissues. Examples of carcinomas include, but are not limited to, adenocarcinoma (cancer that begins in glandular (secretory) cells such as cancers of the breast, pancreas, lung, prostate, stomach, gastroesophageal junction, and colon) adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma; transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; and the like. Carcinomas may be found in prostrate, pancreas, colon, brain (usually as secondary metastases), lung, breast, and skin.

Soft tissue tumors are a highly diverse group of rare tumors that are derived from connective tissue. Examples of soft tissue tumors include, but are not limited to, alveolar soft part sarcoma; angiomatoid fibrous histiocytoma; chondromyoxid fibroma; skeletal chondrosarcoma; extraskeletal myxoid chondrosarcoma; clear cell sarcoma; desmoplastic small round-cell tumor; dermatofibrosarcoma protuberans; endometrial stromal tumor; Ewing's sarcoma; fibromatosis (Desmoid); fibrosarcoma, infantile; gastrointestinal stromal tumor; bone giant cell tumor; tenosynovial giant cell tumor; inflammatory myofibroblastic tumor; uterine leiomyoma; leiomyosarcoma; lipoblastoma; typical lipoma; spindle cell or pleomorphic lipoma; atypical lipoma; chondroid lipoma; well-differentiated liposarcoma; myxoid/round cell liposarcoma; pleomorphic liposarcoma; myxoid malignant fibrous histiocytoma; high-grade malignant fibrous histiocytoma; myxofibrosarcoma; malignant peripheral nerve sheath tumor; mesothelioma; neuroblastoma; osteochondroma; osteosarcoma; primitive neuroectodermal tumor; alveolar rhabdomyosarcoma; embryonal rhabdomyosarcoma; benign or malignant schwannoma; synovial sarcoma; Evan's tumor; nodular fasciitis; desmoid-type fibromatosis; solitary fibrous tumor; dermatofibrosarcoma protuberans (DF SP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; pleomorphic adenoma of soft tissue; and neoplasias derived from fibroblasts, myofibroblasts, histiocytes, vascular cells/endothelial cells, and nerve sheath cells.

A sarcoma is a rare type of cancer that arises in cells of mesenchymal origin, e.g., in bone or in the soft tissues of the body, including cartilage, fat, muscle, blood vessels, fibrous tissue, or other connective or supportive tissue. Different types of sarcoma are based on where the cancer forms. For example, osteosarcoma forms in bone, liposarcoma forms in fat, and rhabdomyosarcoma forms in muscle. Examples of sarcomas include, but are not limited to, askin's tumor; sarcoma botryoides; chondrosarcoma; ewing's sarcoma; malignant hemangioendothelioma; malignant schwannoma; osteosarcoma; and soft tissue sarcomas (e.g., alveolar soft part sarcoma; angiosarcoma; cystosarcoma phyllodesdermatofibrosarcoma protuberans (DFSP); desmoid tumor; desmoplastic small round cell tumor; epithelioid sarcoma; extraskeletal chondrosarcoma; extraskeletal osteosarcoma; fibrosarcoma; gastrointestinal stromal tumor (GIST); hemangiopericytoma; hemangiosarcoma (more commonly referred to as “angiosarcoma”); kaposi's sarcoma; leiomyosarcoma; liposarcoma; lymphangiosarcoma; malignant peripheral nerve sheath tumor (MPNST); neurofibrosarcoma; synovial sarcoma; and undifferentiated pleomorphic sarcoma).

A teratoma is a type of germ cell tumor that may contain several different types of tissue (e.g., can include tissues derived from any and/or all of the three germ layers: endoderm, mesoderm, and ectoderm), including, for example, hair, muscle, and bone. Teratomas occur most often in the ovaries in women, the testicles in men, and the tailbone in children.

Melanoma is a form of cancer that begins in melanocytes (cells that make the pigment melanin). Melanoma may begin in a mole (skin melanoma), but can also begin in other pigmented tissues, such as in the eye or in the intestines.

Leukemias are cancers that start in blood-forming tissue, such as the bone marrow, and cause large numbers of abnormal blood cells to be produced and enter the bloodstream. For example, leukemias can originate in bone marrow-derived cells that normally mature in the bloodstream. Leukemias are named for how quickly the disease develops and progresses (e.g., acute versus chronic) and for the type of white blood cell that is affected (e.g., myeloid versus lymphoid). Myeloid leukemias are also called myelogenous or myeloblastic leukemias. Lymphoid leukemias are also called lymphoblastic or lymphocytic leukemia. Lymphoid leukemia cells may collect in the lymph nodes, which can become swollen. Examples of leukemias include, but are not limited to, Acute myeloid leukemia (AML), Acute lymphoblastic leukemia (ALL), Chronic myeloid leukemia (CIVIL), and Chronic lymphocytic leukemia (CLL).

Lymphomas are cancers that begin in cells of the immune system. For example, lymphomas can originate in bone marrow-derived cells that normally mature in the lymphatic system. There are two basic categories of lymphomas. One category of lymphoma is Hodgkin lymphoma (HL), which is marked by the presence of a type of cell called the Reed-Sternberg cell. There are currently 6 recognized types of HL. Examples of Hodgkin lymphomas include nodular sclerosis classical Hodgkin lymphoma (CHL), mixed cellularity CHL, lymphocyte-depletion CHL, lymphocyte-rich CHL, and nodular lymphocyte predominant HL.

The other category of lymphoma is non-Hodgkin lymphomas (NHL), which includes a large, diverse group of cancers of immune system cells. Non-Hodgkin lymphomas can be further divided into cancers that have an indolent (slow-growing) course and those that have an aggressive (fast-growing) course. There are currently 61 recognized types of NHL. Examples of non-Hodgkin lymphomas include, but are not limited to, AIDS-related Lymphomas, anaplastic large-cell lymphoma, angioimmunoblastic lymphoma, blastic NK-cell lymphoma, Burkitt's lymphoma, Burkitt-like lymphoma (small non-cleaved cell lymphoma), chronic lymphocytic leukemia/small lymphocytic lymphoma, cutaneous T-Cell lymphoma, diffuse large B-Cell lymphoma, enteropathy-type T-Cell lymphoma, follicular lymphoma, hepatosplenic gamma-delta T-Cell lymphomas, T-Cell leukemias, lymphoblastic lymphoma, mantle cell lymphoma, marginal zone lymphoma, nasal T-Cell lymphoma, pediatric lymphoma, peripheral T-Cell lymphomas, primary central nervous system lymphoma, transformed lymphomas, treatment-related T-Cell lymphomas, and Waldenstrom's macroglobulinemia.

Brain cancers include any cancer of the brain tissues. Examples of brain cancers include, but are not limited to, gliomas (e.g., glioblastomas, astrocytomas, oligodendrogliomas, ependymomas, and the like), meningiomas, pituitary adenomas, and vestibular schwannomas, primitive neuroectodermal tumors (medulloblastomas).

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, and invasion of surrounding or distant tissues or organs, such as lymph nodes.

As used herein, the phrases “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs, therefore, tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part that is not directly connected to the organ of the original cancerous tumor. Metastasis can also be defined as several steps of a process, such as the departure of cancer cells from an original tumor site, and migration and/or invasion of cancer cells to other parts of the body.

As used herein the phrases “effective amount” and “therapeutically effective amount” refer to a dose of a substance such as a macromolecule-supported compound that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); Goodman & Gilman's The Pharmacological Basis of Therapeutics, 11^{th} Edition (McGraw-Hill, 2006); and Remington: The Science and Practice of Pharmacy, 22^{nd} Edition, (Pharmaceutical Press, London, 2012)).

As used herein, the terms “recipient,” “individual,” “subject,” “host,” and “patient” are used interchangeably and refer to any mammalian subject for whom diagnosis, treatment, or therapy is desired (e.g., humans). “Mammal” for purposes of treatment refers to any animal classified as a mammal, including humans, domestic and farm animals, and zoo, sports, or pet animals, such as dogs, horses, cats, cows, sheep, goats, pigs, camels, etc. In certain embodiments, the mammal is human.

As used herein, the term “administering” refers to parenteral, intravenous, intraperitoneal, intramuscular, intratumoral, intralesional, intranasal, or subcutaneous administration, oral administration, administration as a suppository, topical contact, intrathecal administration, or the implantation of a slow-release device, e.g., a mini-osmotic pump, to the subject.

The terms “about” and “around,” as used herein to modify a numerical value, indicate a close range surrounding the numerical value. Thus, if “X” is the value, “about X” or “around X” indicates a value of from 0.9X to 1.1X, e.g., from 0.95X to 1.05X or from 0.99X to 1.01X. A reference to “about X” or “around X” specifically indicates at least the values X, 0.95X, 0.96X, 0.97X, 0.98X, 0.99X, 1.01X, 1.02X, 1.03X, 1.04X, and 1.05X. Accordingly, “about X” and “around X” are intended to teach and provide written description support for a claim limitation of, e.g., “0.98X.”

### Macromolecule-Supported Compounds

The invention provides a macromolecule-supported compound of formula (I):

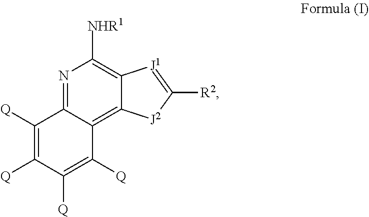

a pharmaceutically acceptable salt thereof, or a quaternary ammonium salt thereof, wherein

R¹ and R² independently are hydrogen or of formula:

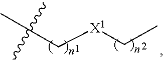

J¹ is CH or N,

J² is CHQ, NQ, O, or S,

each Q independently is Y or Z, wherein exactly one Q is Y,

Y is of formula:

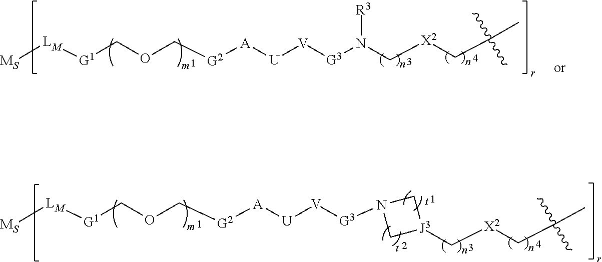

each Z independently is hydrogen or of formula:

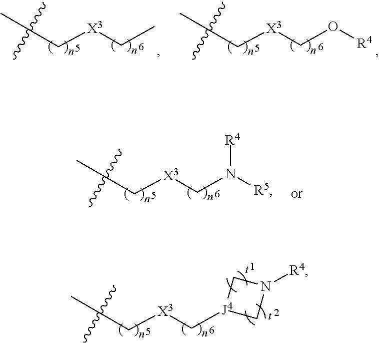

A is optionally present and is NR⁶ or of formula:

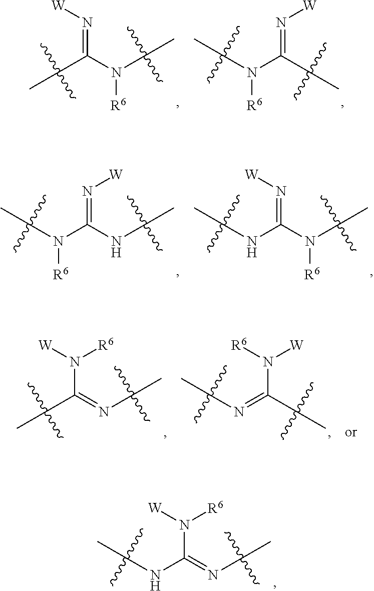

U is optionally present and is CH₂, C(O), CH₂C(O), or C(O)CH₂,

R⁶ and W independently are hydrogen, Ar¹, or of formula:

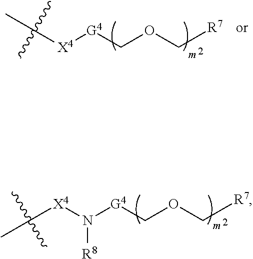

V is optionally present and is of formula:

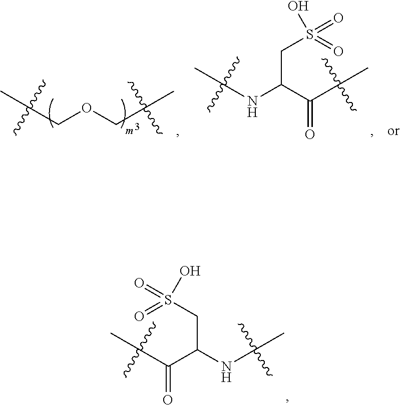

J³ and J⁴ independently are CH or N,

m¹, m², and m³ independently are an integer from 0 to 25, except that at least one of m¹, m², and m³ is a non-zero integer,

n¹, n², n³, n⁴, n⁵, and n⁶ independently are an integer from 0 to 10,

t¹ and t² independently are an integer from 1 to 3,

G¹, G², G³, and G⁴ independently are CH₂, C(O), CH₂C(O), C(O)CH₂, or a bond,

X¹, X², X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

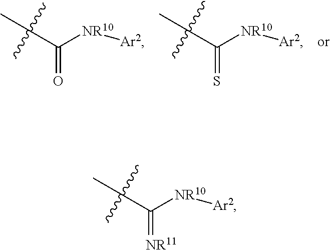

R³, R⁵, R⁷, R⁸, R⁹, R¹⁰, and R¹¹ independently are hydrogen or C₁-C₄ alkyl,

Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (Ia):

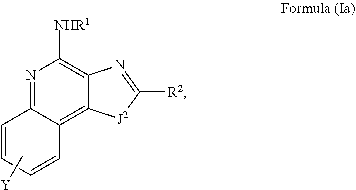

a pharmaceutically acceptable salt thereof, or a quaternary ammonium salt thereof, wherein

R¹ and R² independently are hydrogen or of formula:

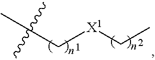

J² is CHZ, NZ, O, or S,

Y is of formula:

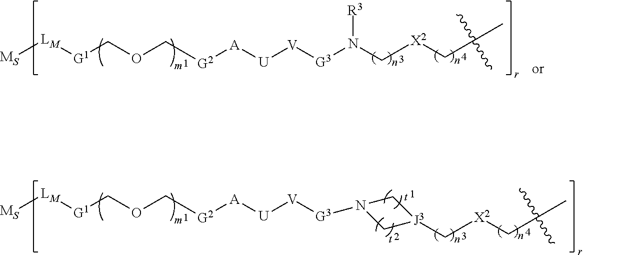

Z is hydrogen or of formula:

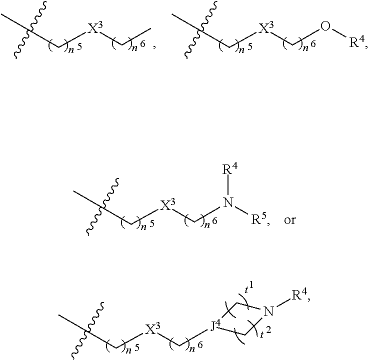

A is optionally present and is NR⁶ or of formula:

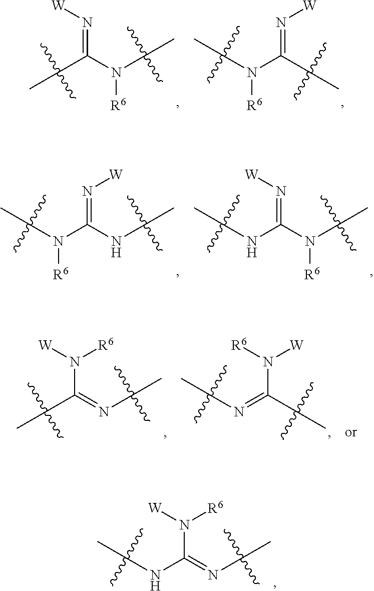

U is optionally present and is CH₂, C(O), CH₂C(O), or C(O)CH₂,

R⁶ and W independently are hydrogen, Ar¹, or of formula:

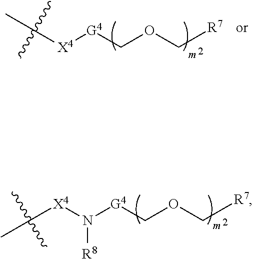

V is optionally present and is of formula:

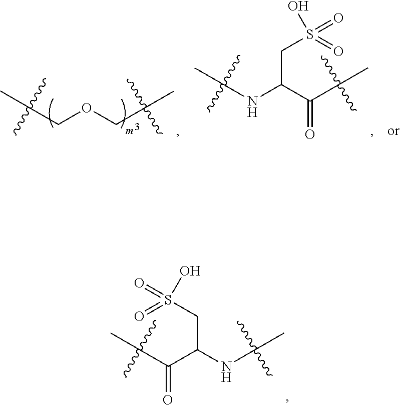

J³ and J⁴ independently are CH or N,

m¹, m², and m³ independently are an integer from 0 to 25, except that at least one of m¹, m², and m³ is a non-zero integer,

n¹, n², n³, n⁴, n⁵, and n⁶ independently are an integer from 0 to 10,

t¹ and t² independently are an integer from 1 to 3,

G¹, G², G³, and G⁴ independently are CH₂, C(O), CH₂C(O), C(O)CH₂, or a bond,

X¹, X², X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

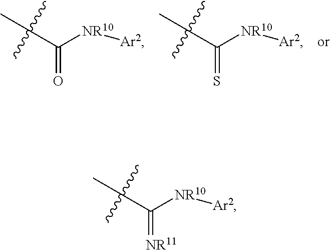

R³, R⁵, R⁷, R⁸, R⁹, R¹⁰, and R¹¹ independently are hydrogen or C₁-C₄ alkyl,

Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and each wavy line (“ [symbol] ”) represents a point of attachment.

In certain embodiments, the macromolecule-supported compound is of formula (Ia₁), (Ia₂), (Ia₃), (Ia₄), (Ia₅), or (Ia₆):

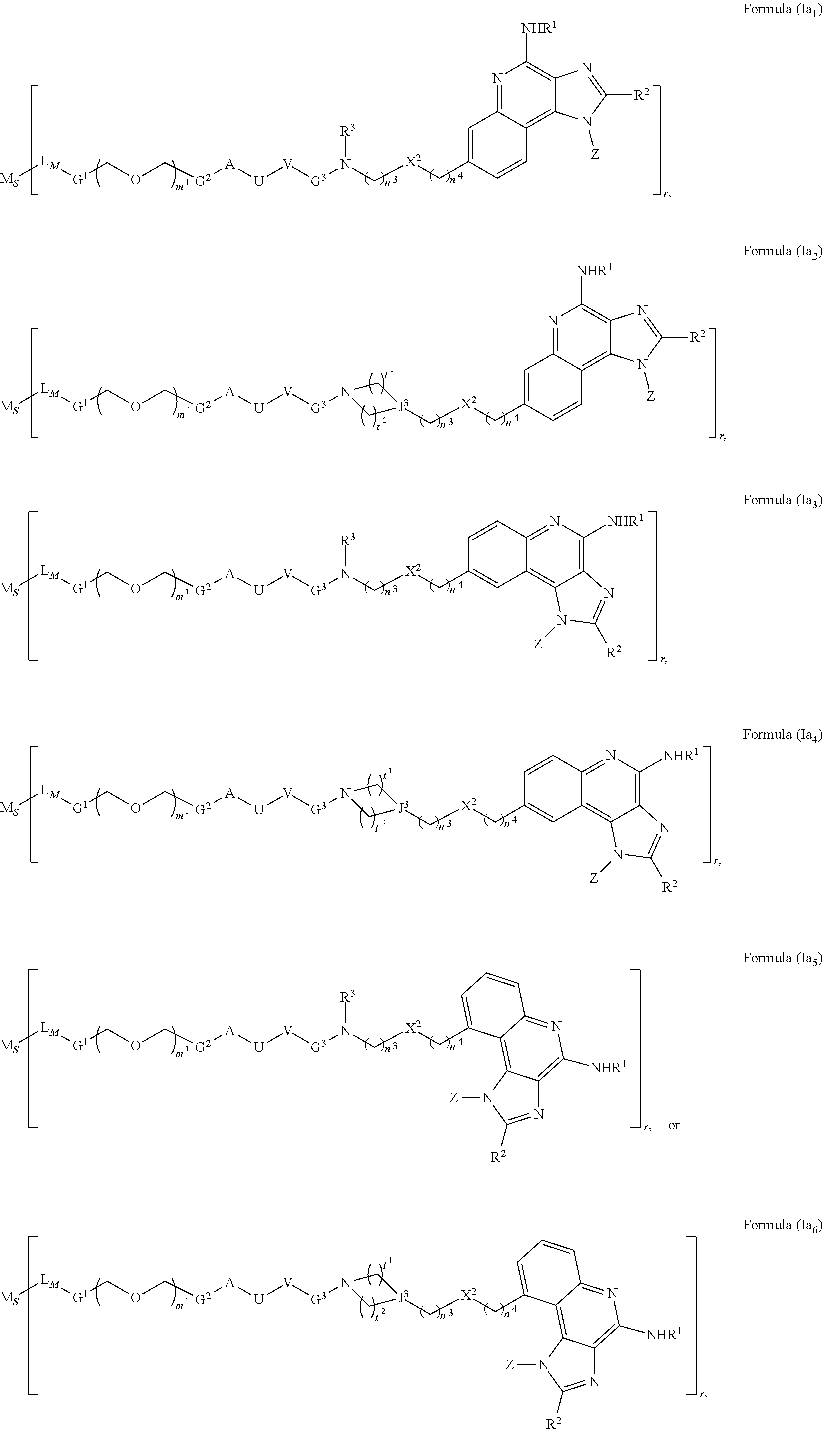

a pharmaceutically acceptable salt thereof, or a quaternary ammonium salt thereof, wherein

R¹ and R² independently are hydrogen or of formula:

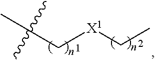

Z is hydrogen or of formula:

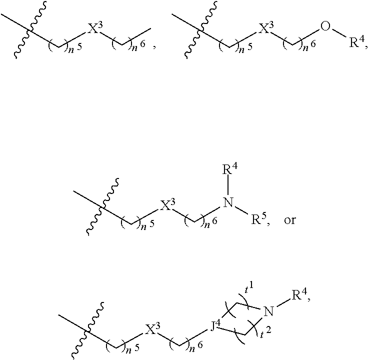

A is optionally present and is NR⁶ or of formula:

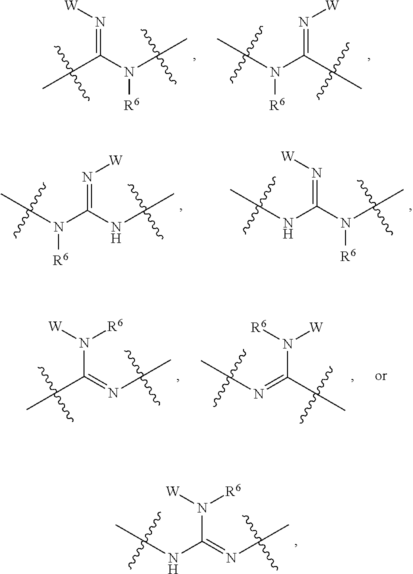

U is optionally present and is CH₂, C(O), CH₂C(O), or C(O)CH₂,

R⁶ and W independently are hydrogen, Ar¹, or of formula:

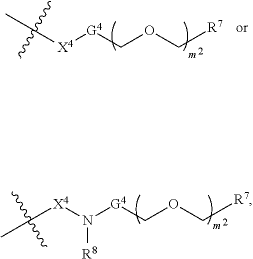

V is optionally present and is of formula:

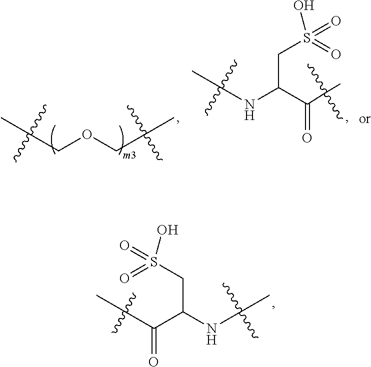

J³ and J⁴ independently are CH or N,

m¹, m², and m³ independently are an integer from 0 to 25, except that at least one of m¹, m², and m³ is a non-zero integer,

n¹, n², n³, n⁴, n⁵, and n⁶ independently are an integer from 0 to 10, t¹ and t² independently are an integer from 1 to 3,

G¹, G², G³, and G⁴ independently are CH₂, C(O), CH₂C(O), C(O)CH₂, or a bond,

X¹, X², X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

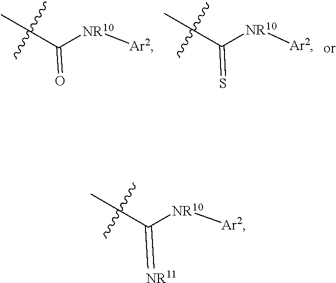

R³, R⁵, R⁷, R⁸, R⁹, R¹⁰, and R¹¹ independently are hydrogen or C₁-C₄ alkyl,

Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (Iaa), (Iab), (Iac), (Iad), (Iae), or (Iaf):

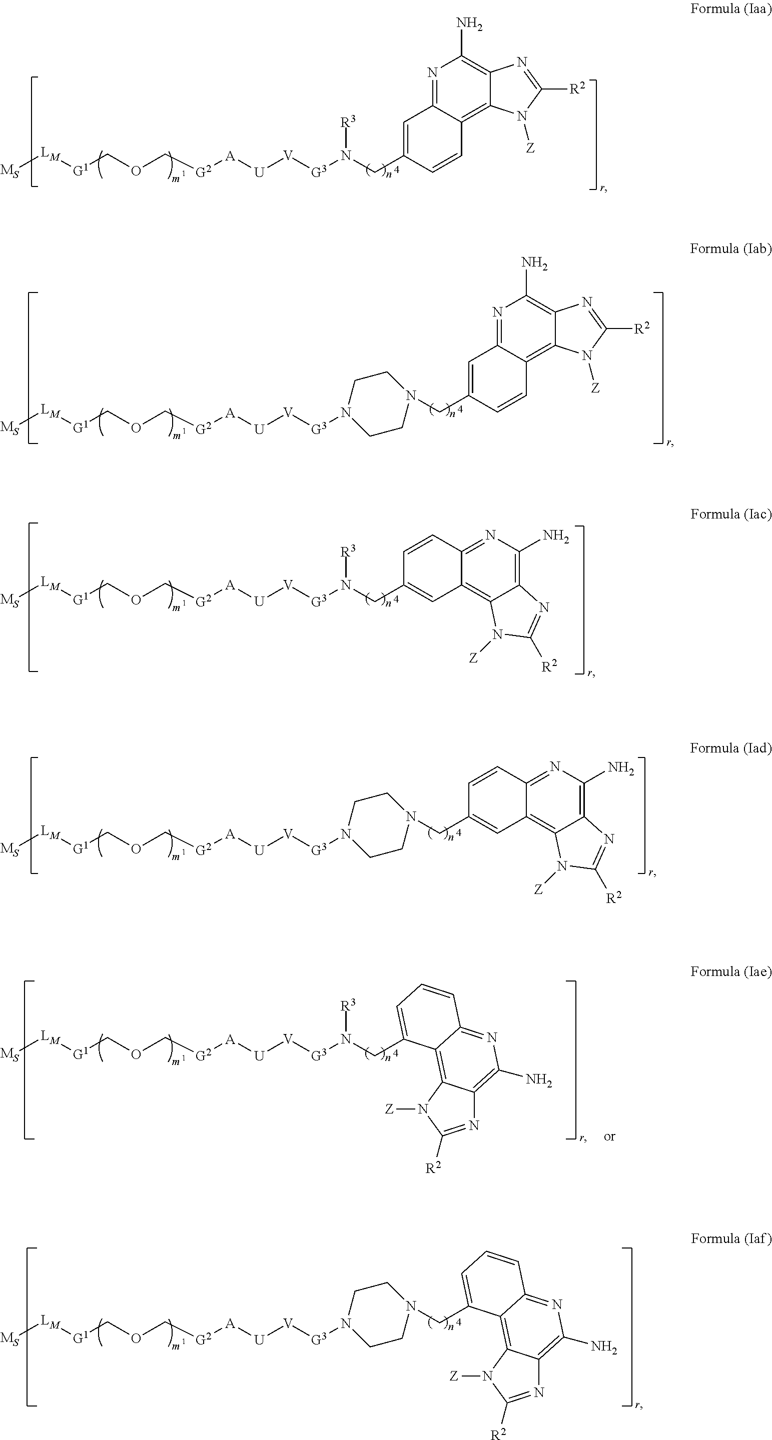

a pharmaceutically acceptable salt thereof, or a quaternary ammonium salt thereof, wherein

R² is of formula:

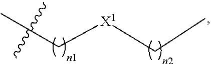

Z is hydrogen or of formula:

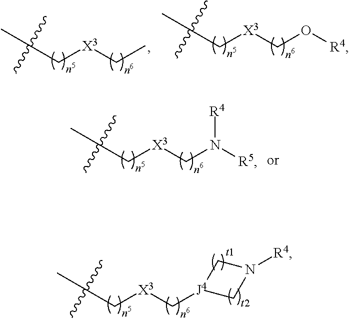

A is optionally present and is NR⁶ or of formula:

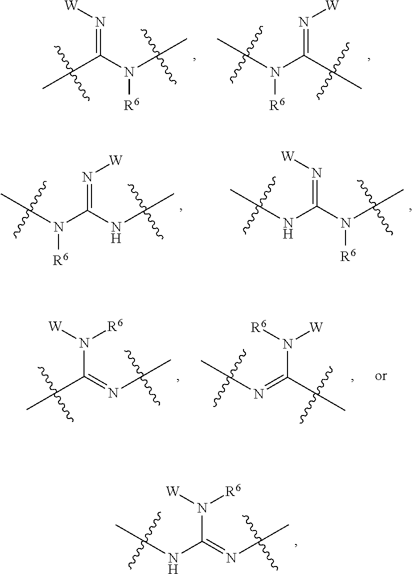

U is optionally present and is CH₂, C(O), CH₂C(O), or C(O)CH₂,

R⁶ and W independently are hydrogen, Ar¹, or of formula:

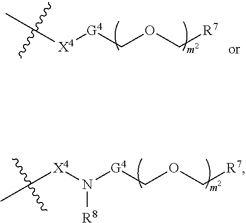

V is optionally present and is of formula:

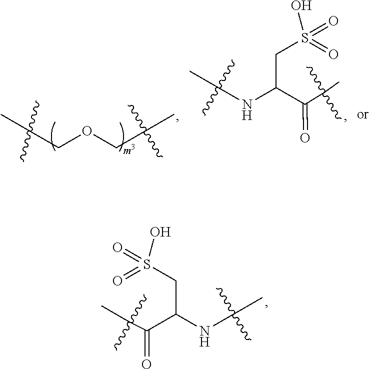

J⁴ is CH or N,

m¹, m², and m³ independently are an integer from 0 to 25, except that at least one of m¹, m², and m³ is a non-zero integer,

n¹, n², n⁴, n⁵, and n⁶ independently are an integer from 0 to 10,

G¹, G², G³, and G⁴ independently are CH₂, C(O), CH₂C(O), C(O)CH₂, or a bond,

X¹, X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

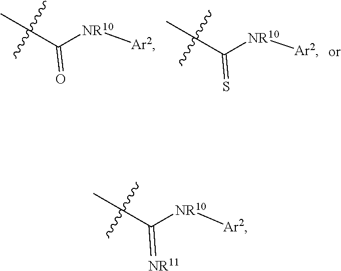

R³, R⁵, R⁷, R⁸, R⁹, R¹⁰, and R¹¹ independently are hydrogen or C₁-C₄ alkyl,

Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (Ib):

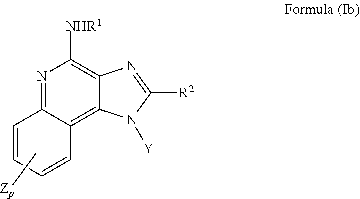

a pharmaceutically acceptable salt thereof, or a quaternary ammonium salt thereof, wherein

R¹ and R² independently are hydrogen or of formula:

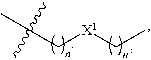

Y is of formula:

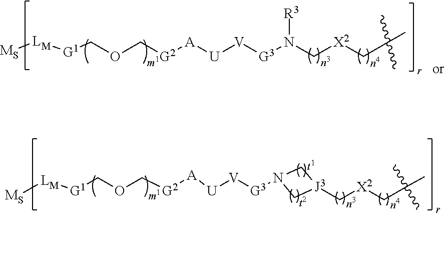

each Z independently is hydrogen or of formula:

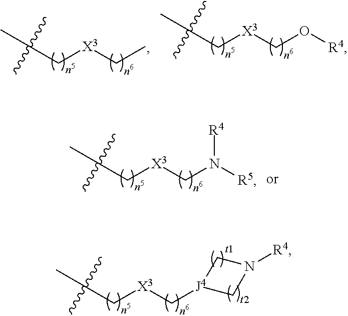

A is optionally present and is NR⁶ or of formula:

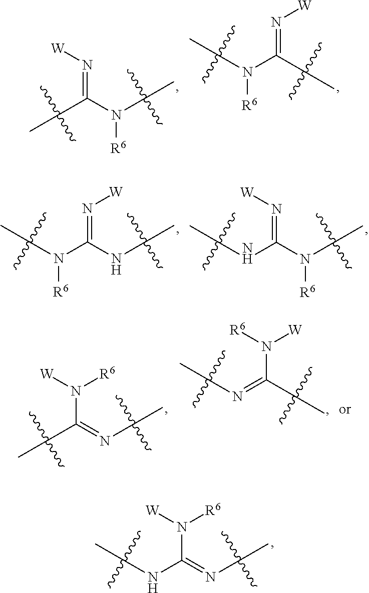

U is optionally present and is CH₂, C(O), CH₂C(O), or C(O)CH₂,

R⁶ and W independently are hydrogen, Ar¹, or of formula:

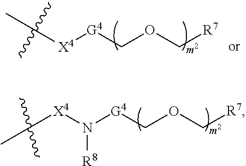

V is optionally present and is of formula:

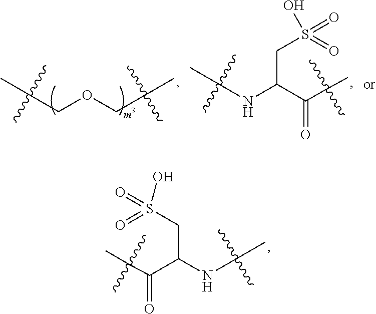

J³ and J⁴ independently are CH or N,

m¹, m², and m³ independently are an integer from 0 to 25, except that at least one of m¹, m², and m³ is a non-zero integer,

n¹, n², n³, n⁴, n⁵, and n⁶ independently are an integer from 0 to 10,

p is an integer from 1 to 4,

t¹ and t² independently are an integer from 1 to 3,

G¹, G², G³, and G⁴ independently are CH₂, C(O), CH₂C(O), C(O)CH₂, or a bond,

X¹, X², X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

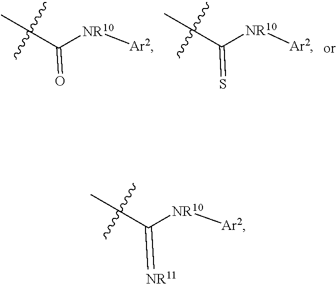

R³, R⁵, R⁷, R⁸, R⁹, R¹⁰, and R¹¹ independently are hydrogen or C₁-C₄ alkyl,

Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (Ib₁), (Ib₂), (Ib₃), or (Ib₄):

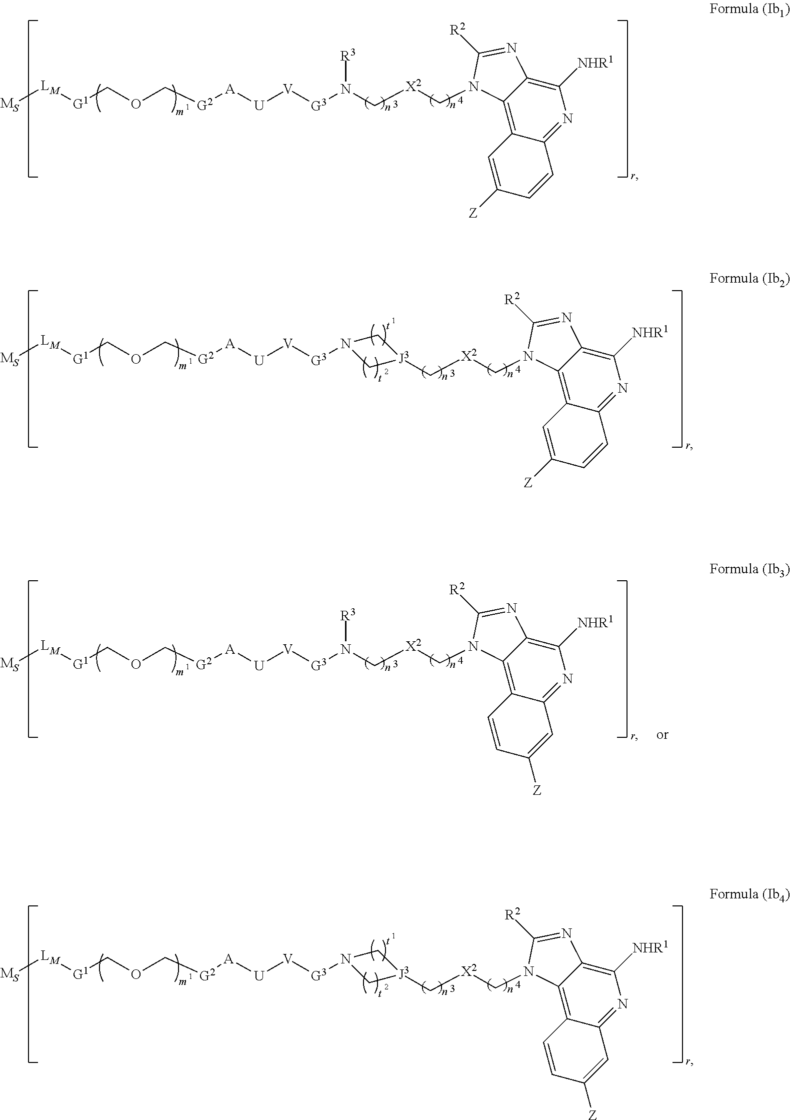

a pharmaceutically acceptable salt thereof, or a quaternary ammonium salt thereof, wherein

R¹ and R² independently are hydrogen or of formula:

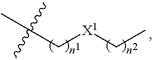

Z is hydrogen or of formula:

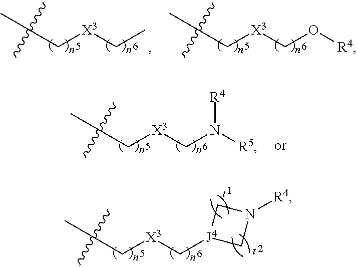

A is optionally present and is NR⁶ or of formula:

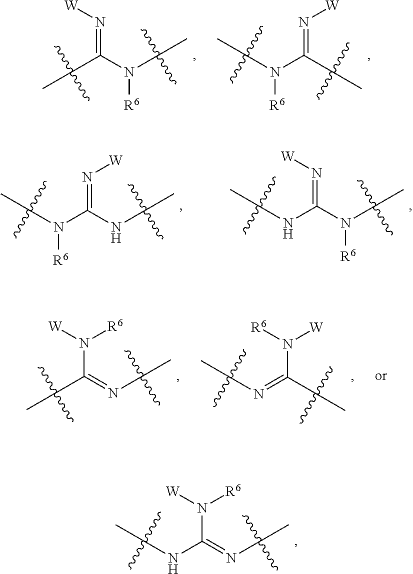

U is optionally present and is CH₂, C(O), CH₂C(O), or C(O)CH₂,

R⁶ and W independently are hydrogen, Ar¹, or of formula:

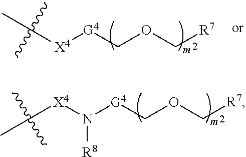

V is optionally present and is of formula:

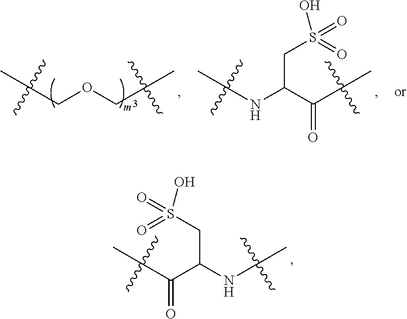

J³ and J⁴ independently are CH or N,

m¹, m², and m³ independently are an integer from 0 to 25, except that at least one of m¹, m², and m³ is a non-zero integer,

n¹, n², n³, n⁴, n⁵, and n⁶ independently are an integer from 0 to 10,

t¹ and t² independently are an integer from 1 to 3,

G¹, G², G³, and G⁴ independently are CH₂, C(O), CH₂C(O), C(O)CH₂, or a bond,

X¹, X², X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

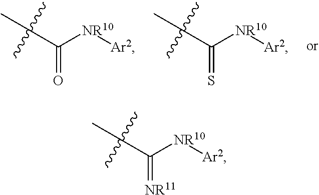

R³, R⁵, R⁷, R⁸, R⁹, R¹⁰, and R¹¹ independently are hydrogen or C₁-C₄ alkyl,

Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (Iba), (Ibb), (Ibc), or (Ibd):

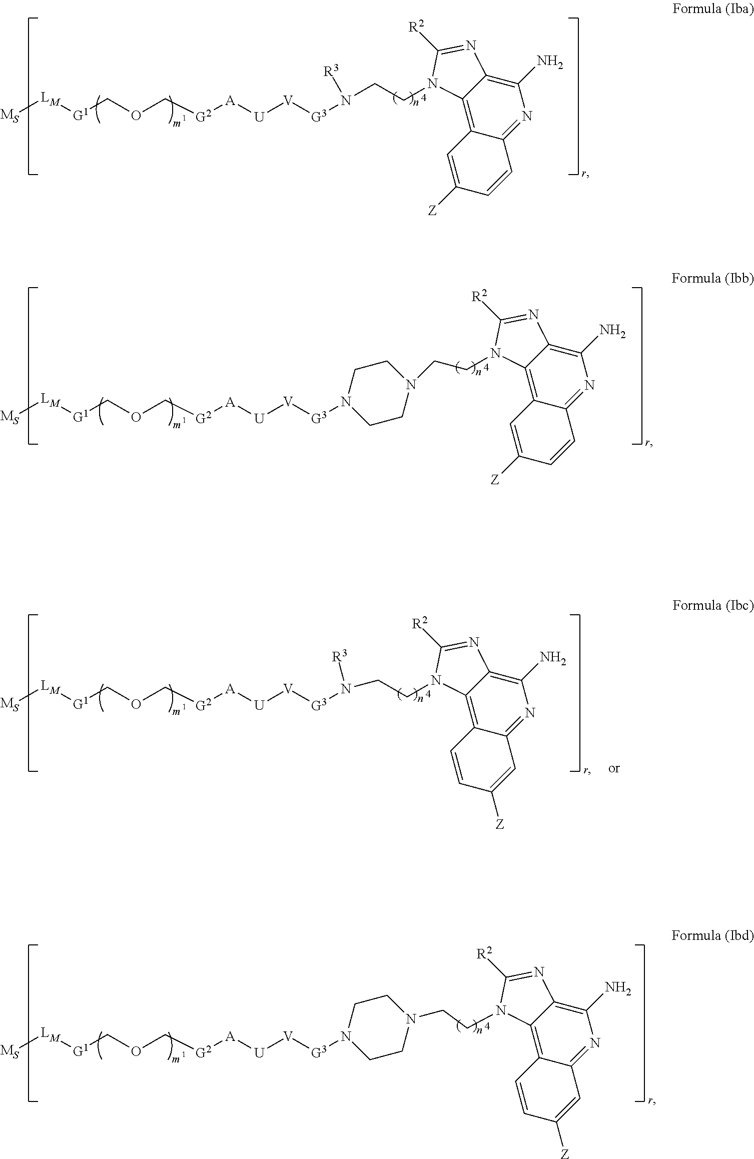

a pharmaceutically acceptable salt thereof, or a quaternary ammonium salt thereof, wherein

R² is of formula:

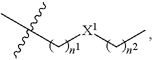

Z is hydrogen or of formula:

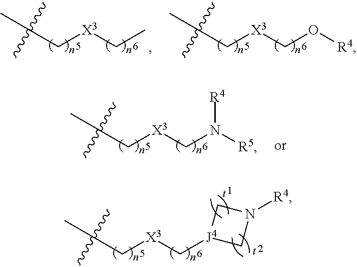

A is optionally present and is NR⁶ or of formula:

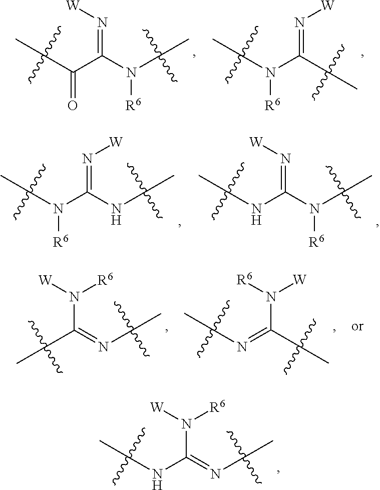

U is optionally present and is CH₂, C(O), CH₂C(O), or C(O)CH₂,

R⁶ and W independently are hydrogen, Ar¹, or of formula:

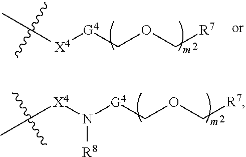

V is optionally present and is of formula:

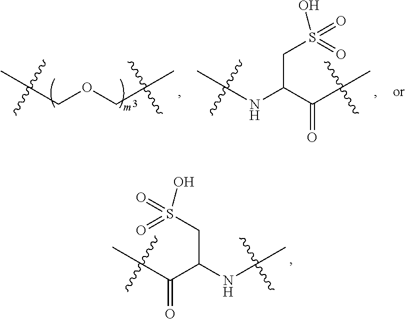

J³ and J⁴ independently are CH or N,

m¹, m², and m³ independently are an integer from 0 to 25, except that at least one of m¹, m², and m³ is a non-zero integer,

n¹, n², n⁴, n⁵, and n⁶ independently are an integer from 0 to 10,

G¹, G², G³, and G⁴ independently are CH₂, C(O), CH₂C(O), C(O)CH₂, or a bond,

X¹, X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

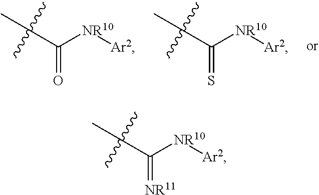

R³, R⁵, R⁷, R⁸, R⁹, R¹⁰, and R¹¹ independently are hydrogen or C₁-C₄ alkyl,

Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (Ic):

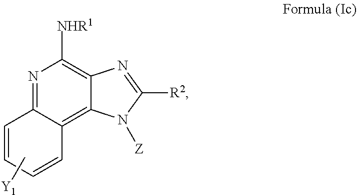

a pharmaceutically acceptable salt thereof, or a quaternary ammonium salt thereof, wherein

R¹ and R² independently are hydrogen or of formula:

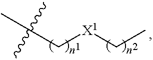

Y₁ is of formula:

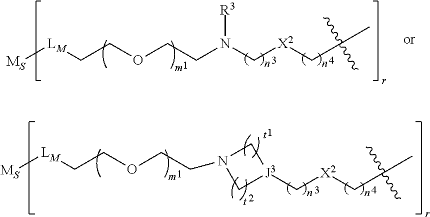

Z is hydrogen or of formula:

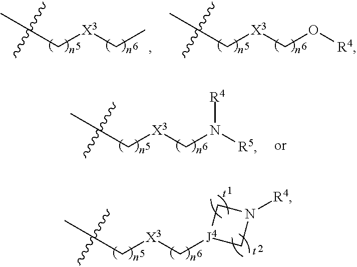

J³ and J⁴ independently are CH or N,

m¹ is an integer from 1 to 25,

n¹, n², n³, n⁴, n⁵, and n⁶ independently are an integer from 0 to 10,

t¹ and t² independently are an integer from 1 to 3,

X¹, X², and X³ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

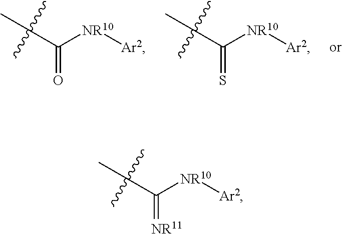

R³, R⁵, R⁹, R¹⁰, and R¹¹ independently are hydrogen or C₁-C₄ alkyl,

Ar² is an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (Id):

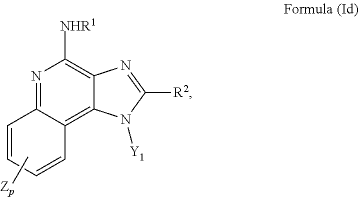

a pharmaceutically acceptable salt thereof, or a quaternary ammonium salt thereof, wherein

R¹ and R² independently are hydrogen or of formula:

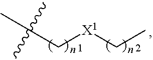

Y₁ is of formula:

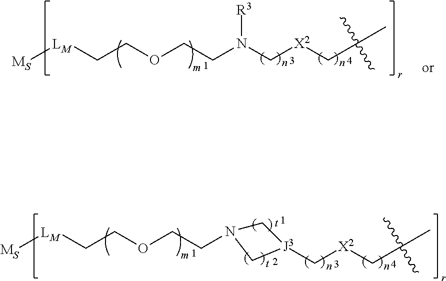

each Z independently is hydrogen or of formula:

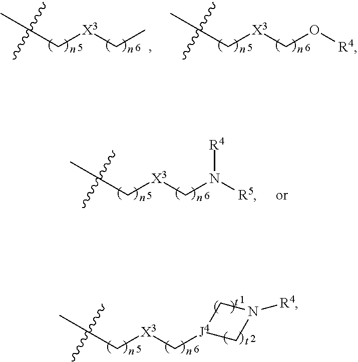

J³ and J⁴ independently are CH or N,

m¹ is an integer from 1 to 25,

n¹, n², n³, n⁴, n⁵, and n⁶ independently are an integer from 0 to 10,

p is an integer from 1 to 4,

t¹ and t² independently are an integer from 1 to 3,

X¹, X², and X³ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

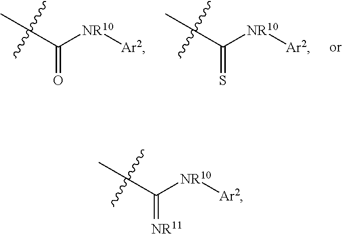

R³, R⁵, R⁹, R¹⁰, and R¹¹ independently are hydrogen or C₁-C₄ alkyl,

Ar² is an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (Ie):

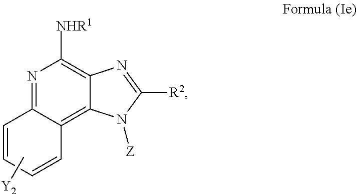

a pharmaceutically acceptable salt thereof, or a quaternary ammonium salt thereof, wherein

R¹ and R² independently are hydrogen or of formula:

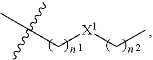

Y₂ is of formula:

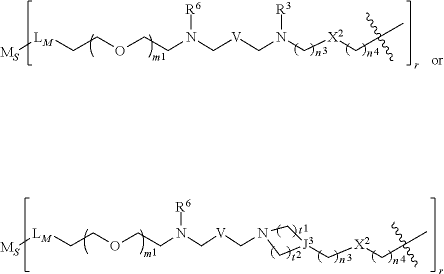

Z is hydrogen or of formula:

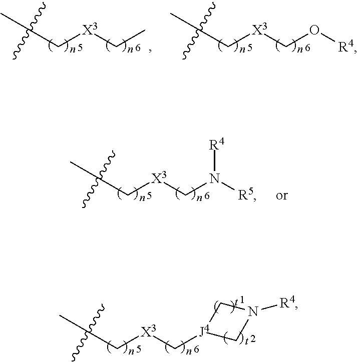

R⁶ is hydrogen, Ar¹, or of formula:

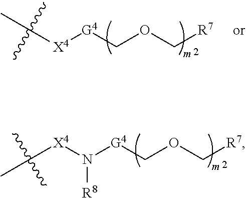

V is optionally present and is of formula:

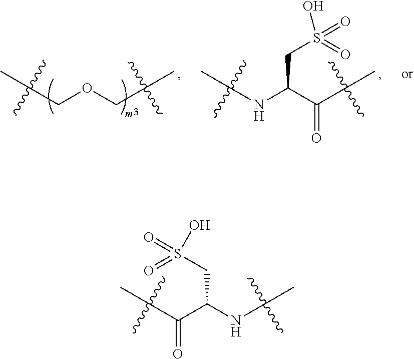

J³ and J⁴ independently are CH or N,

m¹, m², and m³ independently are an integer from 0 to 25, except that at least one of m¹, m², and m³ is a non-zero integer,

n¹, n², n³, n⁴, n⁵, and n⁶ independently are an integer from 0 to 10,

t¹ and t² independently are an integer from 1 to 3,

G⁴ is CH₂, C(O), CH₂C(O), C(O)CH₂, or a bond,

X¹, X², X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

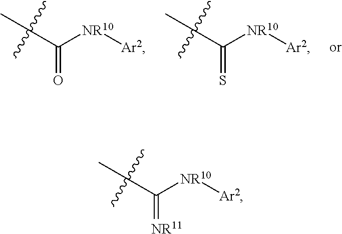

R³, R⁵, R⁷, R⁸, R⁹, R¹⁰, and R¹¹ independently are hydrogen or C₁-C₄ alkyl,

Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (If)

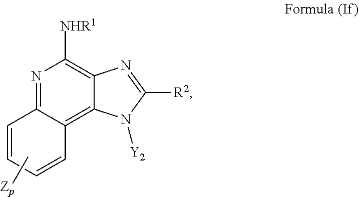

a pharmaceutically acceptable salt thereof, or a quaternary ammonium salt thereof, wherein

R¹ and R² independently are hydrogen or of formula:

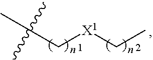

Y₂ is of formula:

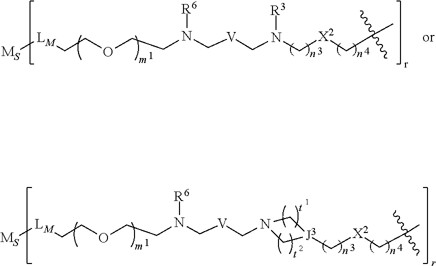

each Z independently is hydrogen or of formula:

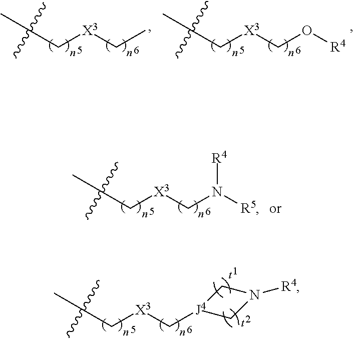

R⁶ is hydrogen, Ar¹, or of formula:

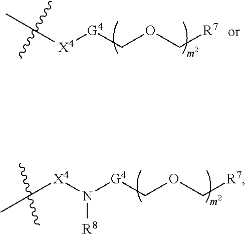

V is optionally present and is of formula:

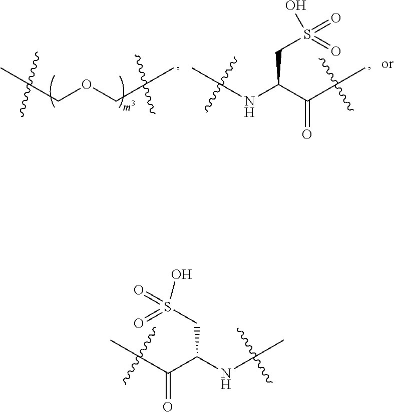

J³ and J⁴ independently are CH or N,

m¹, m², and m³ independently are an integer from 0 to 25, except that at least one of m¹, m², and m³ is a non-zero integer,

p is an integer from 1 to 4,

t¹ and t² independently are an integer from 1 to 3,

G⁴ is CH₂, C(O), CH₂C(O), C(O)CH₂, or a bond,

X¹, X², X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

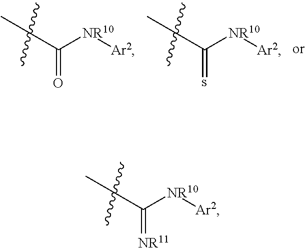

R³, R⁵, R⁷, R⁸, R⁹, R¹⁰, and R¹¹ independently are hydrogen or C₁-C₄ alkyl,

Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (Ig):

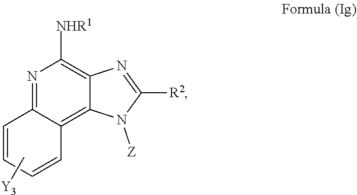

a pharmaceutically acceptable salt thereof, or a quaternary ammonium salt thereof, wherein

R¹ and R² independently are hydrogen or of formula:

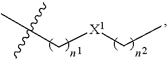

Y₃ is of formula:

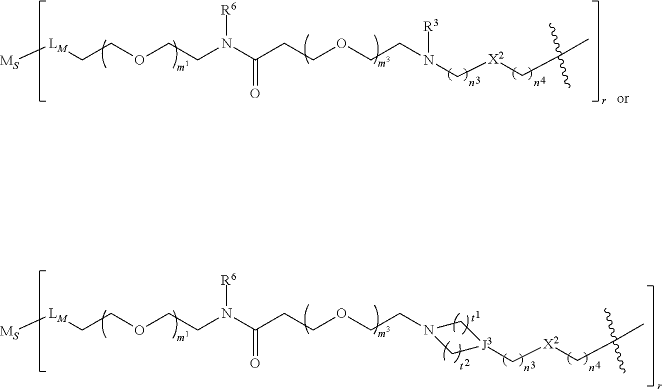

Z is hydrogen or of formula:

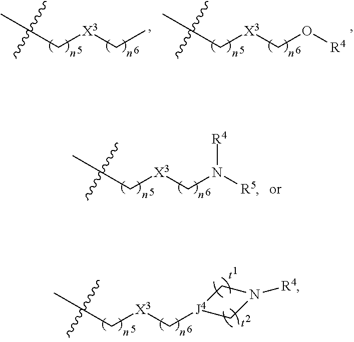

R⁶ is hydrogen, Ar¹, or of formula:

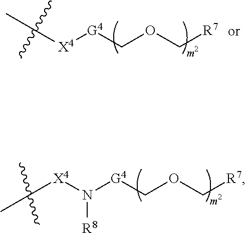

J³ and J⁴ independently are CH or N,

m¹, m², and m³ independently are an integer from 0 to 25, except that at least one of m¹, m², and m³ is a non-zero integer,

n¹, n², n³, n⁴, n⁵, and n⁶ independently are an integer from 0 to 10,

t¹ and t² independently are an integer from 1 to 3,

G⁴ is CH₂, C(O), CH₂C(O), C(O)CH₂, or a bond,

X¹, X², X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

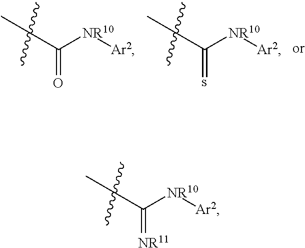

R³, R⁵, R⁷, R⁸, R⁹, R¹⁰, and R¹¹ independently are hydrogen or C₁-C₄ alkyl,

Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (Ih):

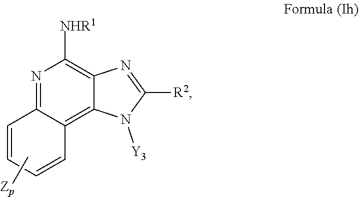

a pharmaceutically acceptable salt thereof, or a quaternary ammonium salt thereof, wherein

R¹ and R² independently are hydrogen or of formula:

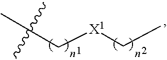

Y₃ is of formula:

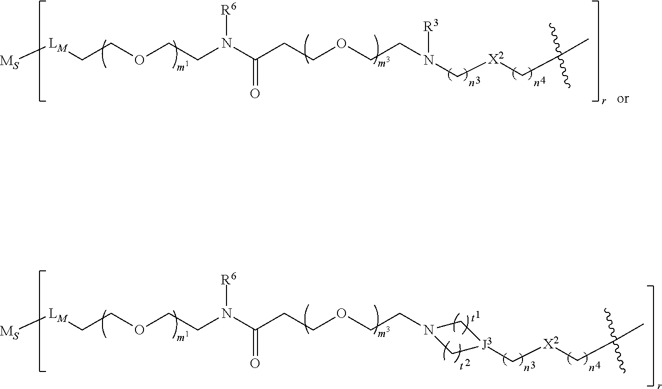

each Z independently is hydrogen or of formula:

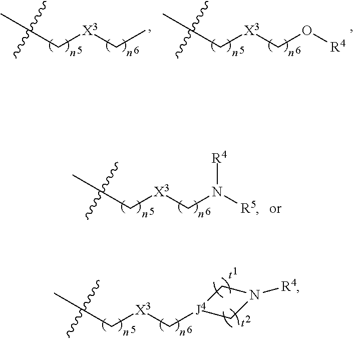

R⁶ is hydrogen, Ar¹, or of formula:

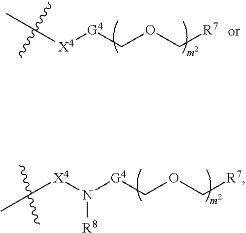

J³ and J⁴ independently are CH or N,

m¹, m², and m³ independently are an integer from 0 to 25, except that at least one of m¹, m², and m³ is a non-zero integer,

n¹, n², n³, n⁴, n⁵, and n⁶ independently are an integer from 0 to 10,

p is an integer from 1 to 4,

t¹ and t² independently are an integer from 1 to 3,

G⁴ is CH₂, C(O), CH₂C(O), C(O)CH₂, or a bond,

X¹, X², X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

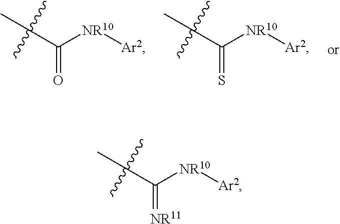

R³, R⁵, R⁷, R⁸, R⁹, R¹⁰, and R¹¹ independently are hydrogen or C₁-C₄ alkyl,

Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (Ii):

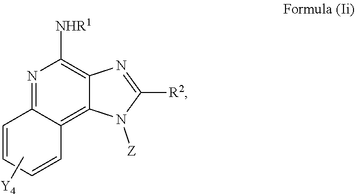

a pharmaceutically acceptable salt thereof, or a quaternary ammonium salt thereof, wherein

R¹ and R² independently are hydrogen or of formula:

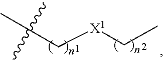

Y₄ is of formula:

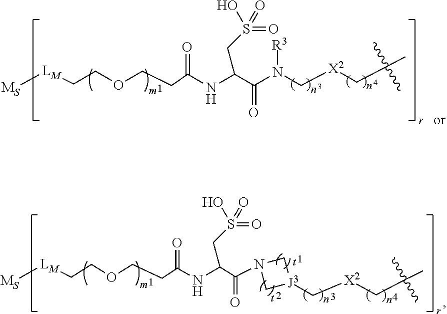

Z is hydrogen or of formula:

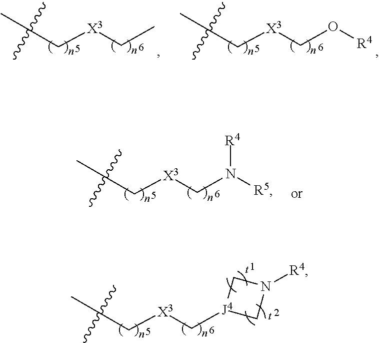

J³ and J⁴ independently are CH or N,

m¹ is an integer from 1 to 25,

n¹, n², n³, n⁴, n⁵, and n⁶ independently are an integer from 0 to 10,

t¹ and t² independently are an integer from 1 to 3,

X¹, X², and X³ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

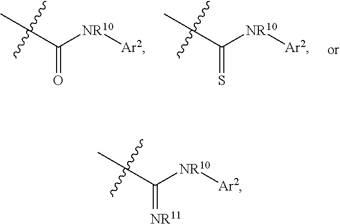

R³, R⁵, R⁹, R¹⁰, and R¹¹ independently are hydrogen or C₁-C₄ alkyl,

Ar² is an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (Ij):

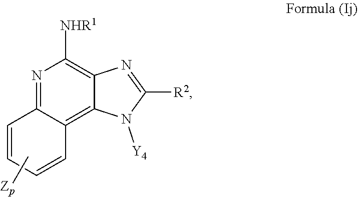

a pharmaceutically acceptable salt thereof, or a quaternary ammonium salt thereof, wherein

R¹ and R² independently are hydrogen or of formula:

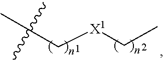

Y₄ is of formula:

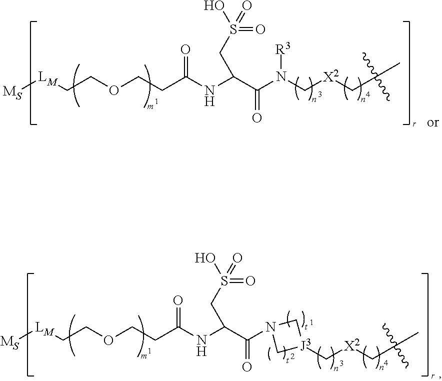

each Z independently is hydrogen or of formula:

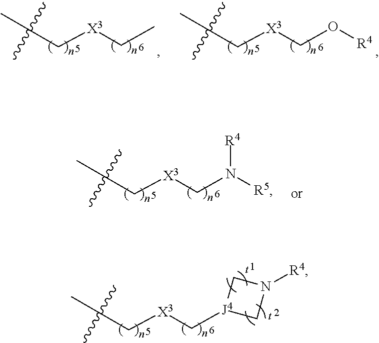

J³ and J⁴ independently are CH or N,

m¹ is an integer from 1 to 25,

n¹, n², n³, n⁴, n⁵, and n⁶ independently are an integer from 0 to 10,

p is an integer from 1 to 4,

t¹ and t² independently are an integer from 1 to 3,

X¹, X², and X³ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

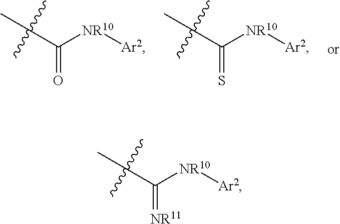

R³, R⁵, R⁹, R¹⁰, and R¹¹ independently are hydrogen or C₁-C₄ alkyl,

Ar² is an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (Ik):

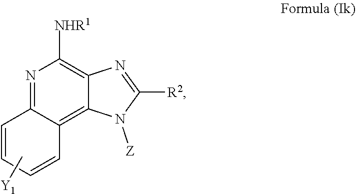

a pharmaceutically acceptable salt thereof, or a quaternary ammonium salt thereof, wherein

R¹ and R² independently are hydrogen or of formula:

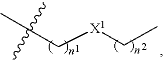

Y₅ is of formula:

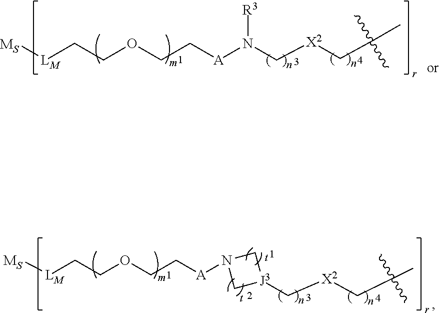

Z is hydrogen or of formula:

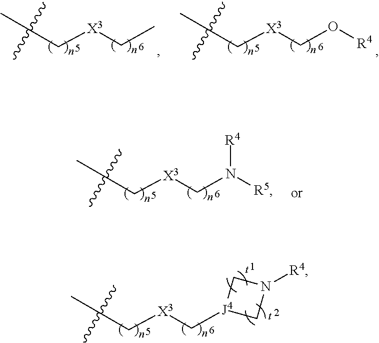

A is NR⁶ or of formula:

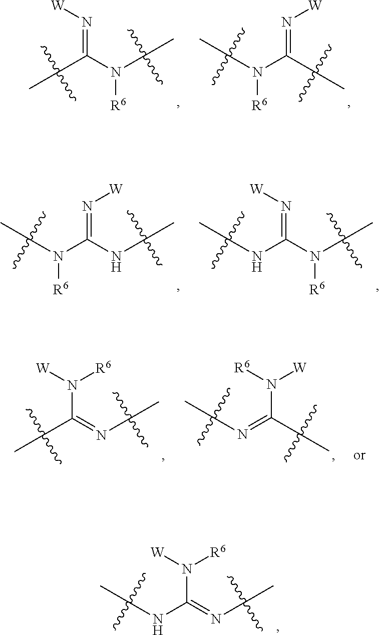

R⁶ and W independently are hydrogen, Ar¹, or of formula:

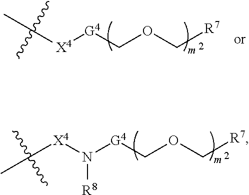

J³ and J⁴ independently are CH or N,

m¹ and m² independently are an integer from 0 to 25, except that at least one of m¹ and m² is a non-zero integer,

n¹, n², n³, n⁴, n⁵, and n⁶ independently are an integer from 0 to 10,

t¹ and t² independently are an integer from 1 to 3,

G⁴ is CH₂, C(O), CH₂C(O), C(O)CH₂, or a bond,

X¹, X², X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

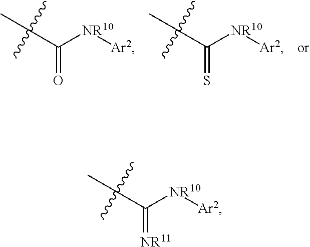

R³, R⁵, R⁷, R⁸, R⁹, R¹⁰, and R¹¹ independently are hydrogen or C₁-C₄ alkyl,

Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (Im):

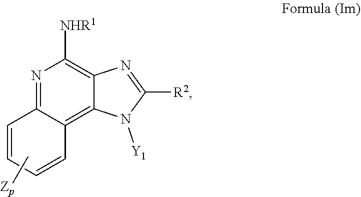

a pharmaceutically acceptable salt thereof, or a quaternary ammonium salt thereof, wherein

R¹ and R² independently are hydrogen or of formula:

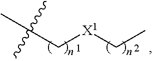

Y₅ is of formula:

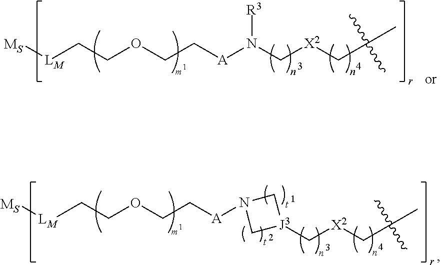

each Z independently is hydrogen or of formula:

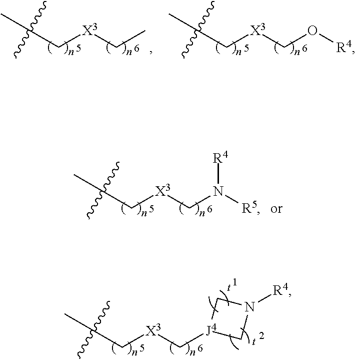

A is NR⁶ or of formula:

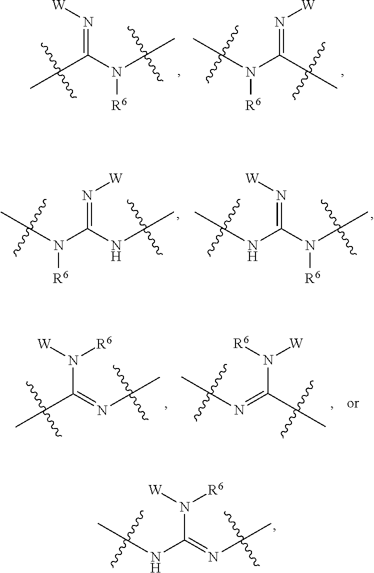

R⁶ and W independently are hydrogen, Ar¹, or of formula:

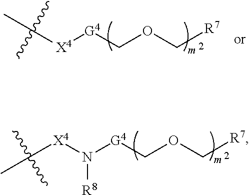

J³ and J⁴ independently are CH or N,

m¹ and m² independently are an integer from 0 to 25, except that at least one of m¹ and m² is a non-zero integer,

n¹, n², n³, n⁴, n⁵, and n⁶ independently are an integer from 0 to 10,

p is an integer from 1 to 4,

t¹ and t² independently are an integer from 1 to 3,

G⁴ is CH₂, C(O), CH₂C(O), C(O)CH₂, or a bond,

X¹, X², X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

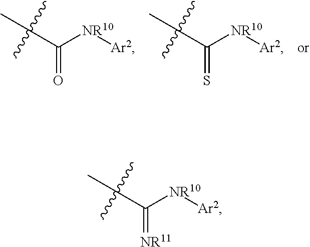

R³, R⁵, R⁷, R⁸, R⁹, R¹⁰, and R¹¹ independently are hydrogen or C₁-C₄ alkyl,

Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

The invention further provides a macromolecule-supported compound of formula (II):

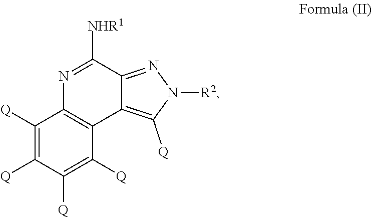

a pharmaceutically acceptable salt thereof, or a quaternary ammonium salt thereof, wherein

R¹ and R² independently are hydrogen or of formula:

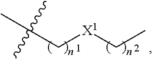

each Q independently is Y or Z, wherein exactly one Q is Y,

Y is of formula:

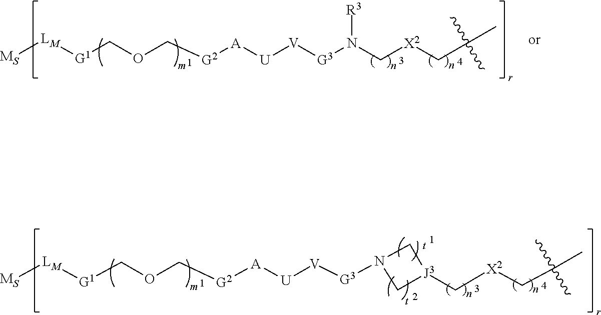

each Z independently is hydrogen or of formula:

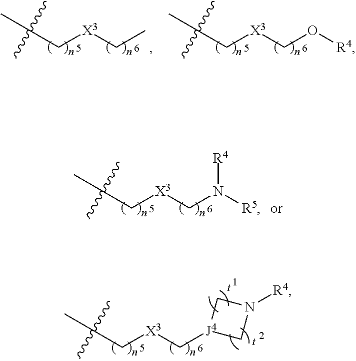

A is optionally present and is NR⁶ or of formula:

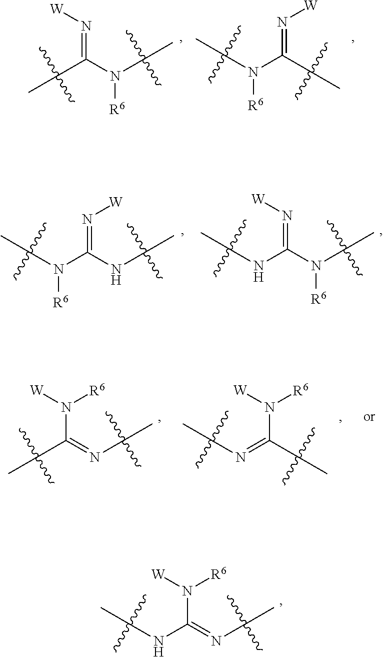

U is optionally present and is CH₂, C(O), CH₂C(O), or C(O)CH₂,

R⁶ and W independently are hydrogen, Ar¹, or of formula:

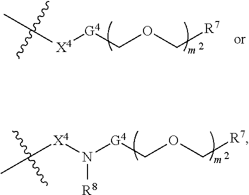

V is optionally present and is of formula:

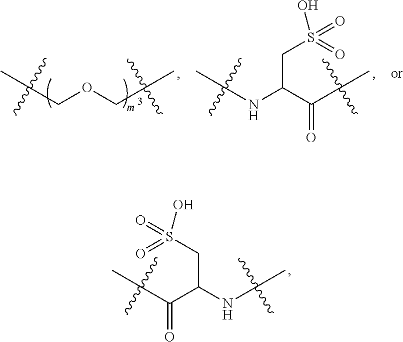

J³ and J⁴ independently are CH or N,

m¹, m², and m³ independently are an integer from 0 to 25, except that at least one of m¹, m², and m³ is a non-zero integer,

n¹, n², n³, n⁴, n⁵, and n⁶ independently are an integer from 0 to 10,

t¹ and t² independently are an integer from 1 to 3,

G¹, G², G³, and G⁴ independently are CH₂, C(O), CH₂C(O), C(O)CH₂, or a bond,

X¹, X², X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

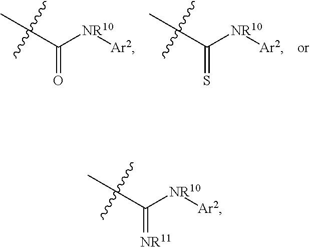

R³, R⁵, R⁷, R⁸, R⁹, R¹⁰, and R¹¹ independently are hydrogen or C₁-C₄ alkyl,

Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (IIa):

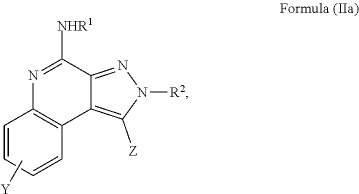

a pharmaceutically acceptable salt thereof, or a quaternary ammonium salt thereof, wherein

R¹ and R² independently are hydrogen or of formula:

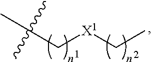

Y is of formula:

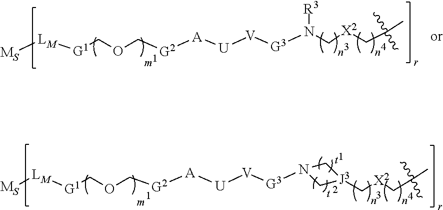

Z is hydrogen or of formula:

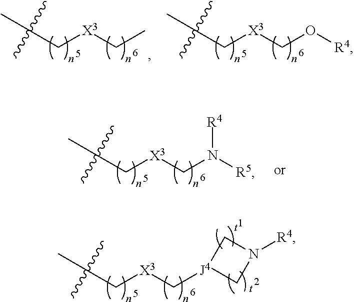

A is optionally present and is NR⁶ or of formula:

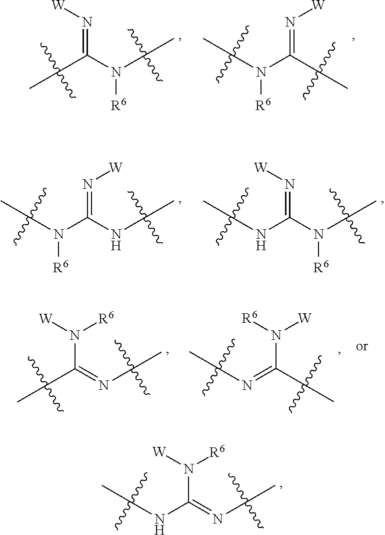

U is optionally present and is CH₂, C(O), CH₂C(O), or C(O)CH₂,

R⁶ and W independently are hydrogen, Ar¹, or of formula:

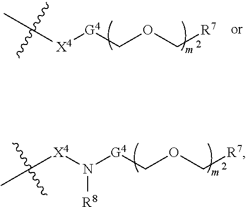

V is optionally present and is of formula:

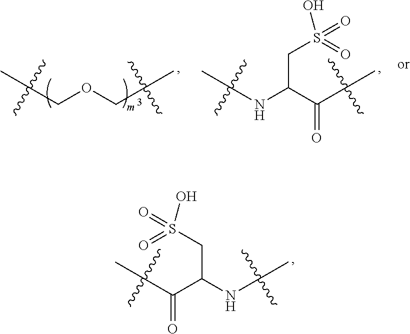

J³ and J⁴ independently are CH or N,

m¹, m², and m³ independently are an integer from 0 to 25, except that at least one of m¹, m², and m³ is a non-zero integer,

n¹, n², n³, n⁴, n⁵, and n⁶ independently are an integer from 0 to 10,

t¹ and t² independently are an integer from 1 to 3,

G¹, G², G³, and G⁴ independently are CH₂, C(O), CH₂C(O), C(O)CH₂, or a bond,

X¹, X², X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

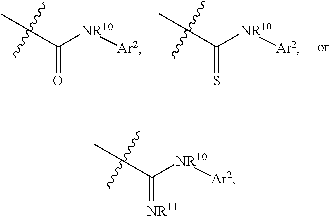

R³, R⁵, R⁷, R⁸, R⁹, R¹⁰, and R¹¹ independently are hydrogen or C₁-C₄ alkyl,

Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (IIa₁), (IIa₂), (IIa₃), (IIa₄), (IIa₅), or (IIa₆):

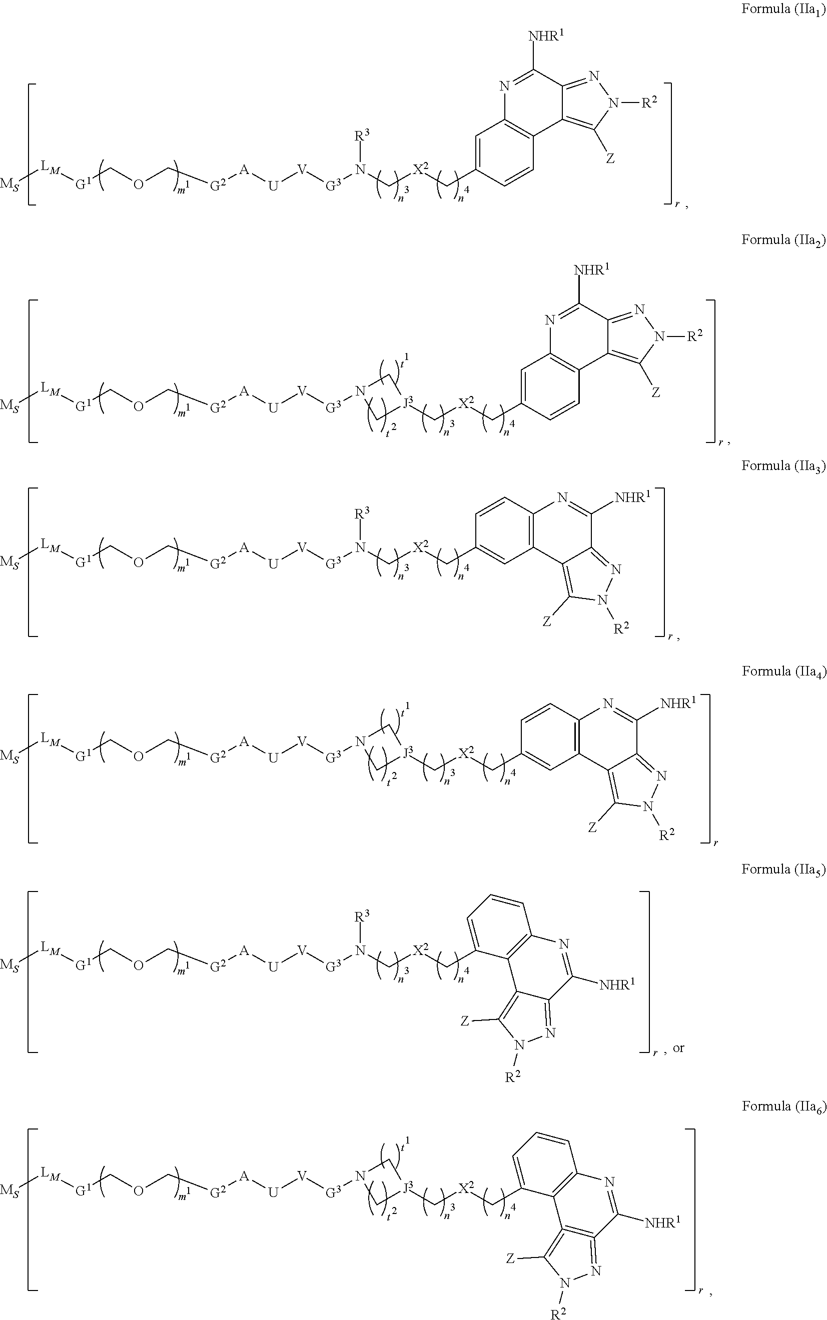

a pharmaceutically acceptable salt thereof, or a quaternary ammonium salt thereof, wherein

R¹ and R² independently are hydrogen or of formula:

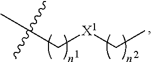

Z is hydrogen or of formula:

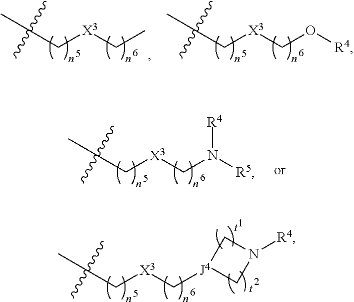

A is optionally present and is NR⁶ or of formula:

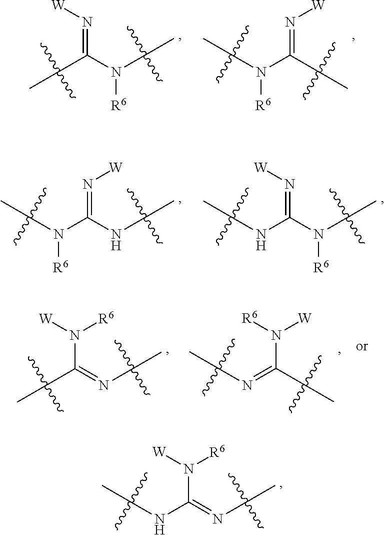

U is optionally present and is CH₂, C(O), CH₂C(O), or C(O)CH₂,

R⁶ and W independently are hydrogen, Ar¹, or of formula:

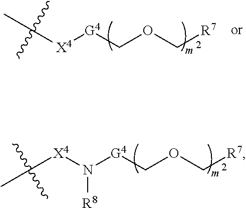

V is optionally present and is of formula:

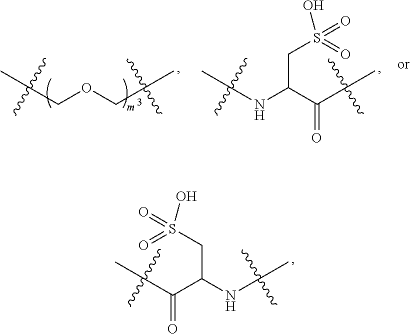

J³ and J⁴ independently are CH or N,

m¹, m², and m³ independently are an integer from 0 to 25, except that at least one of m¹, m², and m³ is a non-zero integer,

n¹, n², n³, n⁴, n⁵, and n⁶ independently are an integer from 0 to 10,

t¹ and t² independently are an integer from 1 to 3,

G¹, G², G³, and G⁴ independently are CH₂, C(O), CH₂C(O), C(O)CH₂, or a bond,

X¹, X², X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

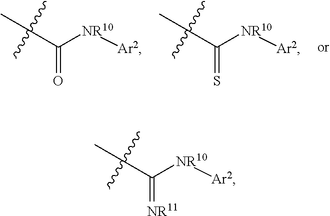

R³, R⁵, R⁷, R⁸, R⁹, R¹⁰, and R¹¹ independently are hydrogen or C₁-C₄ alkyl,

Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (IIaa), (IIab), (IIac), (IIad), (IIae), or (IIaf):

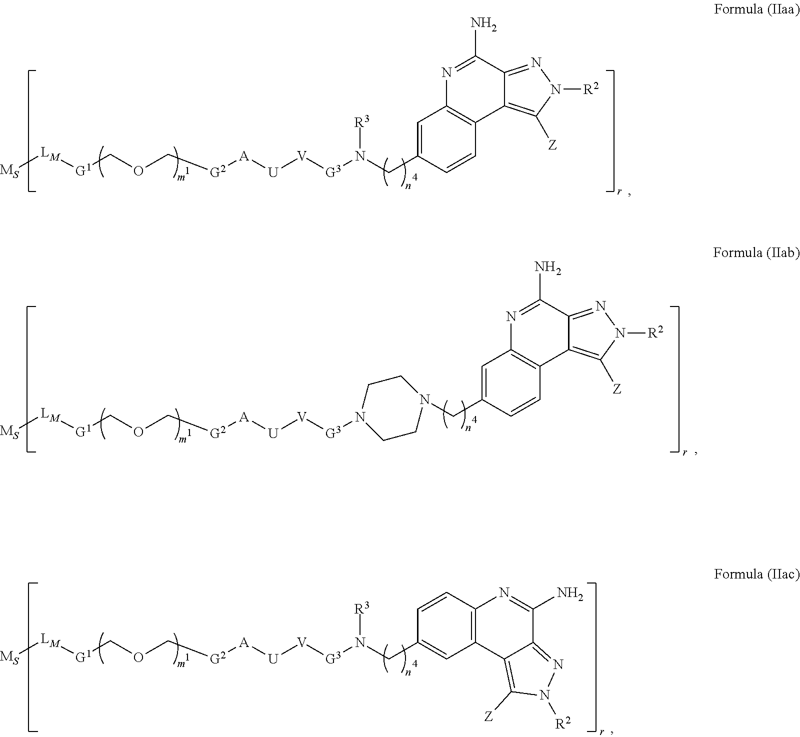

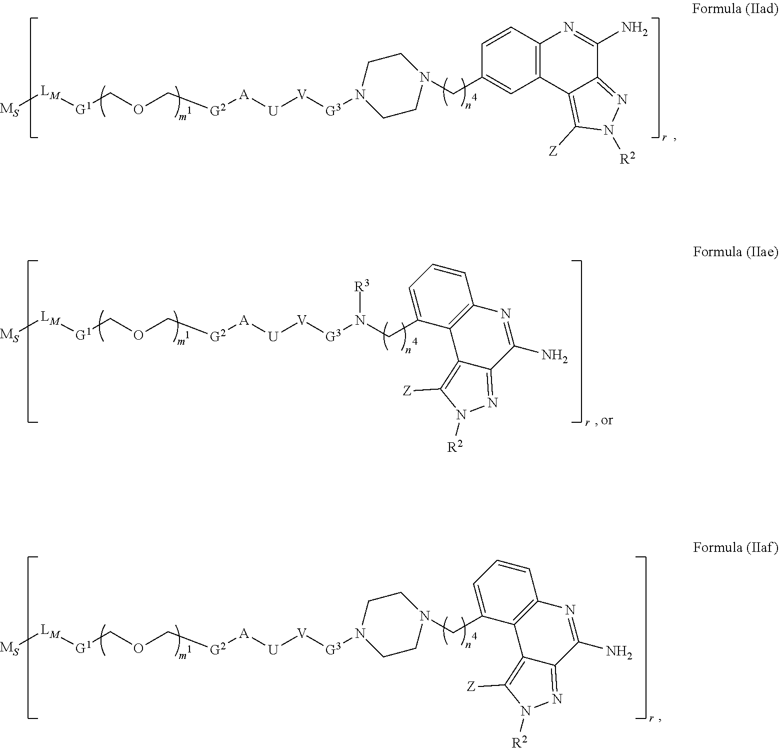

a pharmaceutically acceptable salt thereof, or a quaternary ammonium salt thereof, wherein

R² is of formula:

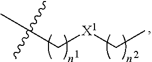

Z is hydrogen or of formula:

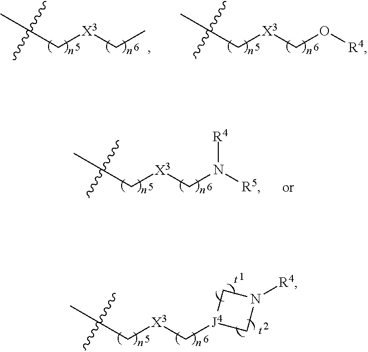

A is optionally present and is NR⁶ or of formula:

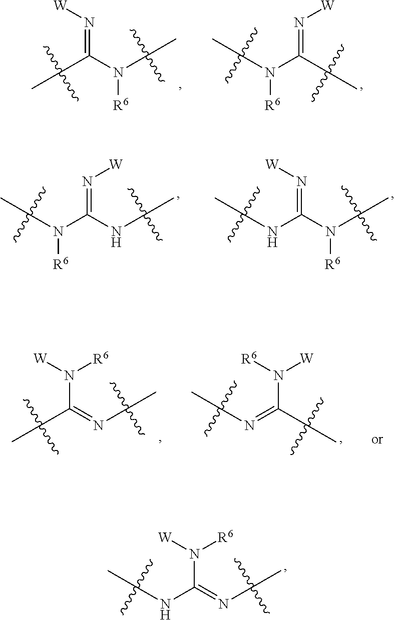

U is optionally present and is CH₂, C(O), CH₂C(O), or C(O)CH₂,

R⁶ and W independently are hydrogen, Ar¹, or of formula:

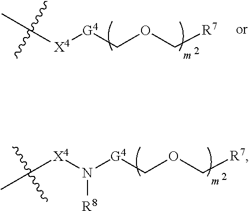

V is optionally present and is of formula:

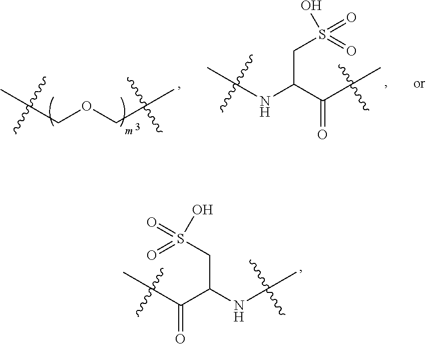

J⁴ is CH or N,

m¹, m², and m³ independently are an integer from 0 to 25, except that at least one of m¹, m², and m³ is a non-zero integer,

n¹, n², n⁴, n⁵, and n⁶ independently are an integer from 0 to 10,

G¹, G², G³, and G⁴ independently are CH₂, C(O), CH₂C(O), C(O)CH₂, or a bond,

X¹, X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

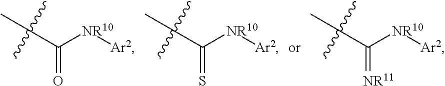

R³, R⁵, R⁷, R⁸, R⁹, R¹⁰, and R¹¹ independently are hydrogen or C₁-C₄ alkyl,

Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (IIb):

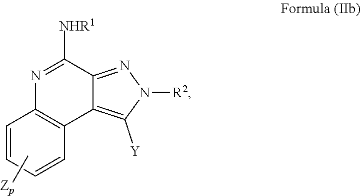

a pharmaceutically acceptable salt thereof, or a quaternary ammonium salt thereof, wherein

R¹ and R² independently are hydrogen or of formula:

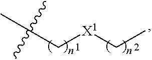

Y is of formula:

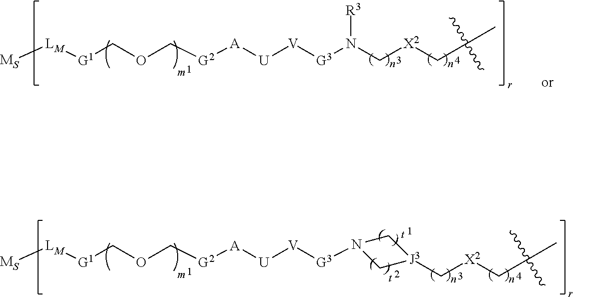

each Z independently is hydrogen or of formula:

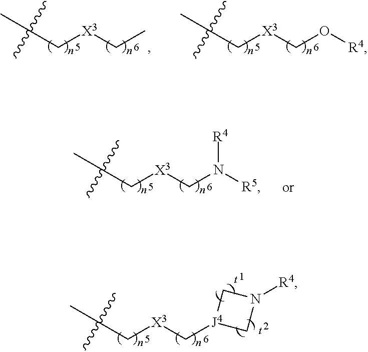

A is optionally present and is NR⁶ or of formula:

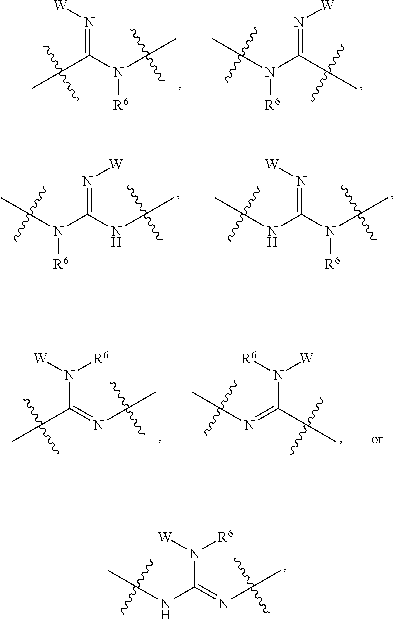

U is optionally present and is CH₂, C(O), CH₂C(O), or C(O)CH₂,

R⁶ and W independently are hydrogen, Ar¹, or of formula:

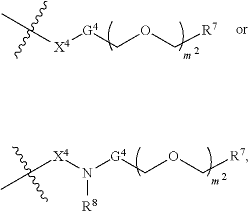

V is optionally present and is of formula:

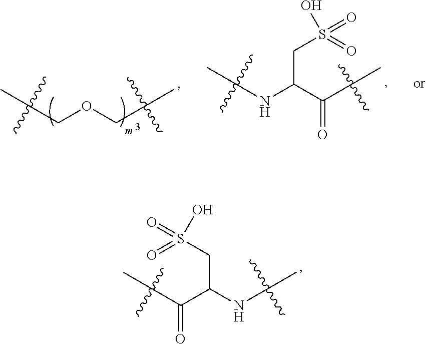

J³ and J⁴ independently are CH or N,

m¹, m², and m³ independently are an integer from 0 to 25, except that at least one of m¹, m², and m³ is a non-zero integer,

n¹, n², n³, n⁴, n⁵, and n⁶ independently are an integer from 0 to 10,

p is an integer from 1 to 4,

t¹ and t² independently are an integer from 1 to 3,

G¹, G², G³, and G⁴ independently are CH₂, C(O), CH₂C(O), C(O)CH₂, or a bond,

X¹, X², X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

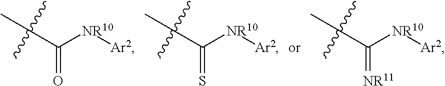

R³, R⁵, R⁷, R⁸, R⁹, R¹⁰, and R¹¹ independently are hydrogen or C₁-C₄ alkyl,

Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (IIb₁), (IIb₂), (IIb₃), or (IIb₄):

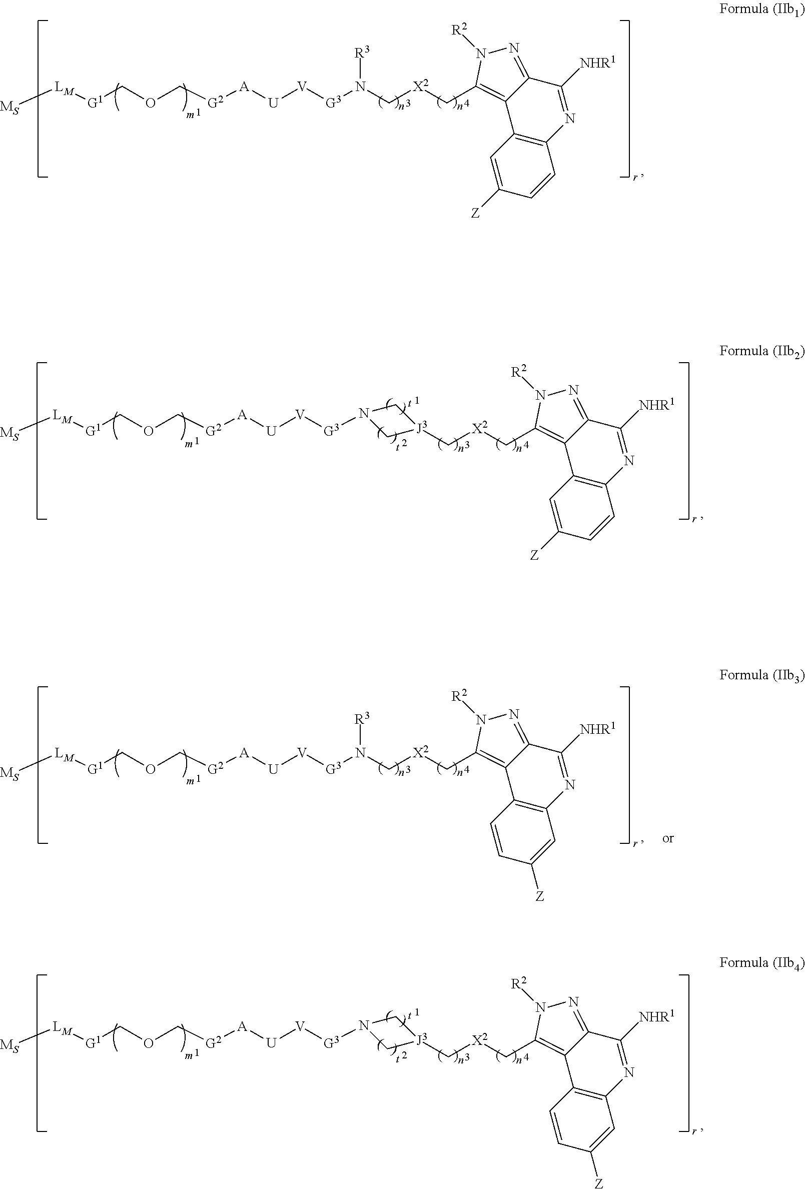

a pharmaceutically acceptable salt thereof, or a quaternary ammonium salt thereof, wherein

R¹ and R² independently are hydrogen or of formula:

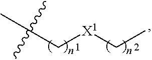

Z is hydrogen or of formula:

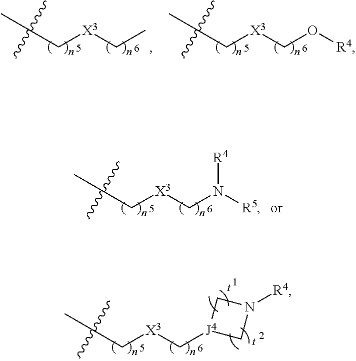

A is optionally present and is NR⁶ or of formula:

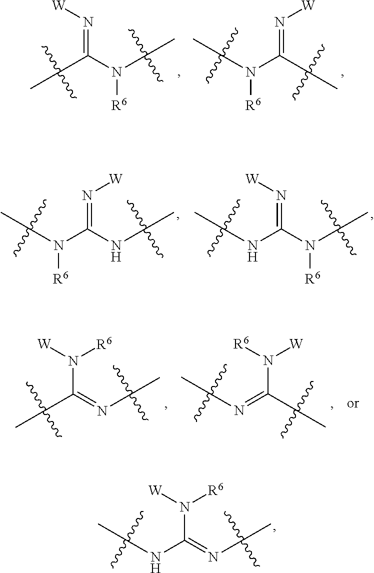

U is optionally present and is CH₂, C(O), CH₂C(O), or C(O)CH₂,

R⁶ and W independently are hydrogen, Ar¹, or of formula:

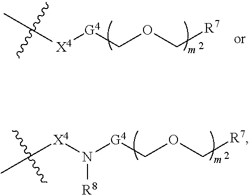

V is optionally present and is of formula:

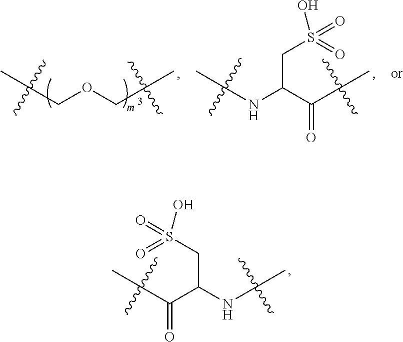

J³ and J⁴ independently are CH or N,

m¹, m², and m³ independently are an integer from 0 to 25, except that at least one of m¹, m², and m³ is a non-zero integer,

n¹, n², n³, n⁴, n⁵, and n⁶ independently are an integer from 0 to 10,

t¹ and t² independently are an integer from 1 to 3,

G¹, G², G³, and G⁴ independently are CH₂, C(O), CH₂C(O), C(O)CH₂, or a bond,

X¹, X², X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

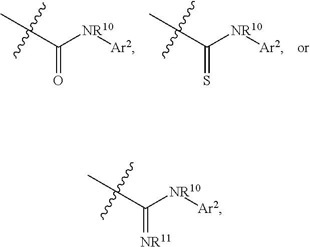

R⁴ is hydrogen, C₁-C₄ alkyl,

R³, R⁵, R⁷, R⁸, R⁹, R¹⁰, and R¹¹ independently are hydrogen or C₁-C₄ alkyl,

Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (IIba), (IIbb), (IIbc), or (IIbd):

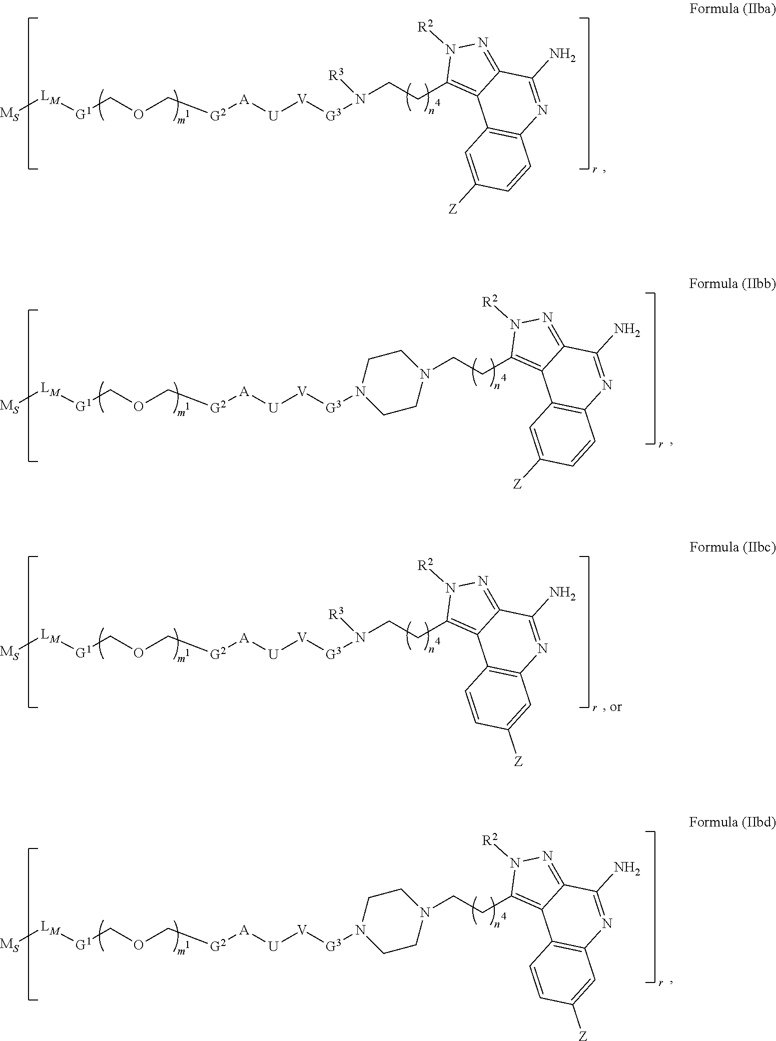

a pharmaceutically acceptable salt thereof, or a quaternary ammonium salt thereof, wherein

R² is of formula:

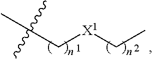

Z is hydrogen or of formula:

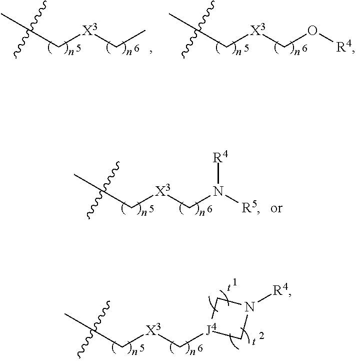

A is optionally present and is NR⁶ or of formula:

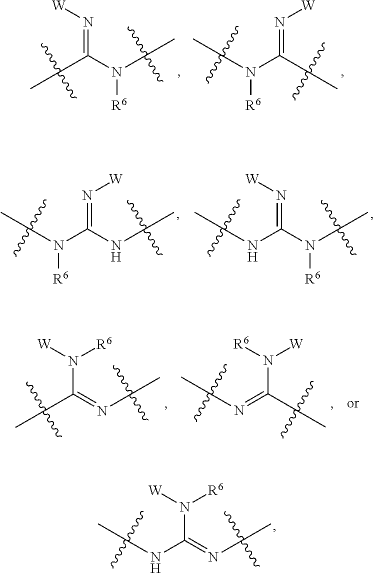

U is optionally present and is CH₂, C(O), CH₂C(O), or C(O)CH₂,

R⁶ and W independently are hydrogen, Ar¹, or of formula:

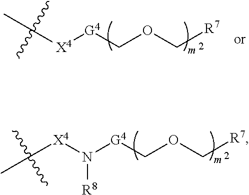

V is optionally present and is of formula:

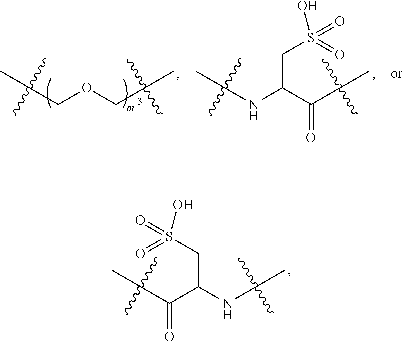

J³ and J⁴ independently are CH or N,

m¹, m², and m³ independently are an integer from 0 to 25, except that at least one of m¹, m², and m³ is a non-zero integer,

n¹, n², n⁴, n⁵, and n⁶ independently are an integer from 0 to 10,

G¹, G², G³, and G⁴ independently are CH₂, C(O), CH₂C(O), C(O)CH₂, or a bond,

X¹, X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

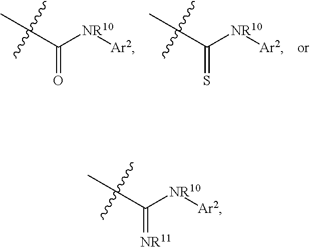

R³, R⁵, R⁷, R⁸, R⁹, R¹⁰, and R¹¹ independently are hydrogen or C₁-C₄ alkyl,

Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (IIc):

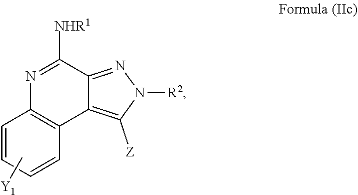

a pharmaceutically acceptable salt thereof, or a quaternary ammonium salt thereof, wherein

R¹ and R² independently are hydrogen or of formula:

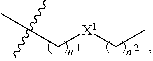

Y₁ is of formula:

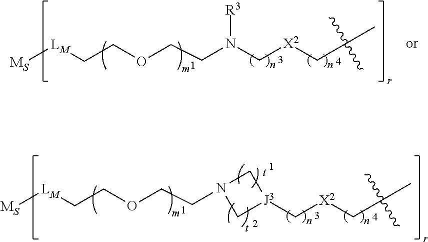

Z is hydrogen or of formula:

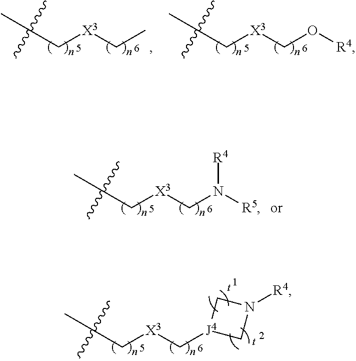

J³ and J⁴ independently are CH or N,

m¹ is an integer from 1 to 25,

n¹, n², n³, n⁴, n⁵, and n⁶ independently are an integer from 0 to 10,

t¹ and t² independently are an integer from 1 to 3,

X¹, X², and X³ are each optionally present and independently are O, NH⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

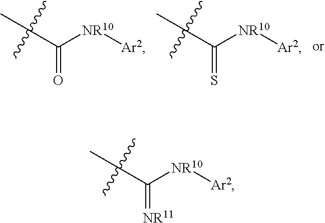

R³, R⁵, R⁹, R¹⁰, and R¹¹ independently are hydrogen or C₁-C₄ alkyl,

Ar² is an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (IId):

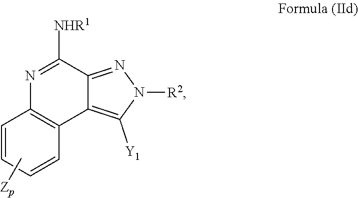

a pharmaceutically acceptable salt thereof, or a quaternary ammonium salt thereof, wherein

R¹ and R² independently are hydrogen or of formula:

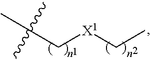

Y₁ is of formula:

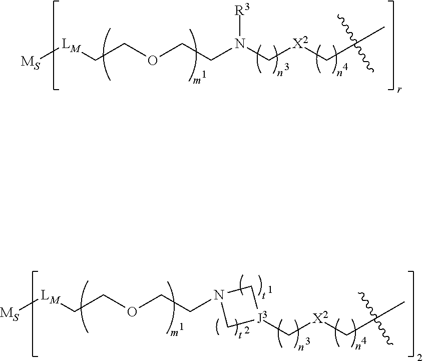

each Z independently is hydrogen or of formula:

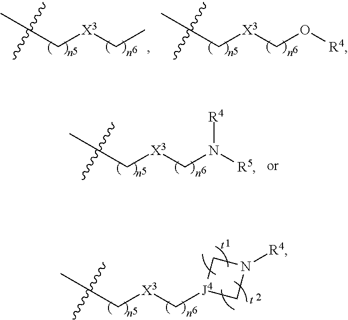

J³ and J⁴ independently are CH or N,

m¹ is an integer from 1 to 25,

n¹, n², n³, n⁴, n⁵, and n⁶ independently are an integer from 0 to 10,

p is an integer from 1 to 4,

t¹ and t² independently are an integer from 1 to 3,

X¹, X², and X³ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

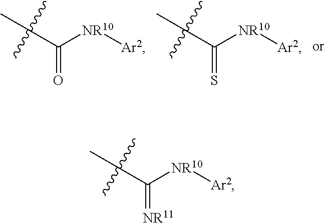

R³, R⁵, R⁹, R¹⁰, and R¹¹ independently are hydrogen or C₁-C₄ alkyl,

Ar² is an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (IIe):

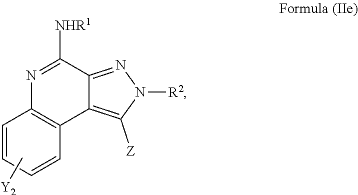

a pharmaceutically acceptable salt thereof, or a quaternary ammonium salt thereof, wherein

R¹ and R² independently are hydrogen or of formula:

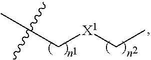

Y₂ is of formula:

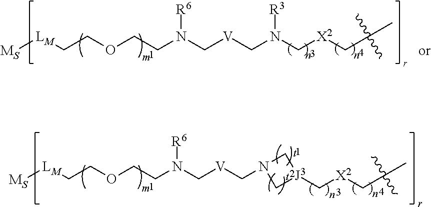

Z is hydrogen or of formula:

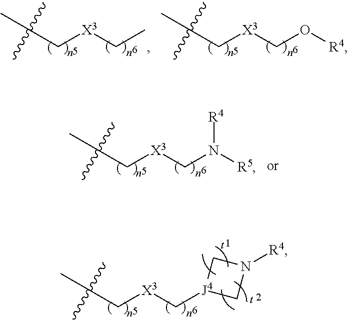

R⁶ is hydrogen, Ar¹, or of formula:

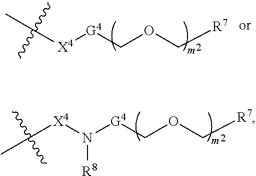

V is optionally present and is of formula:

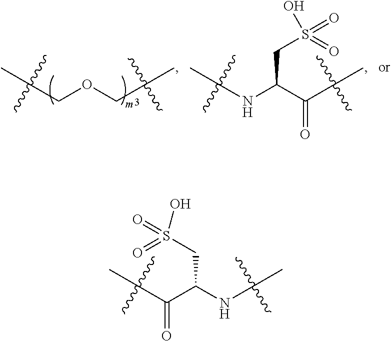

J³ and J⁴ independently are CH or N,

m¹, m², and m³ independently are an integer from 0 to 25, except that at least one of m¹, m², and m³ is a non-zero integer,

n¹, n², n³, n⁴, n⁵, and n⁶ independently are an integer from 0 to 10,

t¹ and t² independently are an integer from 1 to 3,

G⁴ is CH₂, C(O), CH₂C(O), C(O)CH₂, or a bond,

X¹, X², X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

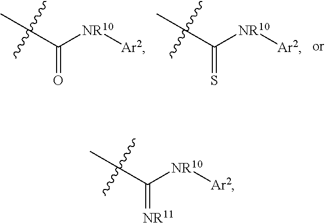

R³, R⁵, R⁷, R⁸, R⁹, R¹⁰, and R¹¹ independently are hydrogen or C₁-C₄ alkyl,

Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (If)

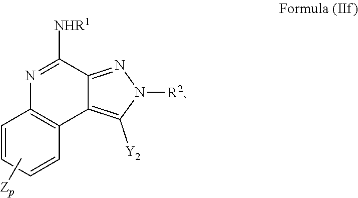

a pharmaceutically acceptable salt thereof, or a quaternary ammonium salt thereof, wherein

R¹ and R² independently are hydrogen or of formula:

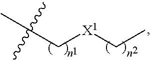

Y₂ is of formula:

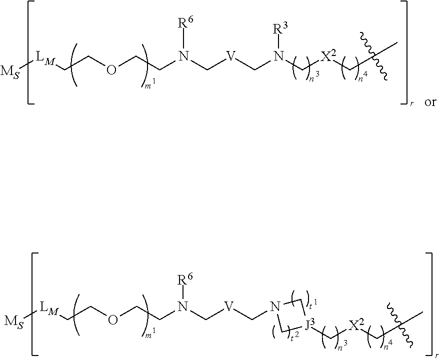

each Z independently is hydrogen or of formula:

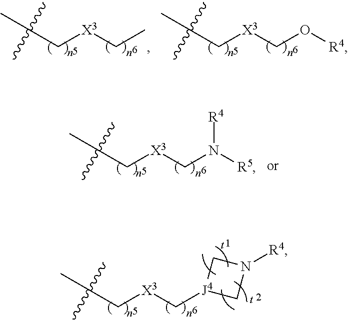

R⁶ is hydrogen, Ar¹, or of formula:

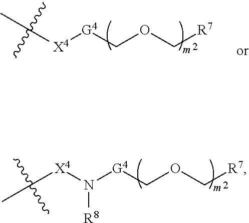

V is optionally present and is of formula:

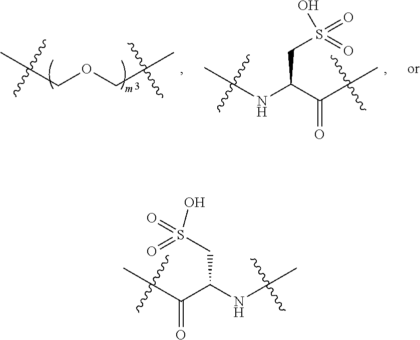

J³ and J⁴ independently are CH or N,

m¹, m², and m³ independently are an integer from 0 to 25, except that at least one of m¹, m², and m³ is a non-zero integer,

p is an integer from 1 to 4,

t¹ and t² independently are an integer from 1 to 3,

G⁴ is CH₂, C(O), CH₂C(O), C(O)CH₂, or a bond,

X¹, X², X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

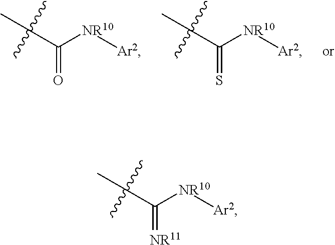

R³, R⁵, R⁷, R⁸, R⁹, R¹⁰, and R¹¹ independently are hydrogen or C₁-C₄ alkyl,

Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (IIg):

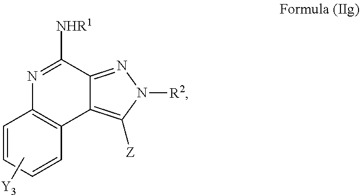

a pharmaceutically acceptable salt thereof, or a quaternary ammonium salt thereof, wherein

R¹ and R² independently are hydrogen or of formula:

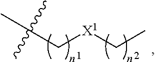

Y₃ is of formula:

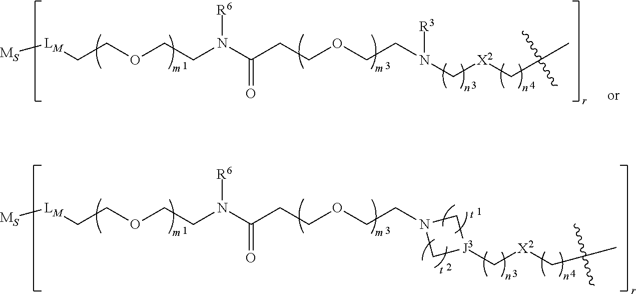

Z is hydrogen or of formula:

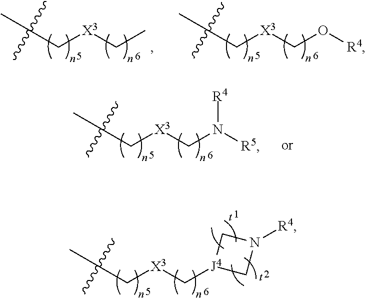

R⁶ is hydrogen, Ar¹, or of formula:

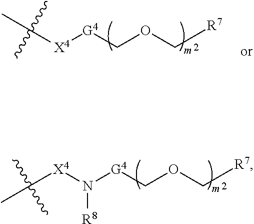

J³ and J⁴ independently are CH or N,

m¹, m², and m³ independently are an integer from 0 to 25, except that at least one of m¹, m², and m³ is a non-zero integer,

n¹, n², n³, n⁴, n⁵, and n⁶ independently are an integer from 0 to 10,

t¹ and t² independently are an integer from 1 to 3,

G⁴ is CH₂, C(O), CH₂C(O), C(O)CH₂, or a bond,

X¹, X², X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

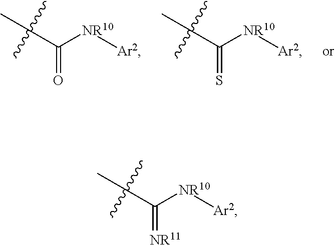

R³, R⁵, R⁷, R⁸, R⁹, R¹⁰, and R¹¹ independently are hydrogen or C₁-C₄ alkyl,

Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (IIh):

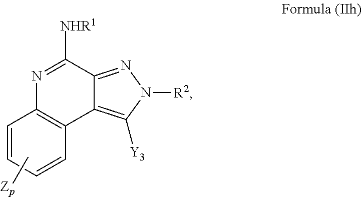

a pharmaceutically acceptable salt thereof, or a quaternary ammonium salt thereof, wherein

R¹ and R² independently are hydrogen or of formula:

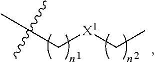

Y₃ is of formula:

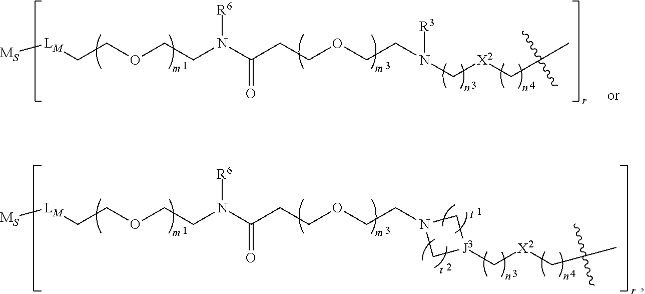

each Z independently is hydrogen or of formula:

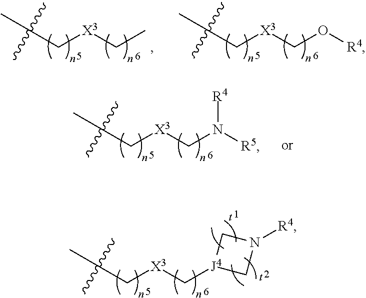

R⁶ is hydrogen, Ar¹, or of formula:

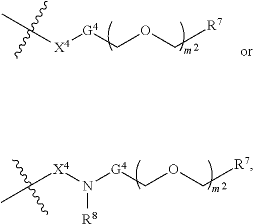

J³ and J⁴ independently are CH or N,

m¹, m², and m³ independently are an integer from 0 to 25, except that at least one of m¹, m², and m³ is a non-zero integer,

n¹, n², n³, n⁴, n⁵, and n⁶ independently are an integer from 0 to 10,

p is an integer from 1 to 4,

t¹ and t² independently are an integer from 1 to 3,

G⁴ is CH₂, C(O), CH₂C(O), C(O)CH₂, or a bond,

X¹, X², X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

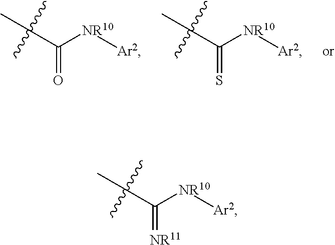

R³, R⁵, R⁷, R⁸, R⁹, R¹⁰, and R¹¹ independently are hydrogen or C₁-C₄ alkyl,

Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (IIi):

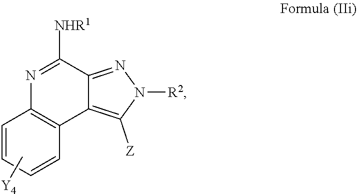

a pharmaceutically acceptable salt thereof, or a quaternary ammonium salt thereof, wherein

R¹ and R² independently are hydrogen or of formula:

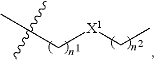

Y₄ is of formula:

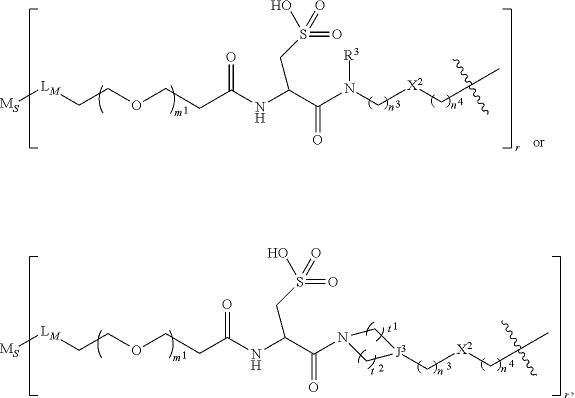

Z is hydrogen or of formula:

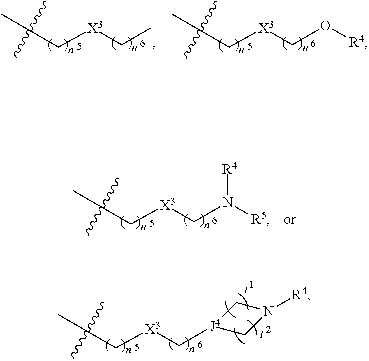

J³ and J⁴ independently are CH or N,

m¹ is an integer from 1 to 25,

n¹, n², n³, n⁴, n⁵, and n⁶ independently are an integer from 0 to 10,

t¹ and t² independently are an integer from 1 to 3,

X¹, X², and X³ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

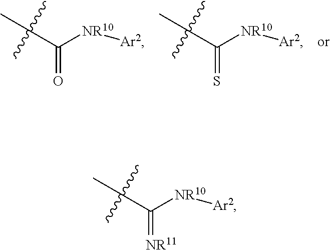

R³, R⁵, R⁹, R¹⁰, and R¹¹ independently are hydrogen or C₁-C₄ alkyl,

Ar² is an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (IIj):

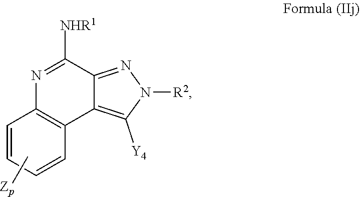

a pharmaceutically acceptable salt thereof, or a quaternary ammonium salt thereof, wherein

R¹ and R² independently are hydrogen or of formula:

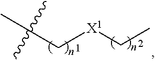

Y₄ is of formula:

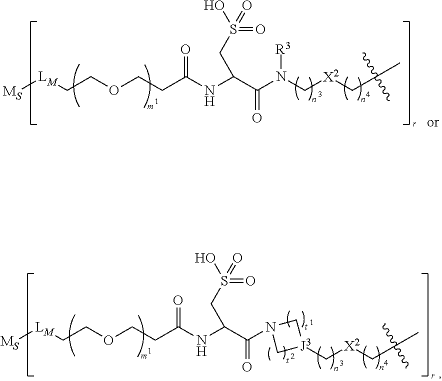

each Z independently is hydrogen or of formula:

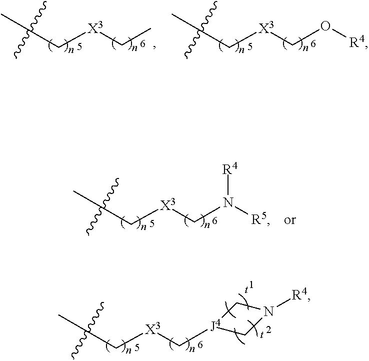

J³ and J⁴ independently are CH or N,

m¹ is an integer from 1 to 25,

n¹, n², n³, n⁴, n⁵, and n⁶ independently are an integer from 0 to 10,

p is an integer from 1 to 4,

t¹ and t² independently are an integer from 1 to 3,

X¹, X², and X³ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

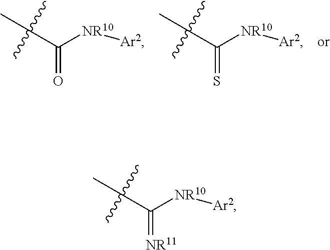

R³, R⁵, R⁹, R¹⁰, and R¹¹ independently are hydrogen or C₁-C₄ alkyl,

Ar² is an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (IIk):

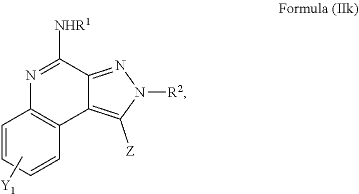

a pharmaceutically acceptable salt thereof, or a quaternary ammonium salt thereof, wherein

R¹ and R² independently are hydrogen or of formula:

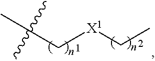

Y₅ is of formula:

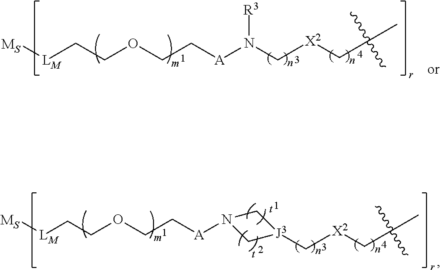

Z is hydrogen or of formula:

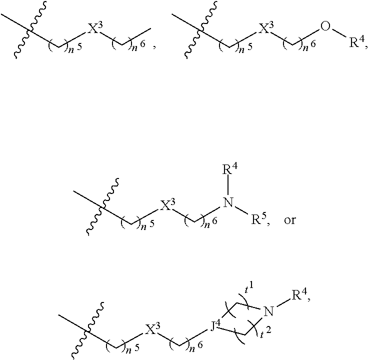

A is NR⁶ or of formula:

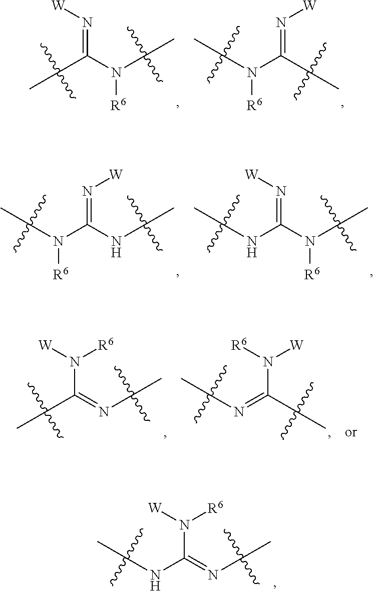

R⁶ and W independently are hydrogen, Ar¹, or of formula:

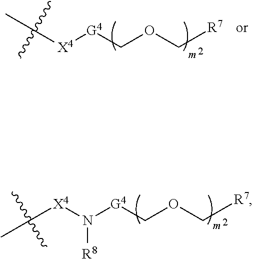

J³ and J⁴ independently are CH or N,

m¹ and m² independently are an integer from 0 to 25, except that at least one of m¹ and m² is a non-zero integer,

n¹, n², n³, n⁴, n⁵, and n⁶ independently are an integer from 0 to 10,

t¹ and t² independently are an integer from 1 to 3,

G⁴ is CH₂, C(O), CH₂C(O), C(O)CH₂, or a bond,

X¹, X², X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

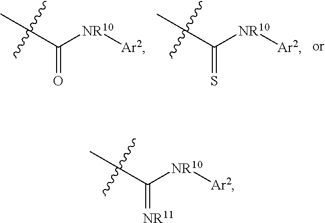

R³, R⁵, R⁷, R⁸, R⁹, R¹⁰, and R¹¹ independently are hydrogen or C₁-C₄ alkyl,

Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments, the macromolecule-supported compound is of formula (IIm):

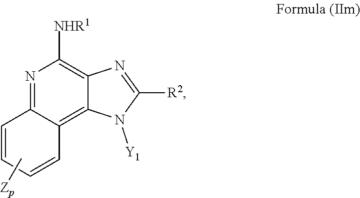

a pharmaceutically acceptable salt thereof, or a quaternary ammonium salt thereof, wherein

R¹ and R² independently are hydrogen or of formula:

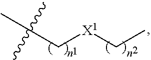

Y₅ is of formula:

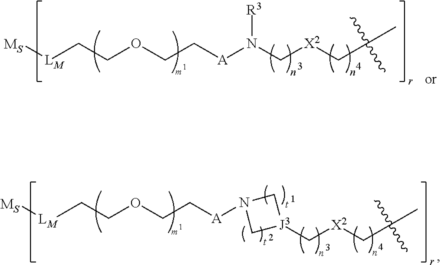

each Z independently is hydrogen or of formula:

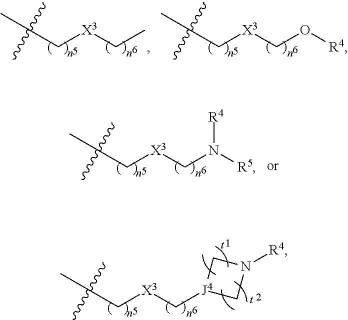

A is NR⁶ or of formula:

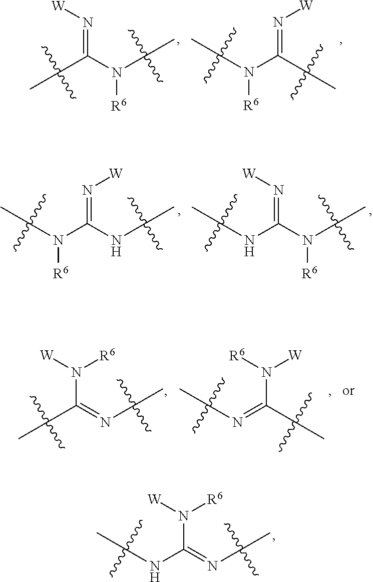

R⁶ and W independently are hydrogen, Ar¹, or of formula:

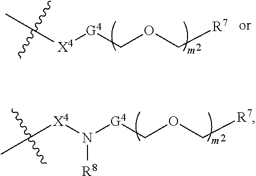

J³ and J⁴ independently are CH or N,

m¹ and m² independently are an integer from 0 to 25, except that at least one of m¹ and m² is a non-zero integer,

n¹, n², n³, n⁴, n⁵, and n⁶ independently are an integer from 0 to 10,

p is an integer from 1 to 4,

t¹ and t² independently are an integer from 1 to 3,

G⁴ is CH₂, C(O), CH₂C(O), C(O)CH₂, or a bond,

X¹, X², X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

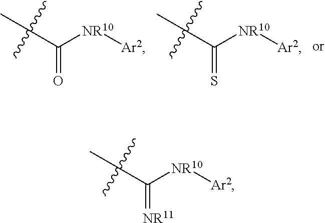

R³, R⁵, R⁷, R⁸, R⁹, R¹⁰, and R¹¹ independently are hydrogen or C₁-C₄ alkyl,

Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

In certain embodiments of the invention, one or more aromatic hydrogen atoms in formulas (I) and (II) can be substituted with a halogen atom (e.g., fluorine, chlorine, bromine, iodine, or combinations thereof).

For variable Y described herein as formulas:

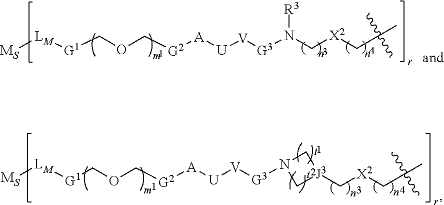

it will be understood that the structures:

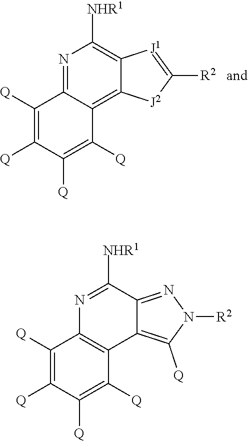

are present within the brackets such that the variable r also applies to said structures.

Generally, the macromolecule-supported compounds of the invention comprise about 1 to about 50 TLR agonists (e.g., about 1 to about 25 or about 1 to about 10), each TLR agonist linked to the macromolecular support, as designated with subscript “r”. In an embodiment, r is 1, such that there is a single TLR agonist linked to the macromolecular support. In some embodiments, r is an integer from about 2 to about 10 (e.g., about 2 to about 9, about 3 to about 9, about 4 to about 9, about 5 to about 9, about 6 to about 9, about 3 to about 8, about 3 to about 7, about 3 to about 6, about 4 to about 8, about 4 to about 7, about 4 to about 6, about 5 to about 6, about 1 to about 6, about 1 to about 4, about 2 to about 4, or about 1 to about 3). Accordingly, the macromolecule-supported compounds can have (i.e., subscript “r” can be) 1, 2, 3, 4, 5, 6, 7, 8, 9, or 10 TLR agonists linked to the macromolecular support. In preferred embodiments, the macromolecule-supported compounds have (i.e., subscript “r” can be) 1, 2, 3, or 4 TLR agonists linked to the macromolecular support. The desirable TLR agonist to macromolecular support ratio (i.e., the value of the subscript “r”) can be determined by a skilled artisan depending on the desired effect of the treatment.

In some embodiments, X¹, X², X³, and X⁴ independently are one or more divalent groups selected from benzene, naphthalene, pyrrole, indole, isoindole, indolizine, furan, benzofuran, benzothiophene, thiophene, pyridine, acridine, naphthyridine, quinolone, isoquinoline, isoxazole, oxazole, benzoxazole, isothiazole, thiazole, benzthiazole, imidazole, thiadiazole, tetrazole, triazole, oxadiazole, benzimidazole, purine, pyrazole, pyrazine, pteridine, quinoxaline, phthalazine, quinazoline, triazine, phenazine, cinnoline, pyrimidine, pyridazine, cyclohexane, decahydronaphthalene, pyrrolidine, octahydroindole, octahydroisoindole, tetrahydrofuran, octahydrobenzofuran, octahydrobenzothiophene, tetrahydrothiophene, piperidine, tetradecahydroacridine, naphthyridine, decahydroquinoline, decahydroisoquinoline, isoxazolidine, oxazolidine, octahydrobenzooxazole, isothiazolidine, thiazolidine, octahydrobenzothiazole, imidazolidine, 1,2,3-thiadiazolidine, tetrazolidine, 1,2,3-triazolidine, 1,2,3-oxadiazolidine, octahydrobenzoimidazole, octahydropurine, pyrazolidine, piperazine, dechydropteridine, decahydroquinoxaline, dechydrophthalazine, dechydroquinazoline, 1,3,5-triazinane, tetradecahydrophenazine, decahydrocinnoline, hexhydropyrimidine, or hexahydropyridazine. In some embodiments, the one or more divalent groups of X¹, X², and X³ are fused. In some embodiments, the one or more divalent groups of X¹, X², and X³ are linked through a bond or —CO—. In certain embodiments, X¹, X², and X³ can be substituted with one or more halogens (e.g., fluorine, chlorine, bromine, or iodine), nitriles, hydroxyls, C₁-C₄ alkyl groups, or a combination thereof.

In certain embodiments, X¹, X², X³, and X⁴ independently are of formula:

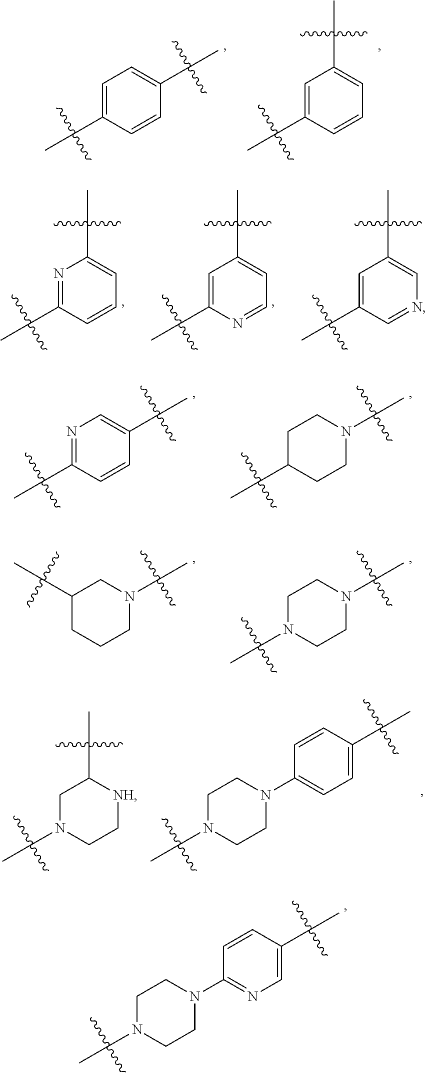

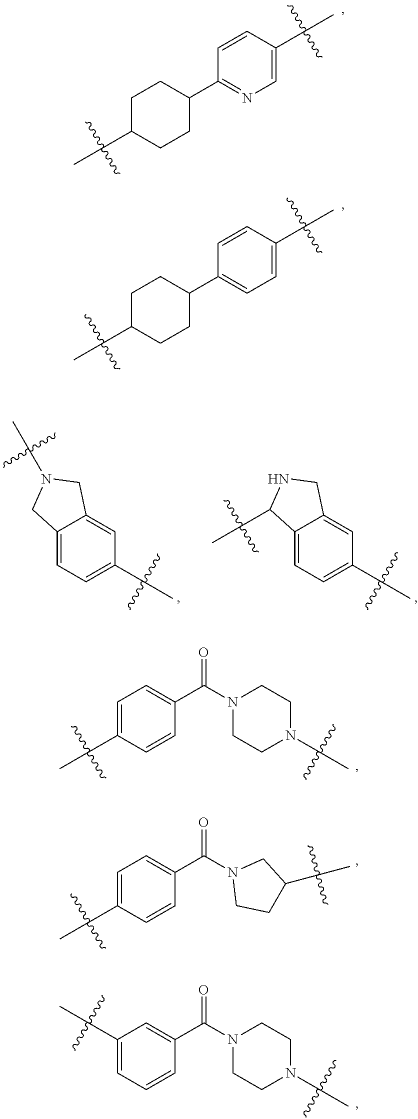

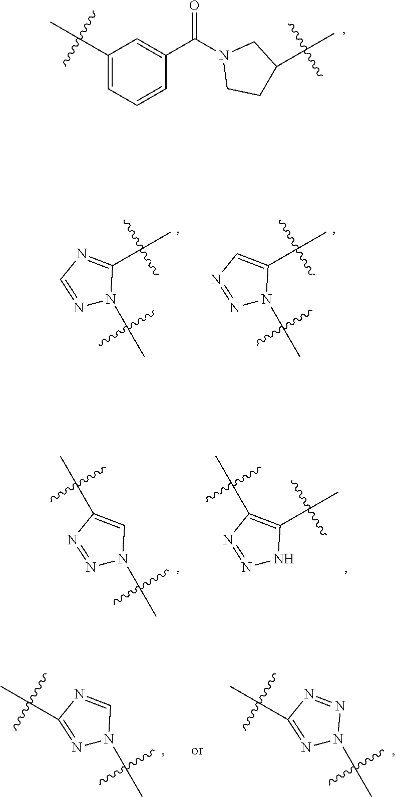

wherein any of the above-referenced structures can be used bilaterally.

Variables Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof. Ar¹ and Ar² can be any suitable aryl or heteroaryl group described herein. In some embodiments, Ar¹ and Ar² independently are a monovalent aryl or heteroaryl group described by the divalent groups of X¹, X², X³, and X⁴, optionally substituted with one or more halogens (e.g., fluorine, chlorine, bromine, or iodine), nitriles, hydroxyls, C₁-C₄ alkyl groups, or a combination thereof.

Variables m¹, m², and m³ independently are an integer from 0 to 25. Typically, at least one of m¹, m², and m³ is a non-zero integer such that at least one of m¹, m², and m³ is an integer from 1 to 25. In certain embodiments, at least one of m¹, m², and m³ is an integer from about 2 to about 25 (e.g., about 2 to about 16, about 6 to about 25, about 6 to about 16, about 8 to about 25, about 8 to about 16, about 6 to about 12, or about 8 to about 12). Accordingly, in some embodiments, the macromolecule-supported compounds of the invention comprise about 2 to about 25 (e.g., about 2 to about 16, about 6 to about 25, about 6 to about 16, about 8 to about 25, about 8 to about 16, about 6 to about 12, or about 8 to about 12) ethylene glycol units, as designated with subscripts “m¹”, “m²” and “m³”. Accordingly, the macromolecule-supported compounds of the invention can comprise at least 2 ethylene glycol groups (e.g., at least 3 ethylene glycol groups, at least 4 ethylene glycol groups, at least 5 ethylene glycol groups, at least 6 ethylene glycol groups, at least 7 ethylene glycol groups, at least 8 ethylene glycol groups, at least 9 ethylene glycol groups, or at least 10 ethylene glycol groups). Accordingly, the macromolecule-supported compound can comprise from about 2 to about 25 ethylene glycol units, for example, from about 6 to about 25 ethylene glycol units, from about 6 to about 16 ethylene glycol units, from about 8 to about 25 ethylene glycol units, from about 8 to about 16 ethylene glycol units, from about 8 to about 12 ethylene glycol units, or from about 8 to about 12 ethylene glycol units. In certain embodiments, the macromolecule-supported compound comprises a di(ethylene glycol) group, a tri(ethylene glycol) group, a tetra(ethylene glycol) group, 5 ethylene glycol groups, 6 ethylene glycol groups, 7 ethylene glycol groups, 8 ethylene glycol groups, 9 ethylene glycol groups, 10 ethylene glycol groups, 11 ethylene glycol groups, 12 ethylene glycol groups, 13 ethylene glycol groups, 14 ethylene glycol groups, 15 ethylene glycol groups, 16 ethylene glycol groups, 24 ethylene glycol groups, or 25 ethylene glycol groups.

Variable p is an integer from 1 to 4 (e.g., 1, 2, 3, or 4). In certain embodiments, p is 1 such that the aryl ring has one Z substituent that is not hydrogen at one of the four available carbons.

Variables t¹ and t² independently are an integer from 1 to 3. In some embodiments, when present, t¹ and t² are 1 and 2, respectively, or t¹ and t² are 2 and 2, respectively. In preferred embodiments, when present, t¹ and t² are 2 and 2, respectively.

The linking moiety (L_{M}) represents the remnants of a chemical species used to conjugate the TLR agonist to the macromolecular support. L_{M} can be any suitable remnants of any conjugation techniques known in the art. One of skill in the art will appreciate that the TLR agonist moieties in the macromolecule-support compound can be covalently bonded to the macromolecular support using various chemistries, and that the linking moieties described above result from the reaction of free functional groups (e.g., amino acid side chains, surface alcohols, thiols, carbonyls, acids, or amines, nucleic acids, etc.), with reagents having reactive linker groups. A wide variety of such reagents are known in the art. Examples of such reagents include, but are not limited to, N-hydroxysuccinimidyl (NETS) esters and N-hydroxysulfosuccinimidyl (sulfo-NHS) esters (amine reactive); carbodiimides (amine and carboxyl reactive); hydroxymethyl phosphines (amine reactive); maleimides (thiol reactive); halogenated acetamides such as N-iodoacetamides (thiol reactive); aryl azides (primary amine reactive); fluorinated aryl azides (reactive via carbon-hydrogen (C—H) insertion); pentafluorophenyl (PFP) esters (amine reactive); tetrafluorophenyl (TFP) esters (amine reactive); imidoesters (amine reactive); isocyanates (hydroxyl reactive); vinyl sulfones (thiol, amine, and hydroxyl reactive); pyridyl disulfides (thiol reactive); and benzophenone derivatives (reactive via C—H bond insertion). Further reagents include but are not limited to those described in Hermanson, Bioconjugate Techniques 2nd Edition, Academic Press, 2008.

Linkers containing maleimide groups, vinyl sulfone groups, pyridyl disulfide groups, and halogenated acetamide groups are particularly useful for covalent bonding to thiol groups in a macromolecular support. Thiol groups in a macromolecular support can be located in cysteine sidechains. Thiol groups may be on the surface of the macromolecular support, present in naturally-occurring, solvent-accessible cysteine residues or in engineered cysteine residues, as described below. In addition, thiol groups can be generated via full or partial reduction of disulfide linkages.

For example, L_{M} can be of formula:

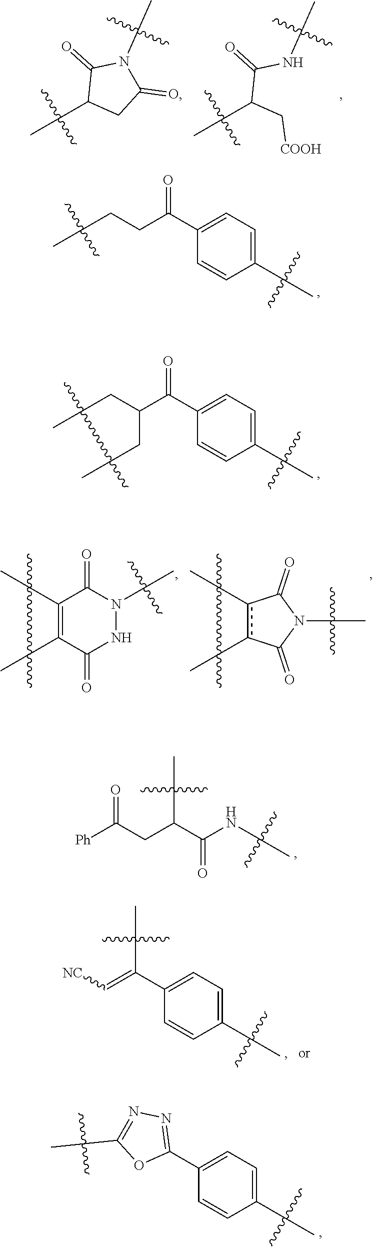

where L_{M} is bound to one or more thiol groups in a macromolecular support, and the one or more thiol groups are, for example, naturally-occurring, solvent-accessible cysteine residues in or on a macromolecular support, present in engineered cysteine residues, generated via full or partial reduction of disulfide linkages between cysteine sidechains (e.g., in an antibody), or appended to lysine sidechains. When the wavy line (“ [symbol] ”) crosses multiple bonds, it will be understood that the L_{M} attaches to the macromolecular support at one or more positions (e.g., thiol groups).

The linking moiety can be derived from N-hydroxysuccinimidyl (NHS) esters or N-hydroxysulfosuccinimidyl (sulfo-NHS) esters; carbodiimides; hydroxymethyl phosphines; aryl azides; pentafluorophenyl (PFP) esters, tetrafluorophenyl (TFP) esters, or derivatives thereof; imidoesters; or vinyl sulfones, such that the linking moiety is attached to a free amine of the macromolecular support. The amine can be present in naturally-occurring solvent-accessible lysine residues in or on the macromolecular support, engineered to be included in a non-naturally occurring lysine residue, or on the surface of the macromolecular support.

For example, L_{M} can be of formula:

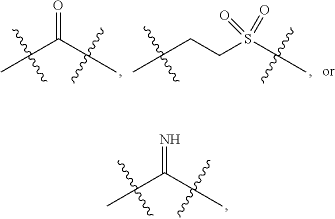

where L_{M} is bound to one or more amine groups in or on the macromolecular support, and the one or more amine groups are naturally-occurring solvent-accessible lysine residues in or on the macromolecular support, engineered to be included in a non-naturally occurring lysine residue, or on the surface of the macromolecular support.

In some embodiments, the macromolecular support can contain an aldehyde, ketone, azide, or alkyne, such that the TLR agonist can be linked via the aldehyde, ketone, azide, or alkyne. For example, L_{M} can be of formula:

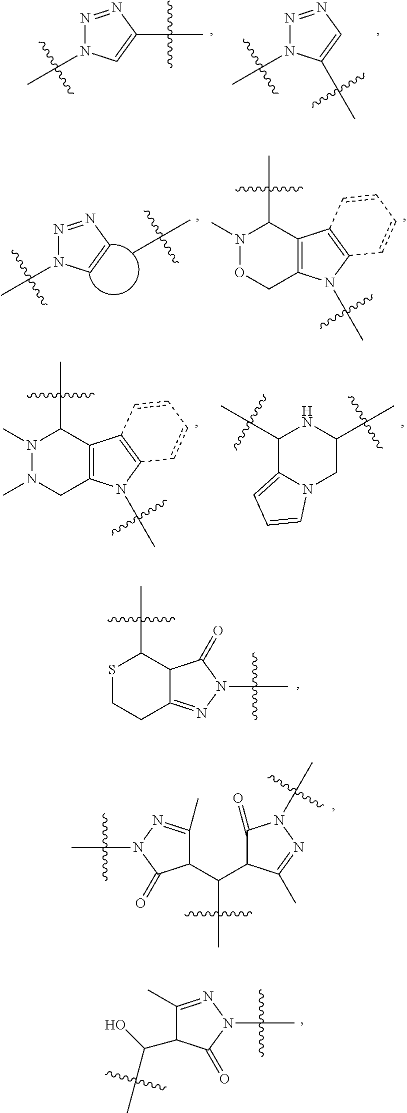

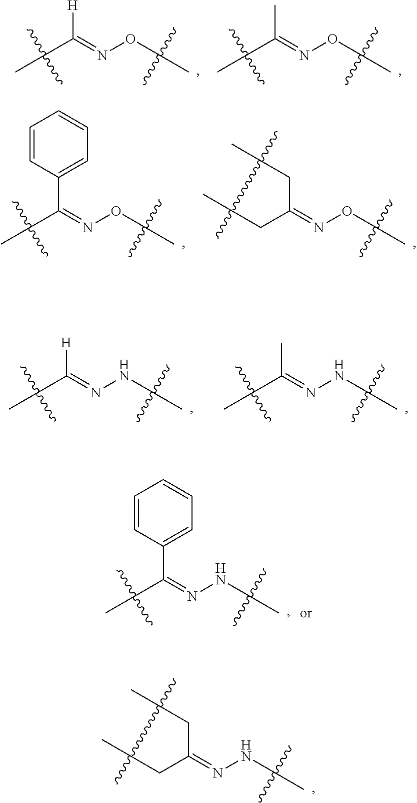

wherein the circle represents a 6 to 10-membered cyclic alkyl structure with the bond representing an attachment to one of the carbon atoms and when the wavy line (“ [symbol] ”) crosses multiple bonds, it will be understood that the L_{M} attaches to the macromolecular support at one or more positions (e.g., thiol groups).

In some embodiments, the TLR agonist is attached to a cysteine residue with the thiol eliminated to form a dehydroalanine residue of the formula:

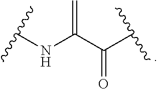 [SMILES: *NC(=C)C(*)=O]

In such instances, L_{M} can be —S—, —NH—, or a bond.

The TLR agonist can be linked to one or more naturally-occurring solvent-accessible tyrosine residues or an engineered non-naturally occurring tyrosine residue such that, for example, L_{M} can be of formula:

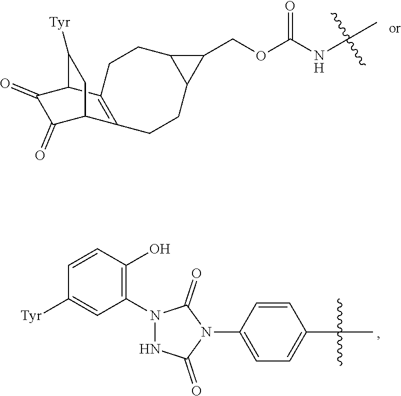

wherein Tyr is a natural or an engineered non-naturally occurring tyrosine residue in or on the macromolecular support.

In certain embodiments, L_{M} is a linking moiety of formula:

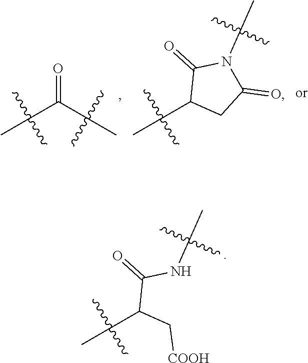

In some embodiments, the invention provides a macromolecule-supported compound of formula:

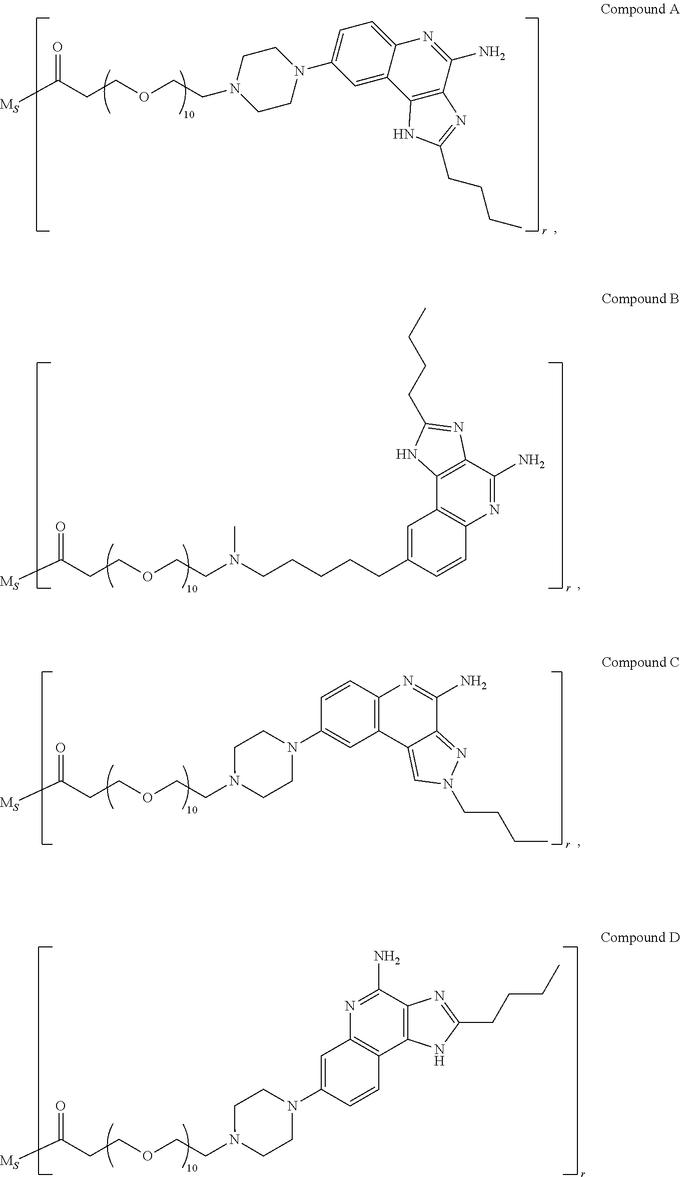

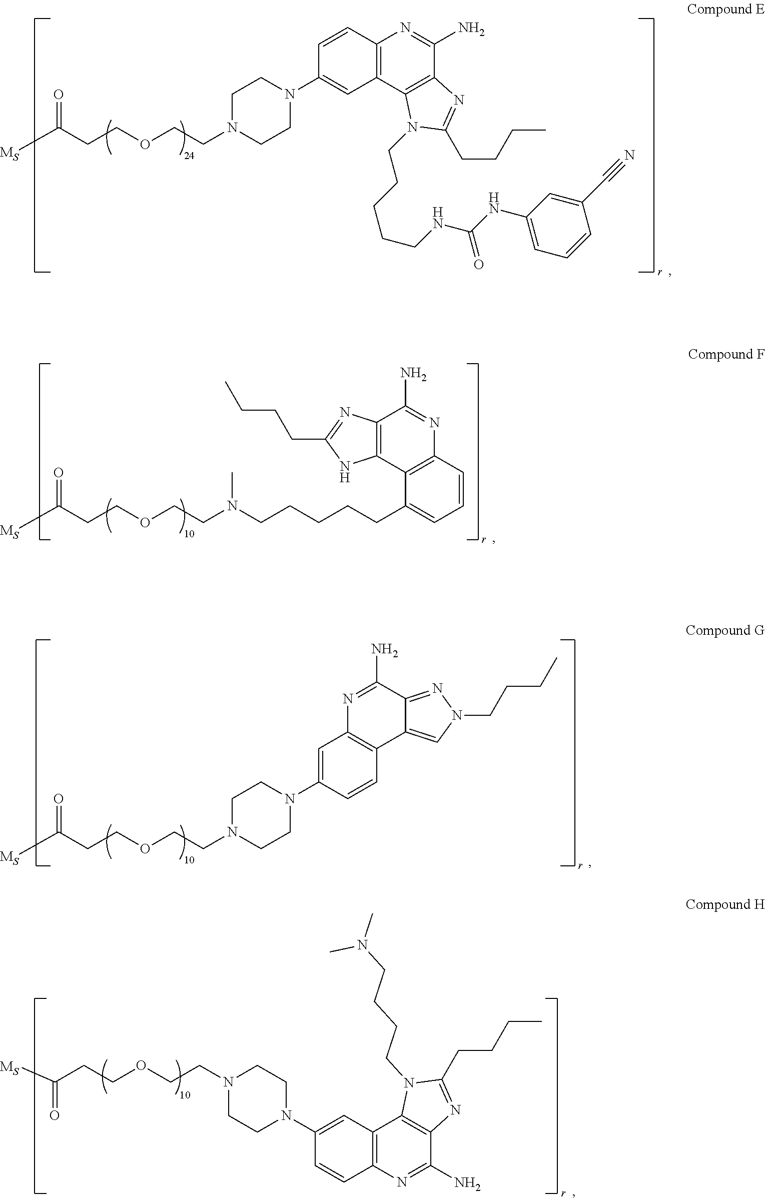

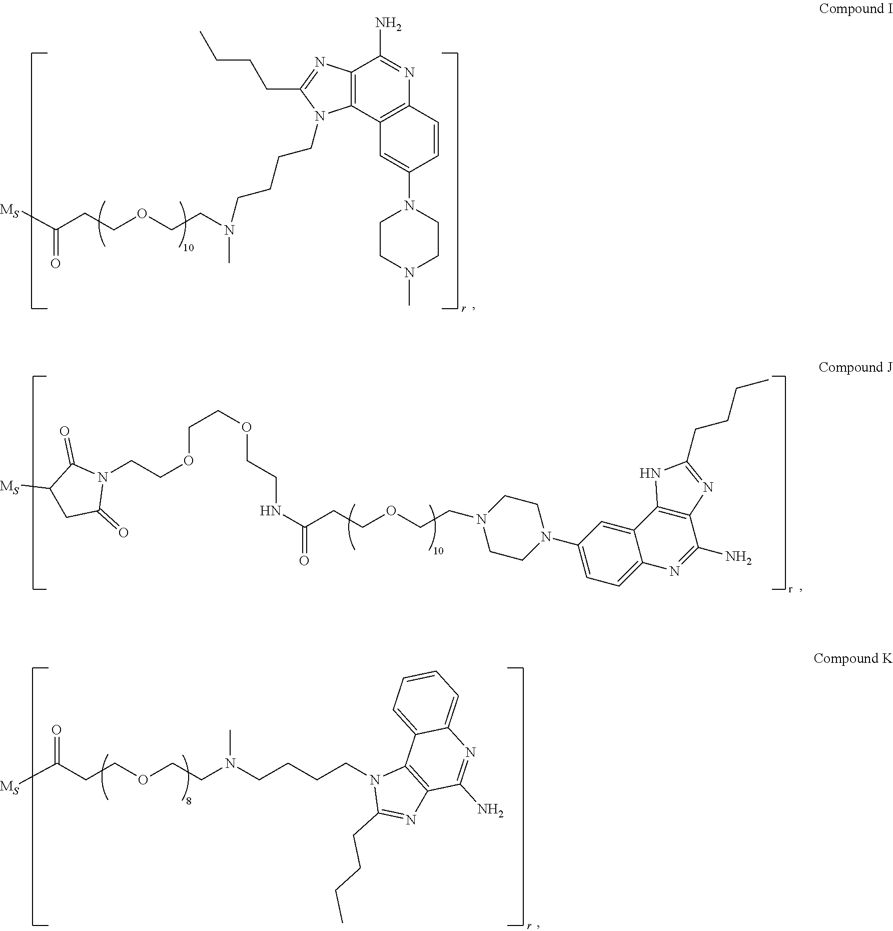

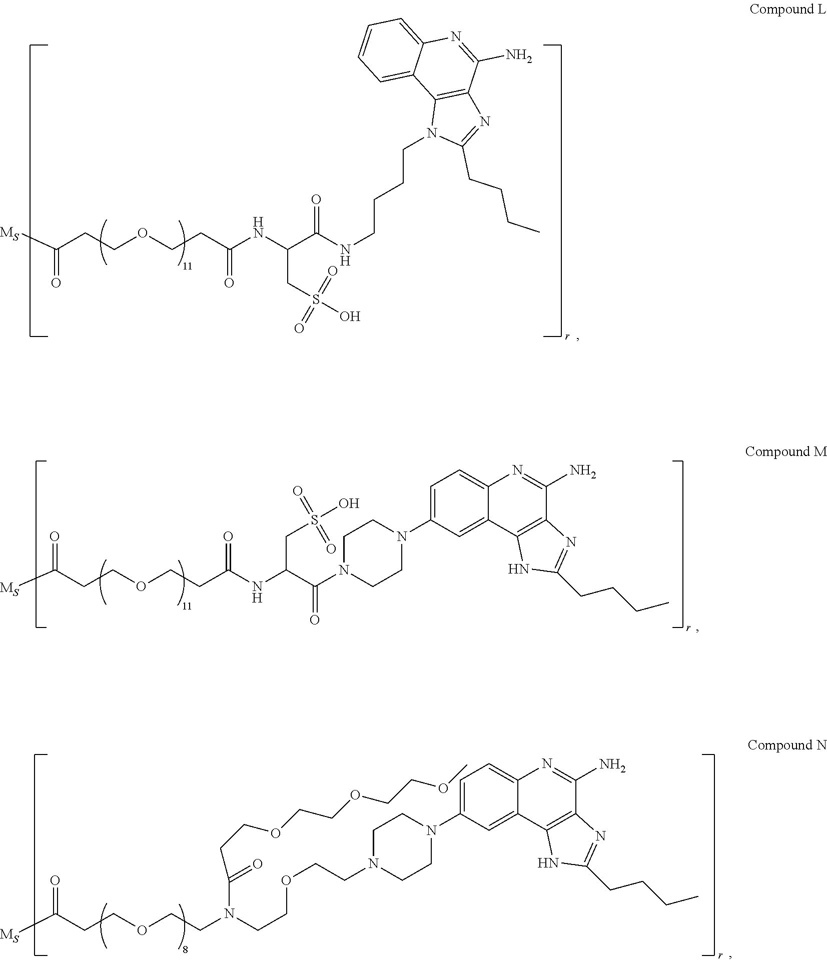

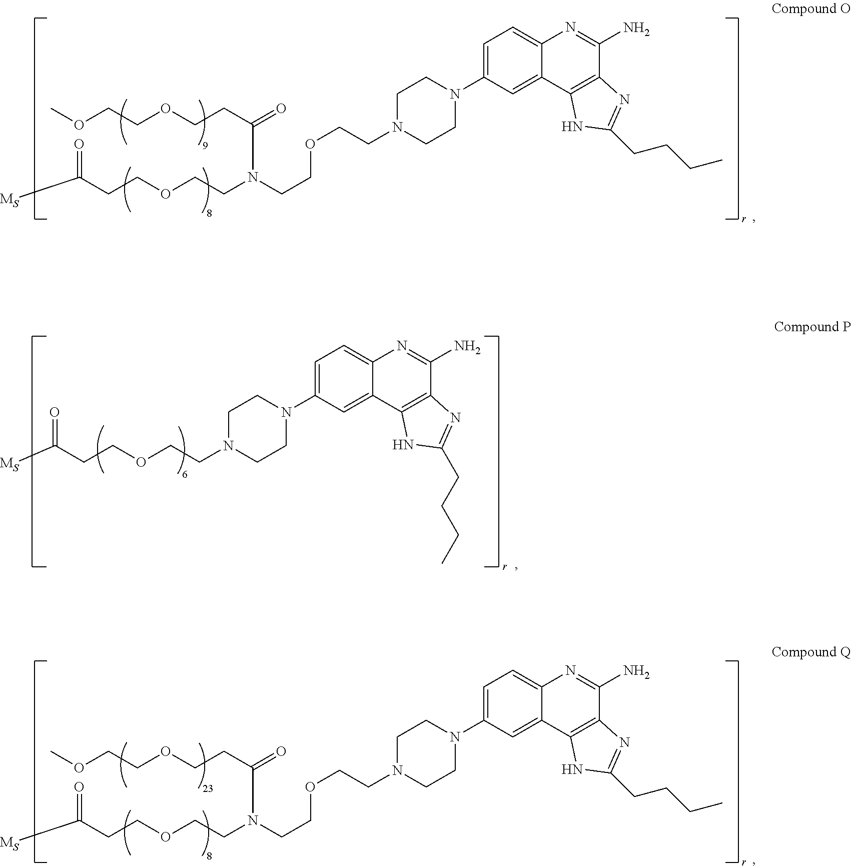

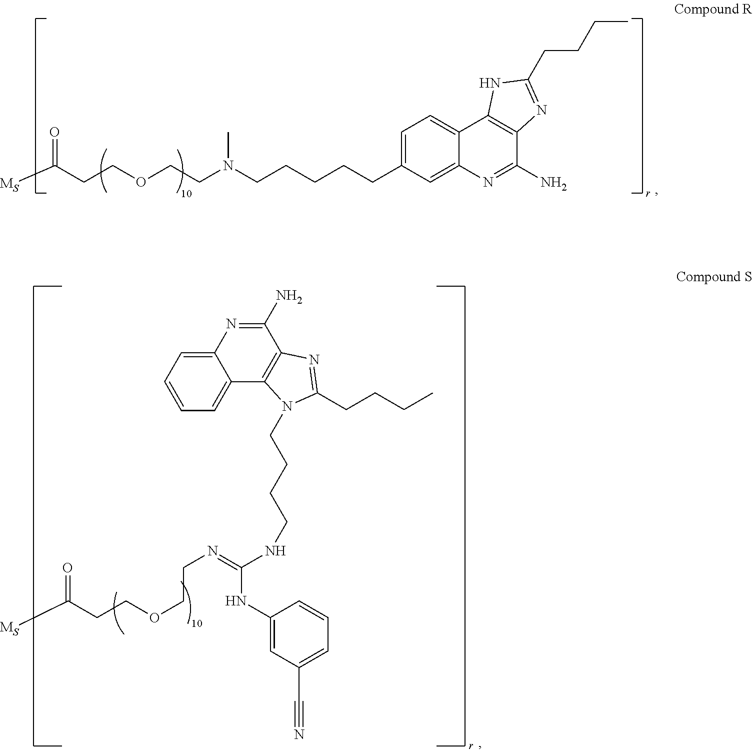

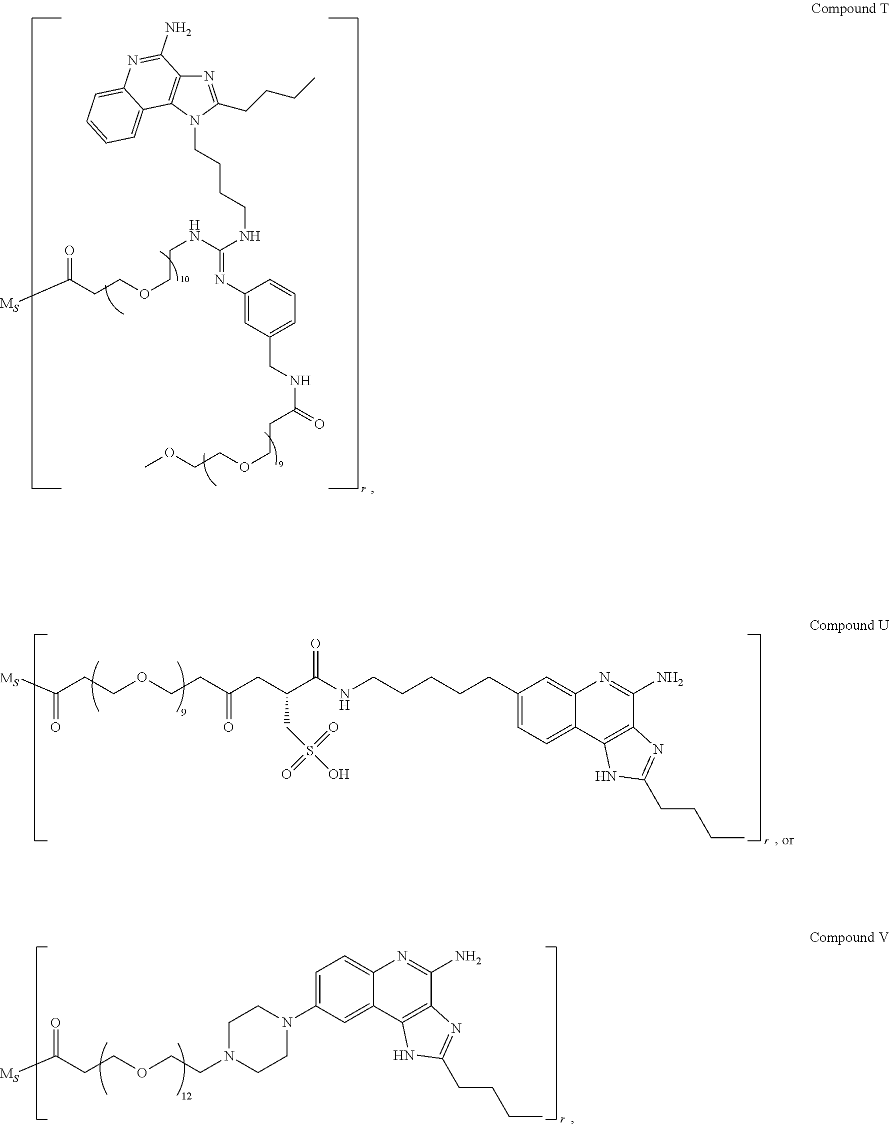

a pharmaceutically acceptable salt thereof, or a quaternary ammonium salt thereof, wherein subscript r is an integer from 1 to 50 and “M_{S}” is a macromolecular support.

In some embodiments, the invention provides a quaternary ammonium salt of a macromolecule-supported compound of formula:

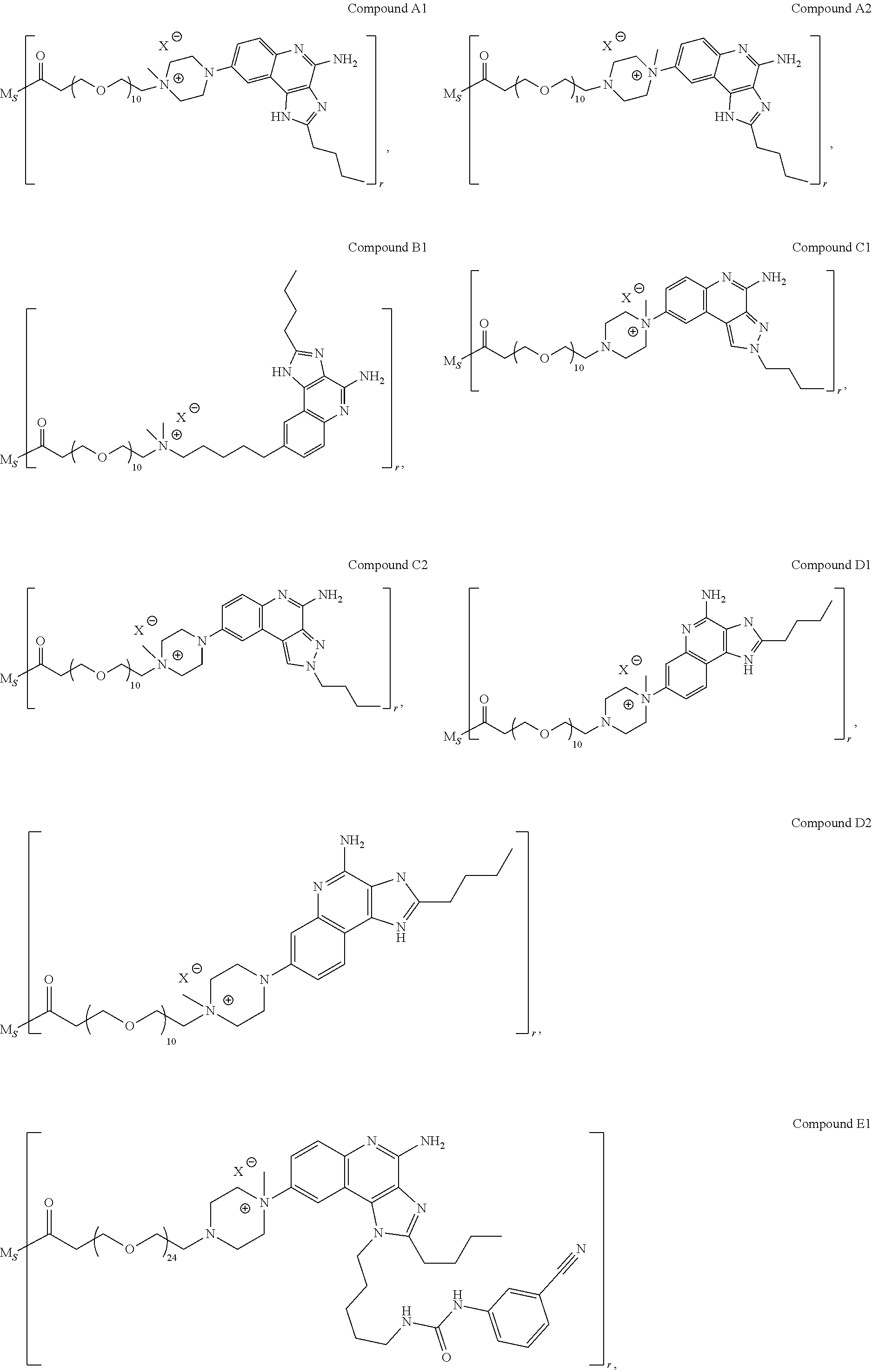

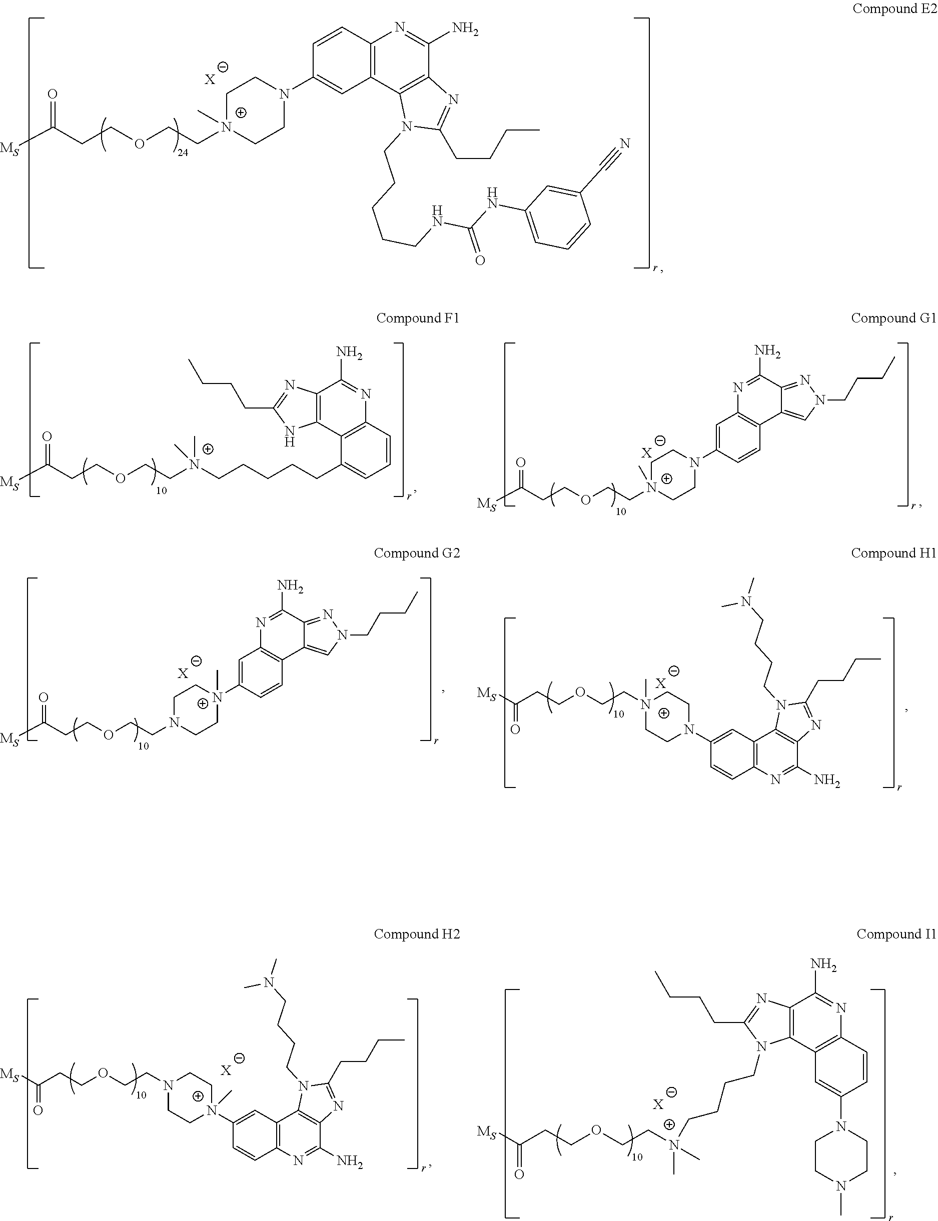

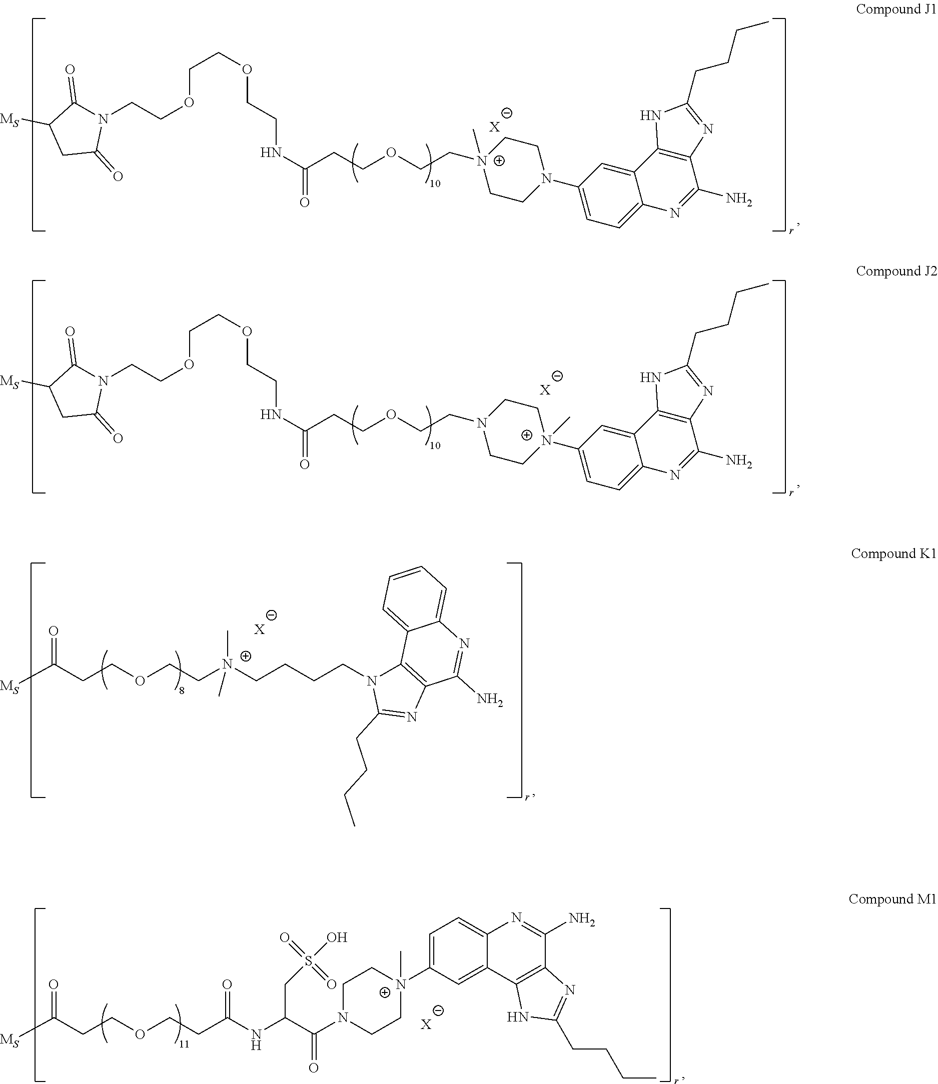

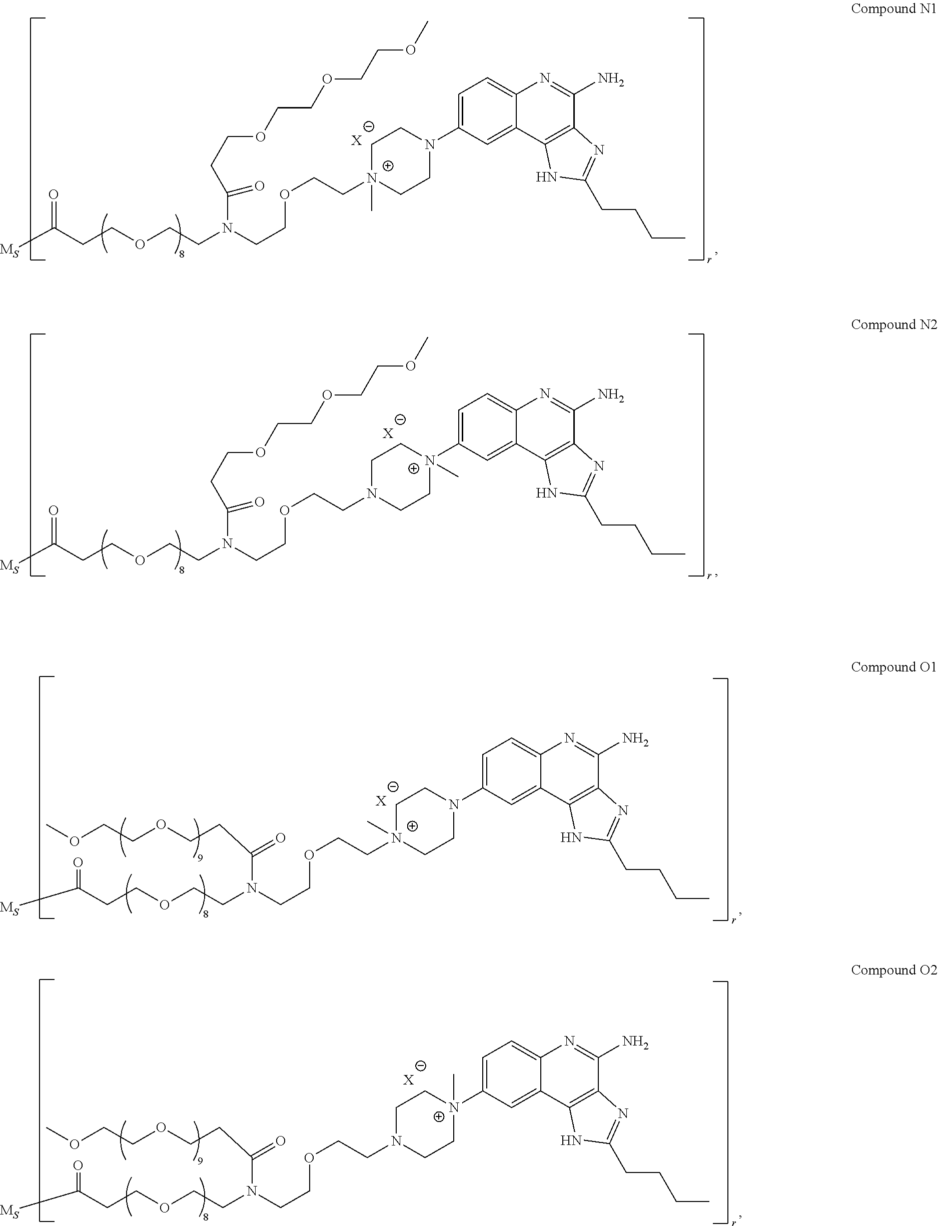

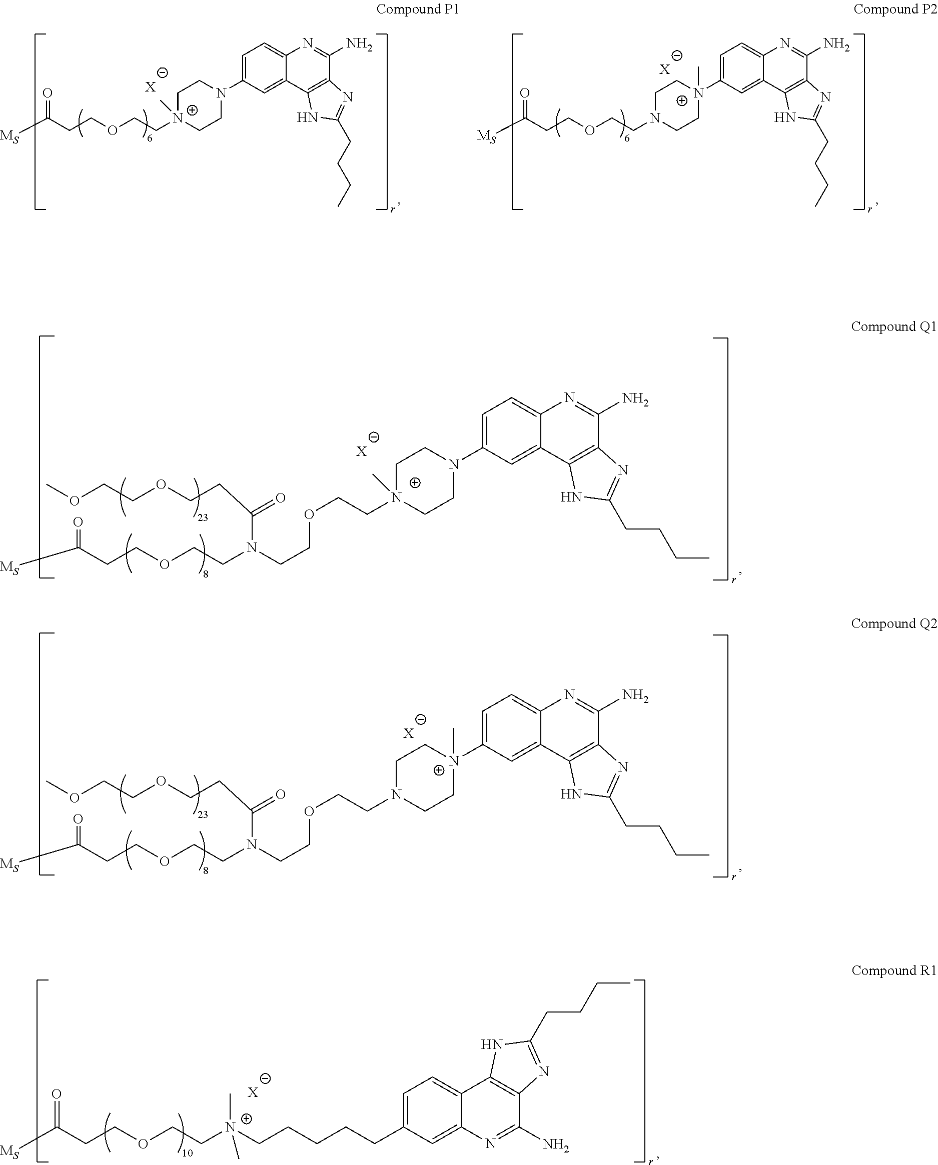

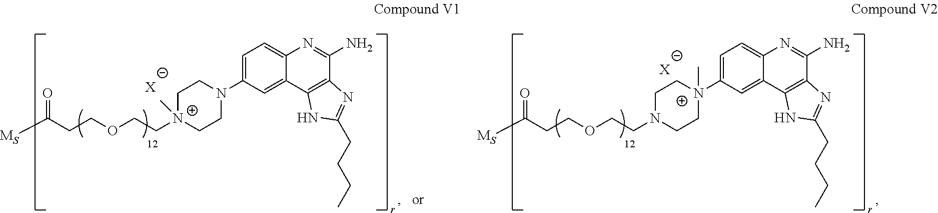

wherein counterion X⁻ is any pharmaceutically acceptable counterion (e.g., chloride, bromide, acetate, formate, nitrate, phosphate, sulfate, tosylate, etc.), subscript r is an integer from 1 to 50 and “M_{S}” is a macromolecular support.

In some embodiments, the macromolecule-supported compound is not of formula:

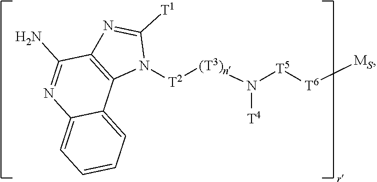

or a pharmaceutically acceptable salt thereof, wherein
“M_{S}” is a macromolecular support;
T¹ is selected from C₁₋₆ alkyl and 2- to 6-membered heteroalkyl, each of which is optionally substituted with one or more members selected from the group consisting of halo, hydroxy, amino, oxo (═O), alkylamino, amido, acyl, nitro, cyano, and alkoxy;
T² is selected from 0 and CH₂;
each T³ is independently CHT⁶, wherein T⁶ is selected from H, OH, and NH₂,
T⁵ is a linker;
T⁴ is selected from H and C₁₋₄ alkyl; or
T⁵, T⁴, and the nitrogen atom to which they are attached form a linker comprising a 5- to 8-membered heterocycle;
T⁶ is an unmodified amino acid sidechain in the macromolecular support or a modified amino acid sidechain of the macromolecular support;
subscript n¹ is an integer from 1 to 12 (i.e., 1, 2, 3, 4, 5, 6, 7, 8, 9, 10, 11, or 12); and
subscript r′ is an integer from 1 to 10 (i.e., 1, 2, 3, 4, 5, 6, 7, 8, 9, or 10).

In some embodiments, the macromolecule-supported compound is not of formula:

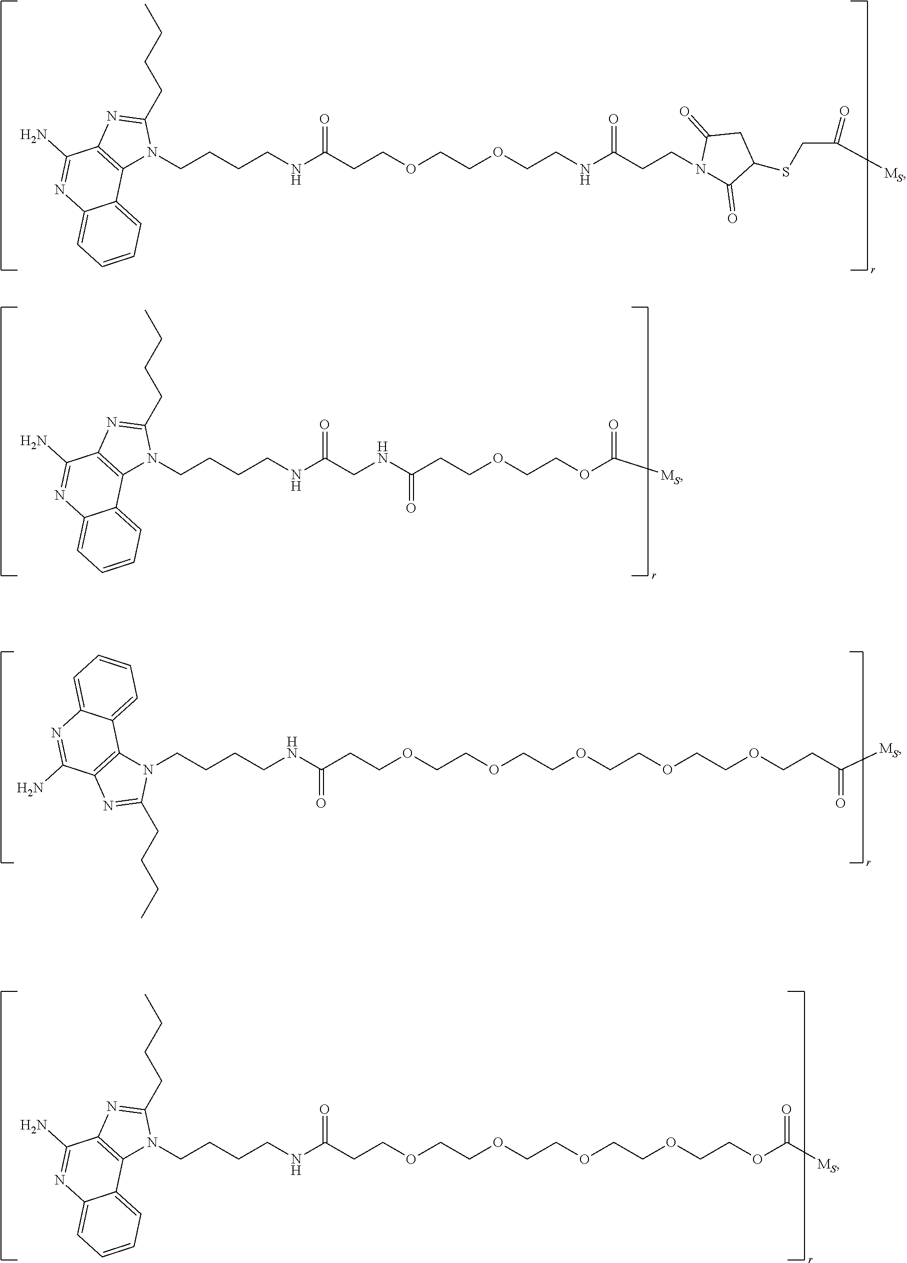

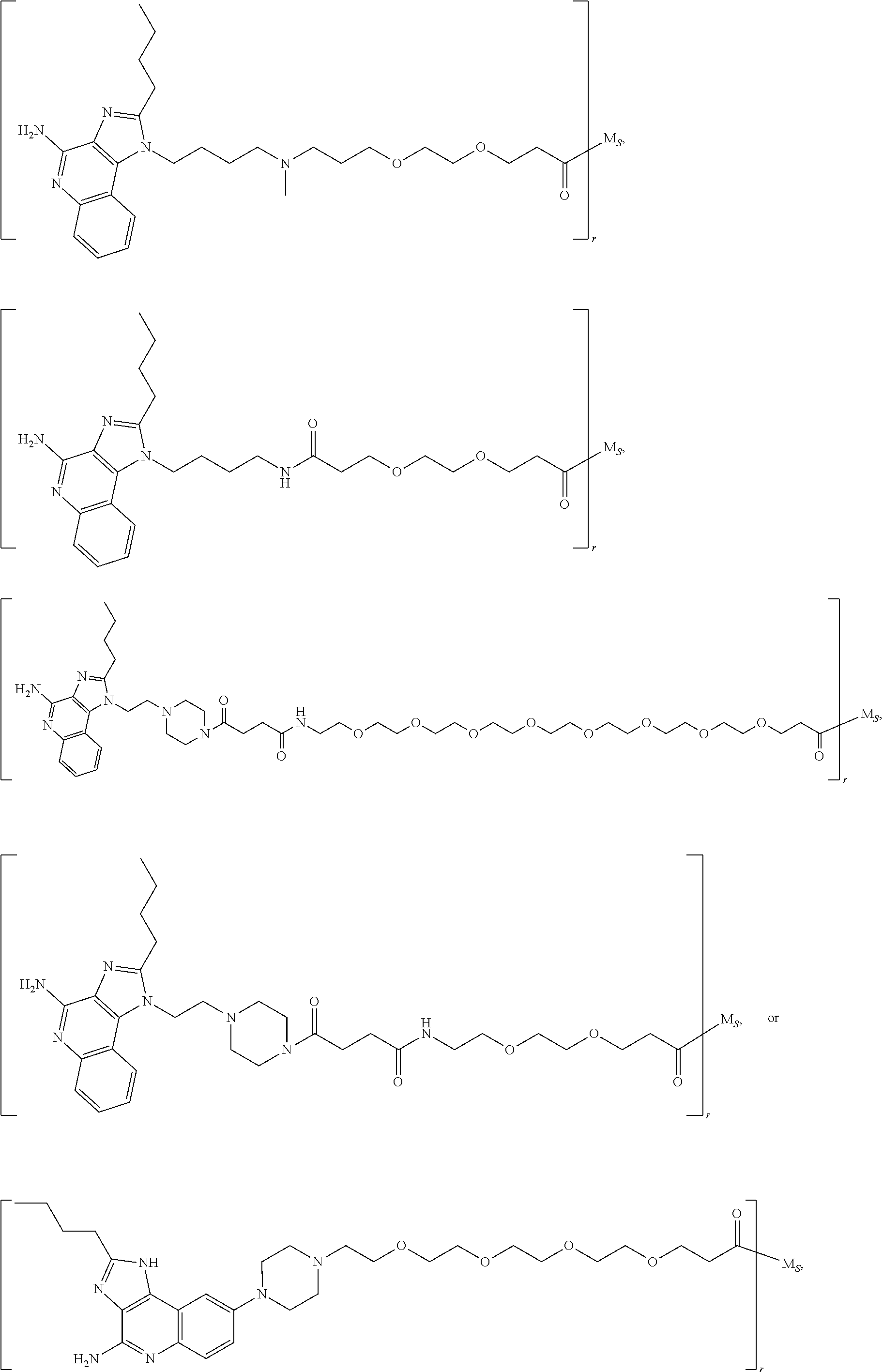

wherein subscript r is an integer from 1 to 50 and “M_{S}” is a macromolecular support.

### TLR Agonists

The macromolecule-supported compound of the invention comprises a TLR agonist. TLRs are type-I transmembrane proteins that are responsible for the initiation of innate immune responses in vertebrates. TLRs recognize a variety of pathogen-associated molecular patterns from bacteria, viruses, and fungi and act as a first line of defense against invading pathogens. TLRs elicit overlapping yet distinct biological responses due to differences in cellular expression and in the signaling pathways that they initiate. Once engaged (e.g., by a natural stimulus or a synthetic TLR agonist), TLRs initiate a signal transduction cascade leading to activation of nuclear factor-κB (NF-κB) via the adapter protein myeloid differentiation primary response gene 88 (MyD88) and recruitment of the IL-1 receptor associated kinase (IRAK). Phosphorylation of IRAK then leads to recruitment of TNF-receptor associated factor 6 (TRAF6), which results in the phosphorylation of the NF-κB inhibitor I-κB. As a result, NF-κB enters the cell nucleus and initiates transcription of genes whose promoters contain NF-κB binding sites, such as cytokines. Additional modes of regulation for TLR signaling include TIR-domain containing adapter-inducing interferon-β (TRIF)-dependent induction of TNF-receptor associated factor 6 (TRAF6) and activation of MyD88 independent pathways via TRIF and TRAF3, leading to the phosphorylation of interferon response factor three (IRF3). Similarly, the MyD88 dependent pathway also activates several IRF family members, including IRF5 and IRF7 whereas the TRIF dependent pathway also activates the NF-κB pathway.

Typically, the TLR agonist moiety described herein is a TLR7 and/or TLR8 agonist. TLR7 and TLR8 are both expressed in monocytes and dendritic cells. In humans, TLR7 is also expressed in plasmacytoid dendritic cells (pDCs) and B cells. TLR8 is expressed mostly in cells of myeloid origin, i.e., monocytes, granulocytes, and myeloid dendritic cells. TLR7 and TLR8 are capable of detecting the presence of “foreign” single-stranded RNA within a cell, as a means to respond to viral invasion. Treatment of TLR8-expressing cells, with TLR8 agonists can result in production of high levels of IL-12, IFN-γ, IL-1, TNF-α, IL-6, and other inflammatory cytokines. Similarly, stimulation of TLR7-expressing cells, such as pDCs, with TLR7 agonists can result in production of high levels of IFN-α and other inflammatory cytokines. TLR7/TLR8 engagement and resulting cytokine production can activate dendritic cells and other antigen-presenting cells, driving diverse innate and acquired immune response mechanisms leading to tumor destruction.

### Macromolecular Support

The macromolecule-supported compound of the invention comprises a macromolecular support. As a singular entity, the macromolecular support can be biologically active or biologically inactive relative to the TLR agonist described herein. However, when used in combination with the TLR agonist, the biological activity of the TLR agonist is enhanced, for example, by providing a targeting effect, by providing beneficial off-target effects (i.e., biological activity other than TLR activity), improved pharmacokinetic properties (e.g., half-life extension), enhanced biological delivery (e.g., tumor penetration), or by providing additional biological stimulation, differentiation, up-regulation, and/or down-regulation. In certain embodiments, the biological effect of the macromolecular support and the TLR agonist is synergistic, i.e., greater than the sum of the biological activity of each of the macromolecular support and TLR agonist as a singular entity.

In some embodiments, the macromolecular support is a resin, bead, probe, tag, well, plate, or any other surface that can be used for therapeutics, diagnostics, or chemical assays. The resin, bead, probe, tag, well, plate, or any other surface can be made of any suitable material so long as the material can be surface modified. For example, the resin, bead, probe, tag, well, plate, or any other surface can polymer-based such as, for example, polyacrylates, polyacrylamides, polystyrenes, polyethylenes, polypropylenes, polyethylene glycols, or polypropylene glycols.

In some embodiments, the macromolecular support is a chemical structure (e.g., a biological structure or an inorganic framework) that can be used for therapeutics, diagnostics, or chemical assays. The macromolecular support can have any suitable structure and size. The macromolecular support can be an organic or inorganic structure having a molecular weight of at least about 200 Da (e.g., at least about 500 Da, at least about 1,000 Da, at least about 2,000 Da, at least about 5,000 Da, or at least about 10,000 Da). For example, the macromolecular support can be a biopolymer (e.g., a glycopolymer, a cellulosic polymer, etc.), a nanoparticle (e.g., a carbon nanotube, a quantum dot, a metal nanoparticle (e.g., silver, gold, titanium dioxide, silicon dioxide, zirconium dioxide, aluminum oxide, or ytterbium trifluoride), etc.), a lipid (e.g., lipid vesicles, micelles, liposomes, etc.), a carbohydrate (e.g., sugar, starch, cellulose, glycogen, etc.), a peptide (e.g., a polypeptide, a protein, a peptide mimetic, a glycopeptide, etc.), an alternative protein scaffold, an antibody construct (e.g., antibody, an antibody-derivative (including Fc fusions, Fab fragments and scFvs), etc.), a nucleotide (e.g., RNA, DNA, antisense, siRNA, an aptamer, etc.), or any combination thereof. In some embodiments, the macromolecular support is a peptide, a nucleotide, a sugar, a lipid, or an antibody. In certain embodiments, the macromolecular support is an immune checkpoint inhibitor.

### Macromolecule-Supported Compound Composition

The invention provides a composition, e.g., a pharmaceutically acceptable composition or formulation, comprising a plurality of macromolecule-supported compounds as described herein and optionally a carrier therefor, e.g., a pharmaceutically acceptable carrier. The macromolecule-supported compounds can be the same or different in the composition, i.e., the composition can comprise macromolecule-supported compounds that have the same number of TLR agonists linked to the same chemical entity of the macromolecule-supported compound, macromolecule-supported compounds that have the same number of TLR agonists linked to different chemical entities of the macromolecule-supported compound, that have different numbers of TLR agonists linked to the same chemical entity of the macromolecule-supported compound, and/or that have different numbers of TLR agonists linked to different chemical entities of the macromolecule-supported compound.

The composition can have any suitable average TLR agonist to macromolecular support ratio (e.g., about 0.1 to about 50, about 1 to about 10, about 1 to about 6, or about 1 to about 4). For example, a composition of macromolecule-supported compounds of the invention can have an average TLR agonist to macromolecular support ratio of about 0.4, 0.6, 0.8, 1, 1.2, 1.4, 1.6, 1.8, 2, 2.2, 2.4, 2.6, 2.8, 3, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7, 7.2, 7.4, 7.6, 7.8, 8, 8.2, 8.4, 8.6, 8.8, 9, 9.2, 9.4, 9.6, 9.8, or 10, or within a range bounded by any two of the aforementioned values. A skilled artisan will recognize that the number of TLR agonist conjugated to the macromolecular support may vary from macromolecule-supported compound to macromolecule-supported compound in a composition comprising multiple macromolecule-supported compounds of the invention, and, thus, the TLR agonist to macromolecule-supported ratio can be measured as an average. The TLR agonist to macromolecule-supported ratio can be assessed by any suitable means, many of which are known in the art.

In some embodiments, the composition further comprises one or more pharmaceutically acceptable excipients. For example, the macromolecule-supported compounds of the invention can be formulated for parenteral administration, such as IV administration or administration into a body cavity or lumen of an organ. Alternatively, the macromolecule-supported compounds can be injected intra-tumorally. Compositions for injection will commonly comprise a solution of the macromolecule-supported compound dissolved in a pharmaceutically acceptable carrier. Among the acceptable vehicles and solvents that can be employed are water and an isotonic solution of one or more salts such as sodium chloride, e.g., Ringer's solution. In addition, sterile fixed oils can conventionally be employed as a solvent or suspending medium. For this purpose, any bland fixed oil can be employed, including synthetic monoglycerides or diglycerides. In addition, fatty acids such as oleic acid can likewise be used in the preparation of injectables. These compositions desirably are sterile and generally free of undesirable matter. These compositions can be sterilized by conventional, well known sterilization techniques. The compositions can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like.

The composition can contain any suitable concentration of the macromolecule-supported compound. The concentration of the macromolecule-supported compound in the composition can vary widely, and will be selected primarily based on fluid volumes, viscosities, body weight, and the like, in accordance with the particular mode of administration selected and the patient's needs. In certain embodiments, the concentration of a macromolecule-supported compound in a solution formulation for injection will range from about 0.1% (w/w) to about 10% (w/w).

### Methods of Using the Macromolecule-Supported Compound

The invention provides a method of recognizing TLR (e.g., TLR7 and/or TLR8) for use in therapeutics, diagnostics, or chemical assays. Without wishing to be bound by any particular theory, TLR has a high affinity for the adjuvant/linker combinations described herein, such that the macromolecule-supported compounds described herein are useful in assessing the presence and/or abundance of TLR. In certain embodiments, the macromolecule-supported compound is used as a chemical assay for TLR engagement and/or activity. In such embodiments, the macromolecular support can be a resin, bead, probe, tag, well, or plate. In certain embodiments, the macromolecule-supported compound is used as a therapeutic or diagnostic for diseases associated with TLR. In such embodiments, the macromolecular support is typically a chemical structure (e.g., a biological structure or an inorganic framework) having a molecular weight of at least about 200 Da (e.g., at least about 500 Da, at least about 1,000 Da, at least about 2,000 Da, at least about 5,000 Da, or at least about 10,000 Da).

The invention also provides a method for treating cancer. The method comprises administering a therapeutically effective amount of a macromolecule-supported compound (e.g., as a composition as described above) to a subject in need thereof. For example, the method can include administering the macromolecule-supported compound to provide a dose of from about 100 ng/kg to about 50 mg/kg to the subject. The macromolecule-supported compound dose can range from about 5 mg/kg to about 50 mg/kg, from about 10 μg/kg to about 5 mg/kg, or from about 100 μg/kg to about 1 mg/kg. The macromolecule-supported compound dose can be about 100, 200, 300, 400, or 500 μg/kg. The macromolecule-supported compound dose can be about 1, 2, 3, 4, 5, 6, 7, 8, 9, or 10 mg/kg. The macromolecule-supported compound dose can also be outside of these ranges, depending on the particular compound as well as the type and severity of the cancer being treated. Frequency of administration can range from a single dose to multiple doses per week, or more frequently. In some embodiments, the macromolecule-supported compound is administered from about once per month to about five times per week. In some embodiments, the macromolecule-supported compound is administered once per week.

In a further aspect, the invention provides a method for curing cancer. The method comprises administering a therapeutically effective amount of a macromolecule-supported compound (e.g., as a composition as described above) to a subject. For example, the methods can include administering the macromolecule-supported compound to provide a dose of from about 100 ng/kg to about 50 mg/kg to the subject. The macromolecule-supported compound dose can range from about 5 mg/kg to about 50 mg/kg, from about 10 μg/kg to about 5 mg/kg, or from about 100 μg/kg to about 1 mg/kg. The macromolecule-supported compound dose can be about 100, 200, 300, 400, or 500 μg/kg. The macromolecule-supported compound dose can be about 1, 2, 3, 4, 5, 6, 7, 8, 9, or 10 mg/kg. The macromolecule-supported compound dose can also be outside of these ranges, depending on the particular conjugate as well as the type and severity of the cancer being cured. Frequency of administration can range from a single dose to multiple doses per week, or more frequently. In some embodiments, the macromolecule-supported compound is administered from about once per month to about five times per week. In some embodiments, the macromolecule-supported compound is administered once per week.

In another aspect, the invention provides a method for preventing cancer. The method comprises administering a therapeutically effective amount of a macromolecule-supported compound (e.g., as a composition as described above) to a subject. In certain embodiments, the subject is susceptible to a certain cancer to be prevented. For example, the methods can include administering the macromolecule-supported compound to provide a dose of from about 100 ng/kg to about 50 mg/kg to the subject. The macromolecule-supported compound dose can range from about 5 mg/kg to about 50 mg/kg, from about 10 μg/kg to about 5 mg/kg, or from about 100 μg/kg to about 1 mg/kg. The macromolecule-supported compound dose can be about 100, 200, 300, 400, or 500 μg/kg. The macromolecule-supported compound dose can be about 1, 2, 3, 4, 5, 6, 7, 8, 9, or 10 mg/kg. The macromolecule-supported compound dose can also be outside of these ranges, depending on the particular conjugate as well as the type and severity of the cancer being treated. Frequency of administration can range from a single dose to multiple doses per week, or more frequently. In some embodiments, the macromolecule-supported compound is administered from about once per month to about five times per week. In some embodiments, the macromolecule-supported compound is administered once per week.

Some embodiments of the invention provide methods for treating cancer as described above, wherein the cancer is a head and neck cancer. Head and neck cancer (as well as head and neck squamous cell carcinoma) refers to a variety of cancers characterized by squamous cell carcinomas of the oral cavity, pharynx and larynx, salivary glands, paranasal sinuses, and nasal cavity, as well as the lymph nodes of the upper part of the neck. Head and neck cancers account for approximately 3 to 5 percent of all cancers in the United States. These cancers are more common in men and in people over age 50. Tobacco (including smokeless tobacco) and alcohol use are the most important risk factors for head and neck cancers, particularly those of the oral cavity, oropharynx, hypopharynx and larynx. Eighty-five percent of head and neck cancers are linked to tobacco use.

In the methods of the invention, the macromolecule-supported compounds can be used to target a number of malignant cells. For example, the macromolecule-supported compounds can be used to target squamous epithelial cells of the lip, oral cavity, pharynx, larynx, nasal cavity, or paranasal sinuses. The macromolecule-supported compounds can be used to target mucoepidermoid carcinoma cells, adenoid cystic carcinoma cells, adenocarcinoma cells, small-cell undifferentiated cancer cells, esthesioneuroblastoma cells, Hodgkin lymphoma cells, and Non-Hodgkin lymphoma cells.

Some embodiments of the invention provide methods for treating cancer as described above, wherein the cancer is breast cancer. Breast cancer can originate from different areas in the breast, and a number of different types of breast cancer have been characterized. For example, the macromolecule-supported compounds of the invention can be used for treating ductal carcinoma in situ; invasive ductal carcinoma (e.g., tubular carcinoma; medullary carcinoma; mucinous carcinoma; papillary carcinoma; or cribriform carcinoma of the breast); lobular carcinoma in situ; invasive lobular carcinoma; inflammatory breast cancer; and other forms of breast cancer.

In some embodiments, the cancer is susceptible to a pro-inflammatory response induced by TLR7 and/or TLR8.

### Examples of Non-Limiting Aspects of the Disclosure

Aspects, including embodiments, of the invention described herein may be beneficial alone or in combination, with one or more other aspects or embodiments. Without limiting the foregoing description, certain non-limiting aspects of the disclosure numbered 1-22 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A macromolecule-supported compound of formula (I) or formula (II):

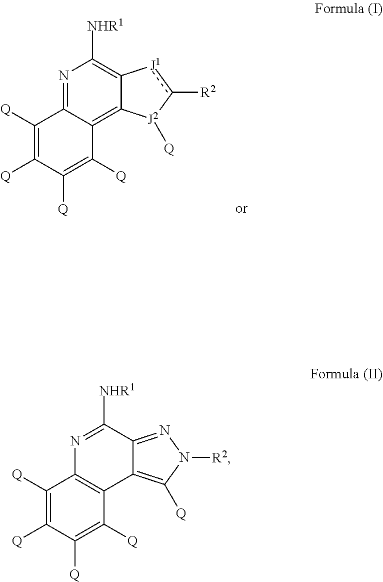

a pharmaceutically acceptable salt thereof, or a quaternary ammonium salt thereof,

wherein

R¹ and R² independently are hydrogen or of formula:

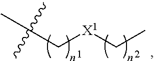

J¹ is CH or N,

J² is CHQ, NQ, O, or S,

each Q independently is Y or Z, wherein exactly one Q is Y,

Y is of formula:

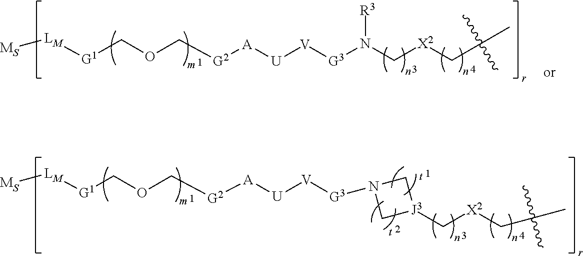

each Z independently is hydrogen or of formula:

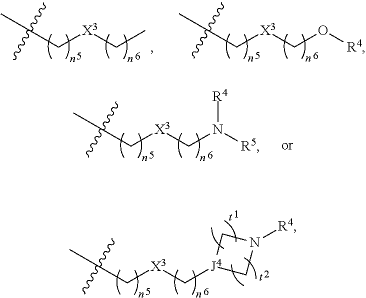

A is optionally present and is NR⁶ or of formula:

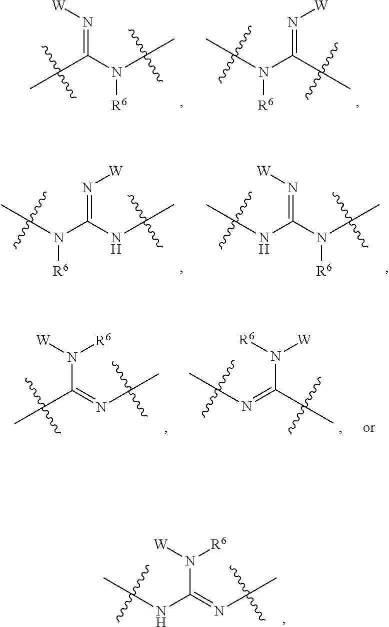

U is optionally present and is CH₂, C(O), CH₂C(O), or C(O)CH₂,

R⁶ and W independently are hydrogen, Ar¹, or of formula:

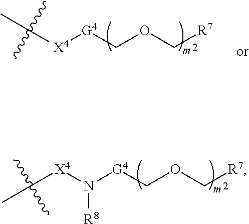

V is optionally present and is of formula:

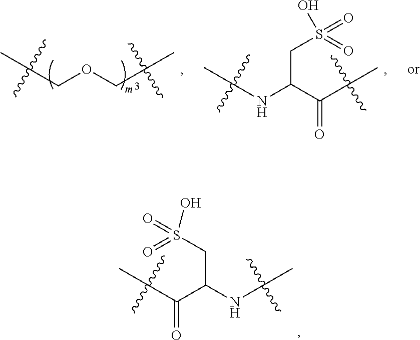

J³ and J⁴ independently are CH or N,

m¹, m², and m³ independently are an integer from 0 to 25, except that at least one of m¹, m², and m³ is a non-zero integer,

n¹, n², n³, n⁴, n⁵, and n⁶ independently are an integer from 0 to 10,

t¹ and t² independently are an integer from 1 to 3,

G¹, G², G³, and G⁴ independently are CH₂, C(O), CH₂C(O), C(O)CH₂, or a bond,

X¹, X², X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

R⁴ is hydrogen, C₁-C₄ alkyl,

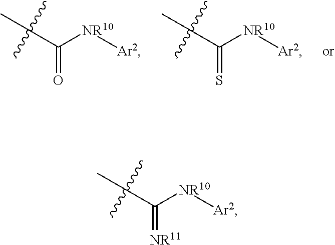

R³, R⁵, R⁷, R⁸, R⁹, R¹⁰, and R¹¹ independently are hydrogen or C₁-C₄ alkyl,

Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,

L_{M} is a linking moiety,

r is an integer from 1 to 50,

“M_{S}” is a macromolecular support, and

each wavy line (“ [symbol] ”) represents a point of attachment.

2. The macromolecule-supported compound of aspect 1, wherein subscript r is an integer from 1 to 25.

3. The macromolecule-supported compound of aspect 2, wherein subscript r is an integer from 1 to 6.

4. The macromolecule-supported compound of any one of aspects 1-3, wherein the macromolecular support is a peptide.

5. The macromolecule-supported compound of any one of aspects 1-3, wherein the macromolecular support is a nucleotide.

6. The macromolecule-supported compound of any one of aspects 1-3, wherein the macromolecular support is carbohydrate.

7. The macromolecule-supported compound of any one of aspects 1-3, wherein the macromolecular support is lipid.

8. The macromolecule-supported compound of any one of aspects 1-3, wherein the macromolecular support is an antibody construct.

9. The macromolecule-supported compound of any one of aspects 1-3, wherein the macromolecular support is a biopolymer.

10. The macromolecule-supported compound of any one of aspects 1-3, wherein the macromolecular support is a nanoparticle.

11. The macromolecule-supported compound of any one of aspects 1-3, wherein the macromolecular support is an immune checkpoint inhibitor.

12. The macromolecule-supported compound of any one of aspects 1-11, wherein the macromolecule-supported compound is of formula:

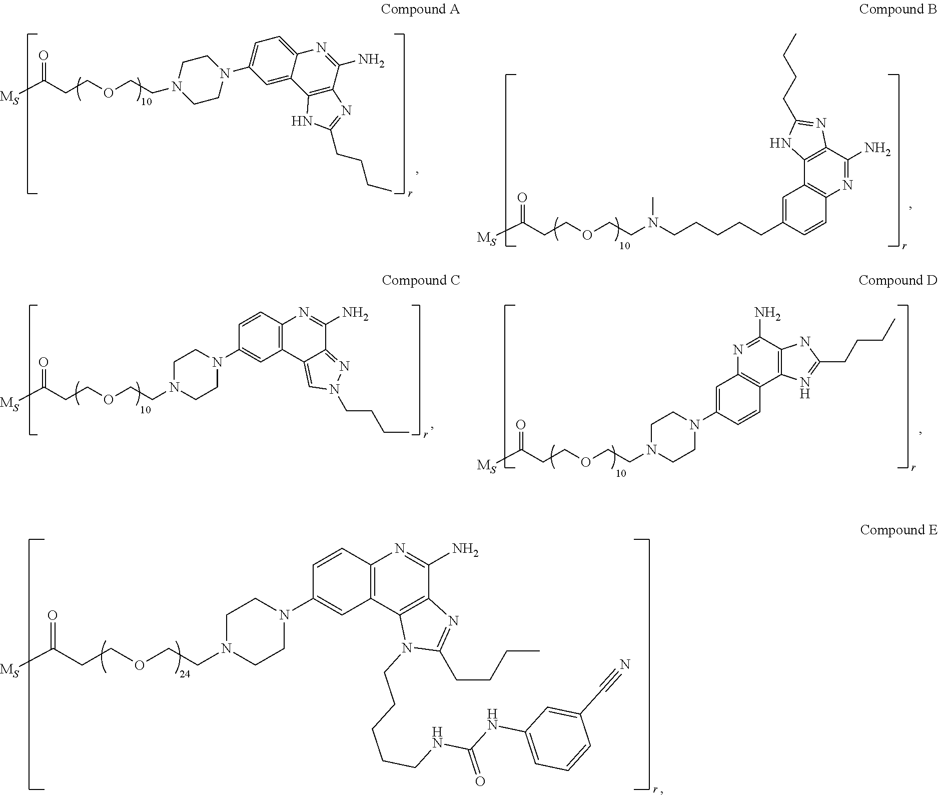

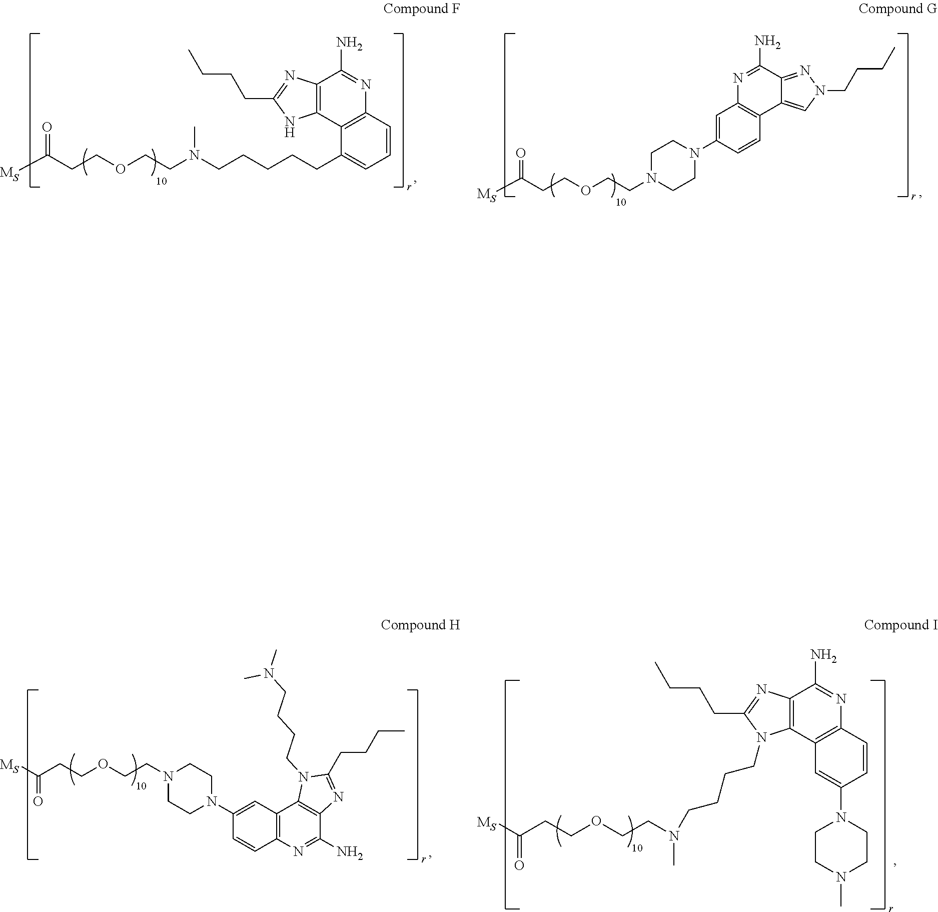

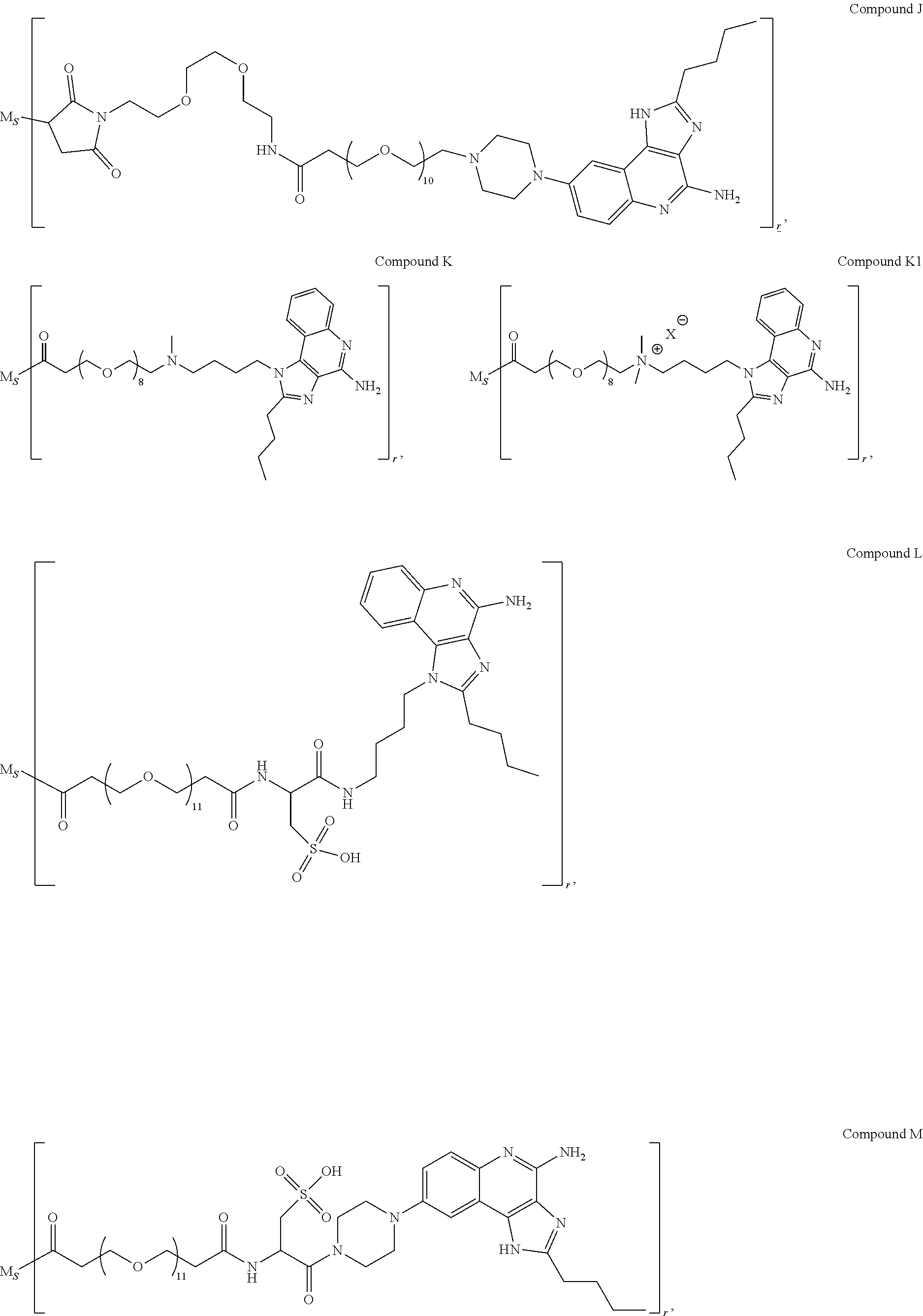

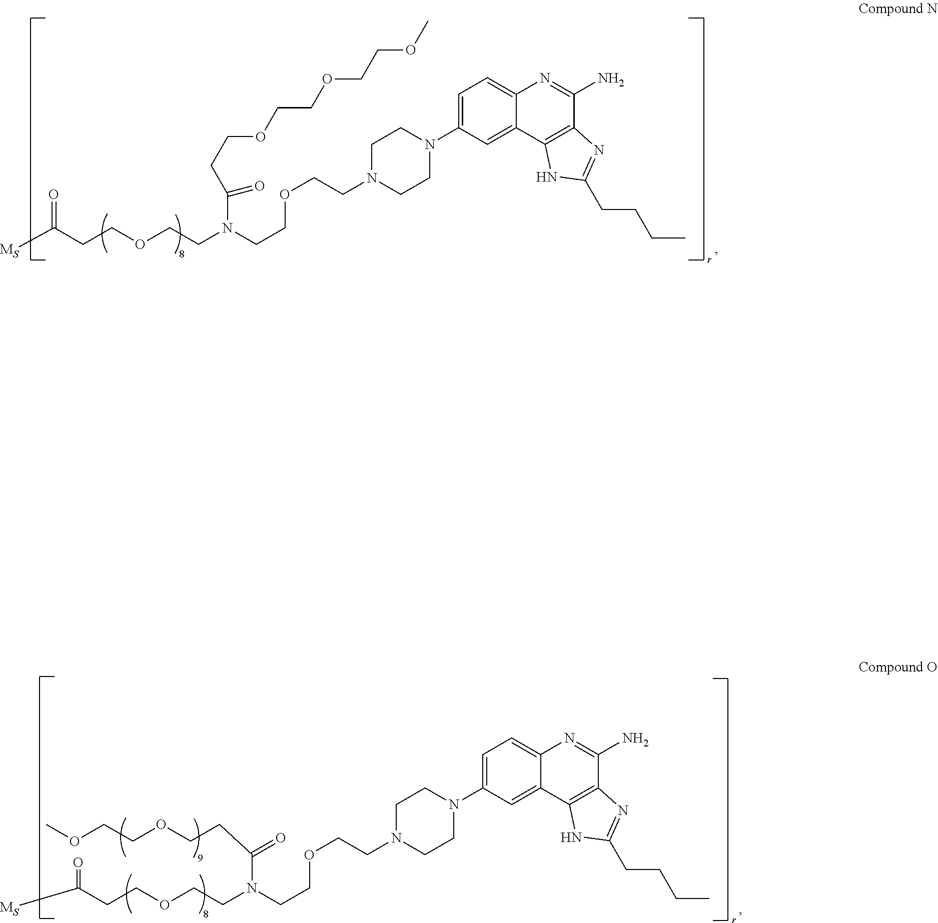

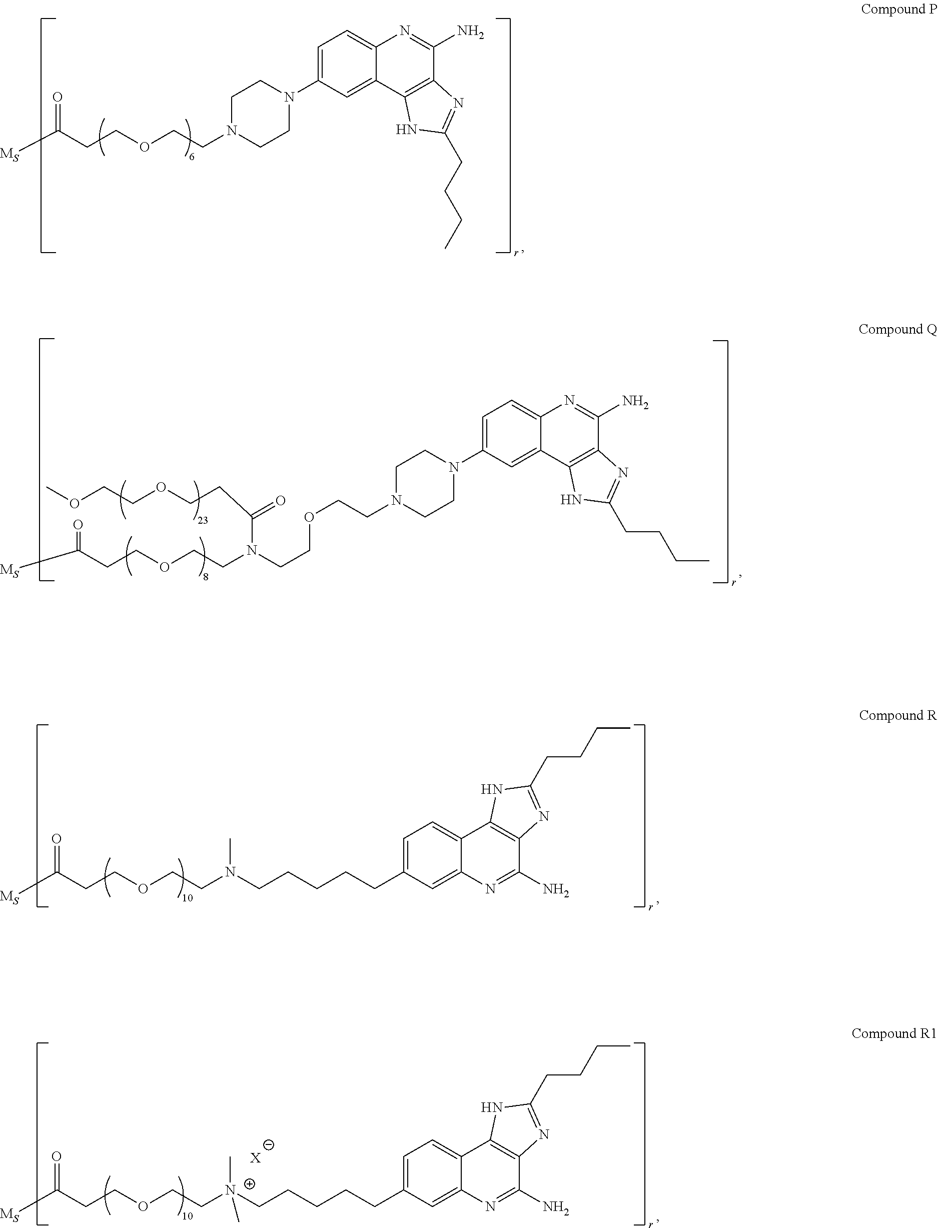

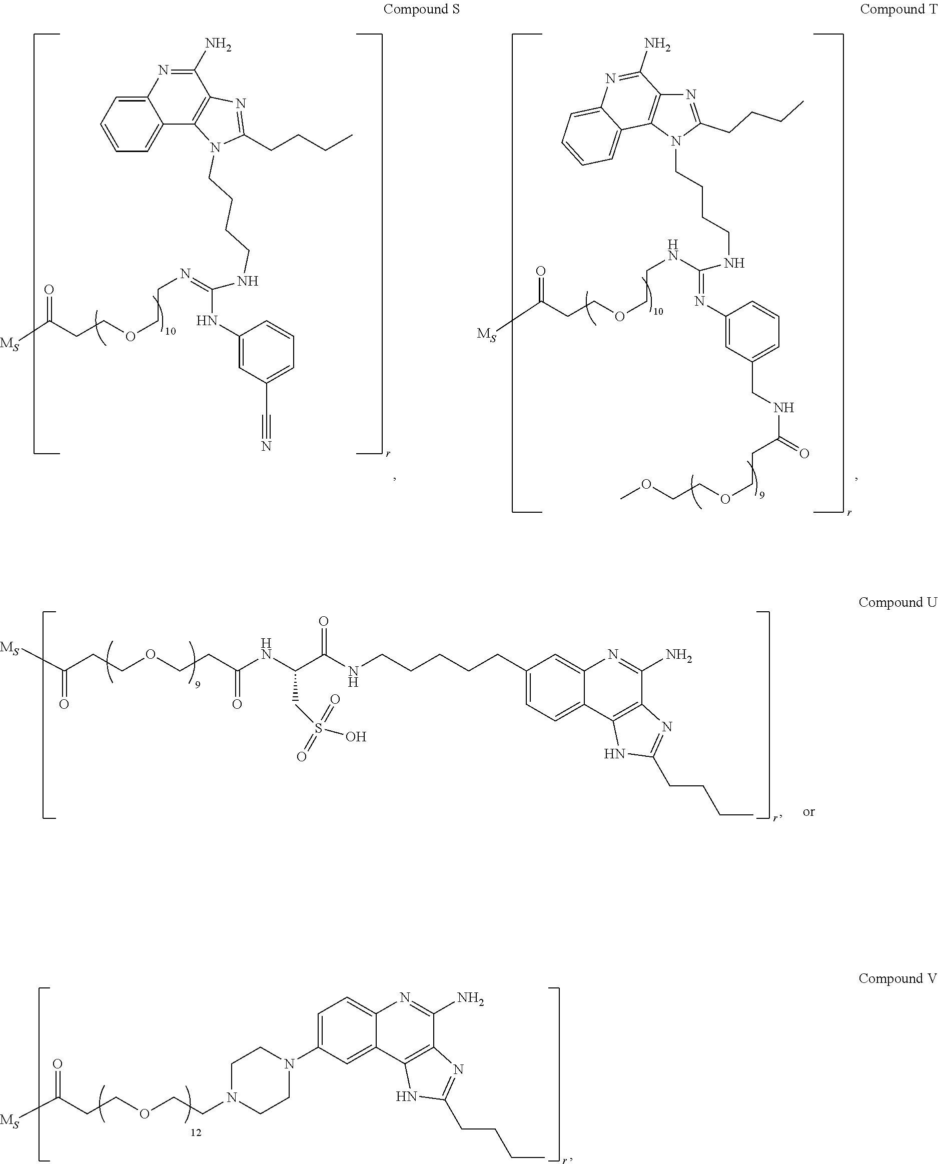

a pharmaceutically acceptable salt thereof, or a quaternary ammonium salt thereof, wherein subscript r is an integer from 1 to 50 and “M_{S}” is macromolecular support.

13. A composition comprising a plurality of macromolecule-supported compounds according to any one of aspects 1-12.

14. The composition of aspect 13, wherein the average TLR agonist to macromolecular support ratio is from about 0.01 to about 50.

15. The composition of aspect 14, wherein the average TLR agonist to macromolecular support ratio is from about 1 to about 10.

16. The composition of aspect 15, wherein the average TLR agonist to macromolecular support ratio is from about 1 to about 6.

17. The composition of aspect 16, wherein the average TLR agonist to macromolecular support ratio is from about 1 to about 4.

18. A method for treating cancer comprising administering a therapeutically effective amount of a macromolecule-supported compound according to any one of aspects 1-13 or a composition according to any one of aspects 14-17 to a subject in need thereof.

19. The method of aspect 18, wherein the cancer is susceptible to a pro-inflammatory response induced by TLR7 and/or TLR8 agonism.

20. Use of a macromolecule-supported compound according to any one of aspects 1-13 or a composition according to any one of aspects 14-17 for treating cancer.

21. Use of a macromolecule-supported compound according to any one of aspects 1-13 or a composition according to any one of aspects 14-17 for a chemical assay for TLR engagement and/or activity.

22. The use according to aspect 21, wherein the chemical assay is for TLR7 and/or TLR8 engagement and/or activity.

### EXAMPLES

The following examples further illustrate the invention but, of course, should not be construed as in any way limiting its scope.

### Example 1: Synthesis of Compound 2

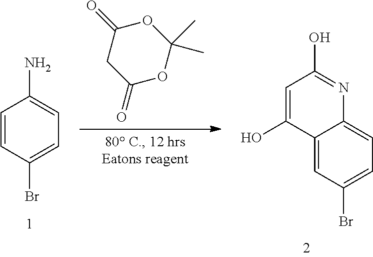

A mixture of 2,2-dimethyl-1,3-dioxane-4,6-dione (41.89 g, 290.66 mmol, 1 eq) and 4-bromoaniline (50 g, 290.66 mmol, 1 eq) was stirred (neat) at 80° C. for 12 hrs. Afterward, the small remaining amount of acetone was removed by vacuum. Eaton's reagent (415.15 g, 1.74 mol, 273.12 mL, 6 eq) was added to the mixture at 80° C. for 12 hrs. Water (1000 mL) was added to this mixture while stirring vigorously. The precipitate was filtered, washed with H₂O, and air dried to provide a solid. The solid was recrystallized from ethanol to afford 6-bromoquinoline-2,4-diol (26 g, 108.31 mmol, 37.26% yield) as off-white solid. ¹H NMR (dimethyl sulfoxide (DMSO)-d6, 400 MHz) δ 11.53 (s, 1H), 11.33 (s, 1H), 7.85 (d, J=2.4 Hz, 1H), 7.75 (dd, J=8.0 Hz, 4.0 Hz, 1H), 7.18-7.24 (m, 1H), 5.75 (s, 1H).

### Example 2: Synthesis of Compound 3

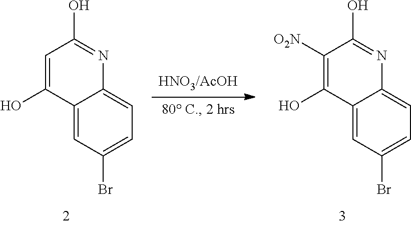 [SMILES: Oc1cc(O)c2cc(Br)ccc2n1 ; "80° C., 2 hrs" ; "HNO3/AcOH" ; O=[N+]([O-])c1c(O)nc2ccc(Br)cc2c1O]

To a solution of nitric acid HNO₃ (13.65 g, 216.62 mmol, 9.75 mL, 2 eq) in AcOH (500 mL) was added 6-bromoquinoline-2,4-diol (26 g, 108.31 mmol, 1 eq) slowly at 15° C. The mixture was stirred at 80° C. for 3 hours. The mixture was cooled and quenched by addition of water (1000 mL). The product was separated by filtration and washed by water (100 mL×3), dried to give desired product. The crude product 6-bromo-3-nitro-quinoline-2,4-diol (30 g, 105.24 mmol, 97.17% yield) was obtained as a yellow solid and used into the next step without further purification. ¹H NMR (DMSO-d₆, 400 MHz) δ 11.92 (s, 1H), 8.13 (s, 1H), 7.76 (d, J=8.4 Hz, 1H), 7.25 (d, J=8.4 Hz, 1H).

### Example 3: Synthesis of Compound 4

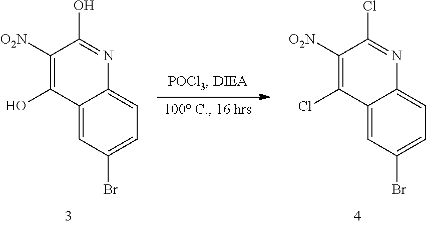 [SMILES: O=[N+]([O-])c1c(O)nc2ccc(Br)cc2c1O ; "100° C., 16 hrs" ; "POCl3, DIEA" ; O=[N+]([O-])c1c(Cl)nc2ccc(Br)cc2c1Cl]

To a mixture of 6-bromo-3-nitro-quinoline-2,4-diol (30 g, 105.24 mmol, 1 eq) in POCl₃ (484.12 g, 3.16 mol, 293.41 mL, 30 eq) was added N,N-diisopropylethylamine (40.81 g, 315.73 mmol, 55.00 mL, 3 eq) slowly at 15° C. The mixture was stirred at 100° C. for 16 hrs. The mixture was concentrated in vacuum. The residue was poured into ice water (2000 mL), filtered and washed with H₂O (500 mL×3), and dried to provide 6-bromo-2,4-dichloro-3-nitro-quinoline (30 g, 93.18 mmol, 88.54% yield) as a yellow solid. ¹H NMR (DMSO-d₆, 400 MHz) δ 8.48 (d, J=2.0 Hz, 1H), 8.25 (dd, J=8.8, 2.0 Hz, 1H), 8.10 (d, J=8.8 Hz, 1H).

### Example 4: Synthesis of Compound 5

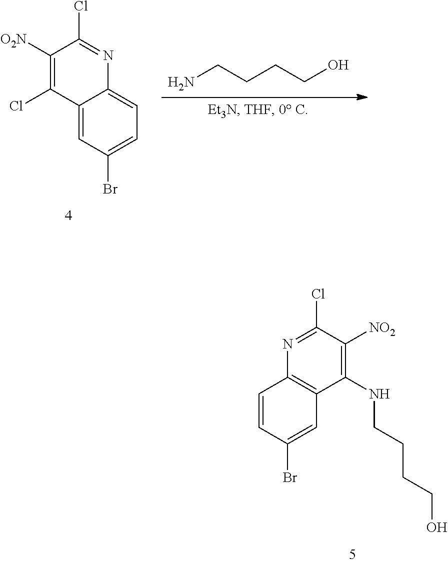

To a mixture of 6-bromo-2,4-dichloro-3-nitro-quinoline (10.00 g, 31.06 mmol, 1 eq) and 4-aminobutan-1-ol (2.77 g, 31.06 mmol, 2.89 mL, 1 eq) in THF (100 mL) was added Et₃N (4.71 g, 46.59 mmol, 6.49 mL, 1.5 eq) in one portion at 0° C. The mixture was stirred at 0° C. for 2 hrs. The mixture was diluted with water (200 mL) and extracted with EtOAc (100 mL×3). The organic layer was washed with brine (100 mL), dried over Na₂SO₄, filtered and concentrated to provide 4-[(6-bromo-2-chloro-3-nitro-4-quinolyl)amino]butan-1-ol (12 g, crude) as a yellow solid. 41 NMR (DMSO-d₆, 400 MHz) δ 8.72-8.85 (m, 1H), 7.90-7.98 (m, 2H), 7.70-7.75 (m, 1H), 4.49 (t, J=3.2 Hz, 1H), 3.36-3.43 (m, 2H), 3.07-3.15 (m, 2H), 1.63-1.73 (m, 2H), 1.39-1.48 (m, 2H).

### Example 5: Synthesis of Compound 6

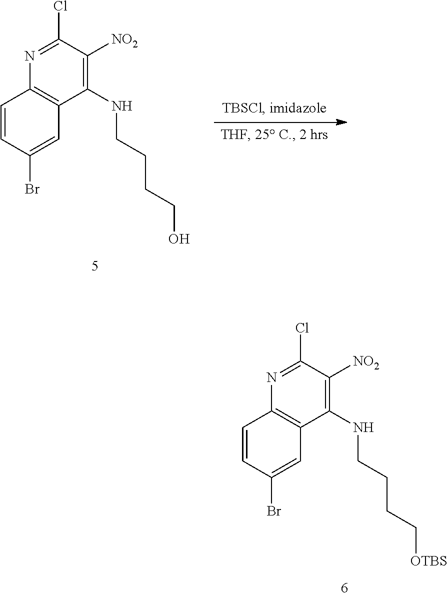 [SMILES: O=[N+]([O-])c1c(Cl)nc2ccc(Br)cc2c1NCCCCO ; "TBSCl, imidazole" ; "THF, 25° C., 2 hrs" ; CC(C)(C)[Si](C)(C)OCCCCNc1c([N+](=O)[O-])c(Cl)nc2ccc(Br)cc12]

To a mixture of 4-[(6-bromo-2-chloro-3-nitro-4-quinolyl)amino]butan-1-ol (12 g, 32.03 mmol, 1 eq) in THF (200 mL) was added imidazole (2.83 g, 41.64 mmol, 1.3 eq) and tert-butyldimethylsilyl chloride (6.28 g, 41.64 mmol, 5.10 mL, 1.3 eq) slowly in portions at 25° C. The mixture was stirred at 25° C. for 2 hrs. The mixture was diluted with water (200 mL) and extracted with EtOAc (200 mL×3). The organic layer was washed with brine (100 mL), dried over Na₂SO₄, filtered and concentrated. The residue was purified by silica gel chromatography (column height: 250 mm, diameter: 100 mm, 100-200 mesh silica gel, petroleum ether/ethyl acetate=10/1, 5/1). Compound 6-bromo-N-[4-[tert-butyl (dimethyl)silyl]oxybutyl]-2-chloro-3-nitro-quinolin-4-amine (15 g, 30.68 mmol, 95.79% yield) was obtained as a yellow solid. ¹H NMR (DMSO-d₆, 400 MHz) δ 8.80 (d, J=2.0 Hz, 1H), 7.92-7.98 (m, 2H), 7.70-7.78 (m, 1H), 3.52-3.59 (m, 2H), 3.09-3.16 (m, 2H), 1.63-1.72 (m, 2H), 1.42-1.52 (m, 2H), 0.81 (s, 9H), −0.02 (s, 6H).

### Example 6: Synthesis of Compound 7

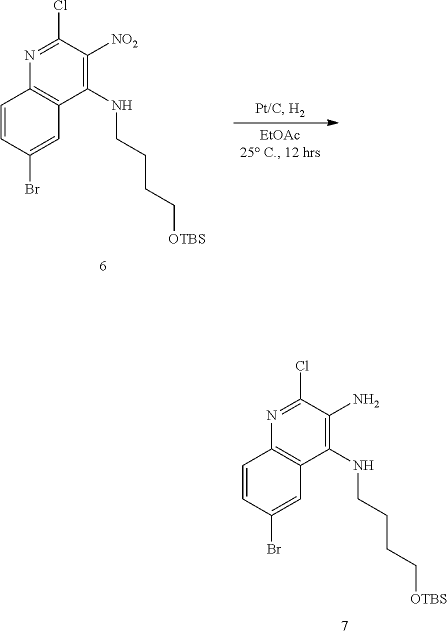 [SMILES: CC(C)(C)[Si](C)(C)OCCCCNc1c([N+](=O)[O-])c(Cl)nc2ccc(Br)cc12 ; "EtOAc25° C., 12 hrs" ; "Pt/C, H2" ; CC(C)(C)[Si](C)(C)OCCCCNc1c(N)c(Cl)nc2ccc(Br)cc12]

To a solution of 6-bromo-N-[4-[tert-butyl(dimethyl)silyl]oxybutyl]-2-chloro-3-nitro-quinolin-4-amine (15 g, 30.68 mmol, 1 eq) in EtOAc (200 mL) was added platinum on carbon (3.87 g, 920.48 μmol, 5% purity, 0.03 eq) under N₂. The suspension was degassed under vacuum and purged with H₂ several times. The mixture was stirred under H₂ (50 psi) at 25° C. for 12 hrs. The mixture was filtered and concentrated. Compound 6-bromo-N⁴-(4-((tert-butyldimethylsilyl)oxy)butyl)-2-chloroquinoline-3,4-diamine (13.6 g, 29.64 mmol, 96.59% yield) was obtained as a yellow oil. ¹H NMR (CDCl₃, 400 MHz) δ 7.92 (d, J=2.0 Hz, 1H), 7.75 (d, J=9.2 Hz, 1H), 7.51-7.55 (m, 1H), 4.17-4.22 (m, 1H), 3.65-3.81 (m, 2H), 3.07-3.37 (m, 2H), 1.53-1.85 (m, 4H), 0.92 (s, 9H), 0.06 (s, 6H).

### Example 7: Synthesis of Compound 8

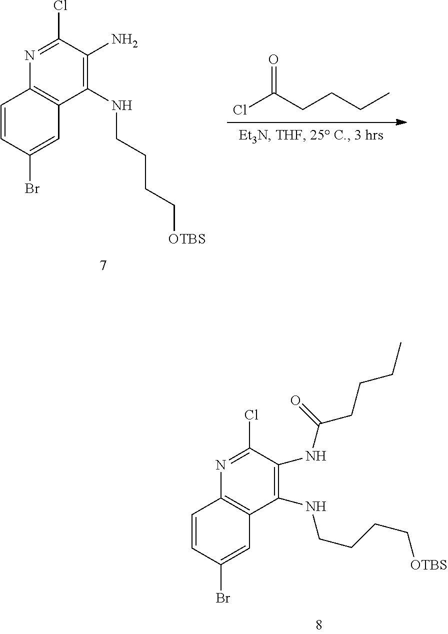

To a mixture of 6-bromo-N⁴-[4-[tert-butyl(dimethyl)silyl]oxybutyl]-2-chloro-quinoline-3,4-diamine (13.6 g, 29.64 mmol, 1 eq) and pentanoyl chloride (6.07 g, 50.38 mmol, 6.11 mL, 1.7 eq) in THF (200 mL) was added Et₃N (4.50 g, 44.45 mmol, 6.19 mL, 1.5 eq) in batches at 0° C. The mixture was stirred at 25° C. for 3 hrs. The mixture was diluted with water (200 mL) and extracted with EtOAc (100 mL×3). The organic layer was washed with brine (100 mL), dried over Na₂SO₄, filtered and concentrated. The residue was purified by flash silica gel chromatography (Teledyne Isco, Lincoln, Nebr.), 20 g SEPAFLASH™ silica flash column, eluent of 0 to about 90% ethyl acetate/petroleum ether gradient at 100 mL/min). Compound N-[6-bromo-4-[4-[tert-butyl (dimethyl) silyl]oxybutylamino]-2-chloro-3-quinolyl]pentanamide (9 g, 16.57 mmol, 55.93% yield) was obtained as a white solid. ¹H NMR (DMSO-d₆, 400 MHz) δ 9.47 (s, 1H), 8.56 (d, J=2.0 Hz, 1H), 7.74-7.81 (m, 1H), 7.60-7.67 (m, 1H), 6.91-6.99 (m, 1H), 3.55-3.61 (m, 2H), 3.39-3.46 (m, 2H), 2.30-2.35 (m, 2H), 1.54-1.65 (m, 4H), 1.43-1.53 (m, 2H), 1.31-1.39 (m, 2H), 0.87-0.95 (m, 3H), 0.83 (s, 9H), 0.00 (s, 6H).

### Example 8: Synthesis of Compound 9

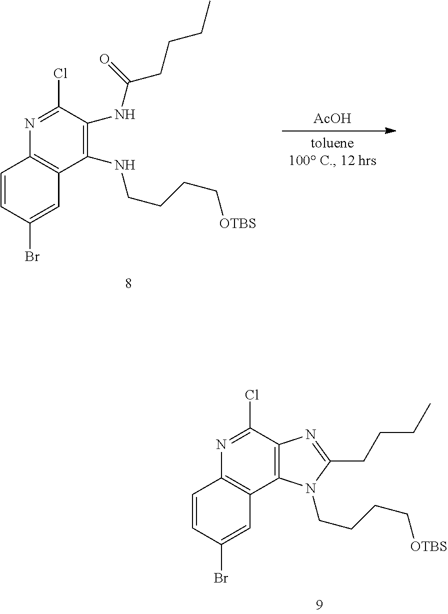 [SMILES: "AcOH" ; CCCCC(=O)Nc1c(Cl)nc2ccc(Br)cc2c1NCCCCO[Si](C)(C)C(C)(C)C ; "toluene100° C., 12 hrs" ; CCCCc1nc2c(Cl)nc3ccc(Br)cc3c2n1CCCCO[Si](C)(C)C(C)(C)C]

To a mixture of N-[6-bromo-4-[4-[tert-butyl(dimethyl)silyl]oxybutylamino]-2-chloro-3-quinolyl]pentanamide (9 g, 16.57 mmol, 1 eq) in toluene (150 mL) was added AcOH (1.99 g, 33.15 mmol, 1.90 mL, 2 eq) in one portion at 25° C. The mixture was stirred at 100° C. for 12 hrs. The reaction mixture was concentrated under reduced pressure to remove the solvent. The residue mixture was washed with EtOAc (50 mL). The mixture was filtered, and the filtrate was concentrated under reduced pressure to provide a residue. The residue was purified by flash silica gel chromatography (Teledyne Isco, 7 g, SEPAFLASH™ silica flash column, eluent of 0 to 100% ethyl acetate/petroleum ether gradient at 100 mL/min). Compound 4-(8-bromo-2-butyl-4-chloro-imidazo[4,5-c]quinolin-1-yl)butoxy-tert-butyl-dimethyl-silane (5 g, 9.52 mmol, 57.46% yield) was obtained as a white solid. ¹H NMR (DMSO-d₆, 400 MHz) δ 8.42 (d, J=4.0 Hz, 1H), 7.97-8.03 (m, 1H), 7.83-7.90 (m, 1H), 4.67 (t, J=8.0 Hz, 2H), 3.61 (t, J=4.0 Hz, 2H), 3.0 (t, J=8.0 Hz, 2H), 1.78-1.94 (m, 4H), 1.52-1.62 (m, 2H), 1.43-1.50 (m, 2H), 0.97 (t, J=8.0 Hz, 3H), 0.72 (s, 9H), −0.06 (s, 6H).

### Example 9: Synthesis of Compound 10

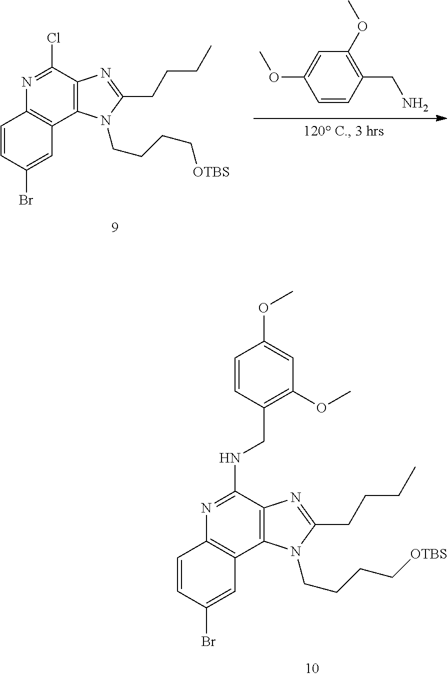

To a mixture of 4-(8-bromo-2-butyl-4-chloro-imidazo[4,5-c]quinolin-1-yl)butoxy-tert-butyl-dimethyl-silane (6.2 g, 11.81 mmol, 1 eq) in (2,4-dimethoxyphenyl) methanamine (19.75 g, 118.10 mmol, 17.79 mL, 10 eq) at 20° C. The mixture was stirred at 120° C. for 3 hrs. The mixture was diluted with water (200 mL) and extracted with EtOAc (100 ml×3). The organic layer was washed with brine (100 mL), dried over Na₂SO₄, filtered and concentrated. The residue was purified by flash silica gel chromatography (Teledyne Isco, 10 g, SEPAFLASH™ silica flash column, eluent of 0 to about 50% ethyl acetate/petroleum ether gradient at 100 mL/min) to provide 8-bromo-2-butyl-1-[4-[tert-butyl(dimethyl)silyl]oxybutyl]-N-[(2,4-dimethoxyphenyl)methyl]imidazo[4,5-c]quinolin-4-amine (8.7 g, crude) as a yellow oil. ¹H NMR (DMSO-d₆, 400 MHz) δ 7.99-8.07 (m, 1H), 7.52-7.57 (m, 1H), 7.44-7.51 (m, 1H), 7.10-7.17 (m, 1H), 6.98-7.06 (m, 1H), 6.56 (d, J=2.0 Hz, 1H), 6.39 (dd, J=8.0 Hz, 2.0 Hz, 1H), 4.63-4.69 (m, 2H), 4.45-4.53 (m, 2H), 3.81-3.85 (m, 3H), 3.67-3.73 (m, 3H), 3.56-3.66 (m, 2H), 2.85-2.95 (m, 2H), 1.74-1.90 (m, 4H), 1.51-1.63 (m, 2H), 1.37-1.48 (m, 2H), 0.89-0.98 (m, 3H), 0.76 (s, 9H), −0.04 (s, 6H).

### Example 10: Synthesis of Compound 11

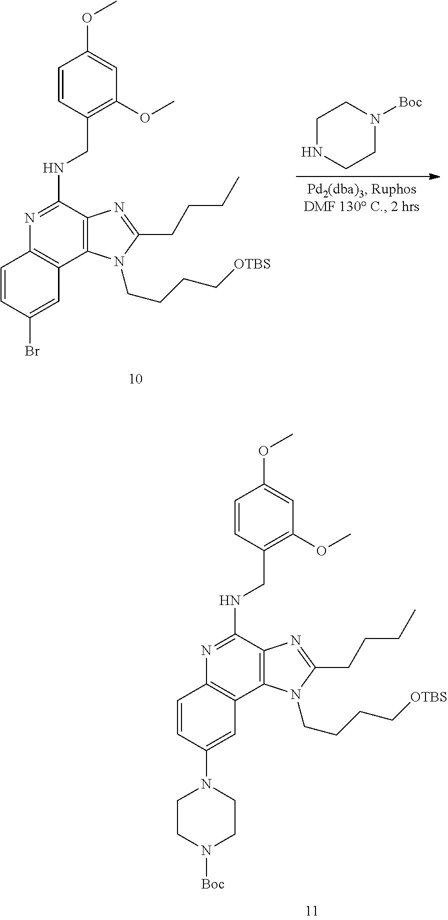

To a mixture of tert-butyl piperazine-1-carboxylate (10.94 g, 58.71 mmol, 5 eq) and 8-bromo-2-butyl-1-[4-[tert-butyl(dimethyl)silyl]oxybutyl]-N-[(2,4-dimethoxyphenyl)methyl]imidazo[4,5-c]quinolin-4-amine (7.7 g, 11.74 mmol, 1 eq) in dimethylformamide (DMF; 100 mL) was added 2-dicyclohexylphosphino-2′,6′-diisopropoxybiphenyl (RuPhos, 547.95 mg, 1.17 mmol, 0.1 eq), Pd₂(dba)₃ (537.64 mg, 587.12 μmol, 0.05 eq) and Cs₂CO₃ (7.65 g, 23.48 mmol, 2 eq) in one portion at 20° C. under N₂. The mixture was stirred at 130° C. for 2 hrs. The mixture was diluted with water (100 mL) and extracted with EtOAc (50 mL×3). The organic layer was washed with brine (50 mL), dried over Na₂SO₄, filtered and concentrated. The crude product was purified by chromatography on a silica gel eluted with petroleum ether:ethyl acetate (from 1/0 to 0/1). Compound tert-butyl 4-[2-butyl-1-[4-[tert-butyl(dimethyl)silyl]oxybutyl]-4-[(2,4-dimethoxyphenyl)methylamino]imidazo[4,5-c]quinolin-8-yl]piperazine-1-carboxylate (8 g, 9.98 mmol, 85.02% yield, 94.980% purity) was obtained as a yellow oil. ¹H NMR (DMSO-d6, 400 MHz) δ 7.49-7.56 (m, 1H), 7.26-7.32 (m, 1H), 7.19-7.23 (m, 1H), 7.15-7.19 (m, 1H), 6.54-6.58 (m, 1H), 6.46-6.53 (m, 1H), 6.37-6.42 (m, 1H), 4.65 (s, 2H), 4.47-4.53 (m, 2H), 3.81-3.85 (m, 3H), 3.68-3.73 (m, 3H), 3.58-3.65 (m, 2H), 3.47-3.54 (m, 4H), 3.10-3.16 (m, 4H), 2.87-2.93 (m, 2H), 1.72-1.95 (m, 6H), 1.60-1.66 (m, 2H), 1.42-1.44 (m, 9H), 0.93-0.97 (m, 3H), 0.78 (s, 9H), 0.00 (s, 6H).

### Example 11: Synthesis of Compound 12

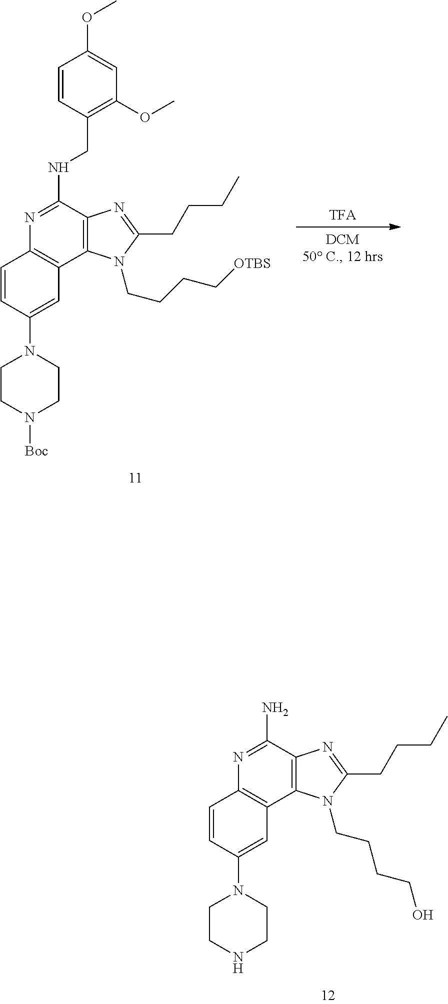 [SMILES: CCCCc1nc2c(NCc3ccc(OC)cc3OC)nc3ccc(N4CCN(C(=O)OC(C)(C)C)CC4)cc3c2n1CCCCO[Si](C)(C)C(C)(C)C ; "DCM50° C., 12 hrs" ; "TFA" ; CCCCc1nc2c(N)nc3ccc(N4CCNCC4)cc3c2n1CCCCO]

To a mixture of tert-butyl 4-[2-butyl-1-[4-[tert-butyl(dimethyl)silyl]oxybutyl]-4-[(2,4-dimethoxyphenyl)methylamino]imidazo[4,5-c]quinolin-8-yl]piperazine-1-carboxylate (8 g, 10.51 mmol, 1 eq) in dichloromethane (DCM; 100 mL) was added TFA (trifluoroacetic acid; 41.95 g, 367.90 mmol, 27.24 mL, 35 eq) in one portion at 20° C. The mixture was stirred at 50° C. for 12 hrs. The mixture was concentrated. Then the residue was diluted with MeOH (50 mL) and the mixture was filtered, the filtrate was concentrated to give the crude product 4-(4-amino-2-butyl-8-piperazin-1-yl-imidazo[4,5-c]quinolin-1-yl)butan-1-ol (8.1 g, crude, TFA) as a yellow solid for next step.

### Example 12: Synthesis of Compound 13

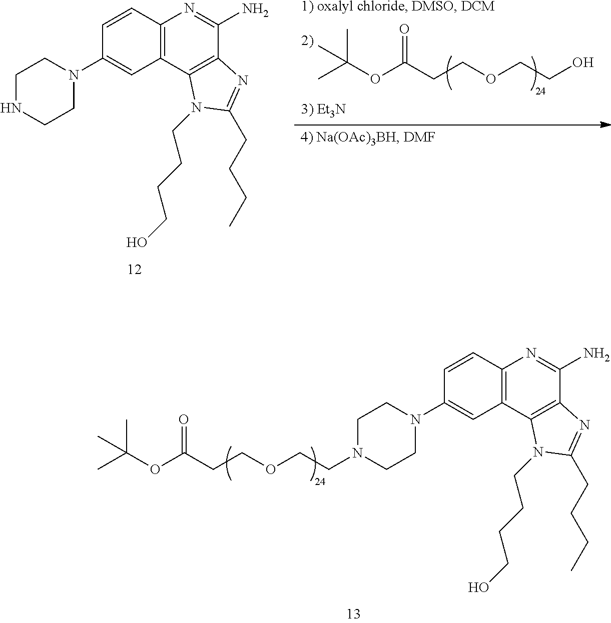

To a solution of oxalyl chloride (127 mg, 86 μL, 1 mmol, 2 eq) in DCM (1 mL) at 80° C. was added dropwise a solution of DMSO (156 mg, 142 μL, 2 mmol, 4 eq) in DCM (1 mL). The mixture was stirred for 15 min at 80° C. To this mixture was added a solution of hydroxyl-PEG24-t-butyl ester (602 mg, 0.5 mmol, 1 eq) in DCM (1 mL). After stirring for 15 min, Et₃N (303 mg, 418 μL) was added and the mixture was stirred at 80° C. for 15 min then removed from the cold bath and allowed to warm to 20° C. over 30 min. To a suspension of 4-(4-amino-2-butyl-8-piperazin-1-yl-imidazo[4,5-c]quinolin-1-yl)butan-1-ol TFA salt and sodium triacetoxyborohydride (212 mg, 1 mmol, 2 eq) in DMF (3 mL) was added the previous mixture slowly at 20° C. The combined mixture was stirred at 20° C. for 45 min. Solvent was removed under reduced pressure and to the remaining was added 3 mL of 10% Na₂CO₃ and stirred vigorously for 15 min. Water (20 mL) was added and the crude product was extracted into DCM (25 mL). The organic layer was washed with brine, dried (Na₂SO₄), filtered and concentrated. The crude material was purified by flash chromatography using a gradient elution of 2-15% MeOH/DCM+1% Et₃N to yield tert-butyl 1-(4-(4-amino-2-butyl-1-(4-hydroxybutyl)-1H-imidazo[4,5-c]quinolin-8-yl)piperazin-1-yl)-3,6,9,12,15,18,21,24,27,30,33,36,39,42,45,48,51,54,57,60,63,66,69,72-tetracosaoxapentaheptacontan-75-oate (490 mg, 0.31 mmol, 62%) as a golden syrup. LC/MS [M+H] 158.98 (calculated); LC/MS [M+H] 1582.27 (observed).

### Example 13: Synthesis of Compound 14

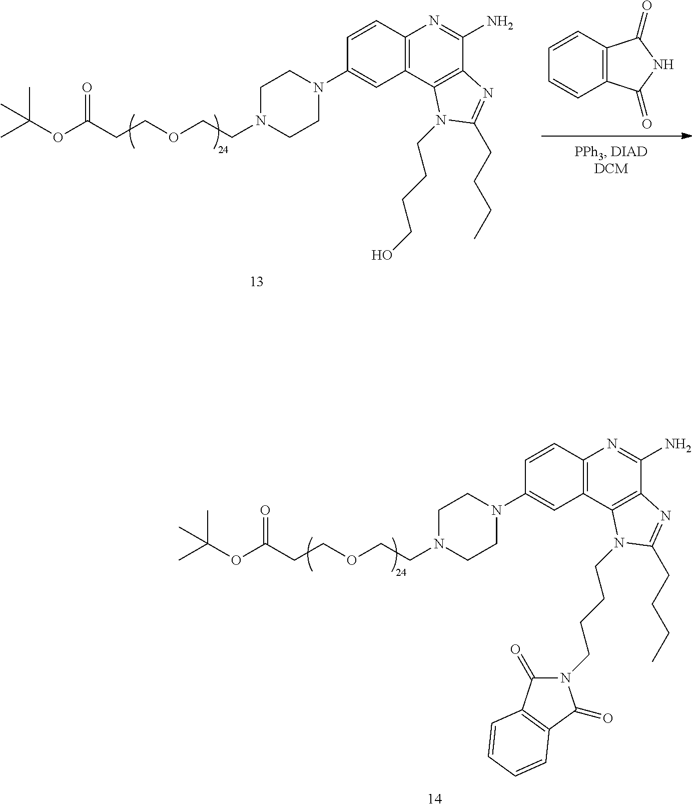

To a solution of triphenylphosphine (0.236 mg, 0.9 mmol, 3 eq) in DCM (5 mL) was added diisopropyl azodicarboxylate (182 mg, 177 μL, 0.9 mmol, 3 eq) dropwise. After 5 min phthalimide (74 mg, 0.5 mmol, 1.7 eq) was added. The mixture was stirred for 5 minutes then a solution of tert-butyl 1-(4-(4-amino-2-butyl-1-(4-hydroxybutyl)-1H-imidazo[4,5-c]quinolin-8-yl)piperazin-1-yl)-3,6,9,12,15,18,21,24,27,30,33,36,39,42,45,48,51,54,57,60,63,66,69,72-tetracosaoxapentaheptacontan-75-oate (475 mg, 0.3 mmol, 1 eq) in DCM (1 mL) was added to the mixture. After 30 min, the solvent was removed under reduced pressure and the crude material was purified by flash chromatography using a gradient elution of 2-10% MeOH/DCM+1% Et₃N to yield tert-butyl 1-(4-(4-amino-2-butyl-1-(4-(1,3-dioxoisoindolin-2-yl)butyl)-1H-imidazo[4,5-c]quinolin-8-yl)piperazin-1-yl)-3,6,9,12,15,18,21,24,27,30,33,36,39,42,45,48,51,54,57,60,63,66,69,72-tetracosaoxapentaheptacontan-75-oate (399 mg, 0.23 mmol, 77%) as a golden syrup. LC/MS [M+H] 1711.01 (calculated); LC/MS [M+H] 1711.25 (observed).

### Example 14: Synthesis of Compound 15

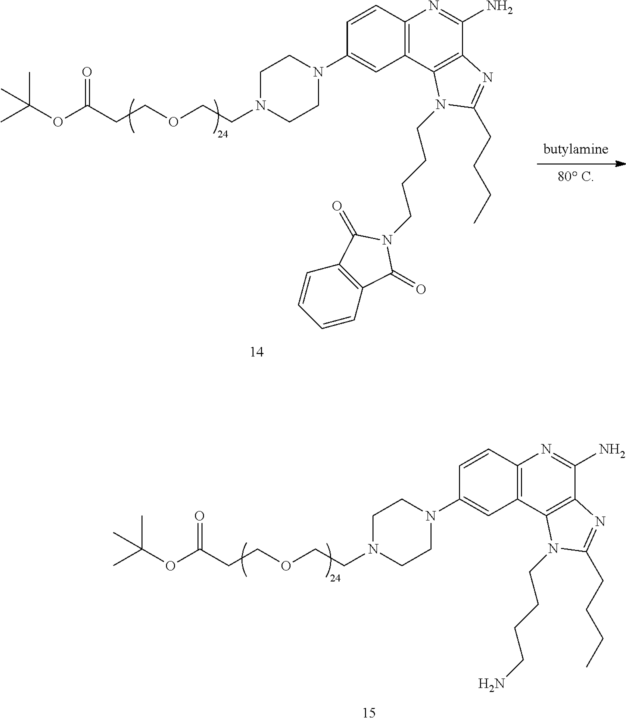

A solution of tert-butyl 1-(4-(4-amino-2-butyl-1-(4-(1,3-dioxoisoindolin-2-yl)butyl)-1H-imidazo[4,5-c]quinolin-8-yl)piperazin-1-yl)-3,6,9,12,15,18,21,24,27,30,33,36,39,42,45,48,51,54,57,60,63,66,69,72-tetracosaoxapentaheptacontan-75-oate (390 mg, 0.23 mmol, 1 eq) was combined with 1-butyl amine (3 mL) and heated at 80° C. in a capped vial in a heating block for 6 h. The solvent was removed and the crude material was purified by flash chromatography using a gradient elution of 2-15% MeOH/DCM+1% Et₃N to yield tert-butyl 1-(4-(4-amino-1-(4-aminobutyl)-2-butyl-1H-imidazo[4,5-c]quinolin-8-yl)piperazin-1-yl)-3,6,9,12,15,18,21,24,27,30,33,36,39,42,45,48,51,54,57,60,63,66,69,72-tetracosaoxapentaheptacontan-75-oate (330 mg, 21 mmol, 92%) as a yellow syrup. LC/MS [M+H] 1581.00 (calculated); LC/MS [M+H] 1581.43 (observed).

### Example 15: Synthesis of Compound 16

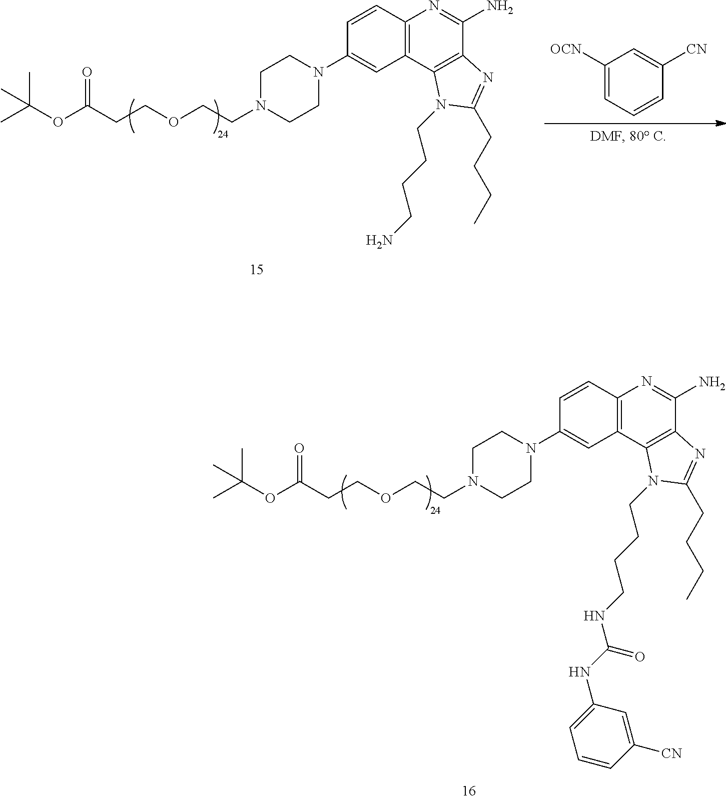

A mixture of yield tert-butyl 1-(4-(4-amino-1-(4-aminobutyl)-2-butyl-1H-imidazo[4,5-c]quinolin-8-yl)piperazin-1-yl)-3,6,9,12,15,18,21,24,27,30,33,36,39,42,45,48,51,54,57,60,63,66,69,72-tetracosaoxapentaheptacontan-75-oate (323 mg, 0.2 mmol, 1 eq) and 3-cyanophenylisocyanate were dissolved in DMF (3 mL) and heated at 80° C. in a capped vial in a heating block for 16 h. The solvent was removed and the crude material was purified by flash chromatography using a gradient elution of 2-10% MeOH/DCM+1% Et₃N to yield tert-butyl 1-(4-(4-amino-2-butyl-1-(4-(3-(3-cyanophenyl)ureido)butyl)-1H-imidazo[4,5-c]quinolin-8-yl)piperazin-1-yl)-3,6,9,12,15,18,21,24,27,30,33,36,39,42,45,48,51,54,57,60,63,66,69,72-tetracosaoxapentaheptacontan-75-oate (240 mg, 0.14 mmol, 70%) as a brownish solid. LC/MS [M+H] 1725.03 (calculated); LC/MS [M+H] 1725.30 (observed).

### Example 16: Synthesis of Compound 17

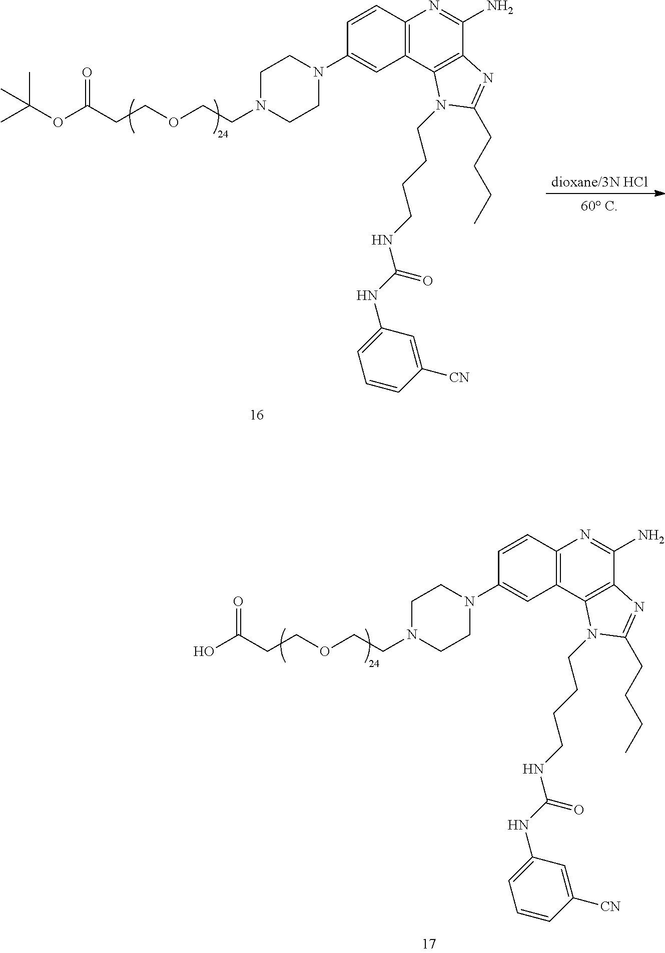

Tert-butyl 1-(4-(4-amino-2-butyl-1-(4-(3-(3-cyanophenyl)ureido)butyl)-1H-imidazo[4,5-c]quinolin-8-yl)piperazin-1-yl)-3,6,9,12,15,18,21,24,27,30,33,36,39,42,45,48,51,54,57,60,63,66,69,72-tetracosaoxapentaheptacontan-75-oate (225 mg, 0.14 mmol) was dissolved in a 1:1 mixture of dioxane and 3 N HCl (5 mL) then heated to 60° C. for 90 min. The solvent was removed and the residue was azeotroped four times with acetonitrile (5 mL). The resulting 1-(4-(4-amino-2-butyl-1-(4-(3-(3-cyanophenyl)ureido)butyl)-1H-imidazo[4,5-c]quinolin-8-yl)piperazin-1-yl)-3,6,9,12,15,18,21,24,27,30,33,36,39,42,45,48,51,54,57,60,63,66,69,72-tetracosaoxapentaheptacontan-75-oic acid HCl salt (220 mg, 0.13 mmol, 95%) was used without further purification.

### Example 17: Synthesis of Compound 18

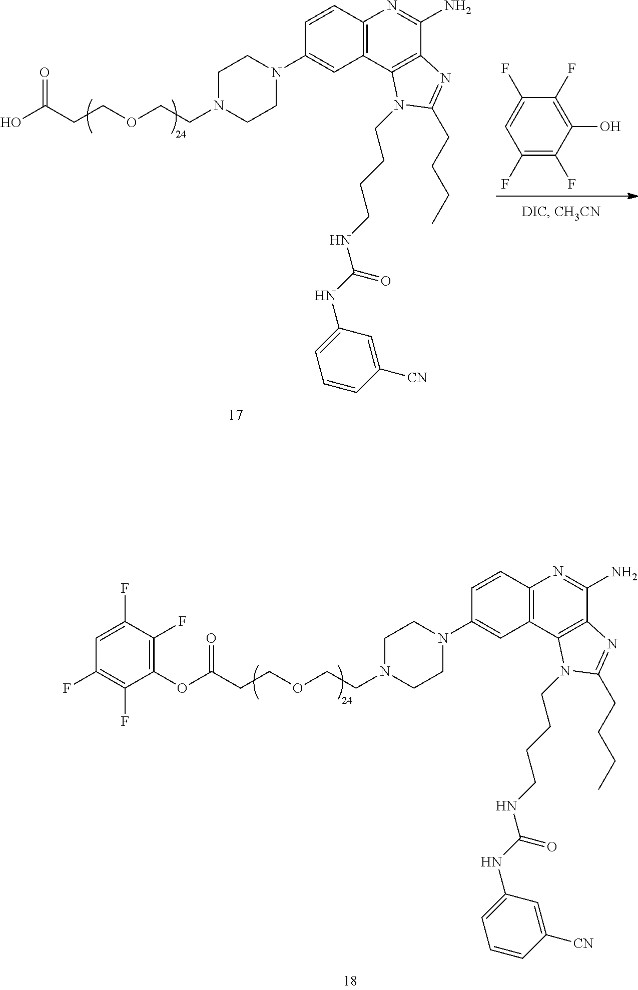

To 1-(4-(4-amino-2-butyl-1-(4-(3-(3-cyanophenyl)ureido)butyl)-1H-imidazo[4,5-c]quinolin-8-yl)piperazin-1-yl)-3,6,9,12,15,18,21,24,27,30,33,36,39,42,45,48,51,54,57,60,63,66,69,72-tetracosaoxapentaheptacontan-75-oic acid HCl salt (220 mg, 0.13 mmol, 1 eq) was added a mixture of 2,3,5,6-tetrafluorophenol (66 mg, 0.4 mmol, 3 eq) and diisopropylcarbodiimide (51 mg, 62 μL, 0.4 mmol, 3 eq) dissolved in acetonitrile (3 mL) and the mixture was stirred at 20° C. for 16 h. The mixture was diluted with water (12 mL) and purified by reverse phase chromatography using a gradient eluent of 30-80% acetonitrile/water+0.1% TFA over 10 min. The pooled fractions were concentrated under reduced pressure and the glassy film was azeotroped with acetonitrile four times (20 mL) to yield 2,3,5,6-tetrafluorophenyl 1-(4-(4-amino-2-butyl-1-(4-(3-(3-cyanophenyl)ureido)butyl)-1H-imidazo[4,5-c]quinolin-8-yl)piperazin-1-yl)-3,6,9,12,15,18,21,24,27,30,33,36,39,42,45,48,51,54,57,60,63,66,69,72-tetracosaoxapentaheptacontan-75-oate (86 mg, 0.04 mmol, 36%) as a glassy film. LC/MS [M+H] 1816.96 (calculated); LC/MS [M+H] 1817.46 (observed).

### Example 18: Synthesis of Compound 19

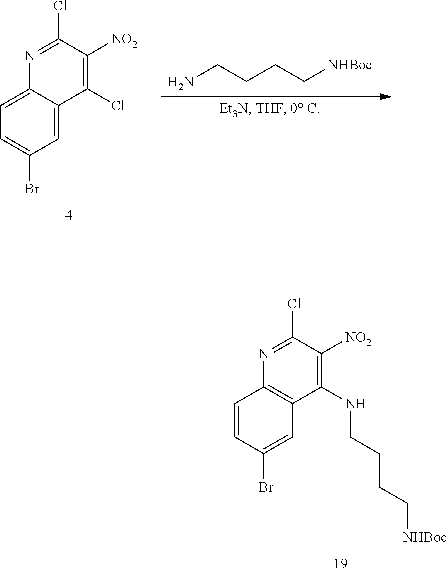

To a mixture of 6-bromo-2,4-dichloro-3-nitro-quinoline (5.5 g, 17.08 mmol, 1 eq) and tert-butyl N-(4-aminobutyl)carbamate (3.54 g, 18.79 mmol, 1.1 eq) in THF (20 mL) was added Et₃N (2.59 g, 25.63 mmol, 3.57 mL, 1.5 eq) slowly in one portion at 0° C. The mixture was stirred at 0° C. for 2 h. TLC showed the reaction was finished and a new spot was detected. The mixture was diluted with ice-water and extracted with EtOAc (50 ml×3). The organic layer was washed with brine (40 ml), dried over Na₂SO₄, filtered and concentrated. The crude product was purified by re-crystallization from petroleum ether (200 mL) at 25° C. to give the tert-butyl N-[4-[(6-bromo-2-chloro-3-nitro-4-quinolyl)amino]butyl]carbamate (6.5 g, 13.7 mmol, 80.3% yield) as a yellow solid. ¹H NMR (DMSO-d₆, 400 MHz) δ=8.80 (d, J=2.0 Hz, 1H), 7.95 (dd, J=2.0, 8.8 Hz, 1H), 7.74 (d, J=8.8 Hz, 1H), 6.78 (br t, J=5.2 Hz, 1H), 3.10 (br t, J=7.2 Hz, 2H), 2.93-2.85 (m, 2H), 1.61 (quin, J=7.4 Hz, 2H), 1.44-1.38 (m, 2H), 1.35 (s, 9H).

### Example 19: Synthesis of Compound 20

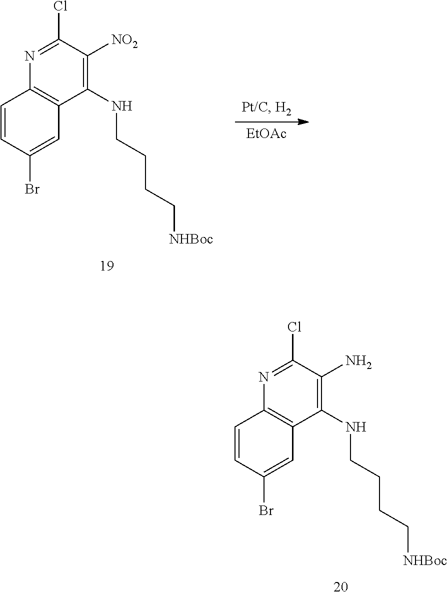 [SMILES: "EtOAc" ; CC(C)(C)OC(=O)NCCCCNc1c([N+](=O)[O-])c(Cl)nc2ccc(Br)cc12 ; "Pt/C, H2" ; CC(C)(C)OC(=O)NCCCCNc1c(N)c(Cl)nc2ccc(Br)cc12]

To a solution of tert-butyl N-[4-[(6-bromo-2-chloro-3-nitro-4-quinolyl)amino] butyl]carbamate (5.5 g, 11.61 mmol, 1 eq) in EtOAc (20 mL) was added platinum on carbon (2.44 g, 5% purity). The suspension was degassed under vacuum and purged with H₂ several times. The mixture was stirred under H₂ (50 psi) at 25° C. for 2 h. LCMS showed the reaction was finished. The mixture was filtered and concentrated. The residue was purified by silica gel chromatography (column height: 250 mm, diameter: 100 mm, 100-200 mesh silica gel, petroleum ether/ethyl acetate=4/1, 1/1) to yield tert-butyl N-[4-[(3-amino-6-bromo-2-chloro-4-quinolyl) amino]butyl]carbamate (3.4 g, 7.6 mmol, 66% yield) as a yellow solid. ¹H NMR (400 MHz, DMSO-d6) δ=8.23 (d, J=2.0 Hz, 1H), 7.62 (d, J=8.8 Hz, 1H), 7.55-7.46 (m, 1H), 6.75 (m, 1H), 5.28 (t, J=6.4 Hz, 1H), 3.13-3.19 (m, 2H), 2.92-2.78 (m, 2H), 1.45-1.50 (m, 2H), 1.43-1.37 (m, 2H), 1.34 (s, 9H).

### Example 20: Synthesis of Compound 21

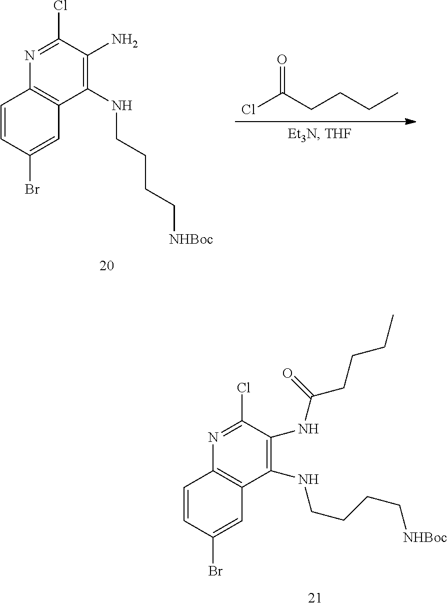

To a mixture of tert-butyl N-[4-[(3-amino-6-bromo-2-chloro-4-quinolyl) amino] butyl]carbamate (1.7 g, 3.83 mmol, 1 eq) and pentanoyl chloride (924 mg, 7.6 mmol, 930 μL, 2 eq) in THF (30 ml) was added Et₃N (582 mg, 5.8 mmol, 800 μL, 1.5 eq) in one portion at 0° C. Then the mixture was stirred at 25° C. for 2 h. LCMS showed the reaction was finished. The mixture was diluted with water (30 ml) and extracted with EtOAc (30 mL×3). The organic layer was washed with brine (40 ml), dried over Na₂SO₄, filtered and concentrated. The residue was purified by silica gel chromatography (column height: 250 mm, diameter: 100 mm, 100-200 mesh silica gel, petroleum ether/ethyl acetate=4/1, 1/1) to yield tert-butyl N-[4-[[6-bromo-2-chloro-3-(pentanoylamino)-4-quinolyl]amino]butyl]carbamate (2 g, 3.79 mmol, 99% yield) as a yellow solid. ¹H NMR (DMSO-d₆, 400 MHz) δ=8.56 (d, J=2.0 Hz, 1H), 7.78 (dd, J=2.0, 8.8 Hz, 1H), 7.63 (d, J=8.8 Hz, 1H), 6.90-6.85 (m, 1H), 6.80-6.78 (m, 1H), 3.43-3.38 (m, 2H), 2.94-2.89 (m, 2H), 2.39-2.29 (m, 2H), 1.64-1.50 (m, 4H), 1.36 (s, 9H), 0.94-0.88 (m, 3H)

### Example 21: Synthesis of Compound 22

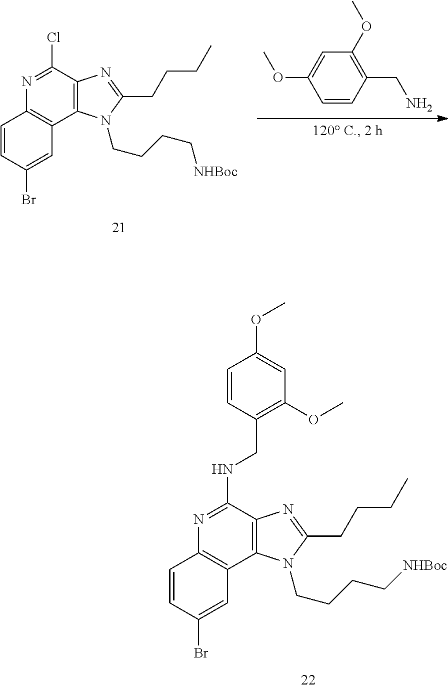

To a mixture of tert-butyl N-[4-(8-bromo-2-butyl-4-chloro-imidazo[4,5-c]quinolin-1-yl)butyl]carbamate (0.8 g, 1.6 mmol, 1 eq) and (2,4-dimethoxyphenyl) methanamine (2.62 g, 15.7 mmol, 2.36 mL, 10 eq) was stirred at 120° C. for 2 hours. The mixture was added 2M HCl adjust to pH-4 and extracted with ethyl acetate (50 mL×3). The combined organic phase was washed with brine (50 mL), dried with anhydrous Na₂SO₄, filtered and concentrated in vacuum. The residue was purified by column chromatography (SiO₂, petroleum ether/ethyl acetate=20/1 to 0:1). Compound tert-butyl N-[4-[8-bromo-2-butyl-4-[(2,4-dimethoxyphenyl) methylamino] imidazo[4,5-c]quinolin-1-yl]butyl]carbamate (0.97 g, 1.51 mmol, 97% yield) was obtained as a yellow solid. ¹H NMR (MeOD, 400 MHz) δ 8.19 (s, 1H), 7.91-7.70 (m, 2H), 7.28 (d, J=8.4 Hz, 1H), 6.59 (s, 1H), 6.51 (d, J=8.4 Hz, 1H), 4.84-4.78 (m, 2H), 4.56 (t, J=7.6 Hz, 2H), 3.83 (s, 3H), 3.78 (s, 3H), 3.17-3.06 (m, 2H), 2.98 (t, J=7.8 Hz, 2H), 1.98-1.86 (m, 4H), 1.70-1.60 (m, 2H), 1.54-1.49 (m, 2H), 1.36 (s, 9H), 1.01 (t, J=7.6 Hz, 2H).

### Example 22: Synthesis of Compound 23

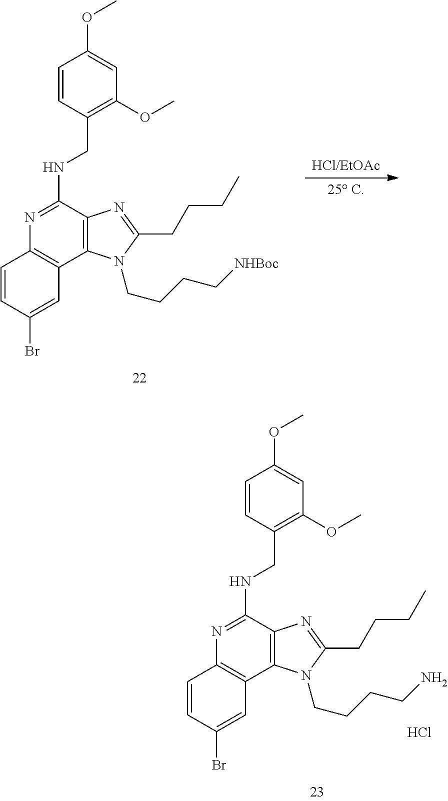 [SMILES: CCCCc1nc2c(NCc3ccc(OC)cc3OC)nc3ccc(Br)cc3c2n1CCCCNC(=O)OC(C)(C)C ; "25° C." ; "HCl/EtOAc" ; CCCCc1nc2c(NCc3ccc(OC)cc3OC)nc3ccc(Br)cc3c2n1CCCCN ; Cl]

A solution of tert-butyl N-[4-[8-bromo-2-butyl-4-[(2,4-dimethoxyphenyl) methylamino]imidazo[4,5-c]quinolin-1-yl]butyl]carbamate (0.47 g, 733.68 μmol, 1 eq) in HCl/EtOAc (4 M, 50 mL) was stirred at 25° C. for 2 hours. The mixture was concentrated under reduced pressure at 45° C. Compound 1-(4-aminobutyl)-8-bromo-2-butyl-N-[(2,4-dimethoxyphenyl)methyl]imidazo[4,5-c]quinolin-4-amine (0.5 g, crude, HCl) was obtained as a yellow solid. LCMS (ESI): mass calcd. for C₂₇H₃₄N₅O₂Br 539.2/541.2, m/z found 540.3/542.3 [M+H]⁺

### Example 23: Synthesis of Compound 24

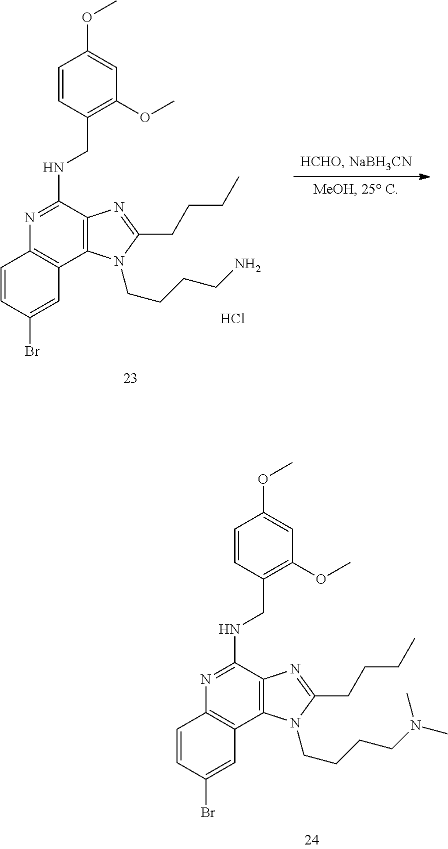

A mixture of 1-(4-aminobutyl)-8-bromo-2-butyl-N-[(2,4-dimethoxyphenyl) methyl]imidazo[4,5-c]quinolin-4-amine (0.4 g, 740.06 μmol, 1 eq) and triethylamine (TEA; 149.77 mg, 1.48 mmol, 206.02 μL, 2 eq) in MeOH (20 mL) was stirred at 25° C. for 30 min then formaldehyde (132.13 mg, 1.63 mmol, 121.22 μL, 2.2 eq) and AcOH (44.44 mg, 740.06 μmol, 42.32 μL, 1 eq) was added to the mixture stirred 30 min at 25° C. NaBH₃CN (186.02 mg, 2.96 mmol, 4 eq) was added to the mixture at 0° C. and stirred at 25° C. for 1.5 hours. The mixture was concentrated under reduced pressure at 45° C. The residue was added water (50 mL) and extracted with ethyl acetate (50 mL×3). The combined organic phase was washed with brine (50 mL), dried with anhydrous Na₂SO₄, filtered and concentrated in vacuum. Compound 8-bromo-2-butyl-N-[(2,4-dimethoxyphenyl)methyl]-1-[4-(dimethylamino)butyl]imidazo[4,5-c]quinolin-4-amine (0.5 g, crude) was obtained as a yellow solid. ¹H NMR (MeOD, 400 MHz) δ 8.06 (m, 1H), 7.71 (d, J=9.0 Hz, 1H), 7.54 (d, J=10.4 Hz, 1H), 7.29 (d, J=8.4 Hz, 1H), 6.56 (d, J=2.4 Hz, 1H), 6.46 (dd, J=2.4, 8.4 Hz, 1H), 4.74 (s, 2H), 4.48-4.46 (m, 2H), 3.86 (s, 3H), 3.77 (s, 3H), 2.96-2.92 (m, 2H), 2.43-2.40 (m, 2H), 2.26 (s, 6H), 1.94-1.81 (m, 4H), 1.70-1.68 (m, 2H), 1.56-1.47 (m, 2H), 1.24 (t, J=7.2 Hz, 3H), 1.01 (t, J=7.2 Hz, 3H).

### Example 24: Synthesis of Compound 25

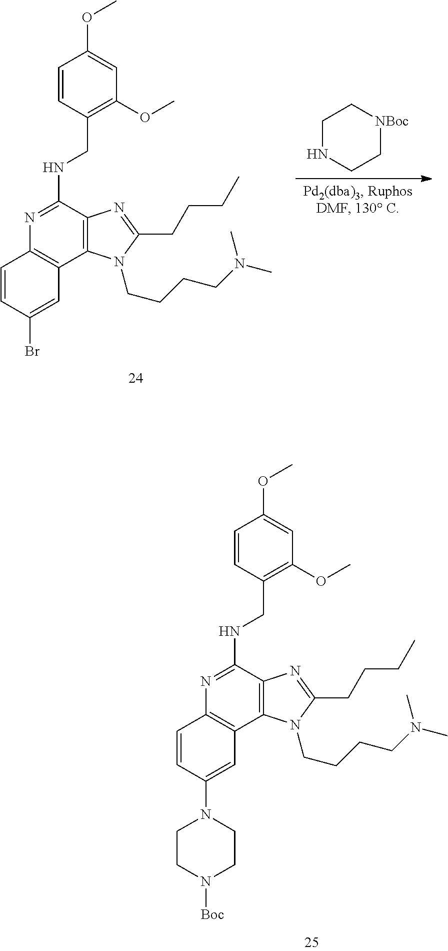

To a mixture of 8-bromo-2-butyl-N-[(2,4-dimethoxyphenyl)methyl]-1-[4-(dimethylamino)butyl]imidazo[4,5-c]quinolin-4-amine (0.48 g, 844.26 μmol, 1 eq) and tert-butyl piperazine-1-carboxylate (786.22 mg, 4.22 mmol, 5 eq) in DMF (50 mL) were added Cs₂CO₃ (550.15 mg, 1.69 mmol, 2 eq), RuPhos (39.40 mg, 84.43 μmol, 0.1 eq) and Pd₂(dba)₃ (38.66 mg, 42.21 μmol, 0.05 eq) in one portion at 25° C. under N₂. The mixture was stirred at 120° C. for 2 hours. The mixture was added H₂O (150 mL) and extracted with ethyl acetate (50 mL×3). The combined organic phase was washed with brine (50 mL×2), dried with anhydrous Na₂SO₄, filtered and concentrated in vacuum. Compound tert-butyl 4-[2-butyl-4-[(2,4-dimethoxyphenyl)methylamino]-1-[4-(dimethylamino)butyl]imidazo[4,5-c]quinolin-8-yl]piperazine-1-carboxylate (0.5 g, crude) was obtained as a yellow solid. LCMS (ESI): mass calcd. for C₃₈H₅₅N₇O₄ 673.4, m/z found 674.4 [M+H]⁺.

### Example 25: Synthesis of Compound 26

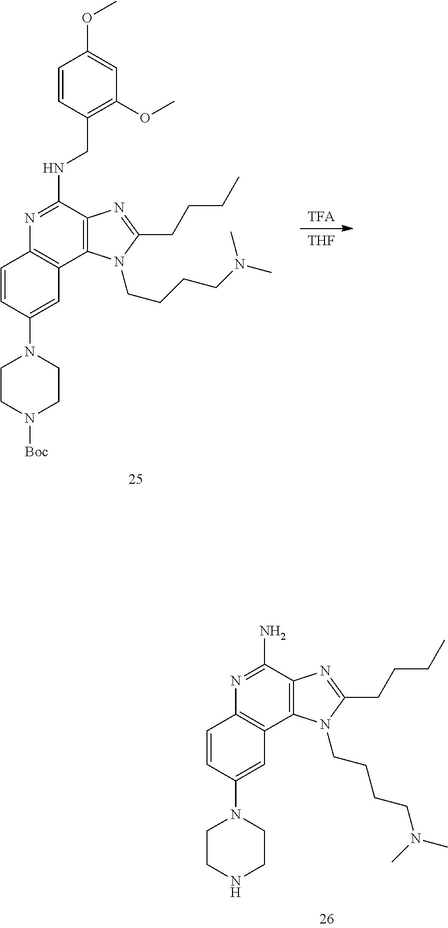 [SMILES: CCCCc1nc2c(NCc3ccc(OC)cc3OC)nc3ccc(N4CCN(C(=O)OC(C)(C)C)CC4)cc3c2n1CCCCN(C)C ; "TFA" ; "THF" ; CCCCc1nc2c(N)nc3ccc(N4CCNCC4)cc3c2n1CCCCN(C)C]

To a solution of tert-butyl 4-[2-butyl-4-[(2,4-dimethoxyphenyl) methylamino]-1-[4-(dimethylamino)butyl]imidazo[4,5-c]quinolin-8-yl]piperazine-1-carboxylate (0.5 g, 741.97 μmol, 1 eq) in DCM (20 mL) was added TFA (2.57 g, 22.51 mmol, 1.67 mL, 30.34 eq) in one portion at 25° C. The mixture was stirred at 40° C. for 12 hours. The mixture was concentrated in reduced pressure at 45° C. The residue was purified by prep-HPLC (column: LUNA™ C18 100×30 5u (Phenomenex, Inc., Torrance, Calif.); mobile phase: [water (0.1% TFA)-ACN]; B %: 5%-30%, 10 min). Compound 2-butyl-1-[4-(dimethylamino)butyl]-8-piperazin-1-yl-imidazo[4,5-c]quinolin-4-amine (0.130 g, 306.90 μmol, 41.36% yield) was obtained as a yellow oil. The compound was used in the subsequent step without further purification.

### Example 26: Synthesis of Compound 27

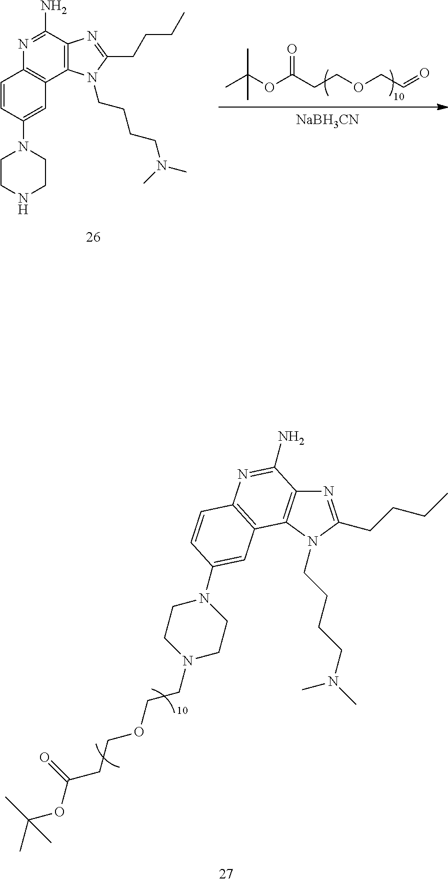

To a solution of 2-butyl-1-[4-(dimethylamino) butyl]-8-piperazin-1-yl-imidazo [4,5-c]quinolin-4-amine (0.13 g, 199.50 μmol, 1 eq, 2TFA) in MeOH (20 mL) was added TEA (40.37 mg, 398.99 μmol, 55.54 μL, 2 eq) at 25° C. and the mixture was stirred for 0.5 hour. Then tert-butyl 3-[2-[2-[2-[2-[2-[2-[2-[2-[2-(2-oxoethoxy) ethoxy]ethoxy]ethoxy]ethoxy]ethoxy]ethoxy]ethoxy]ethoxy]ethoxy]propanoate (116.64 mg, 199.50 μmol, 1 eq), AcOH (11.98 mg, 199.50 μmol, 11.41 μL, 1 eq) and NaBH₃CN (25.07 mg, 398.99 μmol, 2 eq) were added at 25° C. and stirred for 11.5 hours. The mixture was added H₂O (2 mL) and concentrated in reduced pressure at 50° C. Compound tert-butyl 3-[2-[2-[2-[2-[2-[2-[2-[2-[2-[2-[4-[4-amino-2-butyl-1-[4-(dimethylamino)butyl]imidazo[4,5-c]quinolin-8-yl]piperazin-1-yl]ethoxy]ethoxy]ethoxy]ethoxy]ethoxy]ethoxy]ethoxy]ethoxy]ethoxy]ethoxy]propanoate (0.2 g, crude) was obtained as a yellow oil. LC/MS [M+H] 992.66 (calculated); LC/MS [M+H] 993.08 (observed).

### Example 27: Synthesis of Compound 28

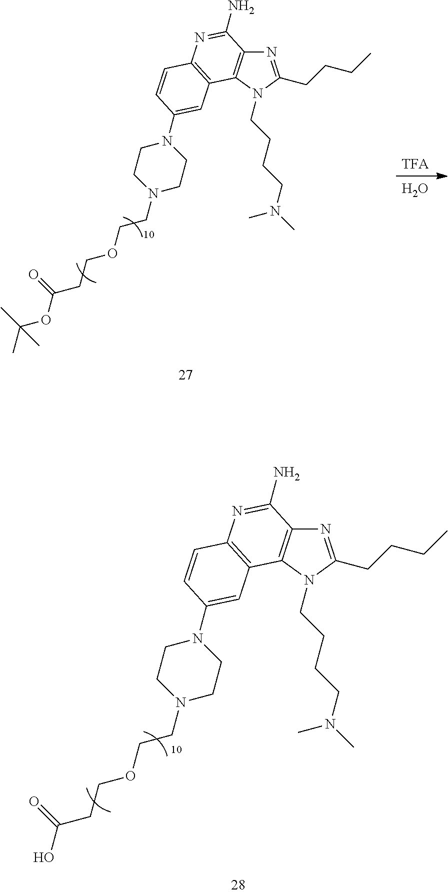

To a solution of tert-butyl 3-[2-[2-[2-[2-[2-[2-[2-[2-[2-[2-[4-[4-amino-2-butyl-1-[4-(dimethylamino)butyl]imidazo[4,5-c]quinolin-8-yl]piperazin-1-yl]ethoxy]ethoxy]ethoxy]ethoxy]ethoxy]ethoxy]ethoxy]ethoxy]ethoxy]ethoxy]propanoate (0.2 g, 201.55 μmol, 1 eq) in H₂O (10 mL) was added TFA (22.98 mg, 201.55 μmol, 14.92 μL, 1 eq) at 60° C. and stirred for 12 hours. The mixture was concentrated under reduced pressure at 50° C. The residue was purified by prep-HPLC (column: Nano-micro KROMASIL′ (Sigma-Aldrich, St. Louis, Mo.) C18 100×30 mm Sum; mobile phase: [water (0.1% TFA)-ACN]; B %: 15%-35%, 10 min). Compound 3-[2-[2-[2-[2-[2-[2-[2-[2-[2-[2-[4-[4-amino-2-butyl-1-[4-(dimethylamino)butyl]imidazo[4,5-c]quinolin-8-yl]piperazin-1-yl]ethoxy]ethoxy]ethoxy]ethoxy]ethoxy]ethoxy]ethoxy]ethoxy]ethoxy]ethoxy]propanoic acid (0.0887 g, 94.75 μmol, 47.01% yield, 100% purity) was obtained as a yellow oil. ¹H NMR (MeOD, 400 MHz) δ 7.76 (d, J=9.2 Hz, 1H), 7.56 (d, J=9.2 Hz, 1H), 7.51 (s, 1H), 4.73 (t, J=7.2 Hz 2H), 4.09-3.78 (m, 6H), 3.76-3.45 (m, 44H), 3.25-3.15 (m, 2H), 3.04 (t, J=7.6 Hz, 2H), 2.88 (s, 6H), 2.52 (t, J=6.0 Hz, 2H), 2.12-1.89 (m, 6H), 1.57-1.53 (m, 2H), 1.04 (t, J=7.2 Hz, 3H). LCMS (ESI): mass calcd. for C₄₁H₈₁N₇O₁₂=935.6, m/z found 936.2 [M+H]⁺.

### Example 28: Synthesis of Compound 29

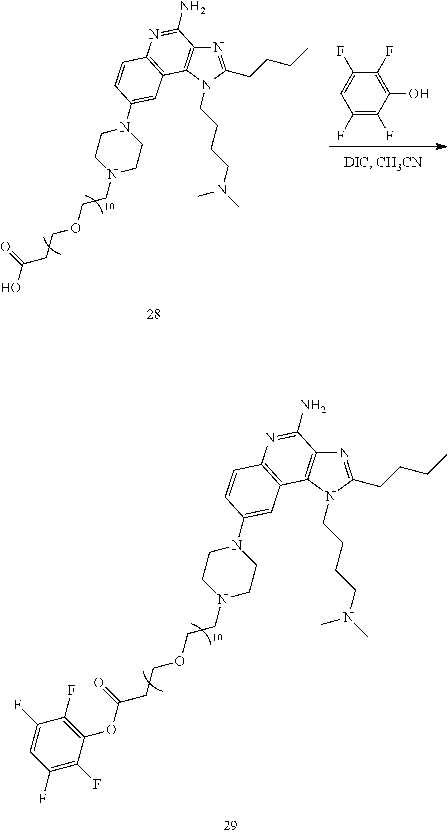

1-(4-(4-amino-2-butyl-1-(4-(dimethylamino)butyl)-1H-imidazo[4,5-c]quinolin-8-yl)piperazin-1-yl)-3,6,9,12,15,18,21,24,27,30-decaoxatritriacontan-33-oic acid HCl was converted into 2,3,5,6-tetrafluorophenyl 1-(4-(4-amino-2-butyl-1-(4-(dimethylamino)butyl)-1H-imidazo[4,5-c]quinolin-8-yl)piperazin-1-yl)-3,6,9,12,15,18,21,24,27,30-decaoxatritriacontan-33-oate in a 46% yield using the procedure described in Example 17. LC/MS [M+H] 1084.60 (calculated); LC/MS [M+H] 1084.86 (observed).

### Example 29: Synthesis of Compound 30

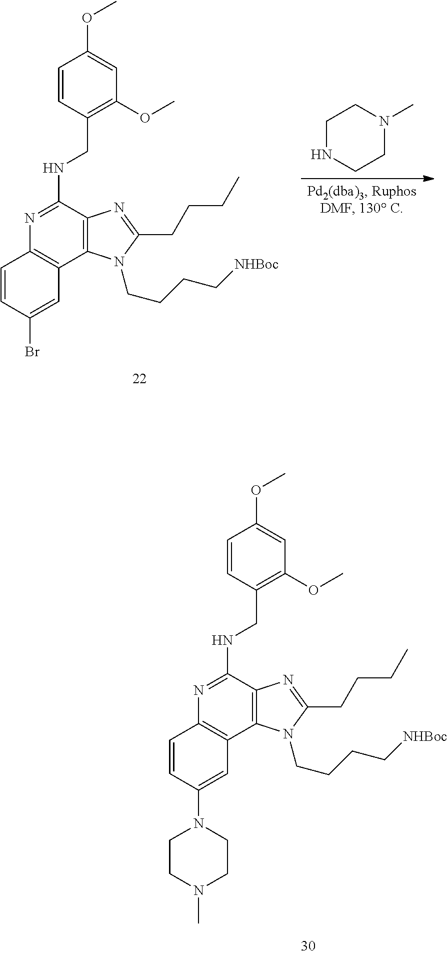

To a mixture of tert-butyl N-[4-[8-bromo-2-butyl-4-[(2,4-dimethoxyphenyl) methylamino]imidazo[4,5-c]quinolin-1-yl]butyl]carbamate (0.5 g, 780.51 μmol, 1 eq) and 1-methylpiperazine (234.53 mg, 2.34 mmol, 259.72 μL, 3 eq) in DMF (20 mL) were added Cs₂CO₃ (508.61 mg, 1.56 mmol, 2 eq), RuPhos (36.42 mg, 78.05 μmol, 0.1 eq) and Pd₂(dba)₃ (35.74 mg, 39.03 μmol, 0.05 eq) in one portion at 25° C. under N₂. The mixture was stirred at 120° C. for 2 hours. To the mixture was added H₂O (80 mL) and extracted with ethyl acetate (50 mL×3). The combined organic phase was washed with brine (30 mL×2), dried with anhydrous Na₂SO₄, filtered and concentrated in vacuum. The residue was purified by column chromatography (SiO₂, ethyl acetate:MeOH=10:1). Compound tert-butyl N-[4-[2-butyl-4-[(2,4-dimethoxyphenyl)methylamino]-8-(4-methylpiperazin-1-yl)imidazo[4,5-c]quinolin-1-yl]butyl]carbamate (0.27 g, 409.18 μmol, 52.42% yield) was obtained as a yellow oil. ¹H NMR (MeOD, 400 MHz) δ 7.77 (d, J=9.2 Hz, 1H), 7.37 (s, 1H), 7.34-7.27 (m, 2H), 6.56 (s, 1H), 6.46 (d, J=8.4 Hz, 1H), 4.72 (s, 2H), 4.56-4.45 (m, 2H), 3.85 (s, 3H), 3.80 (s, 3H), 3.31-3.28 (m, 2H), 2.93 (t, J=7.6 Hz, 1H), 2.70-2.68 (m, 4H), 2.39 (s, 3H), 1.93-1.91 (m, 2H), 1.87-1.77 (m, 2H), 1.64-1.62 (m, 2H), 1.55-1.46 (m, 2H), 1.35 (s, 9H), 1.24 (t, J=7.6 Hz, 2H), 1.01 (t, J=7.2 Hz, 3H).

### Example 30: Synthesis of Compound 31

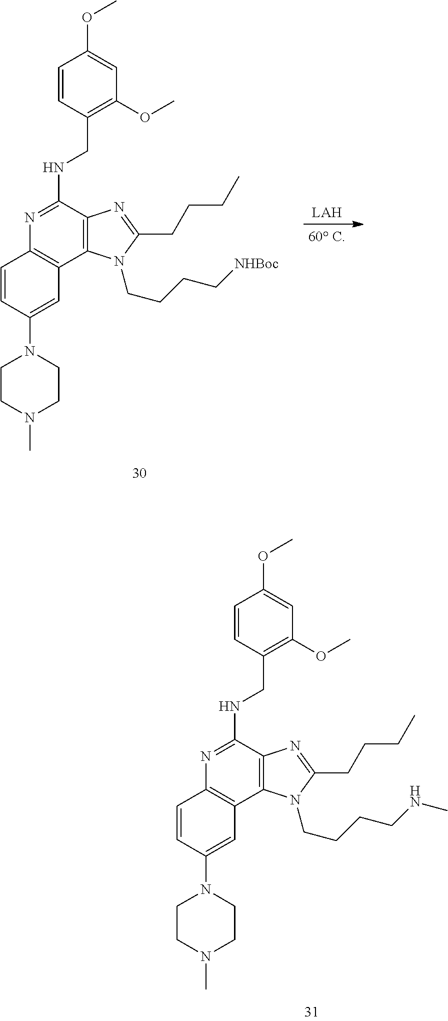 [SMILES: CCCCc1nc2c(NCc3ccc(OC)cc3OC)nc3ccc(N4CCN(C)CC4)cc3c2n1CCCCNC(=O)OC(C)(C)C ; "60° C." ; "LAH" ; CCCCc1nc2c(NCc3ccc(OC)cc3OC)nc3ccc(N4CCN(C)CC4)cc3c2n1CCCCNC]

To a solution of tert-butyl N-[4-[2-butyl-4-[(2,4-dimethoxyphenyl)methylamino]-8-(4-methylpiperazin-1-yl)imidazo[4,5-c]quinolin-1-yl]butyl]carbamate (0.22 g, 333.40 μmol, 1 eq) in THF (20 mL) was added LiAlH₄ (63.27 mg, 1.67 mmol, 5 eq) in portions at 25° C. under N₂. The mixture was stirred at 60° C. for 3 hours. The mixture was added saturated aqueous Na₂SO₄ (2 mL) at 0° C. and dried with anhydrous Na₂SO₄, filtered and concentrated in vacuum. Compound 2-butyl-N-[(2,4-dimethoxyphenyl) methyl]-1-[4-(methylamino)butyl]-8-(4-methylpiperazin-1-yl)imidazo[4,5-c]quinolin-4-amine (0.2 g, crude) was obtained as a yellow oil. ¹H NMR (CDCl₃, 400 MHz) δ 7.82 (d, J=9.2 Hz, 1H), 7.41 (d, J=8.2 Hz, 1H), 7.32 (d, J=2.4 Hz, 1H), 7.24 (d, J=2.4 Hz, 1H), 6.47 (d, J=2.2 Hz, 1H), 6.42 (dd, J=2.4, 8.2 Hz, 1H), 4.83 (s, 2H), 4.43 (t, J=3.6 Hz, 2H), 4.16-4.06 (m, 2H), 3.85 (s, 3H), 3.79 (s, 3H), 3.73-3.63 (m, 2H), 3.31-3.23 (m, 4H), 2.87 (t, J=8.0 Hz, 2H), 2.71-2.64 (m, 4H), 2.41 (d, J=7.2 Hz, 4H), 2.01-1.87 (m, 2H), 1.90-1.80 (m, 2H), 1.76-1.63 (m, 4H), 0.99 (t, J=7.6 Hz, 3H).

### Example 31: Synthesis of Compound 32

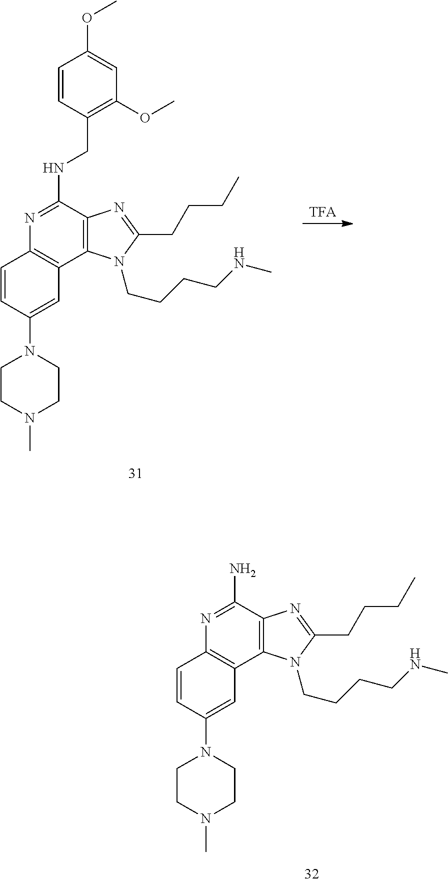 [SMILES: CCCCc1nc2c(NCc3ccc(OC)cc3OC)nc3ccc(N4CCN(C)CC4)cc3c2n1CCCCNC ; "TFA" ; CCCCc1nc2c(N)nc3ccc(N4CCN(C)CC4)cc3c2n1CCCCNC]

To a solution of 2-butyl-N-[(2,4-dimethoxyphenyl) methyl]-1-[4-(methylamino) butyl]-8-(4-methylpiperazin-1-yl)imidazo[4,5-c]quinolin-4-amine (0.2 g, 348.57 μmol, 1 eq) in DCM (20 mL) was added TFA (2.80 g, 24.56 mmol, 1.82 mL, 70.45 eq) in one portion at 25° C. The mixture was stirred at 40° C. for 12 hours. The mixture was concentrated in reduced pressure at 45° C. The residue was purified by prep-HPLC (column: LUNA™ C18 100×30 5u (Phenomenex, Inc.); mobile phase: [water (0.1% TFA)-ACN]; B %: 5%-25%, 10 min). Compound 2-butyl-1-[4-(methylamino) butyl]-8-(4-methylpiperazin-1-yl) imidazo[4,5-c]quinolin-4-amine (0.2 g, crude) was obtained as a yellow oil. LC/MS [M+H] 424.32 (calculated); LC/MS [M+H] 424.43 (observed).

### Example 32: Synthesis of Compound 33

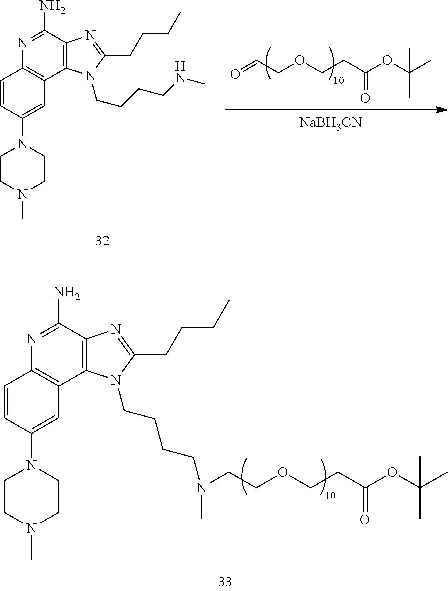

To a solution of 2-butyl-1-[4-(methylamino)butyl]-8-(4-methylpiperazin-1-yl) imidazo[4,5-c]quinolin-4-amine (0.1 g, 153.46 μmol, 1 eq, 2TFA) in MeOH (20 mL) was added TEA (31.06 mg, 306.92 μmol, 42.72 μL, 2 eq) at 25° C. and stirred for 0.5 hour. Then tert-butyl 3-[2-[2-[2-[2-[2-[2-[2-[2-[2-(2-oxoethoxy)ethoxy]ethoxy]ethoxy]ethoxy]ethoxy]ethoxy]ethoxy]ethoxy]ethoxy]propanoate (89.73 mg, 153.46 μmol, 1 eq), AcOH (9.22 mg, 153.46 μmol, 8.78 μL, 1 eq) and NaBH₃CN (19.29 mg, 306.92 μmol, 2 eq) were added at 25° C. and stirred for 11.5 hours. The mixture was added to H₂O (2 mL) and concentrated in reduced pressure at 50° C. Compound tert-butyl 3-[2-[2-[2-[2-[2-[2-[2-[2-[2-[2-[4-[4-amino-2-butyl-8-(4-methylpiperazin-1-yl)imidazo[4,5-c]quinolin-1-yl]butyl-methylamino]ethoxy]ethoxy]ethoxy]ethoxy]ethoxy]ethoxy]ethoxy]ethoxy]ethoxy]ethoxy]propanoate (0.2 g, crude) was obtained as a yellow oil, which was used without purification in the next step.

### Example 33: Synthesis of Compound 34

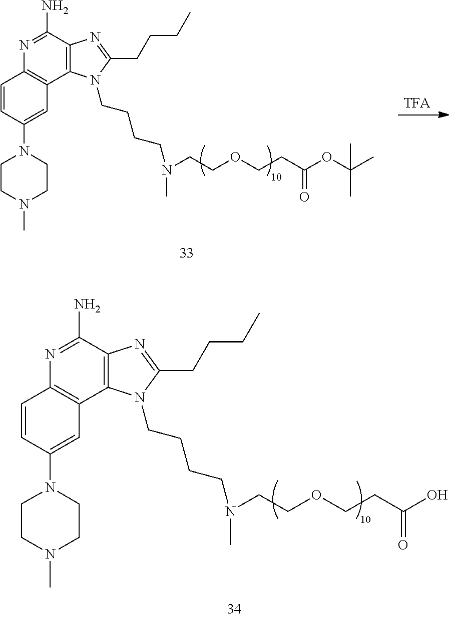

To a solution of tert-butyl 3-[2-[2-[2-[2-[2-[2-[2-[2-[2-[2-[4-[4-amino-2-butyl-8-(4-methylpiperazin-1-yl)imidazo[4,5-c]quinolin-1-yl]butyl-methylamino]ethoxy]ethoxy]ethoxy]ethoxy]ethoxy]ethoxy]ethoxy]ethoxy]ethoxy]ethoxy]propanoate (0.15 g, 151.17 μmol, 1 eq) in H₂O (10 mL) was added TFA (1.54 g, 13.51 mmol, 1 mL, 89.35 eq) at 25° C. and stirred at 60° C. for 12 hours. The mixture was concentrated in reduced pressure at 50° C. The residue was purified by prep-HPLC (column: Nano-micro KROMASIL™ (Sigma-Aldrich) C18 100×30 mm Sum; mobile phase: [water (0.1% TFA)-ACN]; B %: 15%-35%, 10 min). Compound 3-[2-[2-[2-[2-[2-[2-[2-[2-[2-[2-[4-[4-amino-2-butyl-8-(4-methylpiperazin-1-yl)imidazo[4,5-c]quinolin-1-yl]butyl-methylamino]ethoxy]ethoxy]ethoxy]ethoxy]ethoxy]ethoxy]ethoxy]ethoxy]ethoxy]ethoxy]propanoic acid (0.0444 g, 47.43 μmol, 31.37% yield, 100% purity) was obtained as a yellow oil. ¹H NMR (MeOD, 400 MHz) δ 7.77 (d, J=9.2 Hz, 1H), 7.60-7.50 (m, 2H), 4.75 (t, J=7.0 Hz, 2H), 4.00 (d, J=10.4 Hz, 2H), 3.82 (t, J=4.8 Hz, 2H), 3.74-3.70 (m, 4H), 3.65-3.49 (m, 40H), 3.05 (t, J=7.8 Hz, 2H), 3.02 (s, 3H), 2.92 (s, 3H), 2.53 (t, J=6.2 Hz, 2H), 2.09-1.87 (m, 6H), 1.61-1.52 (m, 2H), 1.05 (t, J=7.6 Hz, 3H). LCMS (ESI): mass calcd. for C₄₇H₈₁N₇O₁₂ 935.6, m/z found 936.2 [M+H]⁺.

### Example 34: Synthesis of Compound 35

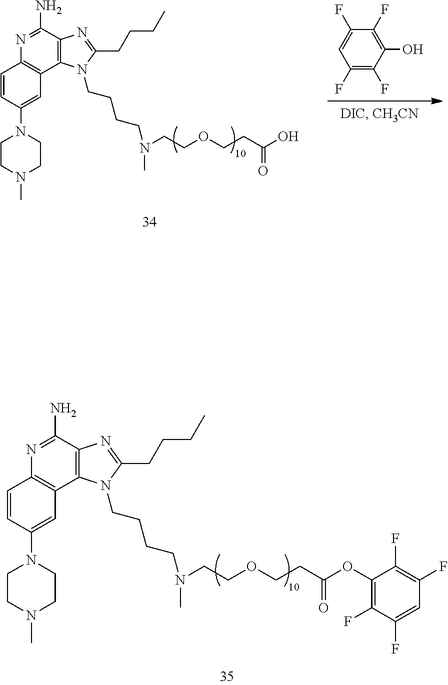

38-(4-amino-2-butyl-8-(4-methylpiperazin-1-yl)-1H-imidazo[4,5-c]quinolin-1-yl)-34-methyl-4,7,10,13,16,19,22,25,28,31-decaoxa-34-azaoctatriacontanoic acid HCl was converted into 2,3,5,6-tetrafluorophenyl 38-(4-amino-2-butyl-8-(4-methylpiperazin-1-yl)-1H-imidazo[4,5-c]quinolin-1-yl)-34-methyl-4,7,10,13,16,19,22,25,28,31-decaoxa-34-azaoctatriacontanoate TFA in a 52% yield using the procedure described in Example 17. LC/MS [M+Na] 1106.58 (calculated); LC/MS [M+Na] 1107.00 (observed).

### Example 35: Synthesis of Compound 36

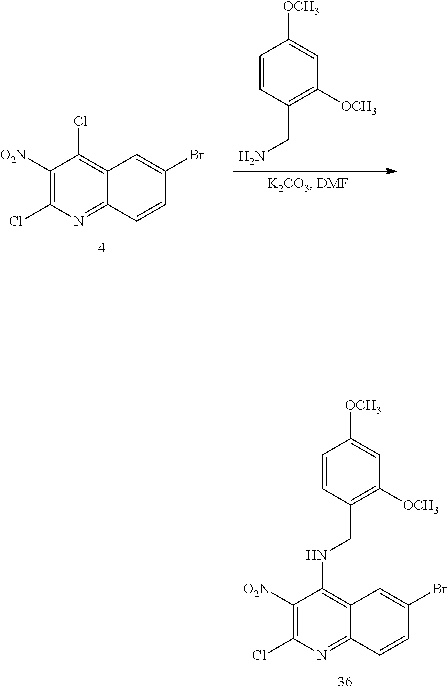

To a solution of 6-bromo-2,4-dichloro-3-nitroquinoline (5.6 g, 17.4 mmol, 1 eq.) and solid K₂CO₃ (3.6 g, 26 mmol, 1.5 eq.) in DMF (100 mL) at room temperature was added neat 2,4-dimethoxybenzylamine (3.5 g, 20.1 mmol, 1.2 eq.). The mixture was stirred for 15 minutes, water (300 mL) was added and the mixture was stirred for 5 additional minutes. The resultant solid was filtered and then dissolved in ethyl acetate (100 mL). The solution was washed with water (100 mL), brine (100 mL), separated, dried (Na₂SO₄), then filtered and concentrated in vacuo. The brown solid was triturated with 1:1 hexanes/diethyl ether (150 mL) and filtered to obtain 6-bromo-2-chloro-4-(2,4-dimethoxybenzyl)amino-3-nitroquinoline (6.9 g, 15.3 mmol, 88%) as a yellow solid. The compound was used without further purification.

### Example 36: Synthesis of Compound 37

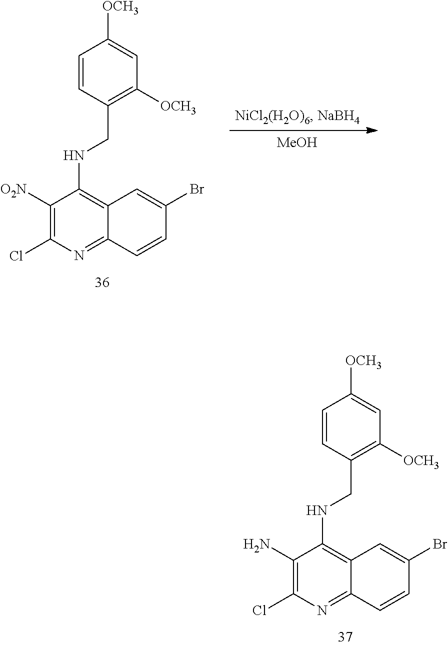

To 6-bromo-2-chloro-4-(2,4-dimethoxybenzyl)amino-3-nitroquinoline (6.9 g, 15.3 mmol, 88%) in methanol (200 mL) at 0° C. was added NiCl₂.6H₂O (0.36 g, 1.5 mmol, 0.1 eq). Sodium borohydride (pellets, 1.42 g, 38 mmol, 2.5 eq.) was added and reaction was stirred for 1 h at 0° C. then warmed to room temperature and allowed to stir for another 15 minutes. Glacial acetic acid (5 mL) was added until a pH of −5 was obtained. The solvent was evaporated in vacuo and the crude solid was redissolved in ethyl acetate (150 mL) then filtered through a bed of diatomaceous earth to remove a black insoluble material. The ethyl acetate was removed in vacuo. The dark brown solid was triturated with ether (75 mL) then filtered to obtain 3-amino-6-bromo-2-chloro-4-(2,4-dimethoxybenzyl)aminoquinoline (5.81 g, 13.7 mmol, 90%) as a tan solid. The compound was used without further purification.

### Example 37: Synthesis of Compound 38

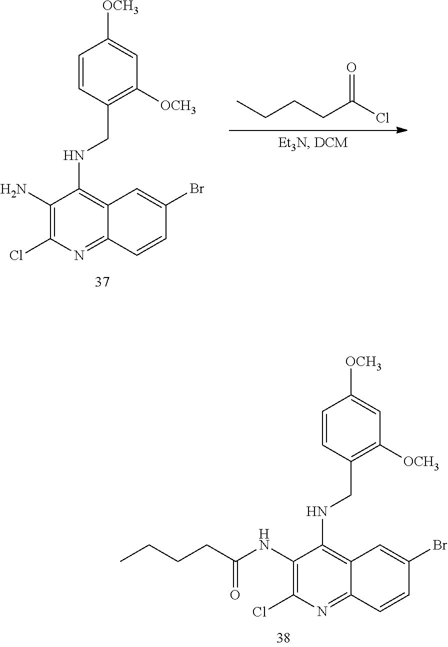

To a solution of 3-amino-6-bromo-2-chloro-4-(2,4-dimethoxybenzyl)aminoquinoline (5.75 g, 13.6 mmol, 1 eq.) in dichloromethane (100 mL) containing triethylamine (2.1 g, 2.8 mL, 20 mmol, 1.5 eq.) stirring at room temperature was added neat valeroyl chloride (2.0 mL, 2.0 g, 16 mmol, 1.2 eq). The mixture was washed with water (150 mL), brine (150 mL), separated, dried (Na₂SO₄), filtered, and concentrated. The solid was triturated with ether, filtered and dried under vacuum. N-(6-bromo-2-chloro-4-((2,4-dimethoxybenzyl)amino)quinolin-3-yl)pentanamide was obtained as a brown solid (5.8 g, 11.4 mmol, 84%). The compound was used without further purification.

### Example 38: Synthesis of Compound 39

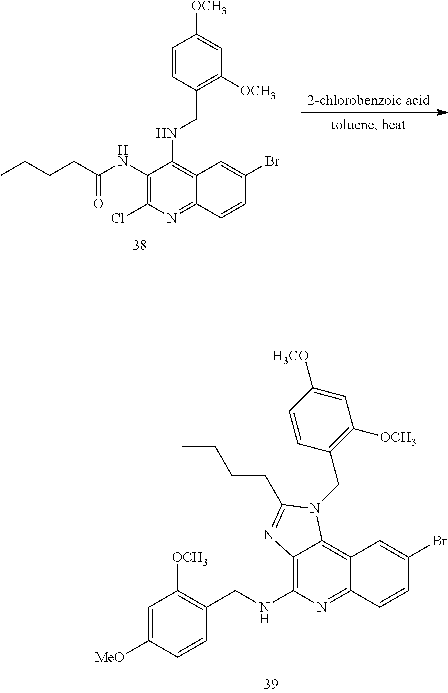 [SMILES: CCCCC(=O)Nc1c(Cl)nc2ccc(Br)cc2c1NCc1ccc(OC)cc1OC ; "2-chlorobenzoic acid" ; "toluene, heat" ; CCCCc1nc2c(NCc3ccc(OC)cc3OC)nc3ccc(Br)cc3c2n1Cc1ccc(OC)cc1OC]

In a 100 mL beaker a mixture of N-(6-bromo-2-chloro-4-((2,4-dimethoxybenzyl)amino)quinolin-3-yl)pentanamide (5.8 g, 11.4 mmol, 1 eq.) and 2-chlorobenzoic (0.90 g, 5.7 mmol. 0.5 eq.) was boiled in 50 mL toluene for 2 hours. Toluene was added to 50 mL each time the volume reached 25 mL. 2,4-dimethoxybenzylamine (9.5 g, 57 mmol, 5 eq.) was added and the reaction was maintained at 120° C. for 2 hours. The reaction was cooled to room temperature and water (80 mL) then acetic acid (3.5 mL) was added. The supernatant was decanted and the crude product was washed with water (80 mL). The wet solid was triturated with methanol (100 mL) to provide 8-bromo-2-butyl-N,1-bis(2,4-dimethoxybenzyl)-1H-imidazo[4,5-c]quinolin-4-amine (4.80 g, 7.7 mmol, 68%) as an off-white solid. The compound was used without further purification.

### Example 39: Synthesis of Compound 40

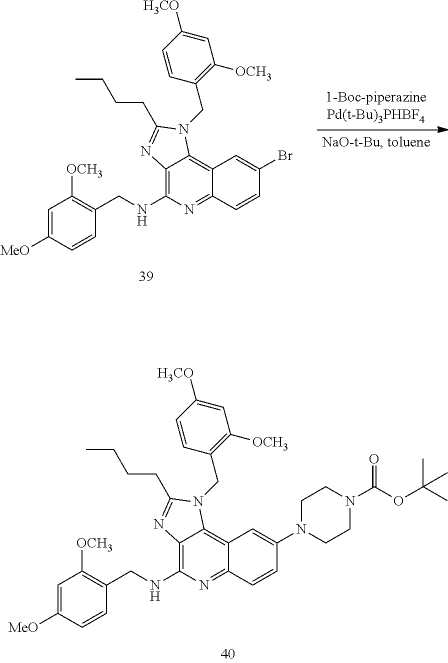 [SMILES: CCCCc1nc2c(NCc3ccc(OC)cc3OC)nc3ccc(Br)cc3c2n1Cc1ccc(OC)cc1OC ; "1-Boc-piperazinePd(t-Bu)3PHBF4" ; "NaO-t-Bu, toluene" ; CCCCc1nc2c(NCc3ccc(OC)cc3OC)nc3ccc(N4CCN(C(=O)OC(C)(C)C)CC4)cc3c2n1Cc1ccc(OC)cc1OC]

A mixture of 8-bromo-2-butyl-N,1-bis(2,4-dimethoxybenzyl)-1H-imidazo[4,5-c]quinolin-4-amine (0.31 g, 0.5 mmol, 1 eq.) and tert-butyl piperazine-1-carboxylate (0.19 g, 1 mmol, 2 eq.) were combined in toluene (2 mL) then degassed with argon. Pd₂dba₃ (45 mg, 0.05 mmol, 0.1 eq.), tri-tert-butylphosphine tetrafluoroborate (29 mg, 0.10 mmol, 0.2 eq) and sodium tert-butoxide (144 mg, 1.5 mmol, 3 eq) were added. The mixture was heated in a capped vial at 110° C. for 30 minutes. The mixture was cooled then partitioned between ethyl acetate (50 mL) and water (50 mL). The organic layer was washed with brine (50 mL), dried with sodium sulfate, filtered, and concentrated in vacuo. The crude product was purified on silica gel (20 g) eluted with 50% ethyl acetate/hexanes to yield tert-butyl 4-(2-butyl-1-(2,4-dimethoxybenzyl)-4-((2,4-dimethoxybenzyl)amino)-1H-imidazo[4,5-c]quinolin-8-yl)piperazine-1-carboxylate (0.28 g, 0.39 mmol, 78%) as an off-white solid. LC/MS [M+H] 725.40 (calculated); LC/MS [M+H] 725.67 (observed).

### Example 40: Synthesis of Compound 41

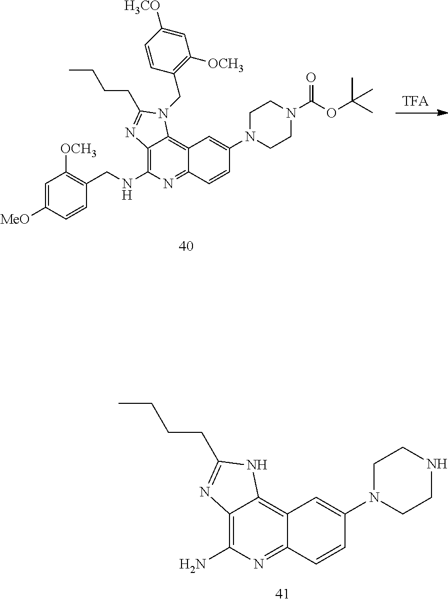

Tert-butyl 4-(2-butyl-1-(2,4-dimethoxybenzyl)-4-((2,4-dimethoxybenzyl)amino)-1H-imidazo[4,5-c]quinolin-8-yl)piperazine-1-carboxylate (0.28 g, 0.39 mmol, 1 eq.) was dissolved in TFA (3 mL) and heated to reflux for 5 min. The TFA was removed in vacuo and the crude product was dissolved in acetonitrile, filtered then concentrated to obtain the TFA salt of 2-butyl-8-(piperazin-1-yl)-1H-imidazo[4,5-c]quinolin-4-amine (0.16 g, 0.37 mmol, 95%) as an off-white solid. LC/MS [M+H] 325.21 (calculated); LC/MS [M+H] 325.51 (observed).

### Example 41: Synthesis of Compound 42

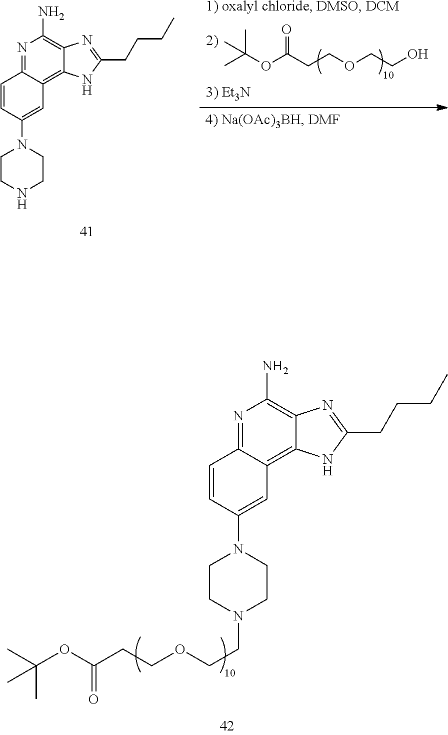

2-butyl-8-(piperazin-1-yl)-1H-imidazo[4,5-c]quinolin-4-amine was converted into tert-butyl 1-(4-(4-amino-2-butyl-1H-imidazo[4,5-c]quinolin-8-yl)piperazin-1-yl)-3,6,9,12,15,18,21,24,27,30-decaoxatritriacontan-33-oate in a 56% yield using the procedure described in Example 12. LC/MS [M+H] 893.55 (calculated); LC/MS [M+H] 893.79 (observed).

### Example 42: Synthesis of Compound 43

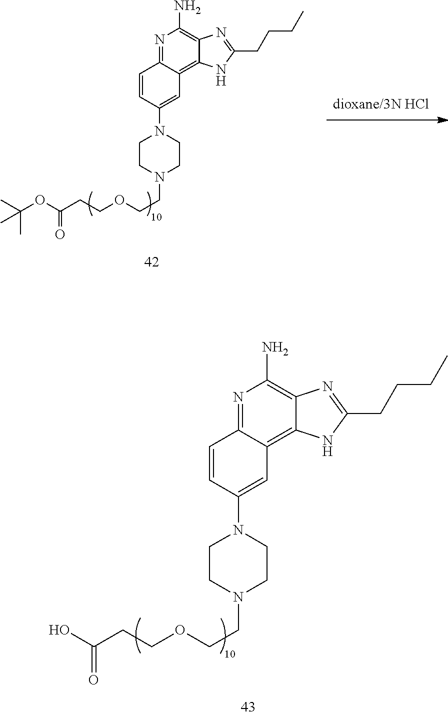

Tert-butyl 1-(4-(4-amino-2-butyl-1H-imidazo[4,5-c]quinolin-8-yl)piperazin-1-yl)-3,6,9,12,15,18,21,24,27,30-decaoxatritriacontan-33-oate was converted into 1-(4-(4-amino-2-butyl-1H-imidazo[4,5-c]quinolin-8-yl)piperazin-1-yl)-3,6,9,12,15,18,21,24,27,30-decaoxatritriacontan-33-oic acid in a 93% yield using the procedure described in Example 16. The compound was used without further purification.

### Example 43: Synthesis of Compound 44

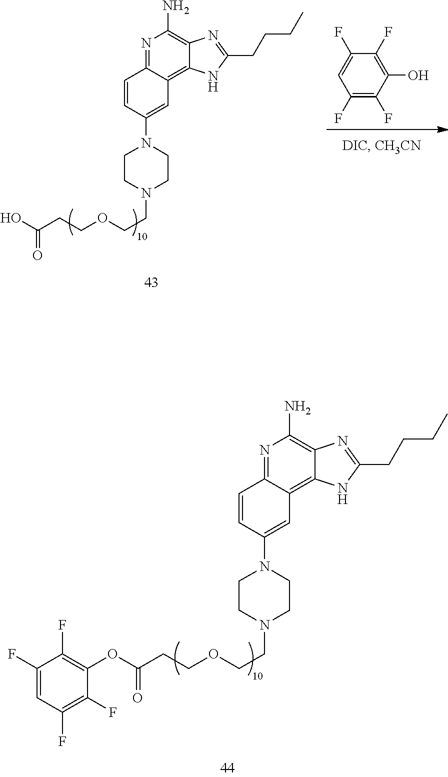

1-(4-(4-amino-2-butyl-1H-imidazo[4,5-c]quinolin-8-yl)piperazin-1-yl)-3,6,9,12,15,18,21,24,27,30-decaoxatritriacontan-33-oic acid was converted into 2,3,5,6-tetrafluorophenyl 1-(4-(4-amino-2-butyl-1H-imidazo[4,5-c]quinolin-8-yl)piperazin-1-yl)-3,6,9,12,15,18,21,24,27,30-decaoxatritriacontan-33-oate in a 54% yield using the procedure described in Example 17. LC/MS [M+H] 985.49 (calculated); LC/MS [M+H] 985.71 (observed).

### Example 44: Synthesis of Compound 46

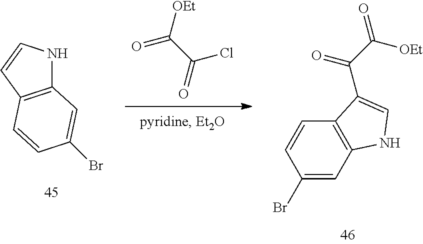 [SMILES: Brc1ccc2cc[nH]c2c1 ; CCOC(=O)C(=O)Cl ; CCOC(=O)C(=O)c1c[nH]c2cc(Br)ccc12]

To a mixture of 6-bromo-1H-indole (5.00 g, 25.50 mmol, 1 eq) and pyridine (2.62 g, 33.16 mmol, 2.68 mL, 1.3 eq) in Et₂O (80 mL) was added ethyl 2-chloro-2-oxo-acetate (4.18 g, 30.61 mmol, 3.43 mL, 1.2 eq) slowly at 0° C. under N₂. The mixture was stirred at 0° C. for 2 hours. A yellow solid precipitated. The mixture was filtered and the cake was washed by H₂O. The crude product was triturated with H₂O at 20° C. for 20 min to provide ethyl 2-(6-bromo-1H-indol-3-yl)-2-oxo-acetate (5.4 g, 18.24 mmol, 71.50% yield) as a yellow solid. ¹H NMR (DMSO-d₆, 400 MHz) δ 12.46 (s, 1H), 8.46 (d, J=3.6 Hz, 1H), 8.10 (d, J=8.8 Hz, 1H), 7.75 (s, 1H), 7.43 (d, J=8.8 Hz, 1H), 4.36 (q, J=7.2 Hz, 2H), 1.33 (t, J=7.2 Hz, 3H).

### Example 45: Synthesis of Compound 47

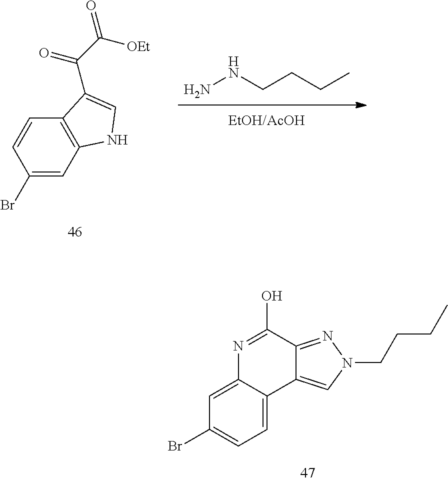

To a mixture of ethyl 2-(6-bromo-1H-indol-3-yl)-2-oxo-acetate (5.4 g, 18.24 mmol, 1 eq) and butylhydrazine (3.41 g, 27.35 mmol, 1.5 eq, HCl) in EtOH (60 mL) was added AcOH (10.95 g, 182.36 mmol, 10.43 mL, 10 eq) at 25° C. under N₂. The mixture was stirred at 90° C. for 16 hours. LCMS showed the reaction was completed. The mixture was concentrated in vacuum. The residue was purified by silica gel chromatography (column height: 250 mm, diameter: 100 mm, 100-200 mesh silica gel, petroleum ether/ethyl acetate=5/1, 1/2) to provide 7-bromo-2-butyl-pyrazolo[3,4-c]quinolin-4-ol (3 g, 9.37 mmol, 51.38% yield) as a brown solid. ¹H NMR (CDCl₃, 400 MHz) δ 11.40 (s, 1H), 8.72 (s, 1H), 7.81 (d, J=8.4 Hz, 1H), 7.50 (s, 1H), 7.34 (dd, J=8.4, 2.0 Hz, 1H), 4.37 (t, J=6.8 Hz, 2H), 1.91-1.84 (m, 2H), 1.32-1.25 (m, 2H), 0.91 (t, J=7.2 Hz, 3H).

### Example 46: Synthesis of Compound 48

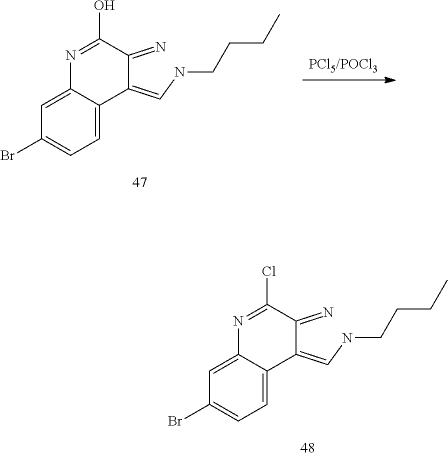 [SMILES: CCCCN/C=C1\C(=N)C(O)=Nc2cc(Br)ccc21 ; CCCCN/C=C1\C(=N)C(Cl)=Nc2cc(Br)ccc21]

To a mixture of 7-bromo-2-butyl-pyrazolo[3,4-c]quinolin-4-ol (2.8 g, 8.74 mmol, 1 eq) in POCl₃ (13.41 g, 87.45 mmol, 8.13 mL, 10 eq) was added PCl₅ (910.52 mg, 4.37 mmol, 0.5 eq) in one portion at 25° C. The mixture was stirred at 100° C. for 1 hour. LCMS showed the reaction was completed. The mixture was concentrated. The residue was poured into ice water (100 mL) and diluted with CH₂C₁₂ (30 mL) and washed with saturated NaHCO₃, dried over Na₂SO₄, filtered, and concentrated. The residue was purified by silica gel chromatography (column height: 250 mm, diameter: 100 mm, 100-200 mesh silica gel, petroleum ether/ethyl acetate=10/1, 3/1) to provide 7-bromo-2-butyl-4-chloro-pyrazolo[3,4-c]quinoline (2.6 g, 7.68 mmol, 87.80% yield) as a yellow oil. ¹H NMR (DMSO-d₆, 400 MHz) δ 8.30 (s, 1H), 8.22 (d, J=2.0 Hz, 1H), 7.85 (d, J=8.4 Hz, 1H), 7.68 (dd, J=8.4, 2.0 Hz, 1H), 4.53 (t, J=7.2 Hz, 2H), 2.08-2.04 (m, 2H), 1.46-1.37 (m, 2H), 0.10 (t, J=7.2 Hz, 3H).

### Example 47: Synthesis of Compound 49

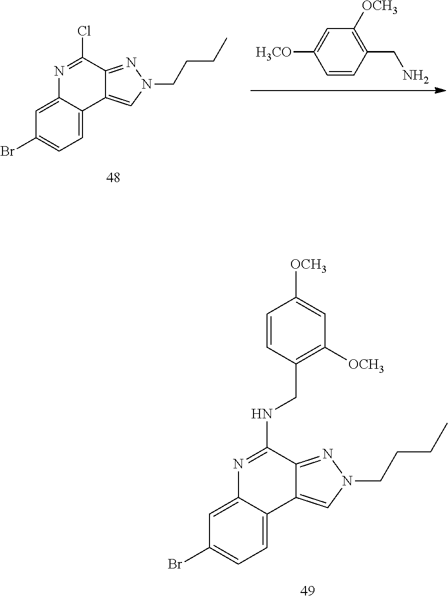 [SMILES: CCCCn1cc2c(n1)c(Cl)nc1cc(Br)ccc12 ; COc1ccc(CN)c(OC)c1 ; CCCCn1cc2c(n1)c(NCc1ccc(OC)cc1OC)nc1cc(Br)ccc12]

To mixture 7-bromo-2-butyl-4-chloro-pyrazolo[3,4-c]quinoline (2.6 g, 7.68 mmol, 1 eq) in 2,4-dimethoxyphenyl)methanamine (6.42 g, 38.39 mmol, 5.78 mL, 5 eq) was stirred at 120° C. for 4 hours. LCMS showed the reaction was completed. The mixture was dissolved in EtOAc/H₂O (10 mL/10 mL) and adjusted pH=3 with aq. HCl (4 M). The aqueous phase was filtered to give 7-bromo-2-butyl-N-[(2,4-dimethoxyphenyl)methyl]pyrazolo[3,4-c]quinolin-4-amine (2.9 g, 6.18 mmol, 80.47% yield) as a yellow solid which was used into the next step without further purification. ¹H NMR (CDCl₃, 400 MHz) δ 9.03 (s, 1H), 8.34 (s, 1H), 8.04 (d, J=8.4 Hz, 1H), 7.64 (s, 1H), 7.20 (d, J=8.4 Hz, 1H), 6.61 (d, J=2.4 Hz, 1H), 6.51 (d, J=8.4 Hz, 1H), 4.89 (d, J=4.2 Hz, 2H), 4.49 (t, J=6.8 Hz, 2H), 3.75 (m, 6H), 1.96-1.89 (m, 2H), 1.35-1.27 (m, 2H), 0.91 (t, J=7.2 Hz, 3H).

### Example 48: Synthesis of Compound 50

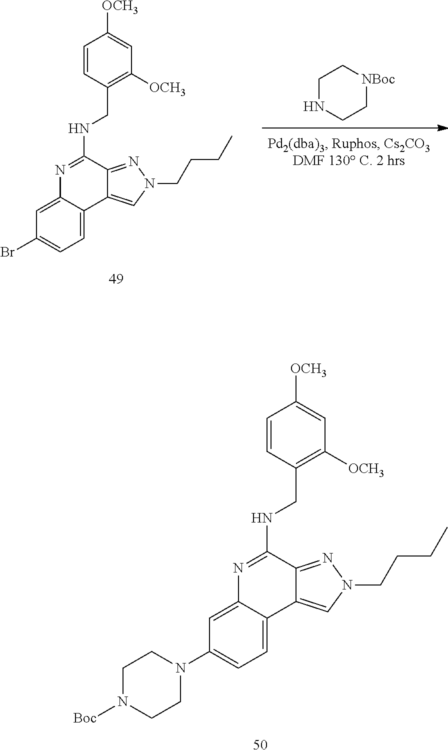 [SMILES: CCCCn1cc2c(n1)c(NCc1ccc(OC)cc1OC)nc1cc(Br)ccc12 ; CC(C)(C)OC(=O)N1CCNCC1 ; CCCCn1cc2c(n1)c(NCc1ccc(OC)cc1OC)nc1cc(N3CCN(C(=O)OC(C)(C)C)CC3)ccc12]

To a mixture of 7-bromo-2-butyl-N-[(2,4-dimethoxyphenyl)methyl]pyrazolo[3,4-c]quinolin-4-amine (0.45 g, 958.73 μmol, 1 eq) and tert-butyl piperazine-1-carboxylate (535.69 mg, 2.88 mmol, 3 eq) in DMF (10 mL) was added Pd₂(dba)₃ (43.90 mg, 47.94 μmol, 0.05 eq), Cs₂CO₃ (624.74 mg, 1.92 mmol, 2 eq) and RuPhos (44.74 mg, 95.87 μmol, 0.1 eq) in one portion at 25° C. under N₂. The mixture was stirred at 140° C. for 2 hours. LCMS showed the reaction was completed. The mixture was cooled to 25° C. and poured into ice water (30 mL) and stirred for 1 min. The aqueous phase was extracted with ethyl acetate (10 mL×3). The combined organic phase was washed with brine (10 mL), dried with anhydrous Na₂SO₄, filtered, and concentrated in vacuum. The residue was purified by silica gel chromatography (petroleum ether/ethyl acetate=10/1, 1/1) to provide tert-butyl 4-[2-butyl-4-[(2,4-dimethoxyphenyl) methylamino]pyrazolo[3,4-c]quinolin-7-yl]piperazine-1-carboxylate (0.45 g, 783.00 μmol, 81.67% yield) as a yellow oil. ¹H NMR (CDCl₃, 400 MHz) δ 7.95 (s, 1H), 7.67 (d, J=8.8 Hz, 1H), 7.40 (d, J=8.0 Hz, 1H), 7.28 (d, J=2.4 Hz, 1H), 6.91 (dd, J=8.8, 2.4 Hz, 1H), 6.49 (d, J=2.4 Hz, 1H), 6.45 (dd, J=8.4, 2.4 Hz, 1H), 5.98 (s, 1H), 4.87 (d, J=4.4 Hz, 2H), 4.33 (t, J=7.6 Hz, 2H), 3.86 (s, 3H), 3.80 (s, 3H), 3.64-3.61 (m, 4H), 3.26-3.23 (m, 4H), 1.99-1.92 (m, 2H), 1.51 (s, 9H), 1.40-1.34 (m, 2H), 0.96 (t, J=7.2 Hz, 3H).

### Example 49: Synthesis of Compound 51

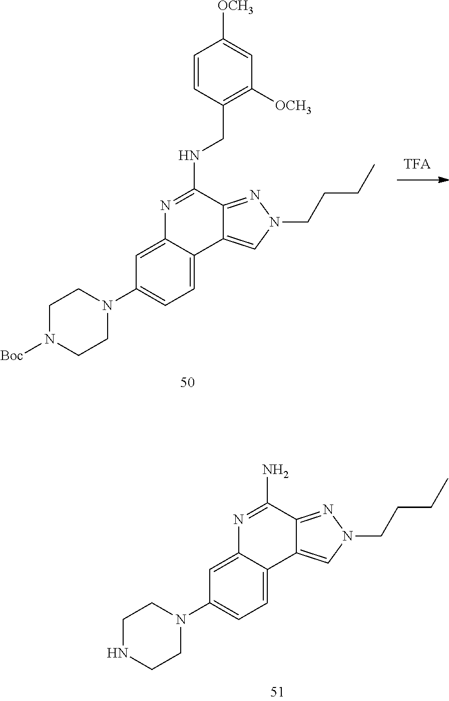 [SMILES: CCCCn1cc2c(n1)c(NCc1ccc(OC)cc1OC)nc1cc(N3CCN(C(=O)OC(C)(C)C)CC3)ccc12 ; CCCCn1cc2c(n1)c(N)nc1cc(N3CCNCC3)ccc12]

To a mixture of tert-butyl 4-[2-butyl-4-[(2,4-dimethoxyphenyl)methylamino] pyrazolo[3,4-c]quinolin-7-yl]piperazine-1-carboxylate (0.2 g, 348.00 μmol, 1 eq) in DCM (20 mL) was added TFA (1.98 g, 17.40 mmol, 1.29 mL, 50 eq) in one portion at 25° C. The mixture was stirred at 50° C. for 36 hours. LCMS and HPLC showed the reaction was completed. The mixture was concentrated and purified by prep-HPLC (column: Nano-micro KROMASIL™ (Sigma-Aldrich) C18 100×30 mm Sum; mobile phase: [water (0.1% TFA)-ACN]; B %: 20%-55%, 10 min) to provide 2-butyl-7-piperazin-1-yl-pyrazolo[3,4-c]quinolin-4-amine (0.088 g, 200.71 μmol, 57.67% yield, TFA) as an off-white solid. ¹H NMR (DMSO-d₆, 400 MHz) δ 9.01 (s, 2H), 8.88 (s, 1H), 7.96 (d, J=8.8 Hz, 1H), 7.23 (dd, J=8.8, 2.4 Hz, 1H), 7.11 (d, J=2.4 Hz, 1H), 4.49 (t, J=7.2 Hz, 2H), 3.45-3.44 (m, 4H), 3.35-3.29 (m, 4H), 1.98-1.90 (m, 2H), 1.36-1.27 (m, 2H), 0.93 (t, J=7.2 Hz, 3H). LCMS (ESI): mass calcd. for C₁₈H₂₄N₆ 324.21, m/z found 325.3 [M+H]⁺.

### Example 50: Synthesis of Compound 52

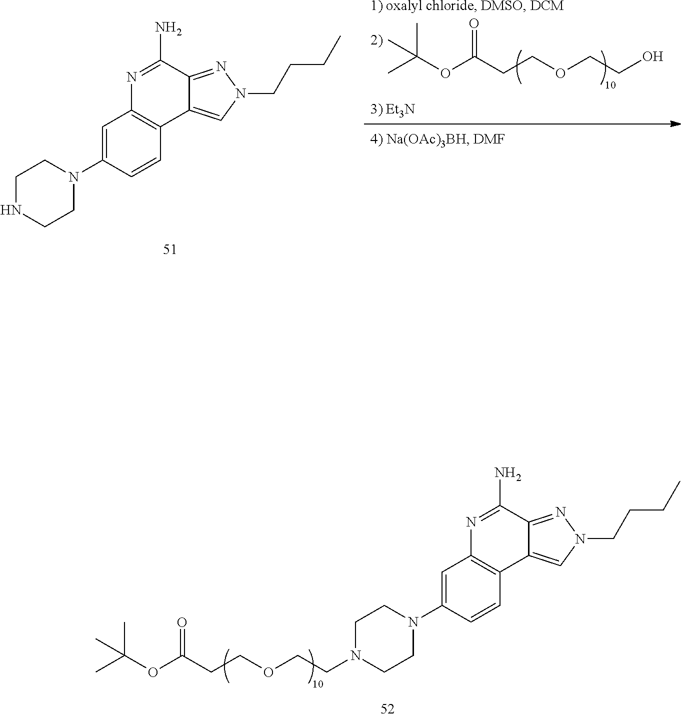

2-butyl-7-(piperazin-1-yl)-2H-pyrazolo[3,4-c]quinolin-4-amine was converted into tert-butyl 1-(4-(4-amino-2-butyl-2H-pyrazolo[3,4-c]quinolin-7-yl)piperazin-1-yl)-3,6,9,12,15,18,21,24,27,30-decaoxatritriacontan-33-oate in a 65% yield using the procedure described in Example 12. LC/MS [M+H] 893.56 (calculated); LC/MS [M+H] 893.82 (observed).

### Example 51: Synthesis of Compound 53

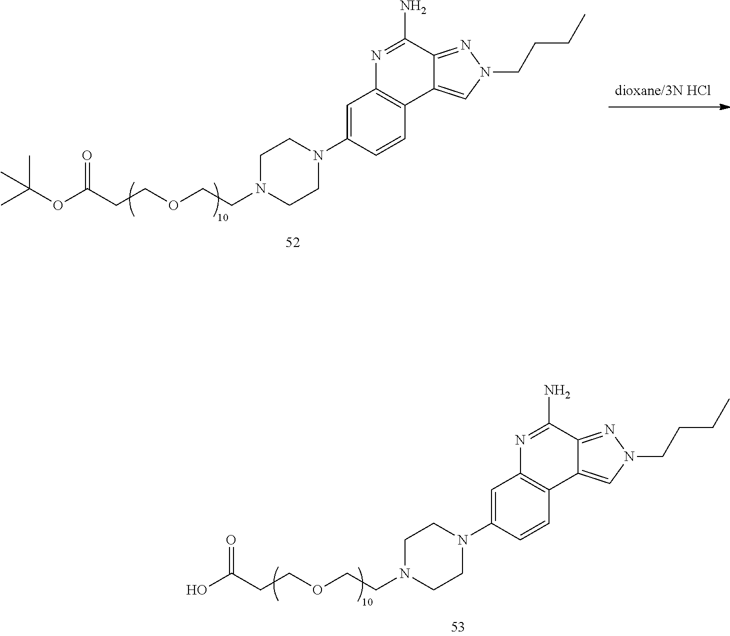

tert-butyl 1-(4-(4-amino-2-butyl-2H-pyrazolo[3,4-c]quinolin-7-yl)piperazin-1-yl)-3,6,9,12,15,18,21,24,27,30-decaoxatritriacontan-33-oate was converted into 1-(4-(4-amino-2-butyl-2H-pyrazolo[3,4-c]quinolin-7-yl)piperazin-1-yl)-3,6,9,12,15,18,21,24,27,30-decaoxatritriacontan-33-oic acid in a 92% yield using the procedure described in Example 16. The compound was used without further purification.

### Example 52: Synthesis of Compound 54

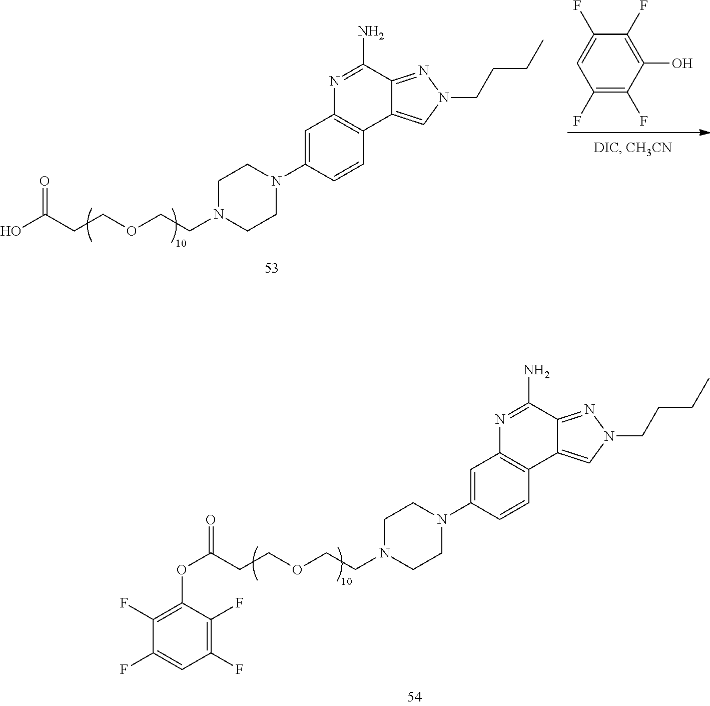

1-(4-(4-amino-2-butyl-2H-pyrazolo[3,4-c]quinolin-7-yl)piperazin-1-yl)-3,6,9,12,15,18,21,24,27,30-decaoxatritriacontan-33-oic acid was converted into 2,3,5,6-tetrafluorophenyl 1-(4-(4-amino-2-butyl-2H-pyrazolo[3,4-c]quinolin-7-yl)piperazin-1-yl)-3,6,9,12,15,18,21,24,27,30-decaoxatritriacontan-33-oate in a 46% yield using the procedure described in Example 17. LC/MS [M+H] 985.49 (calculated); LC/MS [M+H] 985.73 (observed).

### Example 53: Synthesis of Compound 56

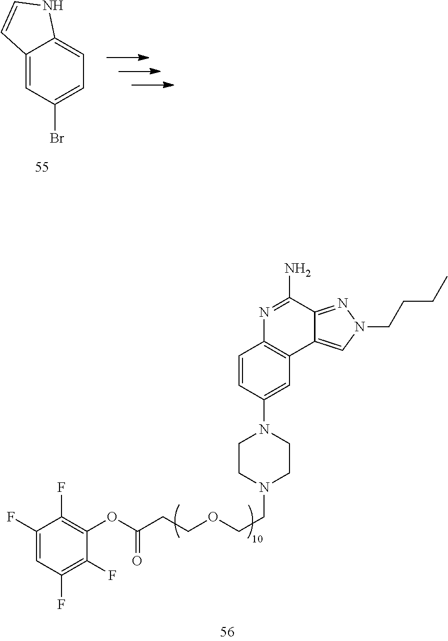

5-bromo-1H-indole was converted into 2,3,5,6-tetrafluorophenyl 1-(4-(4-amino-2-butyl-2H-pyrazolo[3,4-c]quinolin-8-yl)piperazin-1-yl)-3,6,9,12,15,18,21,24,27,30-decaoxatritriacontan-33-oate using the route described in Examples 45-53. LC/MS [M+H] 985.49 (calculated); LC/MS [M+H] 985.73 (observed).

### Example 54: Synthesis of Compound 59

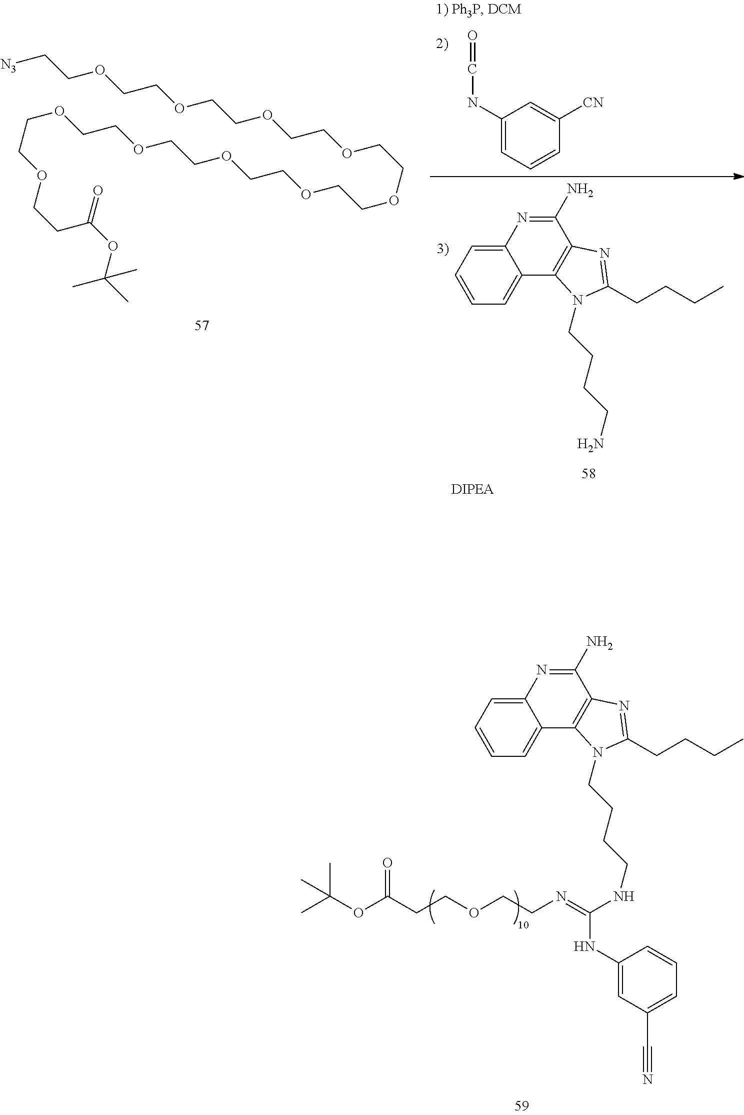

A 4 mL vial was charged with tert-butyl 1-azido-3,6,9,12,15,18,21,24,27,30-decaoxatritriacontan-33-oate (0.077 mmol, 47 mg), triphenylphosphine (0.088 mmol, 23 mg) and 500 μL of anhydrous dichloromethane. The reaction was maintained at 30° C. for 90 min, at which point 3-cyanophenyl isocyanate (0.076 mmol, 11 mg) was added. After 45 min a solution containing 58.HCl (0.077 mmol) and DIEA (0.345 mmol) in 750 mL of 2:1 DCM:DMF was added. This reaction was maintained for 2 h then concentrated under reduced pressure. The crude reaction was purified using reverse phase preparative HPLC utilizing a 25-75% gradient of acetonitrile:water containing 0.1% trifluoroacetic acid. The purified fractions were combined and lyophilized to provide 49.7 mg of the desired product tert-butyl (Z)-40-(4-amino-2-butyl-1H-imidazo[4,5-c]quinolin-1-yl)-35-((3-cyanophenyl)amino)-4,7,10,13,16,19,22,25,28,31-decaoxa-34,36-diazatetracont-34-enoate in 63% yield. LC/MS [M+H] 1023.61 (calculated); LC/MS [M+H] 1024.01 (observed).

### Example 55: Synthesis of Compound 60

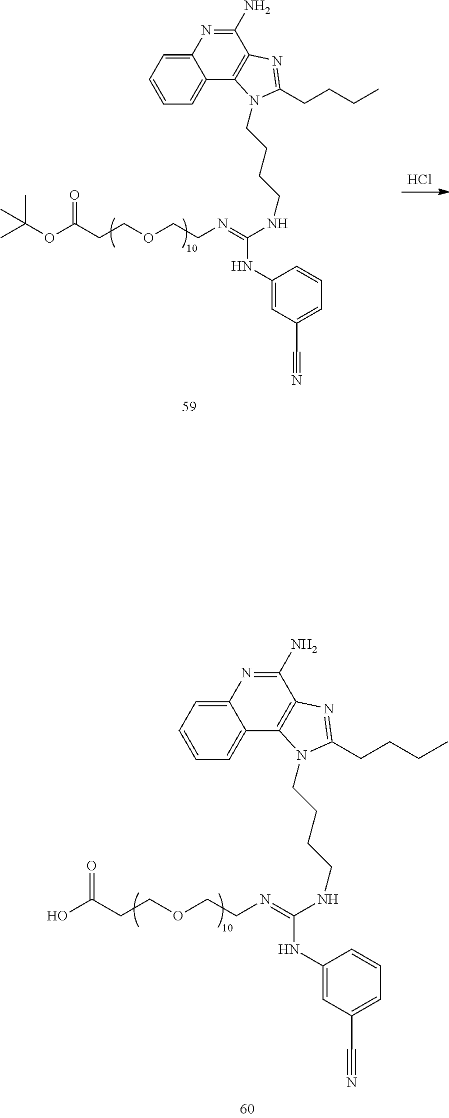

A 4 ml vial was charged with tert-butyl (Z)-40-(4-amino-2-butyl-1H-imidazo[4,5-c]quinolin-1-yl)-35-((3-cyanophenyl)amino)-4,7,10,13,16,19,22,25,28,31-decaoxa-34,36-diazatetracont-34-enoate (0.031 mmol, 32 mg), 1 mL 1,4-dioxane, and 1 mL 4M HCl in 1,4-dioxane. The reaction was stirred for 6 h, diluted with water (8 mL) and purified by reverse phase preparative HPLC utilizing a 25-75% gradient of acetonitrile:water containing 0.1% trifluoroacetic acid. The purified fractions were combined and lyophilized to afford 15.8 mg of the desired product (Z)-40-(4-amino-2-butyl-1H-imidazo[4,5-c]quinolin-1-yl)-35-((3-cyanophenyl)amino)-4,7,10,13,16,19,22,25,28,31-decaoxa-34,36-diazatetracont-34-enoic acid in 53% yield. LC/MS [M+H] 967.55 (calculated); LC/MS [M+H] 967.96 (observed).

### Example 56: Synthesis of Compound 61

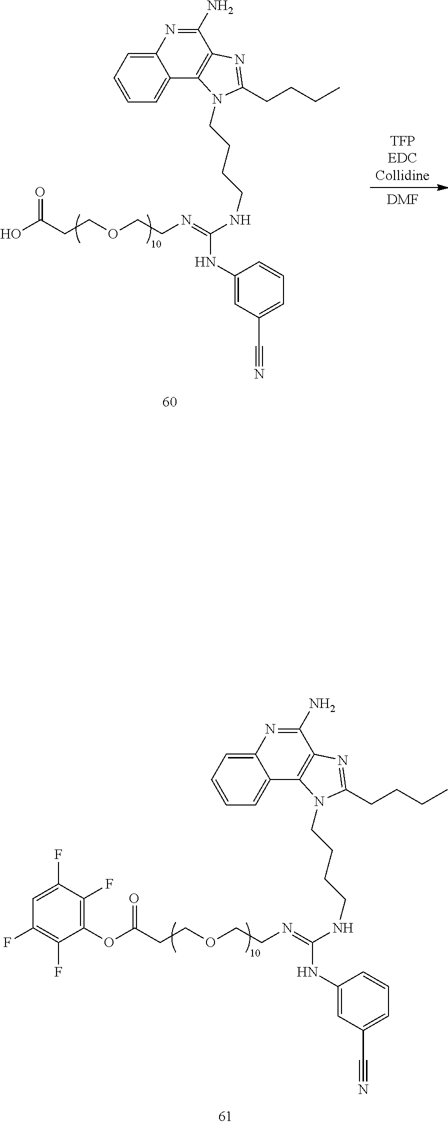

A 4 mL vial was charged with (Z)-40-(4-amino-2-butyl-1H-imidazo[4,5-c]quinolin-1-yl)-35-((3-cyanophenyl)amino)-4,7,10,13,16,19,22,25,28,31-decaoxa-34,36-diazatetracont-34-enoic acid (9.7 μmol, 9.4 mg) and 300 μL of DMF. To this vial was added 2,3,5,6-tetrafluorophenol (29 μmol, 4.1 mg) in 50 μL of DMF followed by 1-hydroxy-7-azabenzotriazole (9.4 μmol, 1.3 mg) and 1-ethyl-3-(3-dimethylaminopropyl)carbodiimide (29 μmol, 6.5 mg). The reaction was heat to 70° C. and stirred for 5 h, after which the reaction was cooled to 20° C., diluted with water (8 mL) and purified by reverse phase preparative HPLC utilizing a 25-75% gradient of acetonitrile:water containing 0.1% trifluoroacetic acid. The purified fractions were combined and lyophilized to afford 5.0 mg of the desired product 2,3,5,6-tetrafluorophenyl (Z)-40-(4-amino-2-butyl-1H-imidazo[4,5-c]quinolin-1-yl)-35-((3-cyanophenyl)amino)-4,7,10,13,16,19,22,25,28,31-decaoxa-34,36-diazatetracont-34-enoate in 46% yield. LC/MS [M+H] 1115.54 (calculated); LC/MS [M+H] 1115.98 (observed).

### Example 57: Synthesis of Compound 63

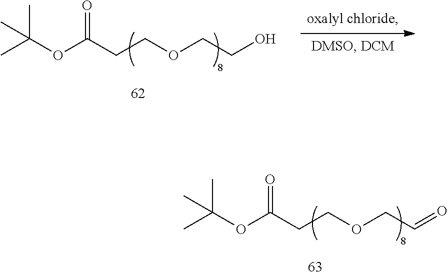

A 20 mL vial was charged with oxalyl chloride (3.02 mmol, 260 μL) and 3 mL anhydrous DCM was cooled to −78° C. in a dry ice-acetone bath. A solution of DMSO (6.05 mmol, 429 μL) in 2 mL anhydrous DCM was added dropwise and the reaction was stirred for 1 h. A solution of tert-butyl 1-hydroxy-3,6,9,12,15,18,21,24-octaoxaheptacosan-27-oate (1.01 mmol, 502 mg) in 2 mL anhydrous DCM was added dropwise and stirred for 20 min. Triethylamine (9.07 mmol, 1.23 mL) was added dropwise and the mixture was stirred for 40 min at −78° C. then warmed to room temperature over 30 min. The reaction was concentrated under reduced pressure and used immediately in the subsequent step.

### Example 58: Synthesis of Compound 64

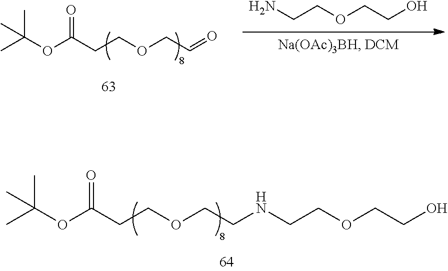

To a 20 ml vial containing crude tert-butyl 1-oxo-3,6,9,12,15,18,21,24-octaoxaheptacosan-27-oate (1.01 mmol) and 2-(2-aminoethoxy)ethan-1-ol (2.37 mmol, 249 mg) in 10 mL anhydrous DCM was added triacetoxyborohydride (5.04 mmol, 1.07 g). The reaction was stirred for 5 h, then quenched with 1 mL of 10% K₂CO₃. The reaction was concentrated under reduced pressure. Purification by reverse phase flash chromatography using a 0-100% MeCN:water+0.1% TFA gradient afforded 276 mg of the desired product tert-butyl 1-hydroxy-3,9,12,15,18,21,24,27,30-nonaoxa-6-azatritriacontan-33-oate in 47% yield. LC/MS [M+H] 586.38 (calculated); LC/MS [M+H] 586.92 (observed).

### Example 59: Synthesis of Compound 65

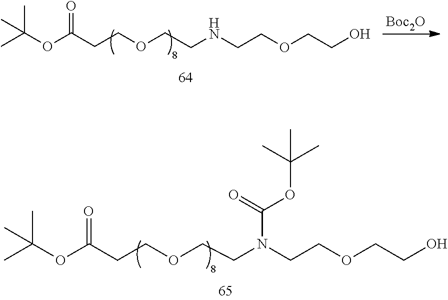

A 20 mL vial was charged with di-tert-butyl dicarbonate (1.39 mmol, 320 μL), tert-butyl 1-hydroxy-3,9,12,15,18,21,24,27,30-nonaoxa-6-azatritriacontan-33-oate (0.47 mmol, 276 mg), sodium bicarbonate (2.36 mmol, 200 mg), 4 mL THF, and 1 mL water. The reaction was stirred for 5 h, then concentrated under reduced pressure. Purification by reverse phase flash chromatography using a 0-100% MeCN:water+0.1% TFA gradient afforded 109 mg of the desired product tert-butyl 6-(tert-butoxycarbonyl)-1-hydroxy-3,9,12,15,18,21,24,27,30-nonaoxa-6-azatritriacontan-33-oate in 34% yield. LC/MS [M+H] 686.42 (calculated); LC/MS [M+H] 686.74 (observed).

### Example 60: Synthesis of Compound 66

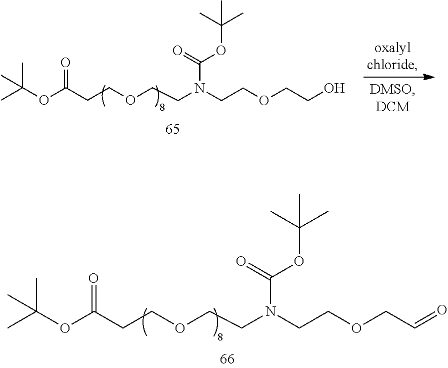

tert-butyl 6-(tert-butoxycarbonyl)-1-oxo-3,9,12,15,18,21,24,27,30-nonaoxa-6-azatritriacontan-33-oate was formed from tert-butyl 6-(tert-butoxycarbonyl)-1-hydroxy-3,9,12,15,18,21,24,27,30-nonaoxa-6-azatritriacontan-33-oate using Swern oxidation conditions according to Example 57. Crude material was carried forward to next step without purification.

### Example 61: Synthesis of Compound 67

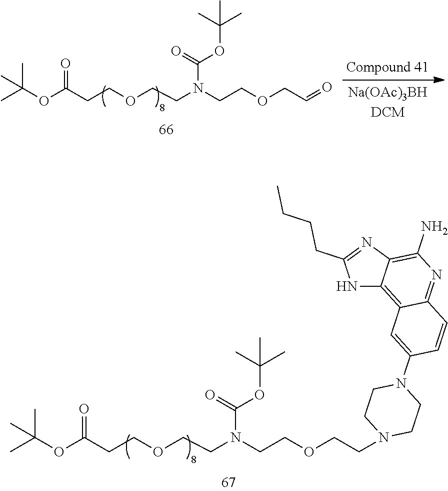

tert-butyl 1-(4-(4-amino-2-butyl-1H-imidazo[4,5-c]quinolin-8-yl)piperazin-1-yl)-6-(tert-butoxycarbonyl)-3,9,12,15,18,21,24,27,30-nonaoxa-6-azatritriacontan-33-oate was formed from tert-Butyl 6-(tert-butoxycarbonyl)-1-oxo-3,9,12,15,18,21,24,27,30-nonaoxa-6-azatritriacontan-33-oate using reductive amination conditions according to Example 58. Crude material was carried forward to next step without purification.

### Example 62: Synthesis of Compound 68

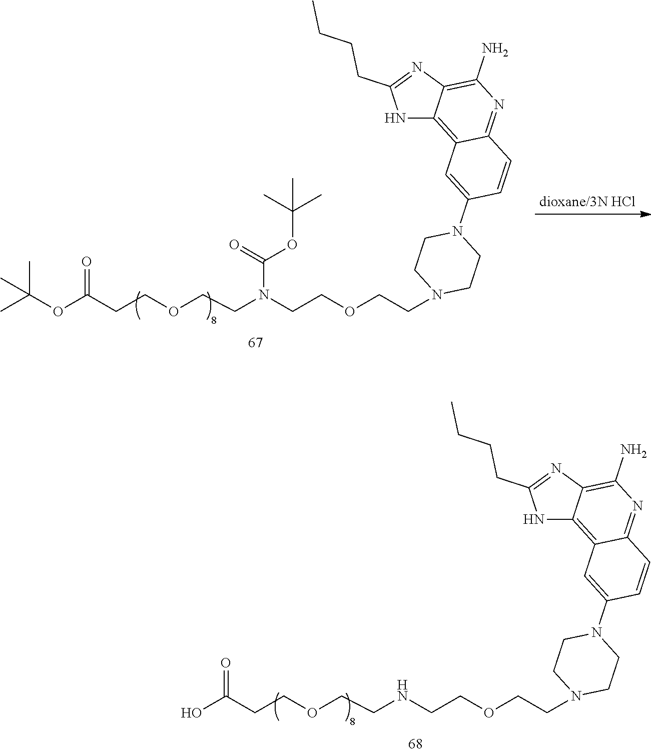

1-(4-(4-amino-2-butyl-1H-imidazo[4,5-c]quinolin-8-yl)piperazin-1-yl)-3,9,12,15,18,21,24,27,30-nonaoxa-6-azatritriacontan-33-oic acid was formed from tert-butyl 1-(4-(4-amino-2-butyl-1H-imidazo[4,5-c]quinolin-8-yl)piperazin-1-yl)-6-(tert-butoxycarbonyl)-3,9,12,15,18,21,24,27,30-nonaoxa-6-azatritriacontan-33-oate using Boc deprotection conditions according to Example 12. Crude material was carried forward to next step without purification. Purification by reverse phase flash chromatography using a 0-100% MeCN:water+0.1% TFA gradient afforded 109 mg of the desired product 1-(4-(4-amino-2-butyl-1H-imidazo[4,5-c]quinolin-8-yl)piperazin-1-yl)-3,9,12,15,18,21,24,27,30-nonaoxa-6-azatritriacontan-33-oic acid in 43% yield over three steps. LC/MS [M+H] 836.51 (calculated); LC/MS [M+H] 836.71 (observed).

### Example 63: Synthesis of Compound 69

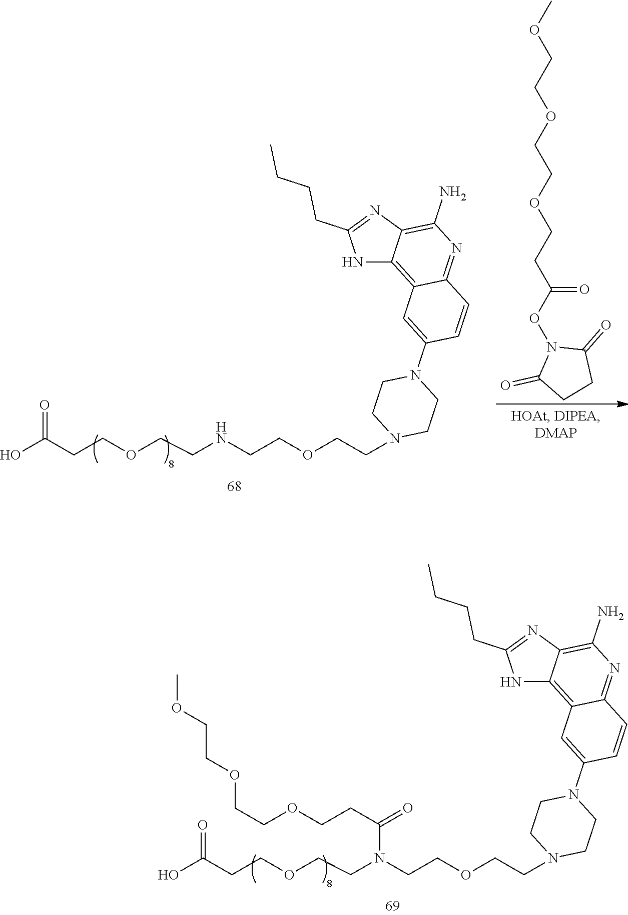

A 4 mL vial was charged with 1-(4-(4-amino-2-butyl-1H-imidazo[4,5-c]quinolin-8-yl)piperazin-1-yl)-3,9,12,15,18,21,24,27,30-nonaoxa-6-azatritriacontan-33-oic acid (0.025 mmol, 22 mg), 2,5-dioxopyrrolidin-1-yl 3-(2-(2-methoxyethoxy)ethoxy)propanoate (0.025 mmol, 6.4 mg), Hunigs base (0.067 mmol, 11.5 μL), 1-hydroxy-7-azabenzotriazole (0.014 mmol, 2 mg), 4-dimethylaminopyridine (0.016 mmol, 2 mg), and 500 μLDMF. The reaction was stirred at 45° C. for 4 h. Purification by reverse phase preparative HPLC utilizing a 25-75% gradient of acetonitrile:water+0.1% trifluoroacetic acid afforded 8.5 mg of the desired 12-(2-(2-(4-(4-amino-2-butyl-1H-imidazo[4,5-c]quinolin-8-yl)piperazin-1-yl)ethoxy)ethyl)-11-oxo-2,5,8,15,18,21,24,27,30,33,36-undecaoxa-12-azanonatriacontan-39-oic acid in 34% yield. LC/MS [M+H] 1010.60 (calculated); LC/MS [M+H] 1010.76 (observed).

### Example 64: Synthesis of Compound 70

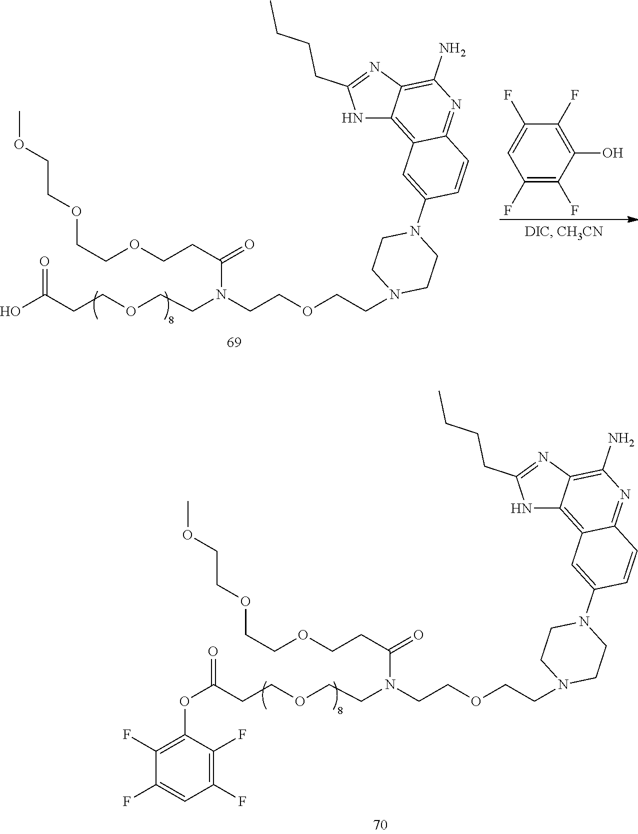

2,3,5,6-tetrafluorophenyl 12-(2-(2-(4-(4-amino-2-butyl-1H-imidazo[4,5-c]quinolin-8-yl)piperazin-1-yl)ethoxy)ethyl)-11-oxo-2,5,8,15,18,21,24,27,30,33,36-undecaoxa-12-azanonatriacontan-39-oate was formed from 12-(2-(2-(4-(4-amino-2-butyl-1H-imidazo[4,5-c]quinolin-8-yl)piperazin-1-yl)ethoxy)ethyl)-11-oxo-2,5,8,15,18,21,24,27,30,33,36-undecaoxa-12-azanonatriacontan-39-oic acid following the procedure of Example 17. The procedure provided 7.7 mg of the desired 2,3,5,6-tetrafluorophenyl 12-(2-(2-(4-(4-amino-2-butyl-1H-imidazo[4,5-c]quinolin-8-yl)piperazin-1-yl)ethoxy)ethyl)-11-oxo-2,5,8,15,18,21,24,27,30,33,36-undecaoxa-12-azanonatriacontan-39-oate in 79% yield. LC/MS [M+H] 1158.60 (calculated); LC/MS [M+H] 1158.88 (observed).

### Example 65: Synthesis of Compound 71

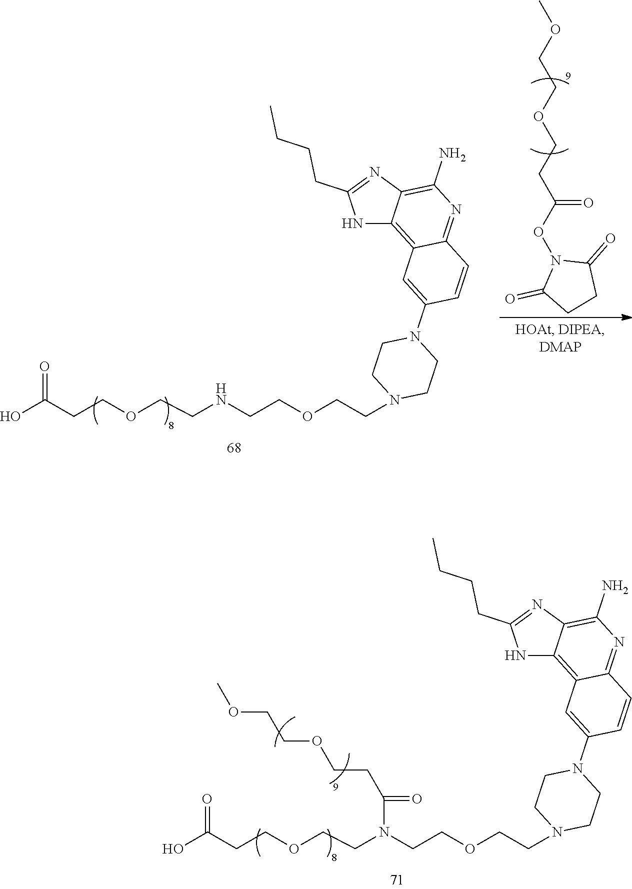

33-(2-(2-(4-(4-amino-2-butyl-1H-imidazo[4,5-c]quinolin-8-yl)piperazin-1-yl)ethoxy)ethyl)-32-oxo-2,5,8,11,14,17,20,23,26,29,36,39,42,45,48,51,54,57-octadecaoxa-33-azahexacontan-60-oic acid was formed from 1-(4-(4-amino-2-butyl-1H-imidazo[4,5-c]quinolin-8-yl)piperazin-1-yl)-3,9,12,15,18,21,24,27,30-nonaoxa-6-azatritriacontan-33-oic acid according to the procedure provided in Example 63. This procedure afforded 14.1 mg of the desired 33-(2-(2-(4-(4-amino-2-butyl-1H-imidazo[4,5-c]quinolin-8-yl)piperazin-1-yl)ethoxy)ethyl)-32-oxo-2,5,8,11,14,17,20,23,26,29,36,39,42,45,48,51,54,57-octadecaoxa-33-azahexacontan-60-oic acid in 49% yield. LC/MS [M+H] 1318.79 (calculated); LC/MS [M+H] 1318.99 (observed).

### Example 66: Synthesis of Compound 72

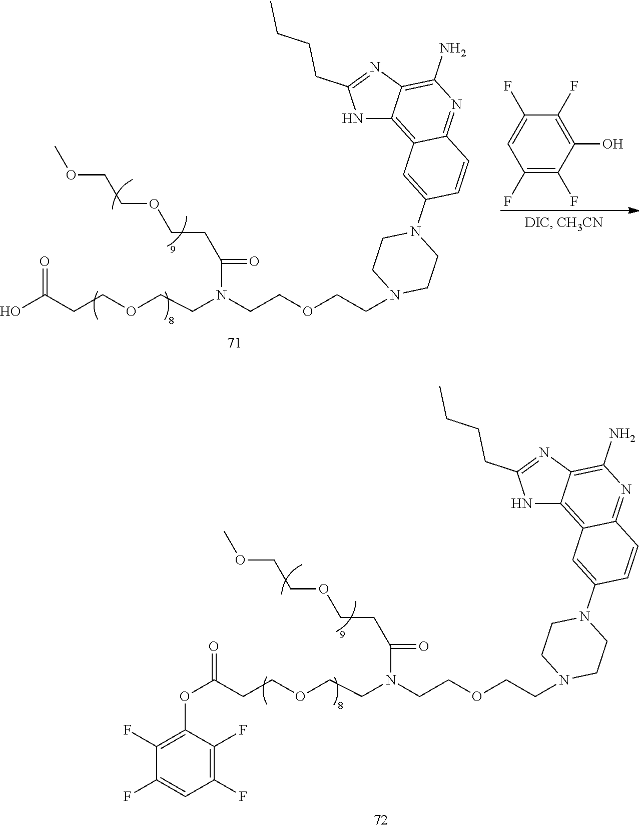

2,3,5,6-tetrafluorophenyl 33-(2-(2-(4-(4-amino-2-butyl-1H-imidazo[4,5-c]quinolin-8-yl)piperazin-1-yl)ethoxy)ethyl)-32-oxo-2,5,8,11,14,17,20,23,26,29,36,39,42,45,48,51,54,57-octadecaoxa-33-azahexacontan-60-oate was formed from 33-(2-(2-(4-(4-amino-2-butyl-1H-imidazo[4,5-c]quinolin-8-yl)piperazin-1-yl)ethoxy)ethyl)-32-oxo-2,5,8,11,14,17,20,23,26,29,36,39,42,45,48,51,54,57-octadecaoxa-33-azahexacontan-60-oic acid according to the procedure provided in Example 64. This procedure provided 11.9 mg of the desired 2,3,5,6-tetrafluorophenyl 33-(2-(2-(4-(4-amino-2-butyl-1H-imidazo[4,5-c]quinolin-8-yl)piperazin-1-yl)ethoxy)ethyl)-32-oxo-2,5,8,11,14,17,20,23,26,29,36,39,42,45,48,51,54,57-octadecaoxa-33-azahexacontan-60-oate in 76% yield. LC/MS [M+H] 1466.78 (calculated); LC/MS [M+H] 1467.12 (observed).

### Example 67: Synthesis of Compound 73

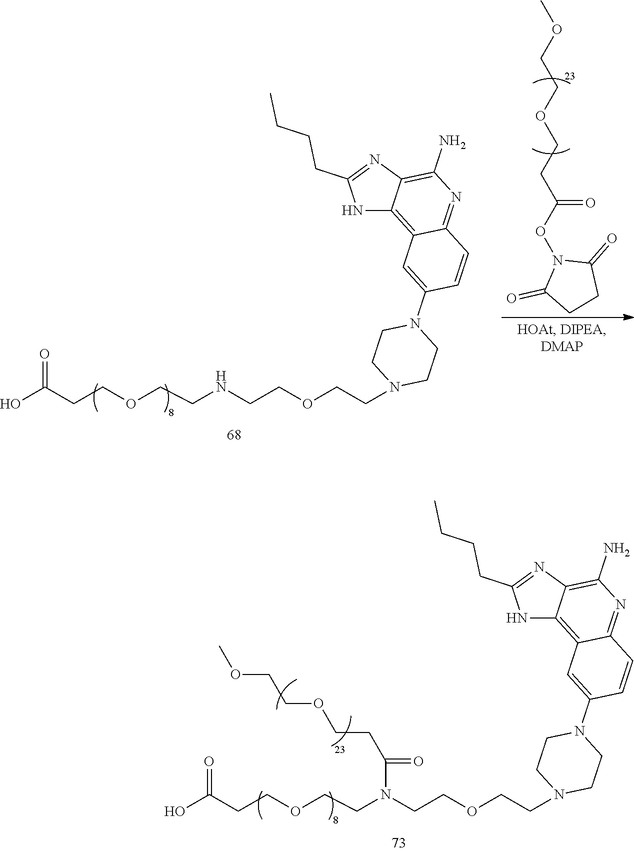

75-(2-(2-(4-(4-amino-2-butyl-1H-imidazo[4,5-c]quinolin-8-yl)piperazin-1-yl)ethoxy)ethyl)-74-oxo-2,5,8,11,14,17,20,23,26,29,32,35,38,41,44,47,50,53,56,59,62,65,68,71,78,81,84,87,90,93,96, 99-dotriacontaoxa-75-azadohectan-102-oic acid was formed from 1-(4-(4-amino-2-butyl-1H-imidazo[4,5-c]quinolin-8-yl)piperazin-1-yl)-3,9,12,15,18,21,24,27,30-nonaoxa-6-azatritriacontan-33-oic acid according to the procedure provided in Example 63. This procedure afforded 23.1 mg of the desired 75-(2-(2-(4-(4-amino-2-butyl-1H-imidazo[4,5-c]quinolin-8-yl)piperazin-1-yl)ethoxy)ethyl)-74-oxo-2,5,8,11,14,17,20,23,26,29,32,35,38,41,44,47,50,53,56,59,62,65,68,71,78,81,84,87,90,93,96,99-dotriacontaoxa-75-azadohectan-102-oic acid in 54% yield. LC/MS [M+H] 1935.15 (calculated); LC/MS [M+H] 1935.30 (observed).

### Example 68: Synthesis of Compound 74

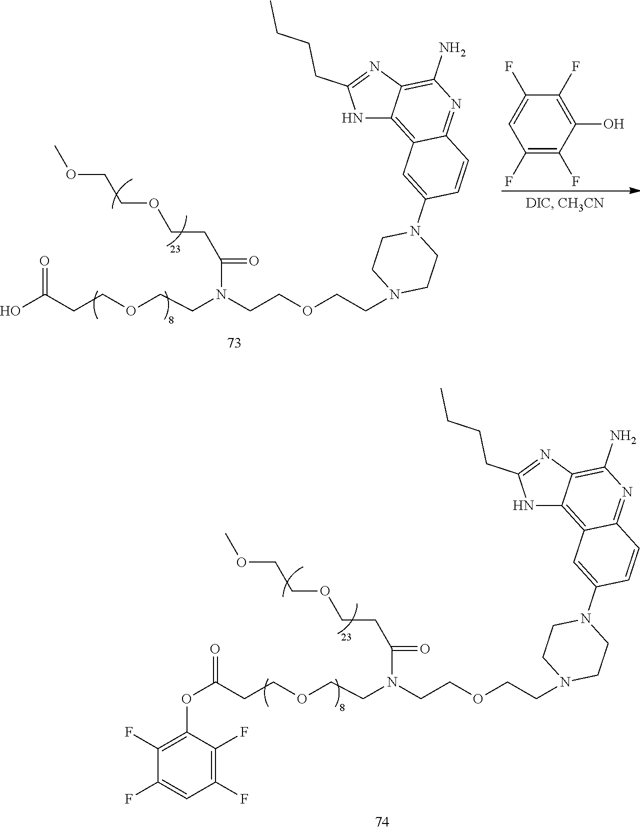

2,3,5,6-tetrafluorophenyl 75-(2-(2-(4-(4-amino-2-butyl-1H-imidazo[4,5-c]quinolin-8-yl)piperazin-1-yl)ethoxy)ethyl)-74-oxo-2,5,8,11,14,17,20,23,26,29,32,35,38,41,44,47,50,53,56,59,62,65,68,71,78,81,84,87,90,93,96,99-dotriacontaoxa-75-azadohectan-102-oate was formed from 75-(2-(2-(4-(4-amino-2-butyl-1H-imidazo[4,5-c]quinolin-8-yl)piperazin-1-yl)ethoxy)ethyl)-74-oxo-2,5,8,11,14,17,20,23,26,29,32,35,38,41,44,47,50,53,56,59,62,65,68,71,78,81,84,87,90,93,96,99-dotriacontaoxa-75-azadohectan-102-oic acid according to the procedure provided in Example 64. This procedure provided 13.4 mg of the desired 2,3,5,6-tetrafluorophenyl 75-(2-(2-(4-(4-amino-2-butyl-1H-imidazo[4,5-c]quinolin-8-yl)piperazin-1-yl)ethoxy)ethyl)-74-oxo-2,5,8,11,14,17,20,23,26,29,32,35,38,41,44,47,50,53,56,59,62,65,68,71,78,81,84,87,90,93,96,99-dotriacontaoxa-75-azadohectan-102-oate in 86% yield. LC/MS [M+H] 2083.15 (calculated); LC/MS [M+H] 2083.31 (observed).

### Example 69: Synthesis of Compound 76

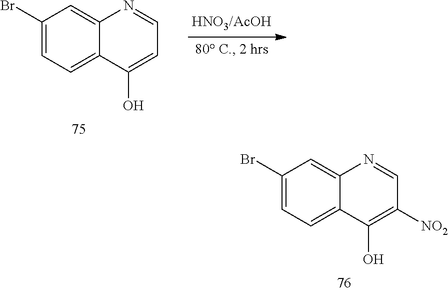 [SMILES: Oc1ccnc2cc(Br)ccc12 ; O=[N+]([O-])c1cnc2cc(Br)ccc2c1O]

7-bromoquinolin-4-ol (9.66 g, 43.11 mmol, 1 equiv.) was converted into 7-bromo-3-nitroquinolin-4-ol (7.46 g, 27.7 mmol, 64%) according to the procedure described in Example 2. LC/MS [M+H] 268.96/270.95 (calculated); LC/MS [M+H] 268.99/271.02 (observed).

### Example 70: Synthesis of Compound 77

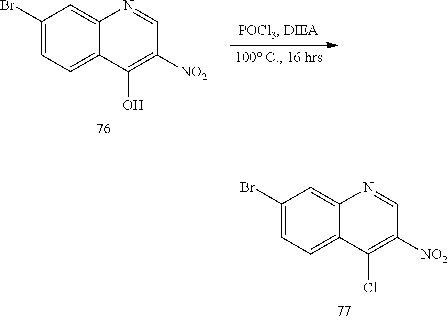 [SMILES: O=[N+]([O-])c1cnc2cc(Br)ccc2c1O ; O=[N+]([O-])c1cnc2cc(Br)ccc2c1Cl]

7-bromo-3-nitroquinolin-4-ol (7.46 g, 27.7 mmol, 1 equiv.) was converted into 7-bromo-4-chloro-3-nitroquinoline (6.88 g, 23.9 mmol, 86%) according to the procedure described in Example 3. LC/MS [M+H] 286.92/288.92 (calculated); LC/MS [M+H] 286.98/288.97 (observed).

### Example 71: Synthesis of Compound 78

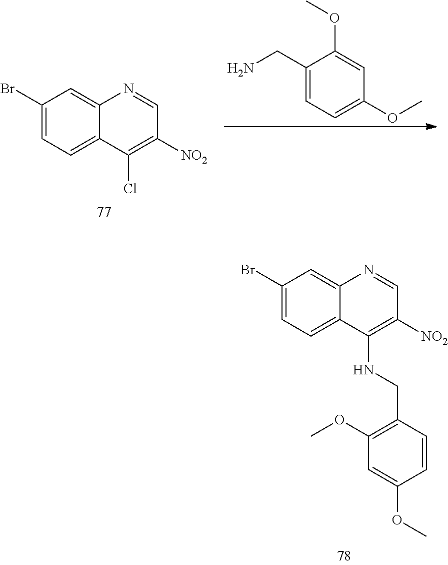 [SMILES: O=[N+]([O-])c1cnc2cc(Br)ccc2c1Cl ; COc1ccc(CN)c(OC)c1 ; COc1ccc(CNc2c([N+](=O)[O-])cnc3cc(Br)ccc23)c(OC)c1]

7-bromo-4-chloro-3-nitroquinoline (2.86 g, 9.95 mmol, 1 equiv.) was converted into 7-bromo-N-(2,4-dimethoxybenzyl)-3-nitroquinolin-4-amine (4.2 g, 10.0 mmol, 100%) according to the procedure described in Example 9. LC/MS [M+H] 418.04/420.04 (calculated); LC/MS [M+H] 418.19/420.16 (observed).

### Example 72: Synthesis of Compound 79

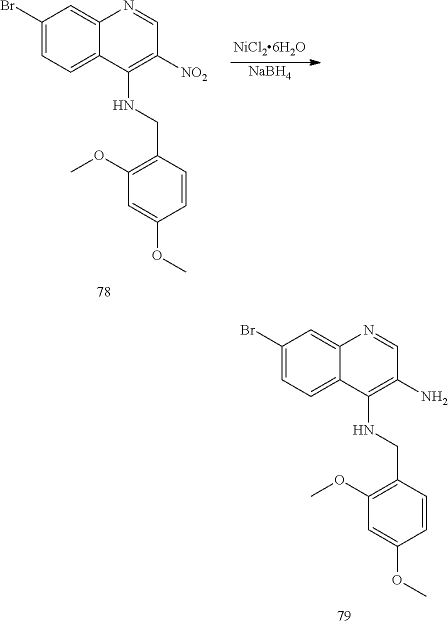 [SMILES: COc1ccc(CNc2c([N+](=O)[O-])cnc3cc(Br)ccc23)c(OC)c1 ; "NaBH4" ; COc1ccc(CNc2c(N)cnc3cc(Br)ccc23)c(OC)c1]

7-bromo-N-(2,4-dimethoxybenzyl)-3-nitroquinolin-4-amine (4.2 g, 10.0 mmol, 1 equiv.) was suspended in acetonitrile (24 ml). Water (4 ml) was added, followed by nickel(II) chloride hexahydrate (0.48 g, 2 mmol, 0.2 equiv.). Sodium borohydride (1.52 g, 40.2 mmol, 4 equiv.) was added to the green suspension and the exothermic reaction was stirred for 30 minutes. The reaction mixture was filtered, concentrated, and purified by flash chromatography to give 7-bromo-N4-(2,4-dimethoxybenzyl)quinoline-3,4-diamine (2.15 g, 5.5 mmol, 55%). LC/MS [M+H] 388.07/390.06 (calculated); LC/MS [M+H] 388.22/390.21 (observed).

### Example 73: Synthesis of Compound 80

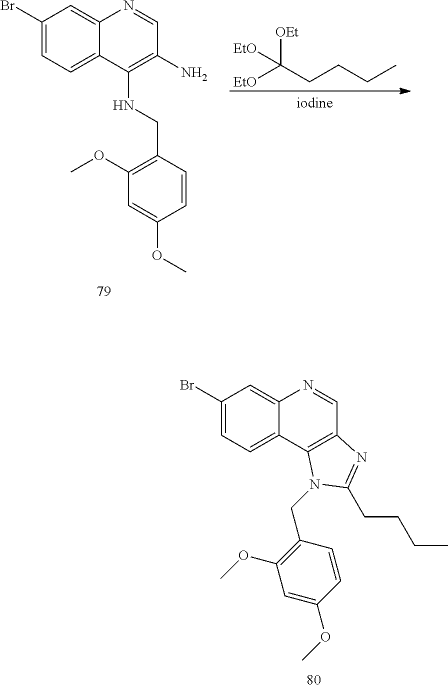 [SMILES: COc1ccc(CNc2c(N)cnc3cc(Br)ccc23)c(OC)c1 ; CCCCC(OCC)(OCC)OCC ; CCCCc1nc2cnc3cc(Br)ccc3c2n1Cc1ccc(OC)cc1OC]

7-bromo-N4-(2,4-dimethoxybenzyl)quinoline-3,4-diamine (2.15 g, 5.53 mmol, 1 equiv.) was dissolved in acetonitrile (25 ml). To the stirring solution were added triethyl orthovalerate (2.57 ml, 11.1 mmol, 2 equiv.) followed by iodine (0.140 g, 0.55 mmol, 0.1 equiv.). The reaction was stirred at room temperature until no starting material was observed by LCMS. The reaction mixture was concentrated, diluted in dichloromethane, and purified by flash chromatography to give 7-bromo-2-butyl-1-(2,4-dimethoxybenzyl)-1H-imidazo[4,5-c]quinoline (2.43 g, 5.3 mmol, 97%). LC/MS [M+H] 454.11/456.11 (calculated); LC/MS [M+H] 454.28/456.23 (observed).

### Example 74: Synthesis of Compound 81

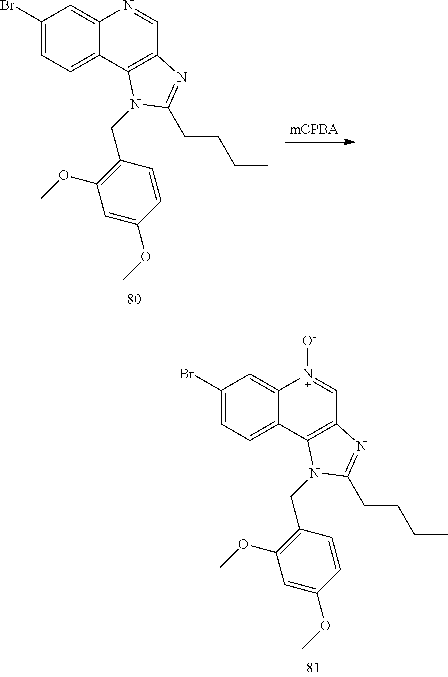 [SMILES: CCCCc1nc2cnc3cc(Br)ccc3c2n1Cc1ccc(OC)cc1OC ; CCCCc1nc2c[n+]([O-])c3cc(Br)ccc3c2n1Cc1ccc(OC)cc1OC]

7-bromo-2-butyl-1-(2,4-dimethoxybenzyl)-1H-imidazo[4,5-c]quinoline (2.7 g, 5.94 mmol, 1 equiv.) was dissolved in 15 ml DCM. To the stirring reaction was added 4-chloroperoxybenzoic acid (4.39 g, 17.83 mmol, 3 equiv.). The reaction was stirred at room temperature and monitored by LCMS. Upon consumption of starting material, the reaction was quenched with 10% aqueous sodium carbonate, extracted with ethyl acetate, concentrated, and purified by flash chromatography to give 7-bromo-2-butyl-1-(2,4-dimethoxybenzyl)-1H-imidazo[4,5-c]quinoline 5-oxide (0.88 g, 1.87 mmol, 31%). LC/MS [M+H] 470.11/472.11 (calculated); LC/MS [M+H] 470.27/472.25 (observed).

### Example 75: Synthesis of Compound 82

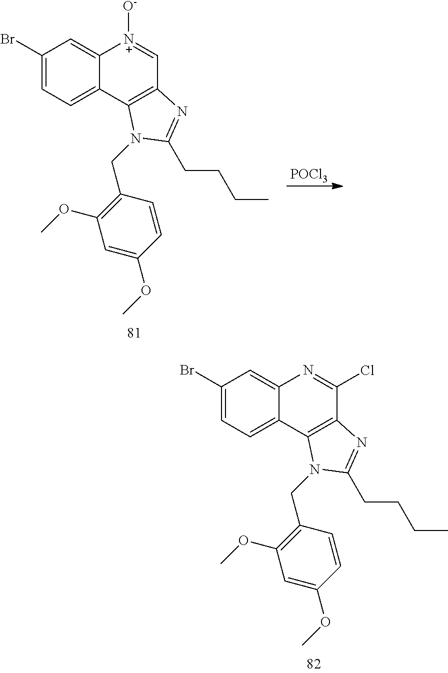 [SMILES: CCCCc1nc2c[n+]([O-])c3cc(Br)ccc3c2n1Cc1ccc(OC)cc1OC ; "POCl3" ; CCCCc1nc2c(Cl)nc3cc(Br)ccc3c2n1Cc1ccc(OC)cc1OC]

7-bromo-2-butyl-1-(2,4-dimethoxybenzyl)-1H-imidazo[4,5-c]quinoline 5-oxide (0.88 g, 1.87 mmol, 1 equiv.) was dissolved in dichloromethane (20 ml) and cooled on ice. Phosphoryl chloride (0.21 ml, 2.2 mmol, 1.2 equiv.) was added dropwise to the rapidly stirring solution, followed by N,N-dimethylformamide (0.072 ml, 0.94 mmol, 0.5 equiv.). After five minutes, the reaction was warmed to ambient temperature and monitored by LCMS. Upon consumption of starting material, the solution was washed with a mixture of ice and 10% aqueous sodium carbonate. The organic and aqueous layers were separated, and the aqueous layer extracted with dichloromethane (15 ml). The combined organic fractions were dried over sodium sulfate, filtered, and concentrated to provide 7-bromo-2-butyl-4-chloro-1-(2,4-dimethoxybenzyl)-1H-imidazo[4,5-c]quinoline as a brown foam (1.02 g, 2.09 mmol, 100%). LC/MS [M+H] 488.07/490.07 (calculated); LC/MS [M+H] 488.22/490.21 (observed).

### Example 76: Synthesis of Compound 83

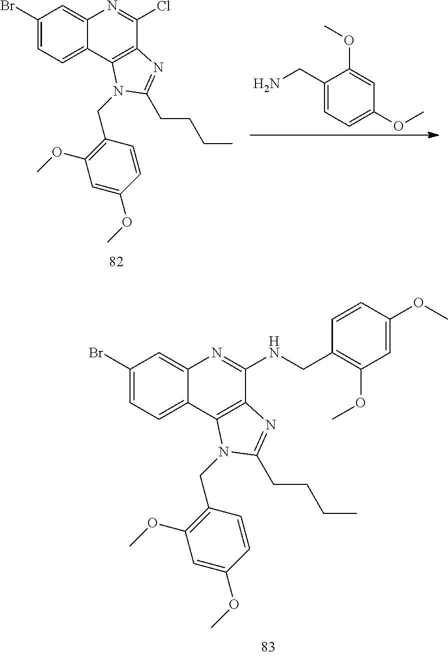 [SMILES: CCCCc1nc2c(Cl)nc3cc(Br)ccc3c2n1Cc1ccc(OC)cc1OC ; COc1ccc(CN)c(OC)c1 ; CCCCc1nc2c(NCc3ccc(OC)cc3OC)nc3cc(Br)ccc3c2n1Cc1ccc(OC)cc1OC]

7-bromo-2-butyl-4-chloro-1-(2,4-dimethoxybenzyl)-1H-imidazo[4,5-c]quinoline (1 g, 2 mmol, 1 equiv.) was converted into 7-bromo-2-butyl-1-(2,4-dimethoxybenzyl)-N-(2,4-dimethoxyphenyl)-1H-imidazo[4,5-c]quinolin-4-amine using the procedure described in Example 21 (0.694 g, 1.12 mmol, 57%). LC/MS [M+H] 619.19/621.32 (calculated); LC/MS [M+H] 619.37/621.32 (observed).

### Example 77: Synthesis of Compound 84

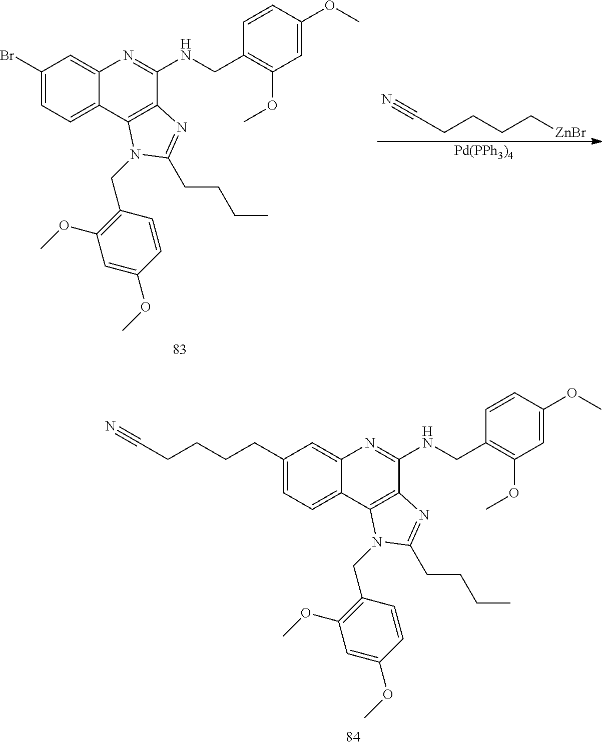

7-bromo-2-butyl-1-(2,4-dimethoxybenzyl)-N-(2,4-dimethoxyphenyl)-1H-imidazo[4,5-c]quinolin-4-amine (0.154 g, 0.25 mmol, 1 equiv.) and Pd(PPH₃)₄ (28.7 mg, 0.0025 mmol, 0.1 equiv.) were combined under dry dinitrogen. Cyanobutylzinc bromide (2.5 ml, 0.5 M in THF, 1.24 mmol, 5 equiv.) was added under dry dinitrogen and the reaction was heated to 75° C. After 30 minutes, another portion of cyanobutylzinc bromide was added (2.5 ml, 0.5 M in THF, 1.24 mmol, 5 equiv.) and the reaction allowed to stir for an additional 90 minutes. The solution was concentrated to a syrup and the crude material purified by flash chromatography to provide a mixture of the desired 5-(2-butyl-1-(2,4-dimethoxybenzyl)-4-((2,4-dimethoxyphenyl)amino)-1H-imidazo[4,5-c]quinolin-7-yl)pentanenitrile along with 2-butyl-1-(2,4-dimethoxybenzyl)-N-(2,4-dimethoxyphenyl)-1H-imidazo[4,5-c]quinolin-4-amine and residual solvent that was carried on as a crude mixture (0.288 g). LC/MS [M+H] 622.34 (calculated); LC/MS [M+H] 622.96 (observed).

### Example 78: Synthesis of Compound 85

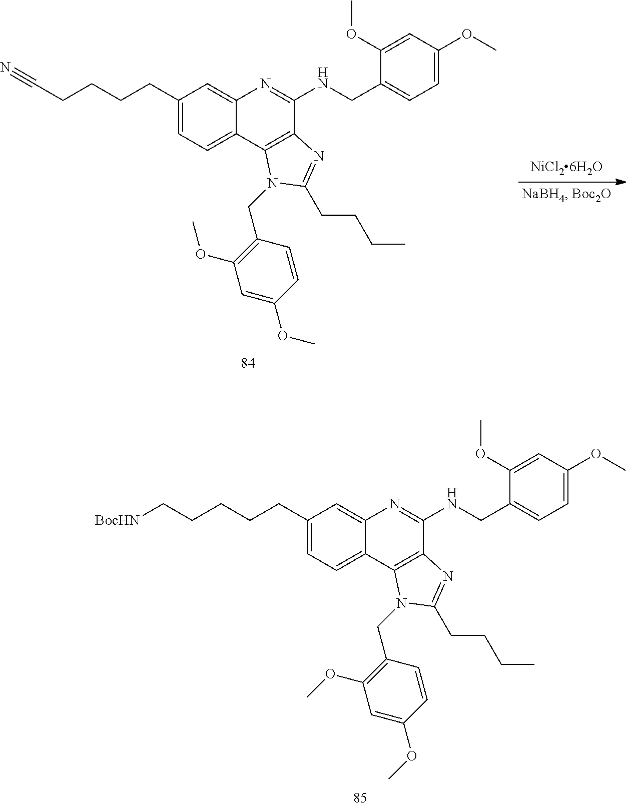 [SMILES: CCCCc1nc2c(NCc3ccc(OC)cc3OC)nc3cc(CCCCC#N)ccc3c2n1Cc1ccc(OC)cc1OC ; CCCCc1nc2c(NCc3ccc(OC)cc3OC)nc3cc(CCCCCNC(=O)OC(C)(C)C)ccc3c2n1Cc1ccc(OC)cc1OC]

5-(2-butyl-1-(2,4-dimethoxybenzyl)-4-((2,4-dimethoxyphenyl)amino)-1H-imidazo[4,5-c]quinolin-7-yl)pentanenitrile (0.69 g, 1.1 mmol, 1 equiv.) was dissolved in methanol (20 ml) and cooled on ice. Nickel(II) chloride hexahydrate (0.053 g, 0.22 mmol, 0.2 equiv.) and Boc anhydride (0.51 ml, 2.22 mmol, 2 equiv.) were added to the stirring mixture. Sodium borohydride (1 g, 26.4 mmol, 23.8 equiv.) was added slowly in portions over 1 hour. The reaction was warmed and allowed to stand at ambient temperature for 1 hour, then concentrated. The crude material was taken up in ethyl acetate and washed with saturated sodium bicarbonate. The organic fraction was dried over sodium sulfate, filtered, concentrated, and then purified by flash chromatography to provide tert-butyl (5-(2-butyl-1-(2,4-dimethoxybenzyl)-4-((2,4-dimethoxyphenyl)amino)-1H-imidazo[4,5-c]quinolin-7-yl)pentyl)carbamate (0.265 g, 0.37 mmol, 33%). LC/MS [M+H] 726.42 (calculated); LC/MS [M+H] 726.64 (observed).

### Example 79: Synthesis of Compound 86

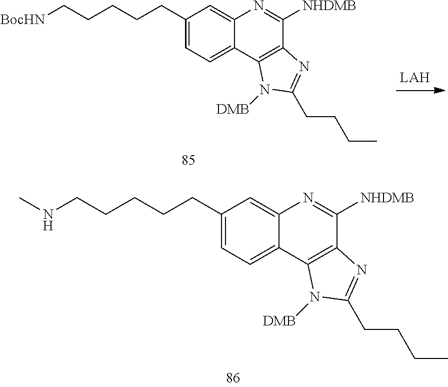

2-butyl-N,1-bis(3,4-dimethylbenzyl)-7-(5-(methylamino)pentyl)-1H-imidazo[4,5-c]quinolin-4-amine was prepared from tert-Butyl (5-(2-butyl-1-(2,4-dimethoxybenzyl)-4-((2,4-dimethoxyphenyl)amino)-1H-imidazo[4,5-c]quinolin-7-yl)pentyl)carbamate (94.3 mg, 0.13 mmol, 1 equiv.) according to the procedure set forth in Example 31. LC/MS [M+H] 640.39 (calculated); LC/MS [M+H] 640.55 (observed).

### Example 80: Synthesis of Compound 87

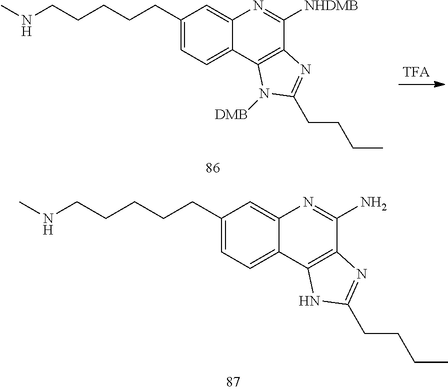

2-butyl-7-(5-(methylamino)pentyl)-1H-imidazo[4,5-c]quinolin-4-amine was prepared from 2-butyl-N,1-bis(3,4-dimethylbenzyl)-7-(5-(methylamino)pentyl)-1H-imidazo[4,5-c]quinolin-4-amine according to the procedure set forth in Example 32. LC/MS [M+H] 340.25 (calculated); LC/MS [M+H] 340.36 (observed).

### Example 81: Synthesis of Compound 88

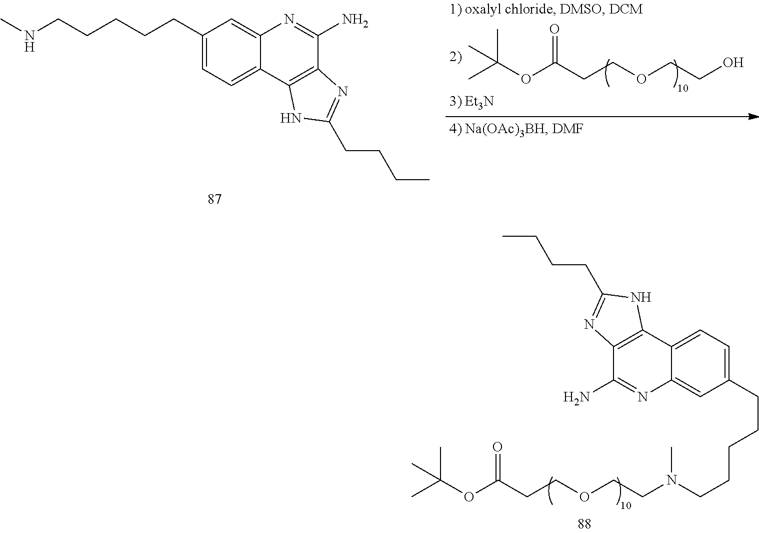

2-butyl-7-(5-(methylamino)pentyl)-1H-imidazo[4,5-c]quinolin-4-amine (50 mg, 0.15 mmol, 1 equiv.) was converted into tert-butyl 39-(4-amino-2-butyl-1H-imidazo[4,5-c]quinolin-7-yl)-34-methyl-4,7,10,13,16,19,22,25,28,31-decaoxa-34-azanonatriacontanoate using the procedure described in Example 12. LC/MS [M+H] 908.60 (calculated); LC/MS [M+H] 908.75 (observed).

### Example 82: Synthesis of Compound 89

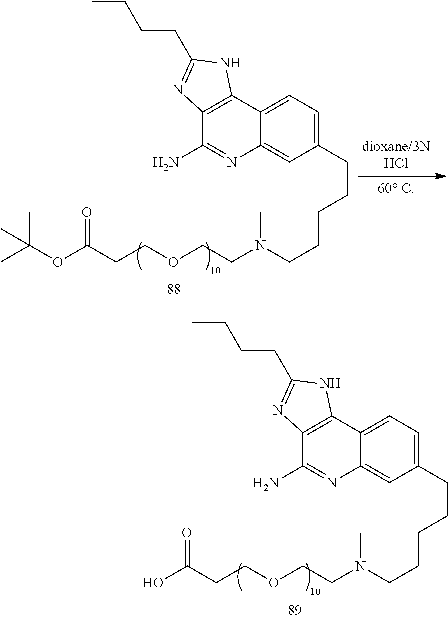

Tert-butyl 39-(4-amino-2-butyl-1H-imidazo[4,5-c]quinolin-7-yl)-34-methyl-4,7,10,13,16,19,22,25,28,31-decaoxa-34-azanonatriacontanoate was converted into 3944-amino-2-butyl-1H-imidazo[4,5-c]quinolin-7-yl)-34-methyl-4,7,10,13,16,19,22,25,28,31-decaoxa-34-azanonatriacontanoic acid (45 mg, 0.15 mmol, 33% from compound 87) using the procedure described in Example 16. LC/MS [M+H] 852.53 (calculated); LC/MS [M+H] 852.75 (observed).

### Example 83: Synthesis of Compound 90

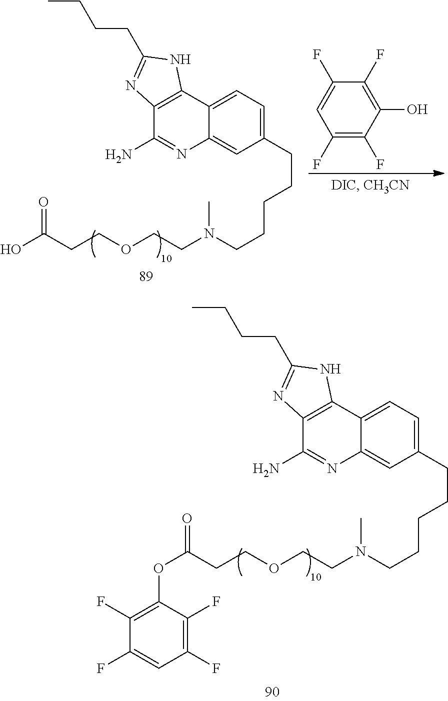

39-(4-amino-2-butyl-1H-imidazo[4,5-c]quinolin-7-yl)-34-methyl-4,7,10,13,16,19,22,25,28,31-decaoxa-34-azanonatriacontanoic acid (45 mg, 0.053 mmol, 1 equiv.) was converted into 2,3,5,6-tetrafluorophenyl 39-(4-amino-2-butyl-1H-imidazo[4,5-c]quinolin-7-yl)-34-methyl-4,7,10,13,16,19,22,25,28,31-decaoxa-34-azanonatriacontanoate (28.5 mg, 0.053 mmol, 54%) according to the procedure described in Example 17. LC/MS [M+H] 1000.53 (calculated); LC/MS [M+H] 1000.72 (observed).

### Example 84: Synthesis of Compound 92

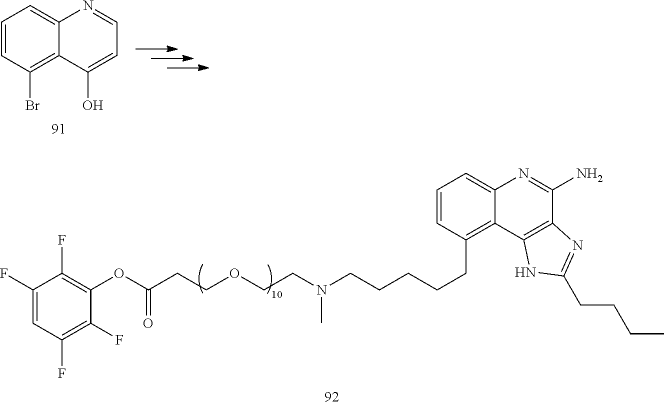

5-bromoquinolin-4-ol was converted into 2,3,5,6-tetrafluorophenyl 39-(4-amino-2-butyl-1H-imidazo[4,5-c]quinolin-9-yl)-34-methyl-4,7,10,13,16,19,22,25,28,31-decaoxa-34-azanonatriacontanoate using the route described in Examples 69-83. LC/MS [M+H] 1000.53 (calculated); LC/MS [M+H] 1000.94 (observed).

### Example 85: Synthesis of Compound 93

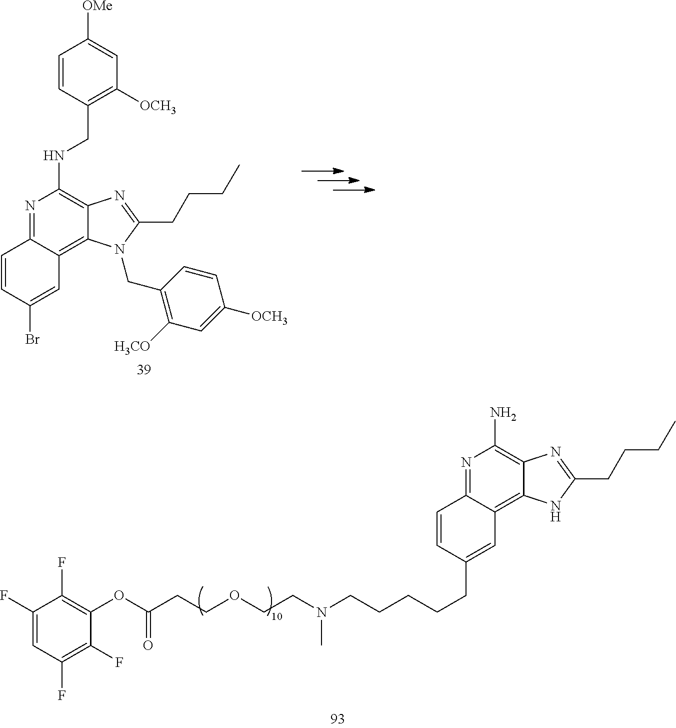

Compound 39 was converted into 2,3,5,6-tetrafluorophenyl 39-(4-amino-2-butyl-1H-imidazo[4,5-c]quinolin-8-yl)-34-methyl-4,7,10,13,16,19,22,25,28,31-decaoxa-34-azanonatriacontanoate using the route described in Examples 77-83. LC/MS [M+H] 1000.53 (calculated); LC/MS [M+H] 1000.92 (observed).

### Example 86: Synthesis of Compound 94

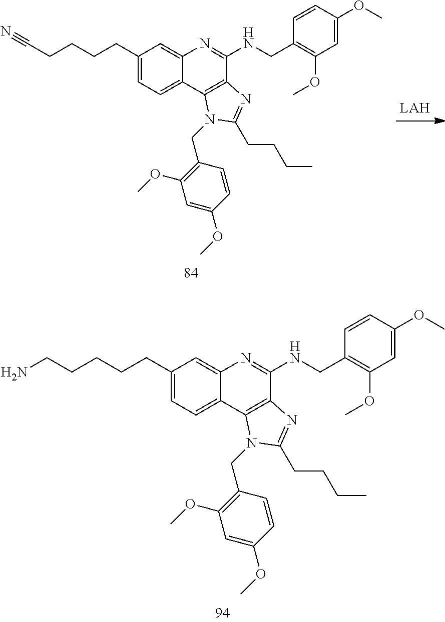 [SMILES: CCCCc1nc2c(NCc3ccc(OC)cc3OC)nc3cc(CCCCC#N)ccc3c2n1Cc1ccc(OC)cc1OC ; CCCCc1nc2c(NCc3ccc(OC)cc3OC)nc3cc(CCCCCN)ccc3c2n1Cc1ccc(OC)cc1OC]

5-(2-butyl-1-(2,4-dimethoxybenzyl)-4-((2,4-dimethoxybenzyl)amino)-1H-imidazo[4,5-c]quinolin-7-yl)pentanenitrile (0.288 g, 0.46 mmol, 1 equiv.) was dissolved in 4 ml dry THF. Lithium aluminum hydride (0.088 g, 2.3 mmol, 5 equiv.) was added and the exothermic reaction stirred at ambient temperature for 1 hour. The reaction was quenched with 0.5 ml saturated NaHCO₃, diluted with 10 ml THF, and precipitate removed by centrifugation. The resulting solution was concentrated and purified by HPLC to provide 7-(5-aminopentyl)-2-butyl-N,1-bis(2,4-dimethoxybenzyl)-1H-imidazo[4,5-c]quinolin-4-amine (0.101 g, 0.16 mmol, 35%). LC/MS [M+H] 626.37 (calculated); LC/MS [M+H] 626.56 (observed).

### Example 87: Synthesis of Compound 95

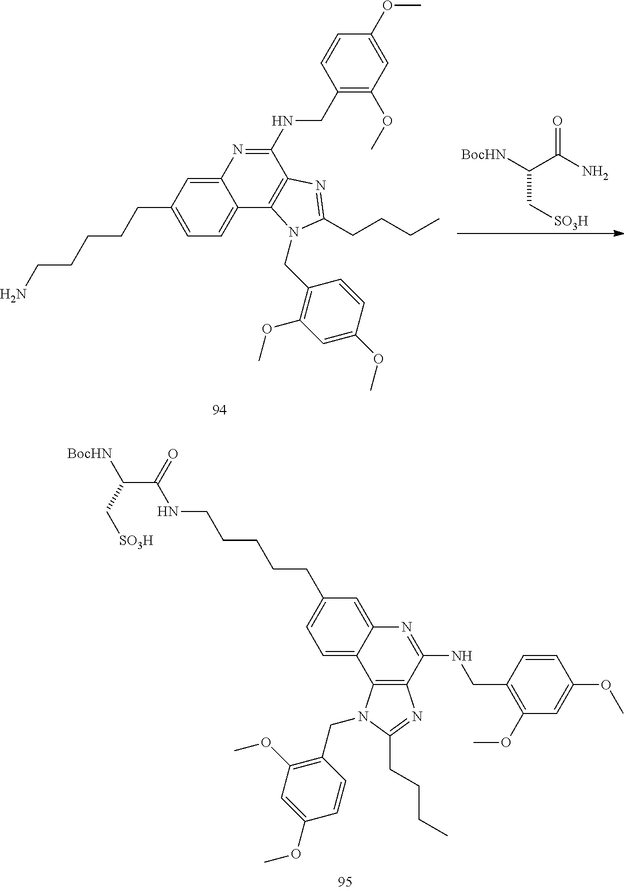 [SMILES: CCCCc1nc2c(NCc3ccc(OC)cc3OC)nc3cc(CCCCCN)ccc3c2n1Cc1ccc(OC)cc1OC ; CC(C)(C)OC(=O)N[C@@H](CS(=O)(=O)O)C(N)=O ; CCCCc1nc2c(NCc3ccc(OC)cc3OC)nc3cc(CCCCCNC(=O)[C@H](CS(=O)(=O)O)NC(=O)OC(C)(C)C)ccc3c2n1Cc1ccc(OC)cc1OC]

7-(5-aminopentyl)-2-butyl-N,1-bis(2,4-dimethoxybenzyl)-1H-imidazo[4,5-c]quinolin-4-amine (23.2 mg, 0.037 mmol, 1 equiv.) was suspended in dry N,N-dimethylformamide (2 ml). Diisopropylamine (0.065 ml, 0.37 mmol, 10 equiv.) was added, followed by (tert-butoxycarbonyl)(sulfo)-D-alanine (0.020 g, 0.074 mmol, 2 equiv.) and HATU (0.049 g, 0.13 mmol, 3.5 equiv.). The reaction was stirred at 50° C. for 30 minutes, then diluted with water and purified by HPLC to provide (R)-2-((tert-butoxycarbonyl)amino)-3-((5-(2-butyl-1-(2,4-dimethoxybenzyl)-4-((2,4-dimethoxybenzyl)amino)-1H-imidazo[4,5-c]quinolin-7-yl)pentyl)amino)-3-oxopropane-1-sulfonic acid (28.3 mg, 0.032 mmol, 87%). LC/MS [M+H] 877.42 (calculated); LC/MS [M+H] 877.59 (observed).

### Example 88: Synthesis of Compound 96

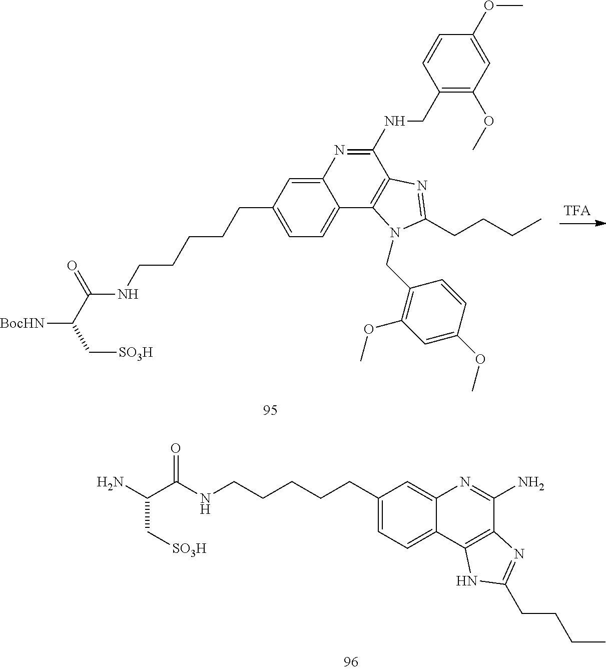 [SMILES: CCCCc1nc2c(NCc3ccc(OC)cc3OC)nc3cc(CCCCCNC(=O)[C@H](CS(=O)(=O)O)NC(=O)OC(C)(C)C)ccc3c2n1Cc1ccc(OC)cc1OC ; CCCCc1nc2c(N)nc3cc(CCCCCNC(=O)[C@@H](N)CS(=O)(=O)O)ccc3c2[nH]1]

(R)-2-((tert-butoxycarbonyl)amino)-3-((5-(2-butyl-1-(2,4-dimethoxybenzyl)-4-((2,4-dimethoxybenzyl)amino)-1H-imidazo[4,5-c]quinolin-7-yl)pentyl)amino)-3-oxopropane-1-sulfonic acid (28.3 mg, 0.032 mmol) was converted into (R)-2-amino-3-((5-(4-amino-2-butyl-1H-imidazo[4,5-c]quinolin-7-yl)pentyl)amino)-3-oxopropane-1-sulfonic acid (15.8 mg, 0.033 mmol, 100%) according to the procedure described in Example 32. LC/MS [M+H] 877.42 (calculated); LC/MS [M+H] 877.59 (observed).

### Example 89: Synthesis of Compound 97

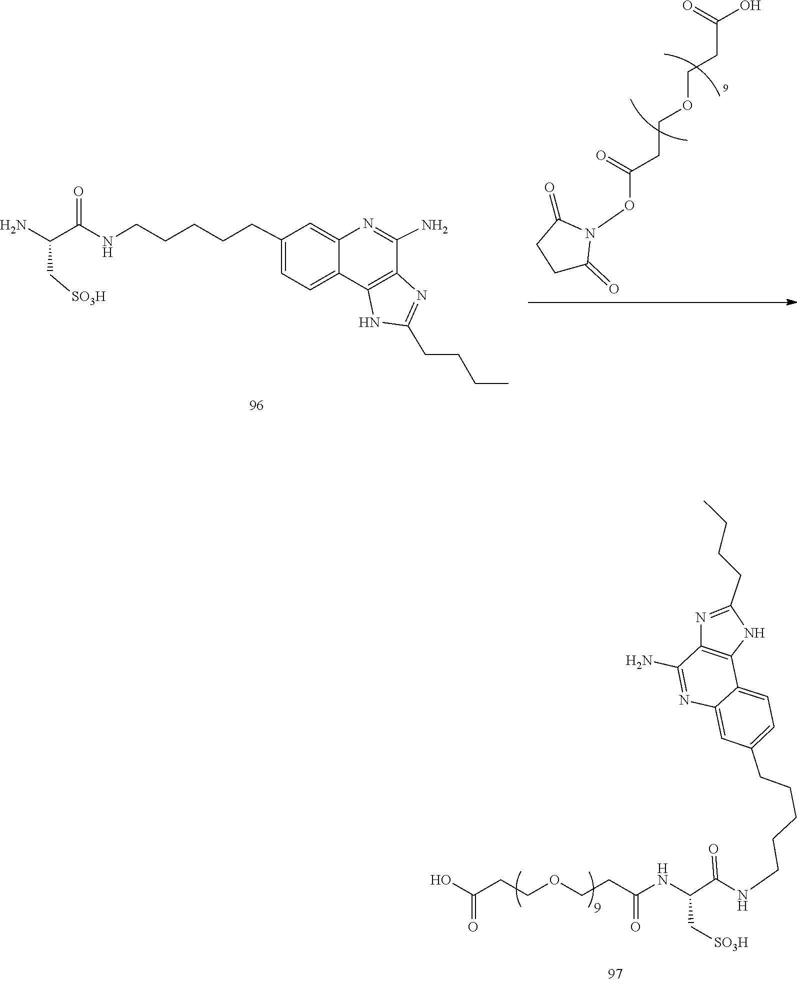

(R)-2-amino-3-((5-(4-amino-2-butyl-1H-imidazo[4,5-c]quinolin-7-yl)pentyl)amino)-3-oxopropane-1-sulfonic acid (15.8 mg, 0.033 mmol) was dissolved in dry N,N-dimethylformamide (1 ml). A solution of 31-((2,5-dioxocyclopentyl)oxy)-31-oxo-4,7,10,13,16,19,22,25,28-nonaoxahentriacontanoic acid (22.3 mg, 0.036 mmol, 1.1 equiv.) in N,N-dimethylformamide (1 ml) was added, followed by diisopropylethylamine (0.01 ml, 0.057 mmol, 1.7 equiv.). The reaction was stirred at 60° C. until complete by LCMS, then purified directly by HPLC to provide (R)-40-(4-amino-2-butyl-1H-imidazo[4,5-c]quinolin-7-yl)-33-((hydroxy(11-oxidaneyl)(oxo)-15-sulfaneyl)methyl)-31,34-dioxo-4,7,10,13,16,19,22,25,28-nonaoxa-32,35-diazatetracontanoic acid (22 mg, 0.023 mmol, 68% yield.). LC/MS [M+H] 973.48 (calculated); LC/MS [M+H] 973.76 (observed).

### Example 90: Synthesis of Compound 98

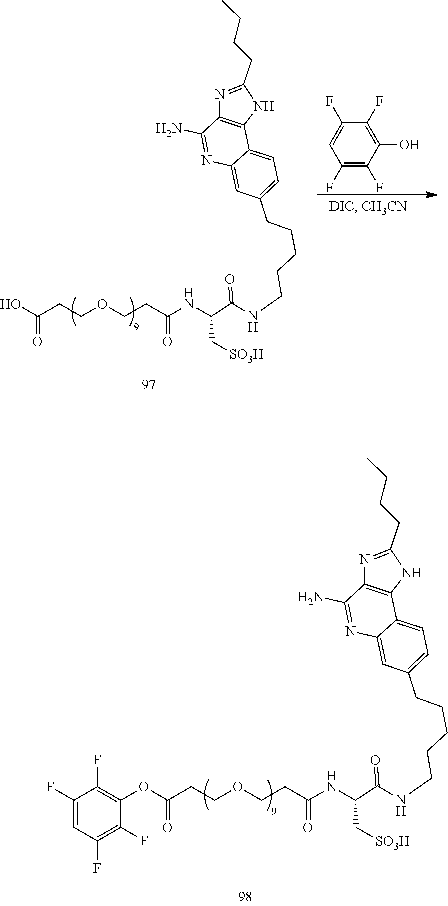

(R)-40-(4-amino-2-butyl-1H-imidazo[4,5-c]quinolin-7-yl)-33-((hydroxy(11-oxidaneyl)(oxo)-15-sulfaneyl)methyl)-31,34-dioxo-4,7,10,13,16,19,22,25,28-nonaoxa-32,35-diazatetracontanoic acid (22 mg, 0.023 mmol) was converted into 2,3,5,6-tetrafluorophenyl (R)-40-(4-amino-2-butyl-1H-imidazo[4,5-c]quinolin-7-yl)-33-((hydroxy(11-oxidaneyl)(oxo)-15-sulfaneyl)methyl)-31,34-dioxo-4,7,10,13,16,19,22,25,28-nonaoxa-32,35-diazatetracontanoate (14.2 mg, 0.013 mmol, 56%) according to the procedure described in Example 17. LC/MS [M+H] 1121.47 (calculated); LC/MS [M+H] 1121.68 (observed).

### Example 91: Synthesis of Compound 99

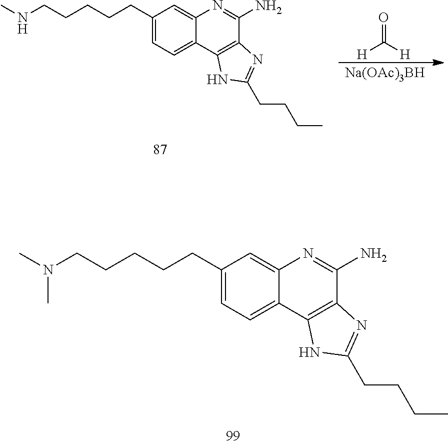

2-butyl-7-(5-(methylamino)pentyl)-1H-imidazo[4,5-c]quinolin-4-amine (0.1 g, 0.29 mmol, 1 equiv.) was dissolved in dry N,N-dimethylformamide (2 ml). Sodium triacetoxyborohydride (0.250 g, 1.17 mmol, 4 equiv.) was added, followed by formaldehyde (0.048 ml, 37% by mass, 0.597 mmol, 2 equiv.). After 15 minutes, the reaction was diluted with water and purified by HPLC to provide 2-butyl-7-(5-(dimethylamino)pentyl)-1H-imidazo[4,5-c]quinolin-4-amine (0.044 g, 0.12 mmol, 42%). LC/MS [M+H] 354.27 (calculated); LC/MS [M+H] 354.40 (observed).

### Example 92: Synthesis of Compound 100

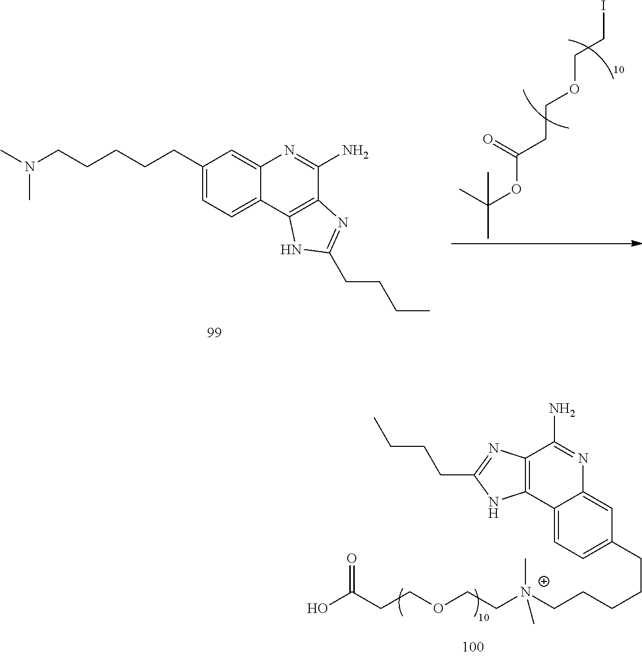

A 20 mL vial was charged with tert-butyl 1-hydroxy-3,6,9,12,15,18,21,24,27,30-decaoxatritriacontan-33-oate (0.4 mmol, 234 mg), triethylamine (0.8 mmol, 111 μL), methanesulfonyl chloride (0.44 mmol, 34 μL), and 7 mL Toluene. The reaction was heated to 60° C. for 90 min. The reaction was cooled to room temperature, filtered through diatomaceous earth, and concentrated under reduced pressure. To this concentrated crude mixture was added potassium iodide and 8 mL acetone. The reaction was heated to 50° C. for 12 h. The reaction was cooled to room temperature, filtered through diatomaceous earth, and concentrated under reduced pressure. The crude material was dissolved in dichloromethane, filtered through diatomaceous earth, concentrated under reduced pressure and used in the subsequent reaction without further purification. 2-butyl-7-(5-(dimethylamino)pentyl)-1H-imidazo[4,5-c]quinolin-4-amine (29.8 mg, 0.084 mmol, 1 equiv.) was dissolved in dry N,N-dimethylformamide (1 ml). Sodium bicarbonate (0.1 g) was added and the suspension stirred at ambient temperature for 5 minutes. tert-Butyl 1-iodo-3,6,9,12,15,18,21,24,27,30-decaoxatritriacontan-33-oate was added as a solution in dry N,N-dimethylformamide (0.5 ml), and the reaction was stirred at 60° C. The reaction solution was filtered, diluted with water, and purified by HPLC. The resulting material was taken up in 0.05 ml trifluoroacetic acid and allowed to stand at ambient temperature for 30 minutes before concentration and purification by HPLC to provide N-(5-(4-amino-2-butyl-1H-imidazo[4,5-c]quinolin-7-yl)pentyl)-32-carboxy-N,N-dimethyl-3,6,9,12,15,18,21,24,27,30-decaoxadotriacontan-1-aminium (14.3 mg, 0.016 mmol, 20%). LC/MS [M+H] 866.55 (calculated); LC/MS [M+H] 866.72 (observed).

### Example 93: Synthesis of Compound 101

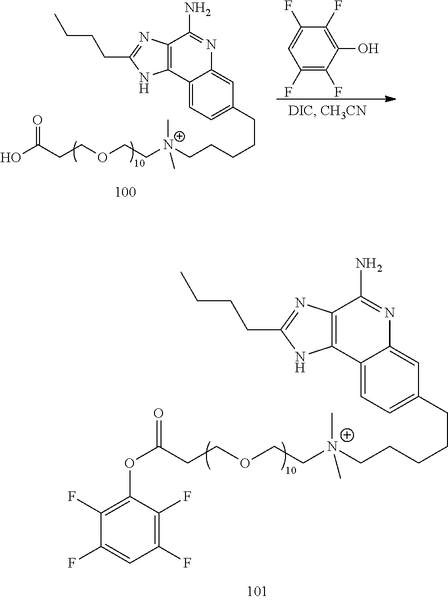

N-(5-(4-amino-2-butyl-1H-imidazo[4,5-c]quinolin-7-yl)pentyl)-32-carboxy-N,N-dimethyl-3,6,9,12,15,18,21,24,27,30-decaoxadotriacontan-1-aminium (14.3 mg, 0.016 mmol) was converted to N-(5-(4-amino-2-butyl-1H-imidazo[4,5-c]quinolin-7-yl)pentyl)-N,N-dimethyl-33-oxo-33-(2,3,5,6-tetrafluorophenoxy)-3,6,9,12,15,18,21,24,27,30-decaoxatritriacontan-1-aminium (11.4 mg, 0.011 mmol, 68%) using the procedure described in Example 17. LC/MS [M+H] 1014.54 (calculated); LC/MS [M+H] 1014.76 (observed).

### Example 94: Synthesis of Compound 102

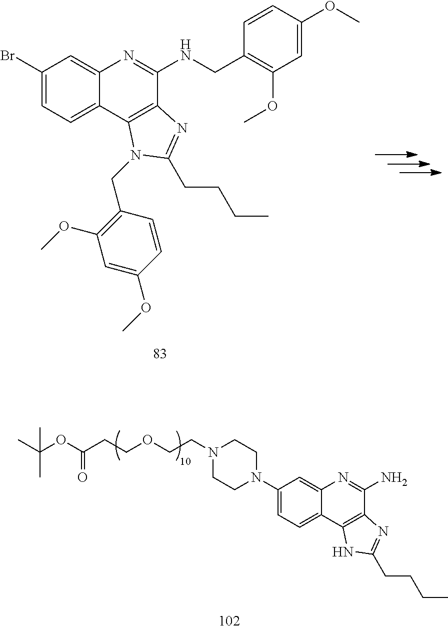

7-bromo-2-butyl-N,1-bis(2,4-dimethoxybenzyl)-1H-imidazo[4,5-c]quinolin-4-amine was converted into tert-butyl 1-(4-(4-amino-2-butyl-1H-imidazo[4,5-c]quinolin-7-yl)piperazin-1-yl)-3,6,9,12,15,18,21,24,27,30-decaoxatritriacontan-33-oate according to the procedures described in Examples 23-27. LC/MS [M+H] 893.56 (calculated); LC/MS [M+H] 893.79.

### Example 95: Synthesis of Compound 103

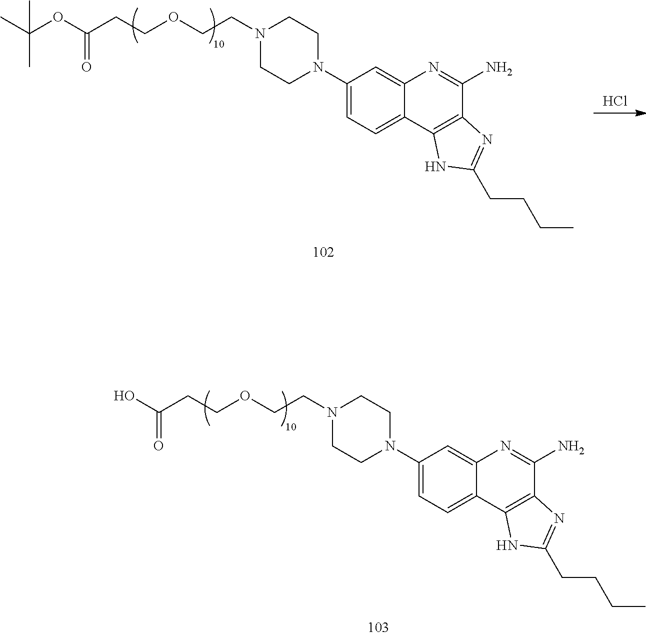

tert-butyl 1-(4-(4-amino-2-butyl-1H-imidazo[4,5-c]quinolin-7-yl)piperazin-1-yl)-3,6,9,12,15,18,21,24,27,30-decaoxatritriacontan-33-oate was converted to 1-(4-(4-amino-2-butyl-1H-imidazo[4,5-c]quinolin-7-yl)piperazin-1-yl)-3,6,9,12,15,18,21,24,27,30-decaoxatritriacontan-33-oic acid according to the procedure set forth in Example 16. LC/MS [M+H] 837.49 (calculated); LC/MS [M+H] 837.84 (observed).

### Example 96: Synthesis of Compound 104

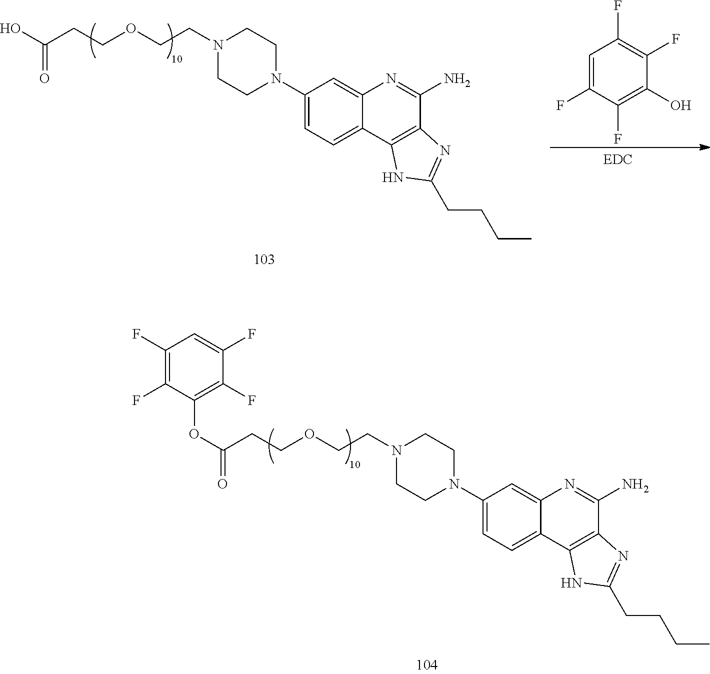

1-(4-(4-amino-2-butyl-1H-imidazo[4,5-c]quinolin-7-yl)piperazin-1-yl)-3,6,9,12,15,18,21,24,27,30-decaoxatritriacontan-33-oic acid was converted to 2,3,5,6-tetrafluorophenyl 1-(4-(4-amino-2-butyl-1H-imidazo[4,5-c]quinolin-7-yl)piperazin-1-yl)-3,6,9,12,15,18,21,24,27,30-decaoxatritriacontan-33-oate according to the procedure set forth in Example 17. LC/MS [M+H] 985.49 (calculated); [M+H] 985.71 (observed).

### Example 97: Synthesis of Compound 105

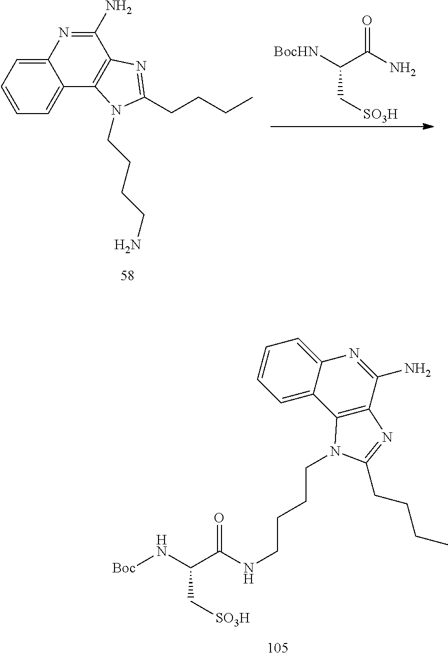 [SMILES: CCCCc1nc2c(N)nc3ccccc3c2n1CCCCN ; CC(C)(C)OC(=O)N[C@@H](CS(=O)(=O)O)C(N)=O ; CCCCc1nc2c(N)nc3ccccc3c2n1CCCCNC(=O)[C@H](CS(=O)(=O)O)NC(=O)OC(C)(C)C]

1-(4-aminobutyl)-2-butyl-1H-imidazo[4,5-c]quinolin-4-amine was converted to (R)-3-((4-(4-amino-2-butyl-1H-imidazo[4,5-c]quinolin-1-yl)butyl)amino)-2-((tert-butoxycarbonyl)amino)-3-oxopropane-1-sulfonic acid according to the procedure described in Example 87. LC/MS [M+H] 563.27 (calculated); LC/MS [M+H] 563.43 (observed).

### Example 98: Synthesis of Compound 106

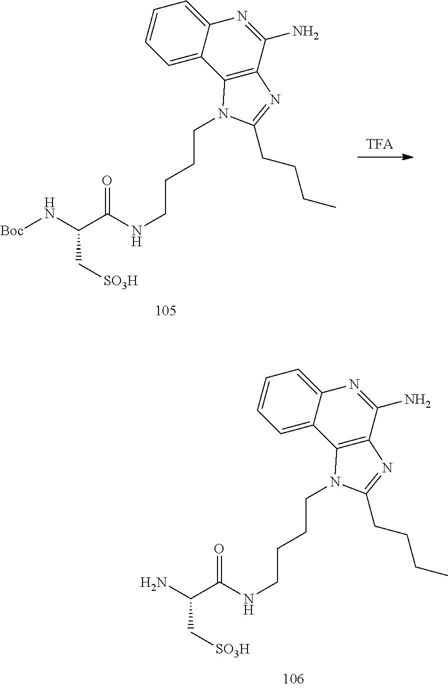 [SMILES: CCCCc1nc2c(N)nc3ccccc3c2n1CCCCNC(=O)[C@H](CS(=O)(=O)O)NC(=O)OC(C)(C)C ; CCCCc1nc2c(N)nc3ccccc3c2n1CCCCNC(=O)[C@@H](N)CS(=O)(=O)O]

(R)-3-((4-(4-amino-2-butyl-1H-imidazo[4,5-c]quinolin-1-yl)butyl)amino)-2-((tert-butoxycarbonyl)amino)-3-oxopropane-1-sulfonic acid was converted to (R)-2-amino-3-((4-(4-amino-2-butyl-1H-imidazo[4,5-c]quinolin-1-yl)butyl)amino)-3-oxopropane-1-sulfonic acid according to the procedure described in Example 88. LC/MS [M+H] 463.21 (calculated); LC/MS [M+H] 463.38 (observed).

### Example 99: Synthesis of Compound 107

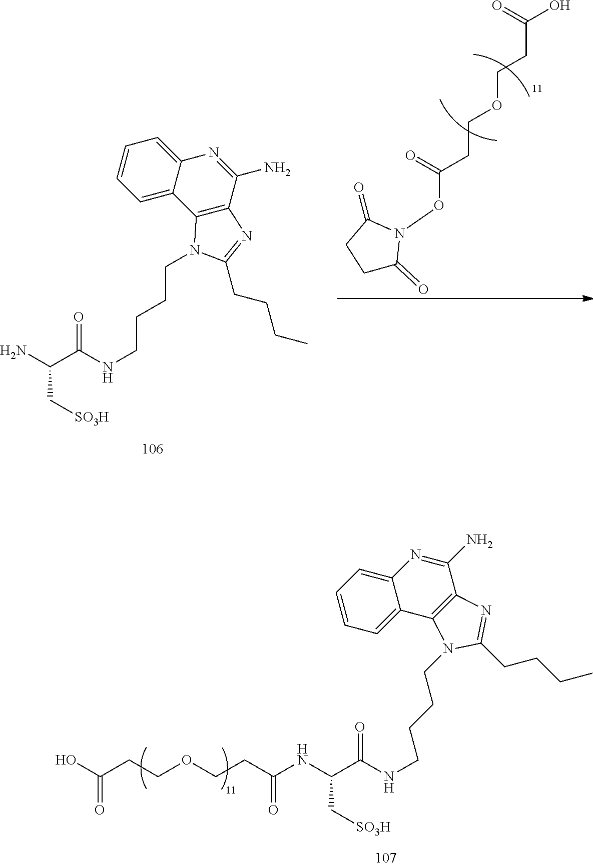

(R)-2-amino-3-((4-(4-amino-2-butyl-1H-imidazo[4,5-c]quinolin-1-yl)butyl)amino)-3-oxopropane-1-sulfonic acid was converted to (R)-45-(4-amino-2-butyl-1H-imidazo[4,5-c]quinolin-1-yl)-37,40-dioxo-39-(sulfomethyl)-4,7,10,13,16,19,22,25,28,31,34-undecaoxa-38,41-diazapentatetracontanoic acid according to the procedure described in Example 89. LC/MS [M+H] 1047.52 (calculated); LC/MS [M+H] 1047.72 (observed).

### Example 100: Synthesis of Compound 108

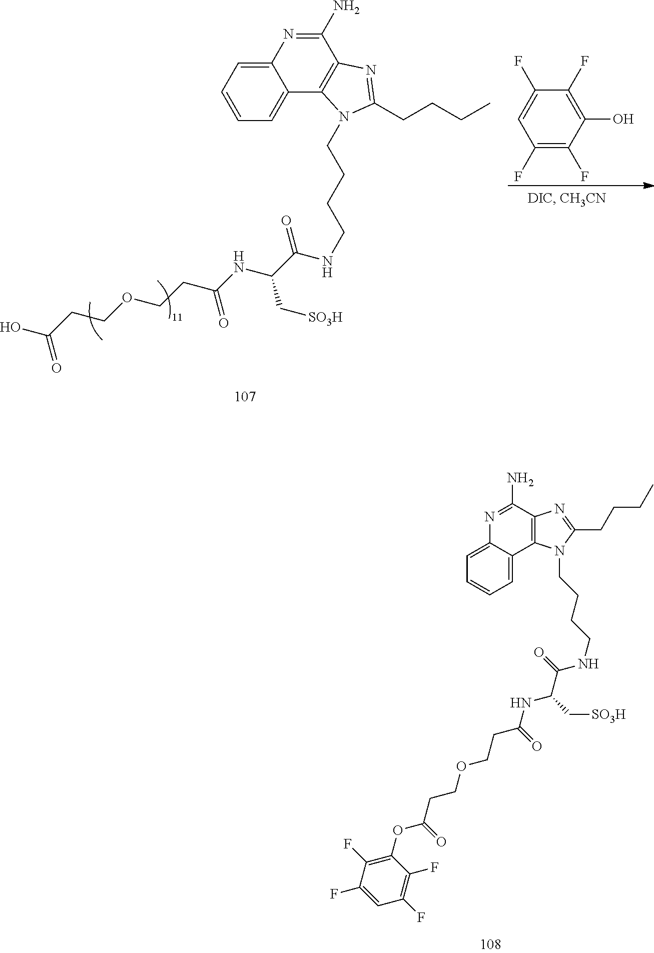

(R)-45-(4-amino-2-butyl-1H-imidazo[4,5-c]quinolin-1-yl)-37,40-dioxo-39-(sulfomethyl)-4,7,10,13,16,19,22,25,28,31,34-undecaoxa-38,41-diazapentatetracontanoic acid was converted to (R)-2-((4-(4-amino-2-butyl-1H-imidazo[4,5-c]quinolin-1-yl)butyl)carbamoyl)-4,40-dioxo-40-(2,3,5,6-tetrafluorophenoxy)-7,10,13,16,19,22,25,28,31,34,37-undecaoxa-3-azatetracontane-1-sulfonic acid according to the procedure described in Example 17. LC/MS [M+H] 1195.51 (calculated); LC/MS [M+H] 1195.73 (observed).

### Example 101: Synthesis of Compound 109

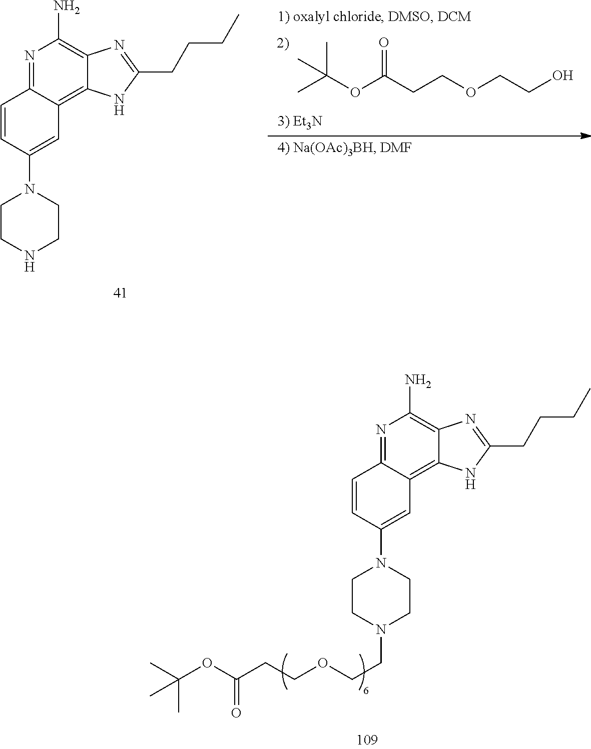

2-butyl-8-(piperazin-1-yl)-1H-imidazo[4,5-c]quinolin-4-amine was converted into tert-butyl 1-(4-(4-amino-2-butyl-1H-imidazo[4,5-c]quinolin-8-yl)piperazin-1-yl)-3,6,9,12,15,18-hexaoxahenicosan-21-oate according to the procedure described in Example 12. LC/MS [M+H] 717.45 (calculated); LC/MS [M+H] 717.75 (observed).

### Example 102: Synthesis of Compound 110

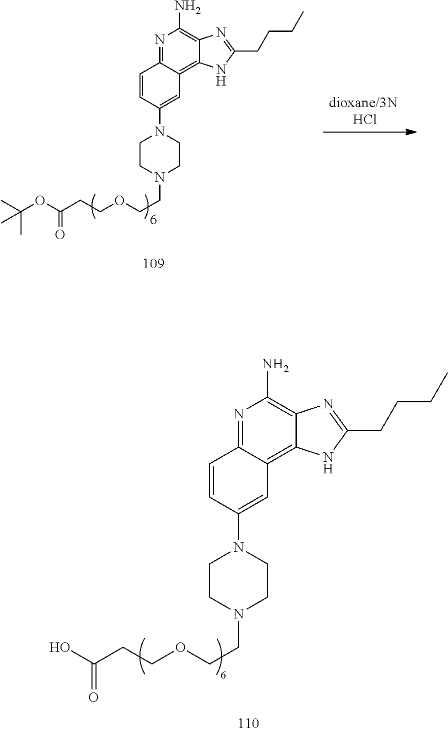

tert-Butyl 1-(4-(4-amino-2-butyl-1H-imidazo[4,5-c]quinolin-8-yl)piperazin-1-yl)-3,6,9,12,15,18-hexaoxahenicosan-21-oate was converted into 1-(4-(4-amino-2-butyl-1H-imidazo[4,5-c]quinolin-8-yl)piperazin-1-yl)-3,6,9,12,15,18-hexaoxahenicosan-21-oic acid according to the procedure described in Example 16. LC/MS [M+H] 661.39 (calculated); LC/MS [M+H] 661.60 (observed).

### Example 103: Synthesis of Compound 111

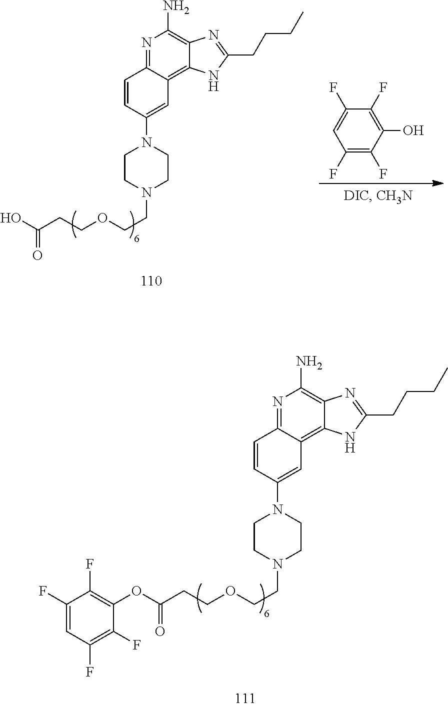

1-(4-(4-amino-2-butyl-1H-imidazo[4,5-c]quinolin-8-yl)piperazin-1-yl)-3,6,9,12,15,18-hexaoxahenicosan-21-oic acid was converted into 2,3,5,6-tetrafluorophenyl 1-(4-(4-amino-2-butyl-1H-imidazo[4,5-c]quinolin-8-yl)piperazin-1-yl)-3,6,9,12,15,18-hexaoxahenicosan-21-oate according to the procedure described in Example 17. LC/MS [M+H] 809.39 (calculated); LC/MS [M+H] 809.62 (observed).

### Example 104: Synthesis of Compound 112

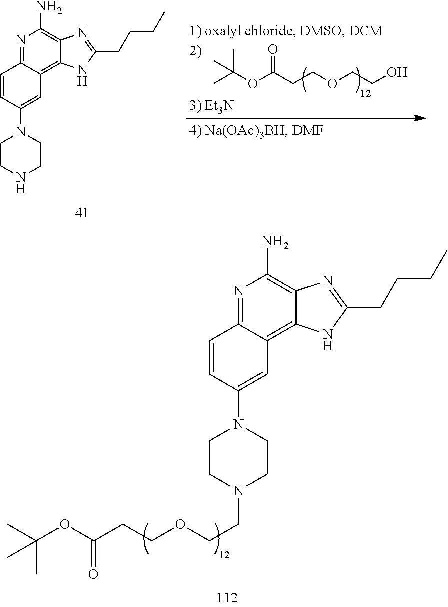

2-butyl-8-(piperazin-1-yl)-1H-imidazo[4,5-c]quinolin-4-amine was converted into tert-butyl 1-(4-(4-amino-2-butyl-1H-imidazo[4,5-c]quinolin-8-yl)piperazin-1-yl)-3,6,9,12,15,18,21,24,27,30,33,36-dodecaoxanonatriacontan-39-oate according to the procedure described in Example 12. LC/MS [M+H] 981.61 (calculated); LC/MS [M+H] 981.86 (observed).

### Example 105: Synthesis of Compound 113

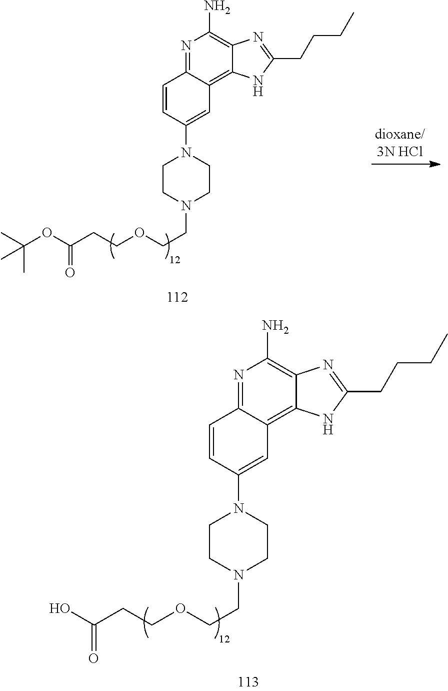

Tert-butyl 1-(4-(4-amino-2-butyl-1H-imidazo[4,5-c]quinolin-8-yl)piperazin-1-yl)-3,6,9,12,15,18,21,24,27,30,33,36-dodecaoxanonatriacontan-39-oate was converted into 1-(4-(4-amino-2-butyl-1H-imidazo[4,5-c]quinolin-8-yl)piperazin-1-yl)-3,6,9,12,15,18,21,24,27,30,33,36-dodecaoxanonatriacontan-39-oic acid according to the procedure described in Example 16. The compound was used without further purification.

### Example 106: Synthesis of Compound 114

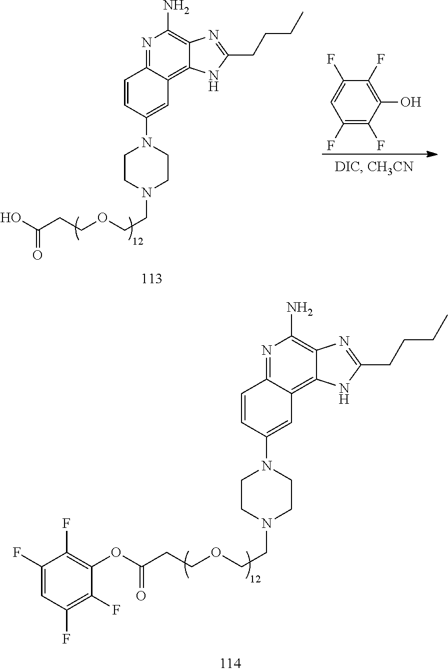

1-(4-(4-amino-2-butyl-1H-imidazo[4,5-c]quinolin-8-yl)piperazin-1-yl)-3,6,9,12,15,18,21,24,27,30,33,36-dodecaoxanonatriacontan-39-oic acid was converted into 2,3,5,6-tetrafluorophenyl 1-(4-(4-amino-2-butyl-1H-imidazo[4,5-c]quinolin-8-yl)piperazin-1-yl)-3,6,9,12,15,18,21,24,27,30,33,36-dodecaoxanonatriacontan-39-oate according to the procedure described in Example 17. LC/MS [M+H] 1073.54 (calculated); LC/MS [M+H] 1073.81 (observed).

### Example 107: Synthesis of Compound 115

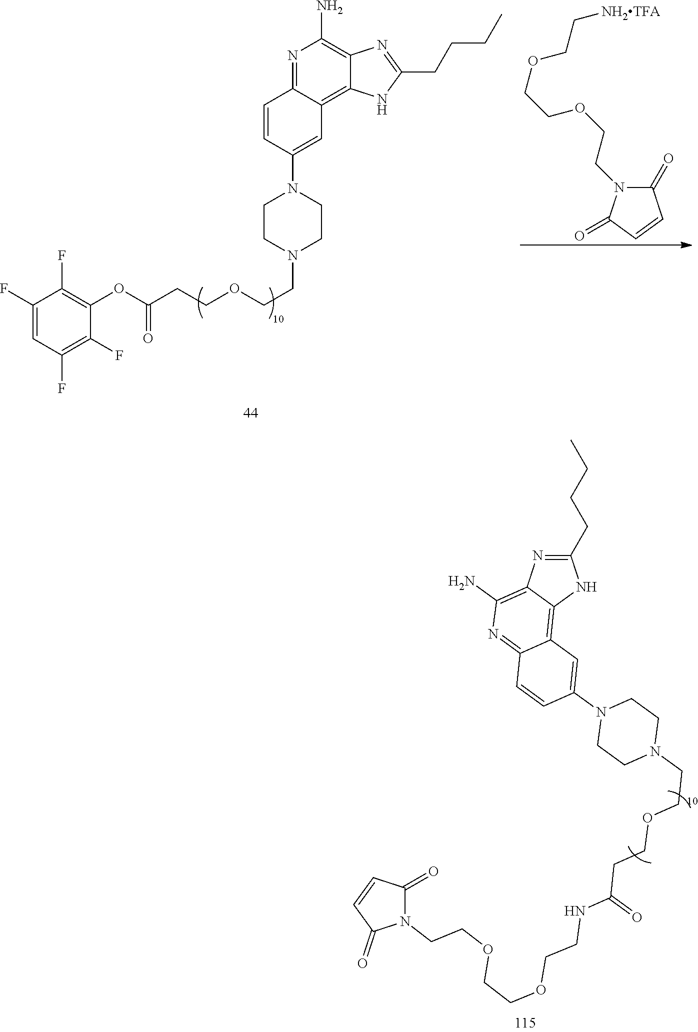

A 4 mL vial was charged with 1-(2-(2-(2-aminoethoxy)ethoxy)ethyl)-1H-pyrrole-2,5-dione (0.089 mmol, 88 mg), 2,4,6-collidine (0.27 mmol, 36 μL) and 800 μL. To this was added a solution of compound 44 (0.098 mmol, 33 mg), HOAT (0.014 mmol, 2 mg), DIPEA (0.20 mmol, 36 μL) in 100 μL DMF. The reaction was stirred for 4 h and then purified by reverse phase preparative HPLC utilizing a 25-75% gradient of acetonitrile:water containing 0.1% trifluoroacetic acid. The purified fractions were combined and lyophilized to afford 46.7 mg of the desired product Compound 115 in 50% yield. LC/MS [M+H] 1047.60 (calculated); LC/MS [M+H] 1048.01 (observed).

### Example 108: Synthesis of Compound 116

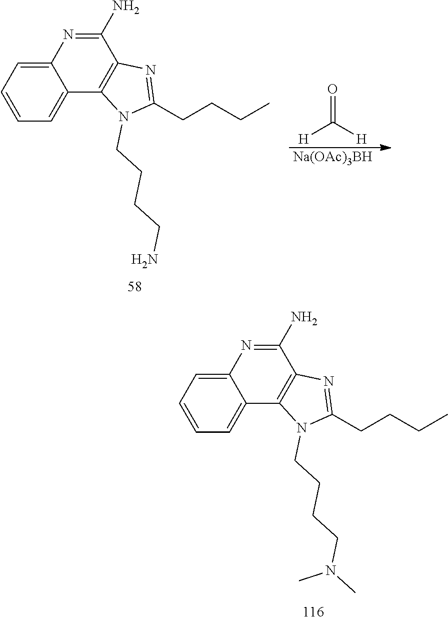 [SMILES: CCCCc1nc2c(N)nc3ccccc3c2n1CCCCN ; C=O ; CCCCc1nc2c(N)nc3ccccc3c2n1CCCCN(C)C ; "Na(OAc)3BH"]

A 4 mL vial was charged with 1-(4-aminobutyl)-2-butyl-1H-imidazo[4,5-c]quinolin-4-amine (0.13 mmol, 44 mg), formaldehyde (0.38 mmol, 31 μL of 37% solution), sodium triacetoxyborohydride (0.65 mmol, 135 mg), and 1 mL THF. Let stir for 1 h then quench with 100 μL of 10% K₂CO₃. The reaction was concentrated under reduced pressure and purified by reverse phase preparative HPLC utilizing a 25-75% gradient of acetonitrile:water containing 0.1% trifluoroacetic acid. The purified fractions were combined and lyophilized to afford 24.5 mg of the desired product 2-butyl-1-(4-(methylamino)butyl)-1H-imidazo[4,5-c]quinolin-4-amine in 57% yield. LC/MS [M+H] 340.25 (calculated); LC/MS [M+H] 340.40 (observed).

### Example 109: Synthesis of Compound 117

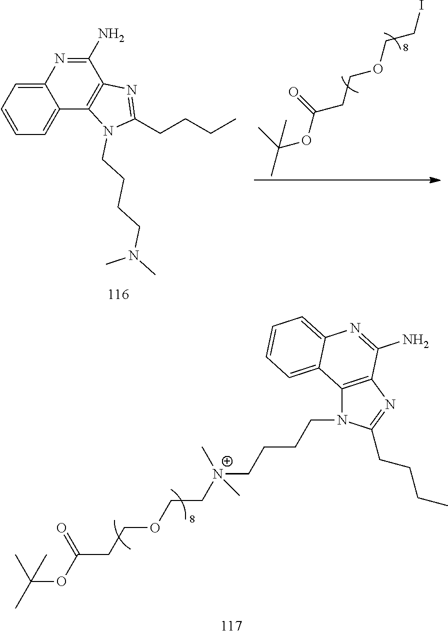

A 20 mL vial was charged with tert-butyl 1-hydroxy-3,6,9,12,15,18,21,24-octaoxaheptacosan-27-oate (0.4 mmol, 200 mg), triethylamine (0.8 mmol, 111 μL), methanesulfonyl chloride (0.44 mmol, 34 μL), and 7 mL Toluene. The reaction was heated to 60° C. for 90 min. The reaction was cooled to room temperature, filtered through diatomaceous earth, and concentrated under reduced pressure. To this concentrated crude mixture was added potassium iodide and 8 mL acetone. The reaction was heated to 50° C. for 12 h. The reaction was cooled to room temperature, filtered through diatomaceous earth, and concentrated under reduced pressure. The crude material was dissolved in dichloromethane, filtered through diatomaceous earth, concentrated under reduced pressure and used in the subsequent reaction without further purification. A 4 mL vial was charged with tert-butyl 1-iodo-3,6,9,12,15,18,21,24-octaoxaheptacosan-27-oate (0.097 mmol, 59 mg), 2-butyl-1-(4-(methylamino)butyl)-1H-imidazo[4,5-c]quinolin-4-amine (0.081 mmol, 27.4 mg), K₂CO₃ (0.16 mmol, 22.3 mg), acetonitrile (700 μL), and water (50 μL). The reaction was heated to 70° C. and stirred for 3 h. The reaction was purified by reverse phase preparative HPLC utilizing a 25-75% gradient of acetonitrile:water containing 0.1% trifluoroacetic acid. The purified fractions were combined and lyophilized to afford 22.2 mg of the desired product tert-butyl 32-(4-amino-2-butyl-1H-imidazo[4,5-c]quinolin-1-yl)-28-methyl-4,7,10,13,16,19,22,25-octaoxa-28-azadotriacontanoate in 34% yield. LC/MS [M+H] 820.54 (calculated); LC/MS [M+H] 820.75 (observed).

### Example 110: Synthesis of Compound 118

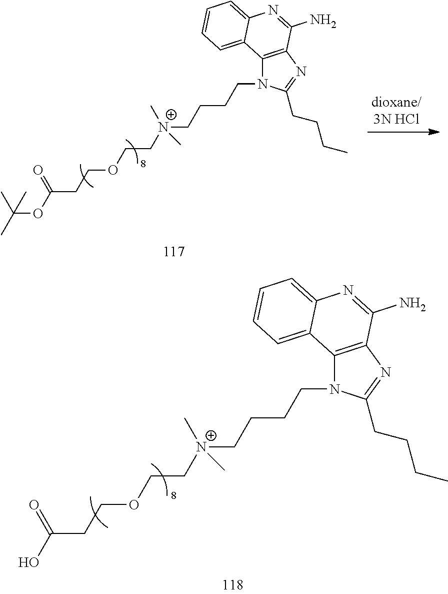

N-(4-(4-amino-2-butyl-1H-imidazo[4,5-c]quinolin-1-yl)butyl)-N,N,29,29-tetramethyl-27-oxo-3,6,9,12,15,18,21,24,28-nonaoxatriacontan-1-aminium was converted into N-(4-(4-amino-2-butyl-1H-imidazo[4,5-c]quinolin-1-yl)butyl)-26-carboxy-N,N-dimethyl-3,6,9,12,15,18,21,24-octaoxahexacosan-1-aminium according to the procedure described in Example 16. Compound was in subsequent step without further purification.

### Example 111: Synthesis of Compound 119

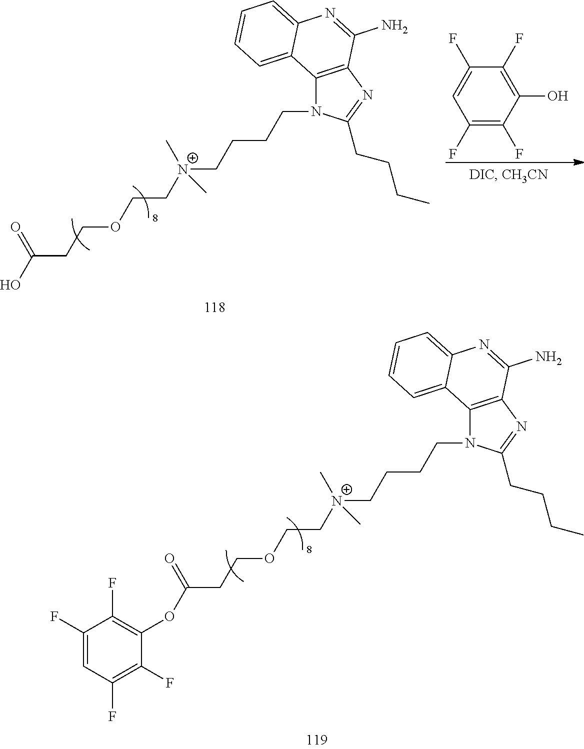

N-(4-(4-amino-2-butyl-1H-imidazo[4,5-c]quinolin-1-yl)butyl)-26-carboxy-N,N-dimethyl-3,6,9,12,15,18,21,24-octaoxahexacosan-1-aminium was converted into N-(4-(4-amino-2-butyl-1H-imidazo[4,5-c]quinolin-1-yl)butyl)-N,N-dimethyl-27-oxo-27-(2,3,5,6-tetrafluorophenoxy)-3,6,9,12,15,18,21,24-octaoxaheptacosan-1-aminium according to the procedure described in Example 17. LC/MS 912.47 [M+H] (calculated); LC/MS [M+H] 912.70 (observed).

### Example 112: Synthesis of Compound 120

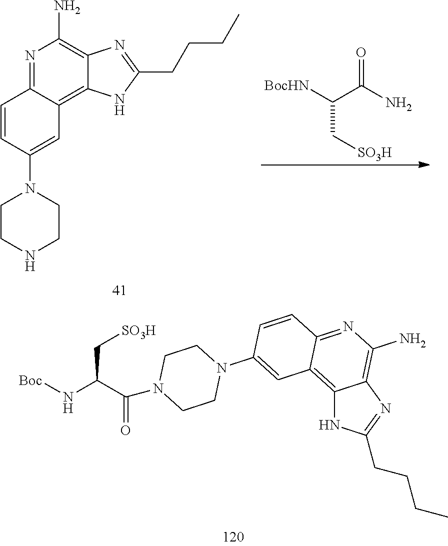 [SMILES: CCCCc1nc2c(N)nc3ccc(N4CCNCC4)cc3c2[nH]1 ; CC(C)(C)OC(=O)N[C@@H](CS(=O)(=O)O)C(N)=O ; CCCCc1nc2c(N)nc3ccc(N4CCN(C(=O)[C@H](CS(=O)(=O)O)NC(=O)OC(C)(C)C)CC4)cc3c2[nH]1]

2-butyl-8-(piperazin-1-yl)-1H-imidazo[4,5-c]quinolin-4-amine was converted to (R)-3-(4-(4-amino-2-butyl-1H-imidazo[4,5-c]quinolin-8-yl)piperazin-1-yl)-2-((tert-butoxycarbonyl)amino)-3-oxopropane-1-sulfonic acid according to the procedure described in Example 87. LC/MS [M+H] 576.26 (calculated); LC/MS [M+H] 576.44 (observed).

### Example 113: Synthesis of Compound 121

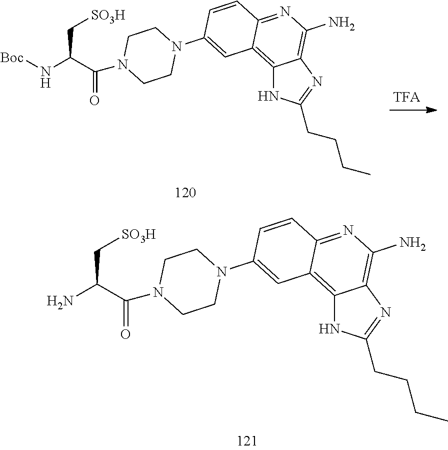 [SMILES: CCCCc1nc2c(N)nc3ccc(N4CCN(C(=O)[C@H](CS(=O)(=O)O)NC(=O)OC(C)(C)C)CC4)cc3c2[nH]1 ; CCCCc1nc2c(N)nc3ccc(N4CCN(C(=O)[C@@H](N)CS(=O)(=O)O)CC4)cc3c2[nH]1]

(R)-3-(4-(4-amino-2-butyl-1H-imidazo[4,5-c]quinolin-8-yl)piperazin-1-yl)-2-((tert-butoxycarbonyl)amino)-3-oxopropane-1-sulfonic acid was converted to (R)-2-amino-3-(4-(4-amino-2-butyl-1H-imidazo[4,5-c]quinolin-8-yl)piperazin-1-yl)-3-oxopropane-1-sulfonic acid according to the procedure described in Example 88. The compound was used in the subsequent step without further purification.

### Example 114: Synthesis of Compound 122

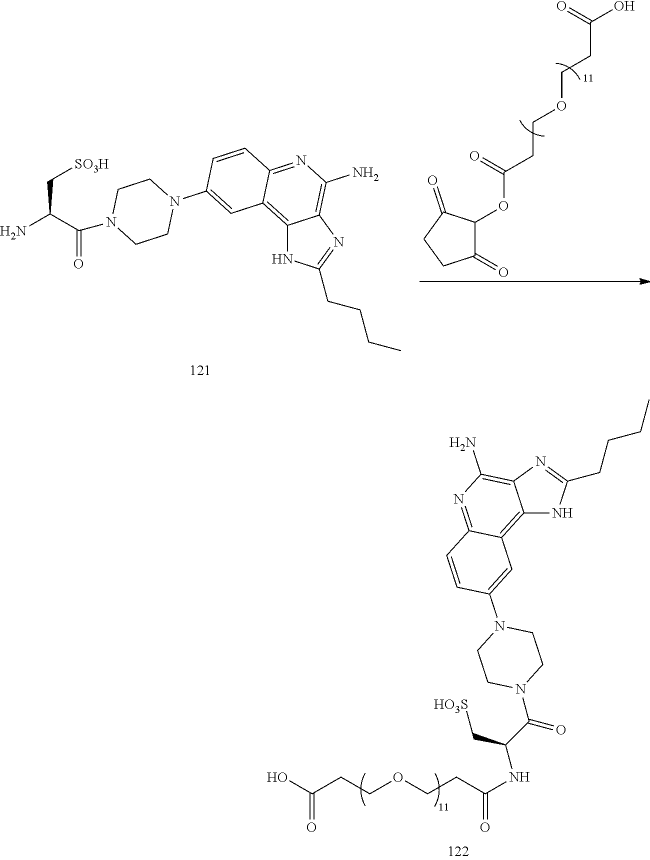

(R)-2-amino-3-(4-(4-amino-2-butyl-1H-imidazo[4,5-c]quinolin-8-yl)piperazin-1-yl)-3-oxopropane-1-sulfonic acid was converted to (R)-1-(4-(4-amino-2-butyl-1H-imidazo[4,5-c]quinolin-8-yl)piperazin-1-yl)-1,4-dioxo-2-(sulfomethyl)-7,10,13,16,19,22,25,28,31,34,37-undecaoxa-3-azatetracontan-40-oic acid according to the procedure described in Example 89. LC/MS [M+H] 1060.51 (calculated); LC/MS [M+H] 1060.73 (observed).

### Example 115: Synthesis of Compound 123

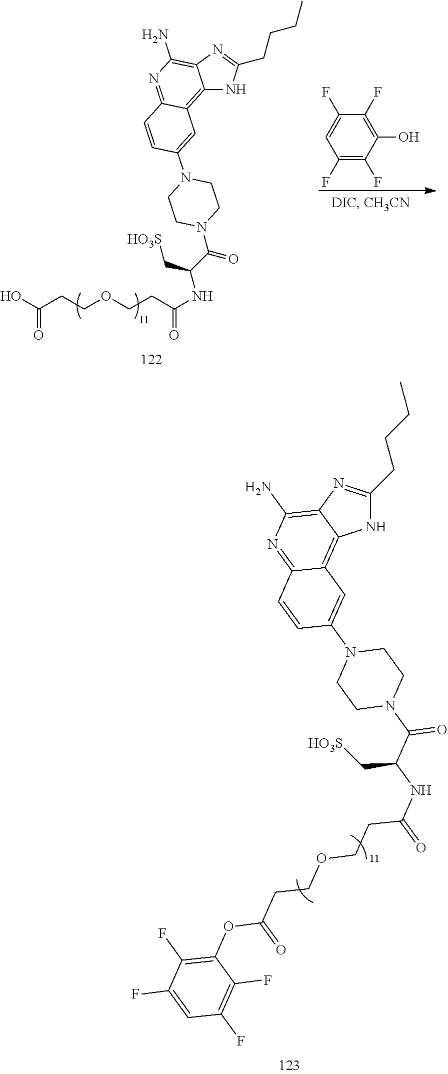

(R)-1-(4-(4-amino-2-butyl-1H-imidazo[4,5-c]quinolin-8-yl)piperazin-1-yl)-1,4-dioxo-2-(sulfomethyl)-7,10,13,16,19,22,25,28,31,34,37-undecaoxa-3-azatetracontan-40-oic acid was converted to (R)-2-(4-(4-amino-2-butyl-1H-imidazo[4,5-c]quinolin-8-yl)piperazine-1-carbonyl)-4,40-dioxo-40-(2,3,5,6-tetrafluorophenoxy)-7,10,13,16,19,22,25,28,31,34,37-undecaoxa-3-azatetracontane-1-sulfonic acid according to the procedure described in Example 17. LC/MS [M+H] 1208.51 (calculated); LC/MS [M+H] 1208.74 (observed).

### Example 116: Synthesis of Compound 124

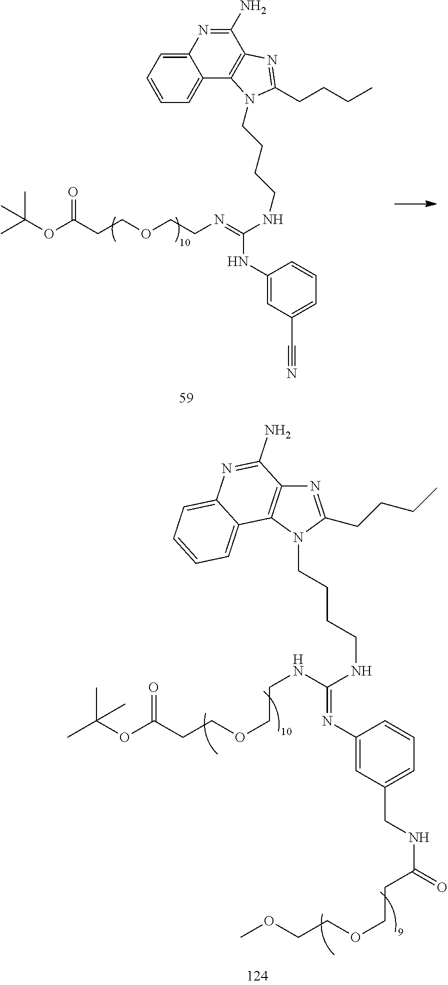

A 4 mL vial was charged with tert-butyl (Z)-40-(4-amino-2-butyl-1H-imidazo[4,5-c]quinolin-1-yl)-35-((3-cyanophenyl)amino)-4,7,10,13,16,19,22,25,28,31-decaoxa-34,36-diazatetracont-34-enoate (0.017 mmol, 17 mg), 22 mg of 10% palladium over carbon, and 1.1 mL of ethanol. The vial was evacuated and backfilled thrice with hydrogen gas. The reaction was stirred for 4 h, filtered through diatomaceous earth, and concentrated under reduced pressure. The crude reaction was dissolved in dichloromethane and concentrated under reduced pressure. To the vial containing the crude reaction mixture was added 2,5-dioxopyrrolidin-1-yl 2,5,8,11,14,17,20,23,26,29-decaoxadotriacontan-32-oate (0.017 mmol, 11 mg), Hunigs base (0.069 mmol, 12 μL) and 300 μLDMF. The reaction was stirred for 4 h. The crude reaction was purified using reverse phase preparative HPLC utilizing a 25-75% gradient of acetonitrile:water containing 0.1% trifluoroacetic acid. The purified fractions were combined and lyophilized to afford 7.1 mg of the desired product tert-butyl (E)-40-(4-amino-2-butyl-1H-imidazo[4,5-c]quinolin-1-yl)-35-((3-(32-oxo-2,5,8,11,14,17,20,23,26,29-decaoxa-33-azatetratriacontan-34-yl)phenyl)imino)-4,7,10,13,16,19,22,25,28,31-decaoxa-34,36-diazatetracontanoate in 28% yield over two steps. LC/MS [M+H] 1509.92 (calculated); LC/MS [M+H] 1510.13 (observed).

### Example 117: Synthesis of Compound 125

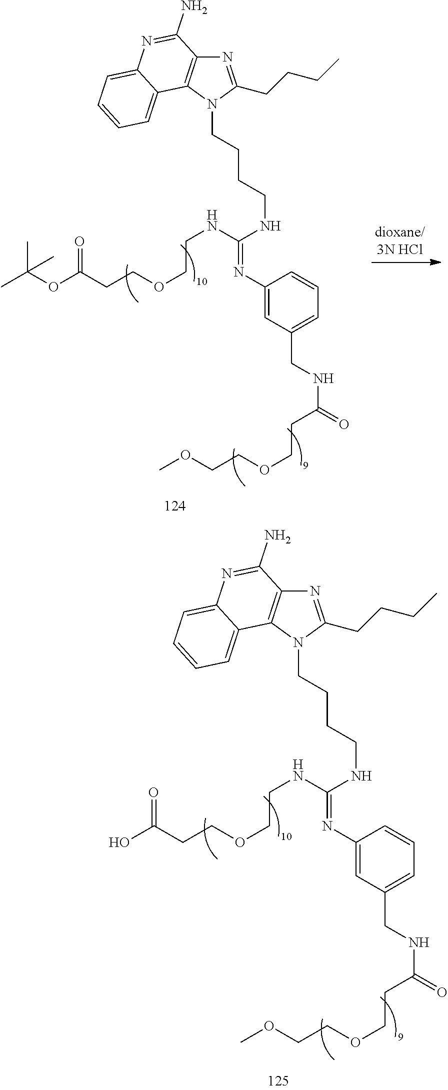

A 4 mL vial was charged with Tert-butyl (E)-40-(4-amino-2-butyl-1H-imidazo[4,5-c]quinolin-1-yl)-35-((3-(32-oxo-2,5,8,11,14,17,20,23,26,29-decaoxa-33-azatetratriacontan-34-yl)phenyl)imino)-4,7,10,13,16,19,22,25,28,31-decaoxa-34,36-diazatetracontanoate (4.7 ?amok 7.1 mg) and 800 μL of 4M HCl in 1,4-dioxane. The reaction was stirred for 4 h then concentrated under reduced pressure. The crude reaction was dissolved in 1 mL Toluene and concentrated under reduced pressure thrice. The crude material was taken on without further purification.

### Example 118: Synthesis of Compound 126

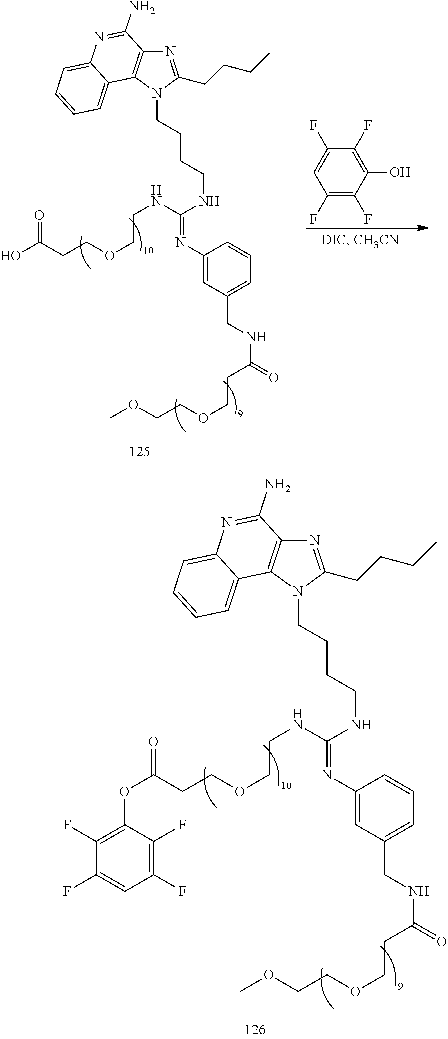

A 4 mL vial was charged with (E)-40-(4-amino-2-butyl-1H-imidazo[4,5-c]quinolin-1-yl)-35-((3-(32-oxo-2,5,8,11,14,17,20,23,26,29-decaoxa-33-azatetratriacontan-34-yl)phenyl)imino)-4,7,10,13,16,19,22,25,28,31-decaoxa-34,36-diazatetracontanoic acid (4.7 μmol), 2,3,5,6-tetrafluorophenol (9.4 μmol, 1.6 mg), 1-ethyl-3-(3-dimethylaminopropyl)carbodiimide (26 wok 5 mg), 2,4,6-collidine (60 wok 8 μL) and 100 μL of DMF. The reaction was stirred for 2 h then purified by reverse phase preparative HPLC utilizing a 25-75% gradient of acetonitrile:water containing 0.1% trifluoroacetic acid. The purified fractions were combined and lyophilized to afford 3.4 mg of the desired product 2,3,5,6-tetrafluorophenyl (E)-40-(4-amino-2-butyl-1H-imidazo[4,5-c]quinolin-1-yl)-35-((3-(32-oxo-2,5,8,11,14,17,20,23,26,29-decaoxa-33-azatetratriacontan-34-yl)phenyl)imino)-4,7,10,13,16,19,22,25,28,31-decaoxa-34,36-diazatetracontanoate 2,5,8,11,14,17,20,23,26,29-decaoxa-33-azatetratriacontan-34-yl)phenyl)imino)-4,7,10,13,16,19,22,25,28,31-decaoxa-34,36-diazatetracontanoic acid in 45% yield over two steps. LC/MS [M+H] 1601.85 (calculated); LC/MS [M+H] 1602.07 (observed).

### Example 119: HEK Reporter Assay

HEK293 reporter cells expressing human TLR7 or human TLR8 were purchased from Invivogen and vendor protocols were followed for cellular propagation and experimentation. Briefly, cells were grown to 80-85% confluence at 5% CO₂ in DMEM supplemented with 10% FBS, Zeocin, and Blasticidin. Cells were then seeded in 96-well flat plates at 4×10⁴ cells/well with substrate containing HEK detection medium and immunostimulatory molecules. Activity was measured using a plate reader at 620-655 nm wavelength.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A macromolecule-supported compound of formula (I) or formula 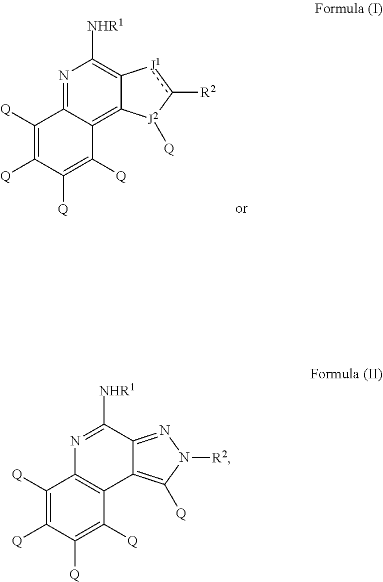
a pharmaceutically acceptable salt thereof, or a quaternary ammonium salt thereof,
wherein
R¹ and R² independently are hydrogen or of formula: 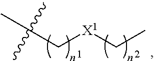
J¹ is CH or N,
J² is CHQ, NQ, O, or S,
each Q independently is Y or Z, wherein exactly one Q is Y,
Y is of formula: 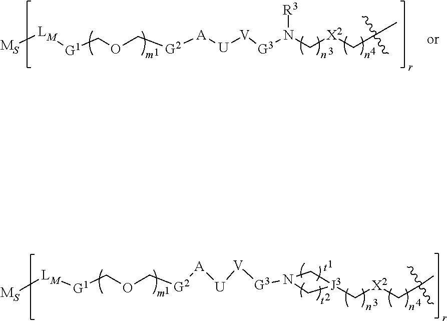
each Z independently is hydrogen or of formula: 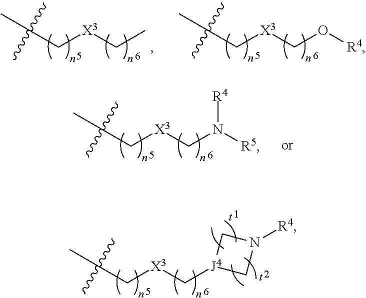
A is optionally present and is NR⁶ or of formula: 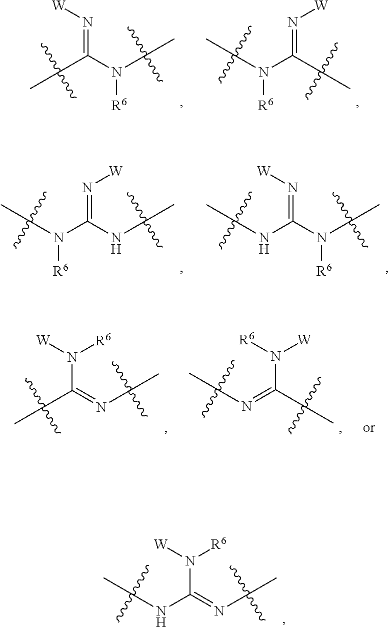
U is optionally present and is CH₂, C(O), CH₂C(O), or C(O)CH₂,
R⁶ and W independently are hydrogen, Ar¹, or of formula: 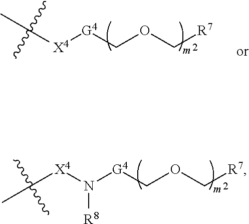
V is optionally present and is of formula: 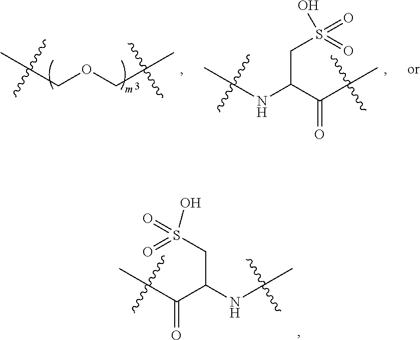
J³ and J⁴ independently are CH or N,
m¹, m², and m³ independently are an integer from 0 to 25, except that at least one of m¹, m², and m³ is a non-zero integer,
n¹, n², n³, n⁴, n⁵, and n⁶ independently are an integer from 0 to 10,
t¹ and t² independently are an integer from 1 to 3,
G¹, G², G³, and G⁴ independently are CH₂, C(O), CH₂C(O), C(O)CH₂, or a bond,
X¹, X², X³, and X⁴ are each optionally present and independently are O, NR⁹, CHR⁹, SO₂, S, or one or two divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,
R⁴ is hydrogen, C₁-C₄ alkyl, 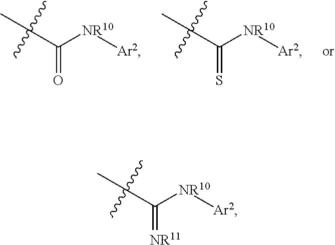
R³, R⁵, R⁷, R⁸, R⁹, R¹⁰, and R¹¹ independently are hydrogen or C₁-C₄ alkyl,
Ar¹ and Ar² independently are an aryl or heteroaryl group, optionally substituted with one or more halogens (e.g., fluorine, chlorine, bromine, or iodine), nitriles, hydroxyls, C₁-C₄ alkyl groups, or a combination thereof,
L_{M} is a linking moiety,
r is an integer from 1 to 50,
“M_{S}” is a macromolecular support, and
each wavy line (“ [symbol] ”) represents a point of attachment.

2. The macromolecule-supported compound of claim 1, wherein
subscript r is an integer from 1 to 25.

3. The macromolecule-supported compound of claim 2, wherein
subscript r is an integer from 1 to 6.

4. The macromolecule-supported compound of claim 1, wherein the macromolecular support is a peptide.

5. The macromolecule-supported compound of claim 1, wherein the macromolecular support is a nucleotide.

6. The macromolecule-supported compound of claim 1, wherein the macromolecular support is carbohydrate.

7. The macromolecule-supported compound of claim 1, wherein the macromolecular support is lipid.

8. The macromolecule-supported compound of claim 1, wherein the macromolecular support is an antibody construct.

9. The macromolecule-supported compound of claim 1, wherein the macromolecular support is a biopolymer.

10. The macromolecule-supported compound of claim 1, wherein the macromolecular support is a nanoparticle.

11. The macromolecule-supported compound of claim 1, wherein the macromolecular support is an immune checkpoint inhibitor.

12. The macromolecule-supported compound of claim 1, wherein the macromolecule-supported compound is of formula: 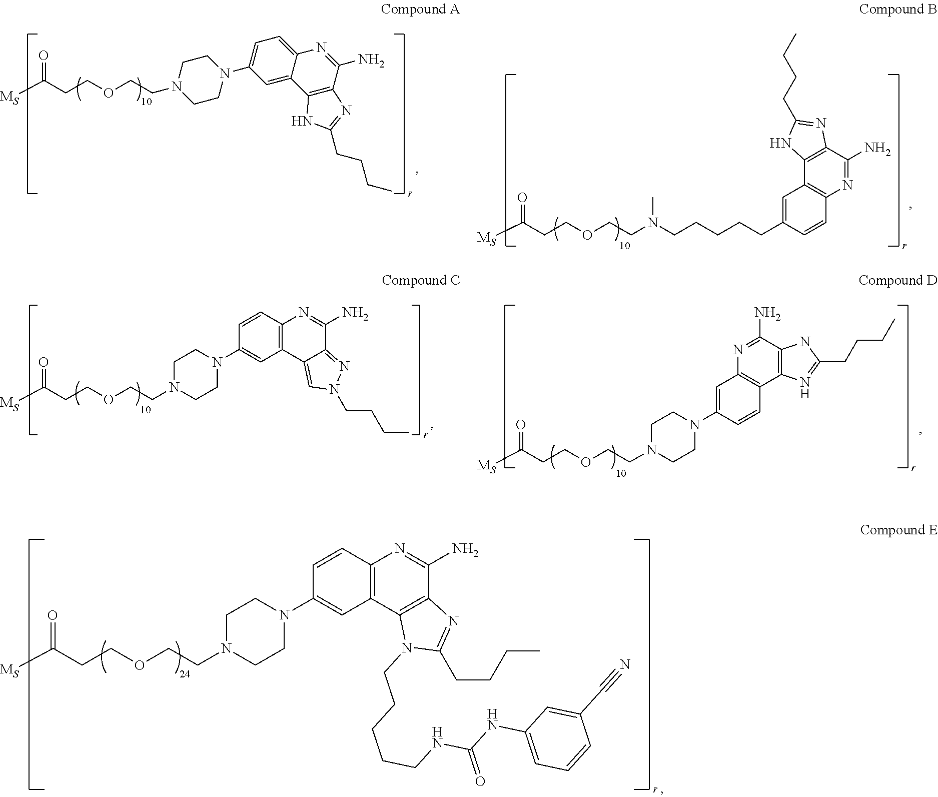 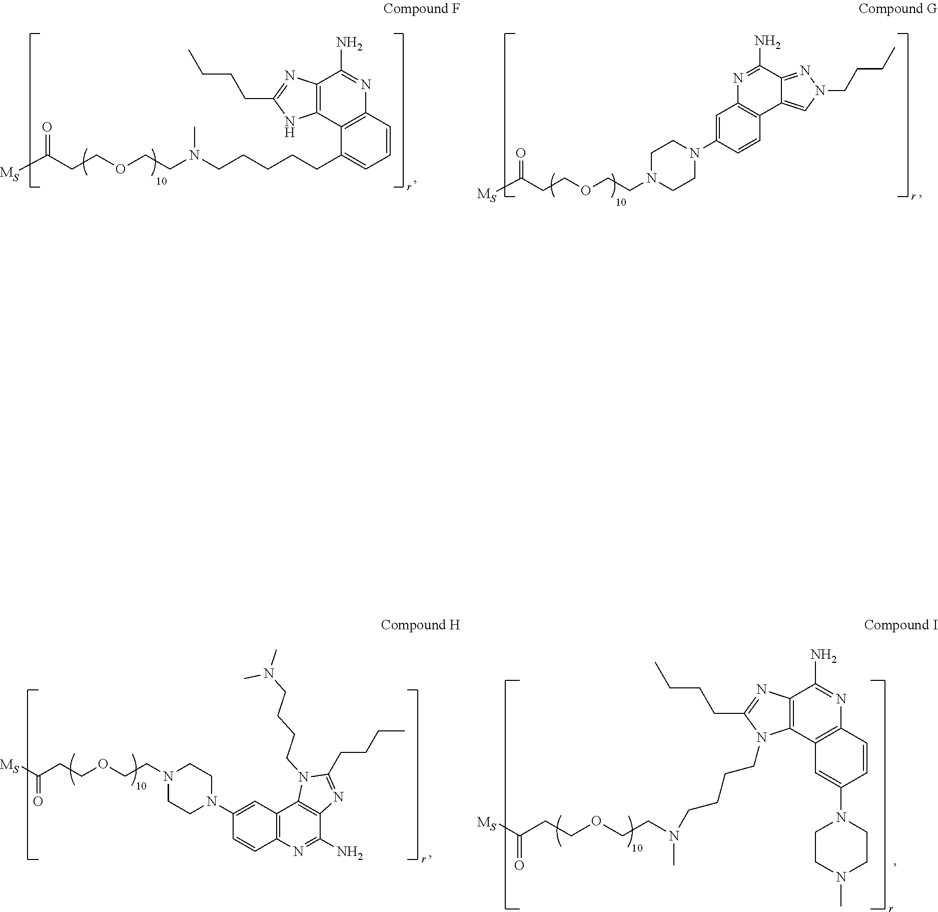 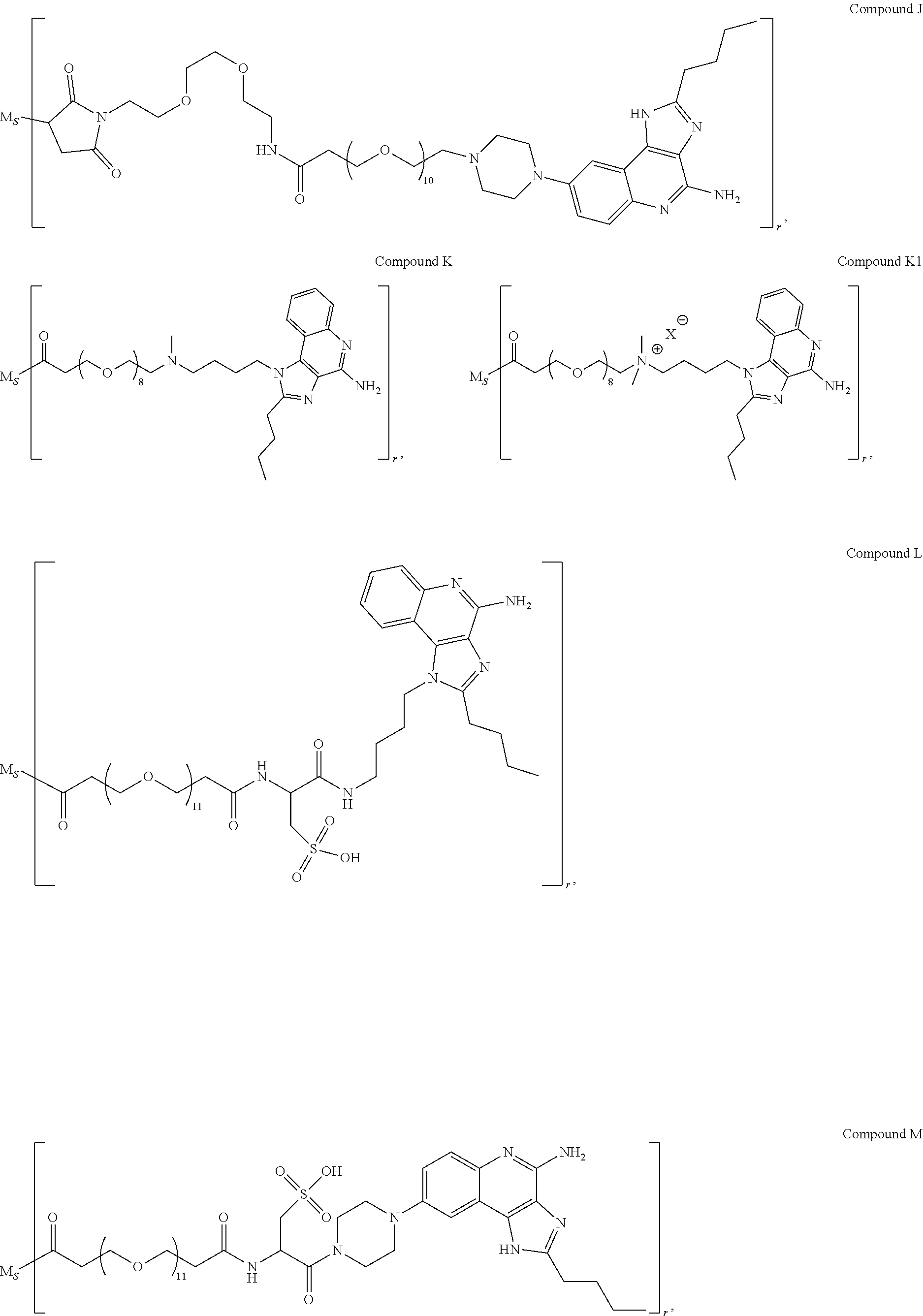 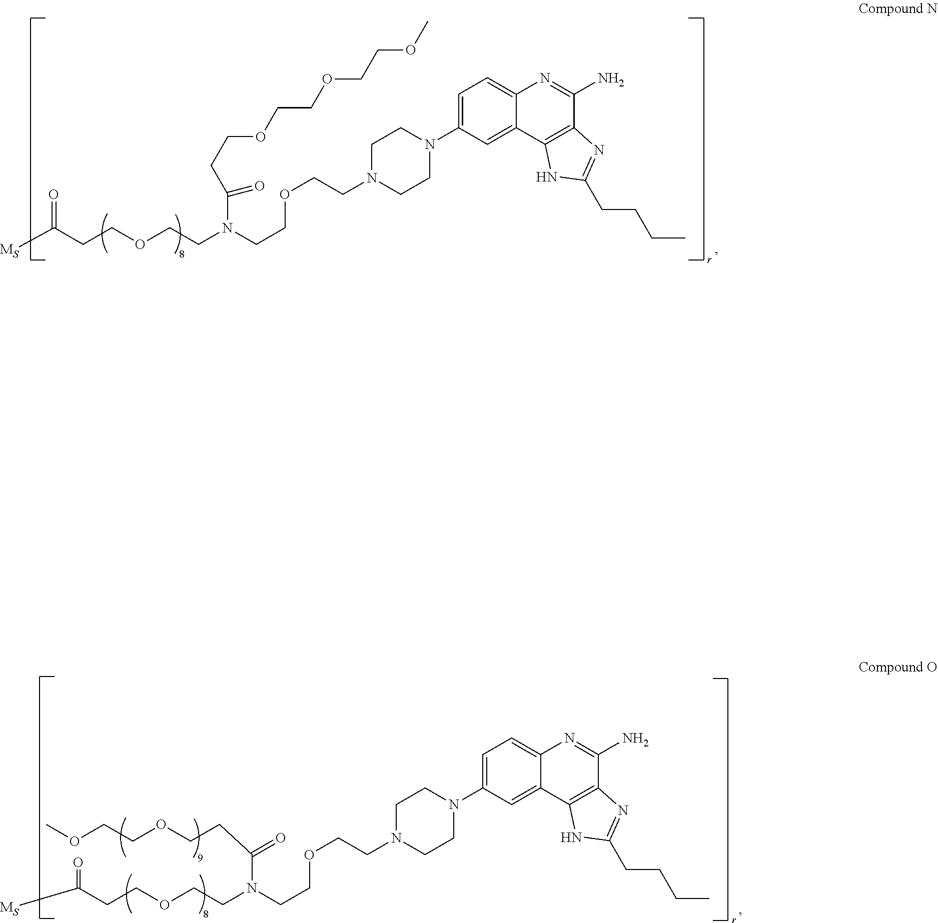 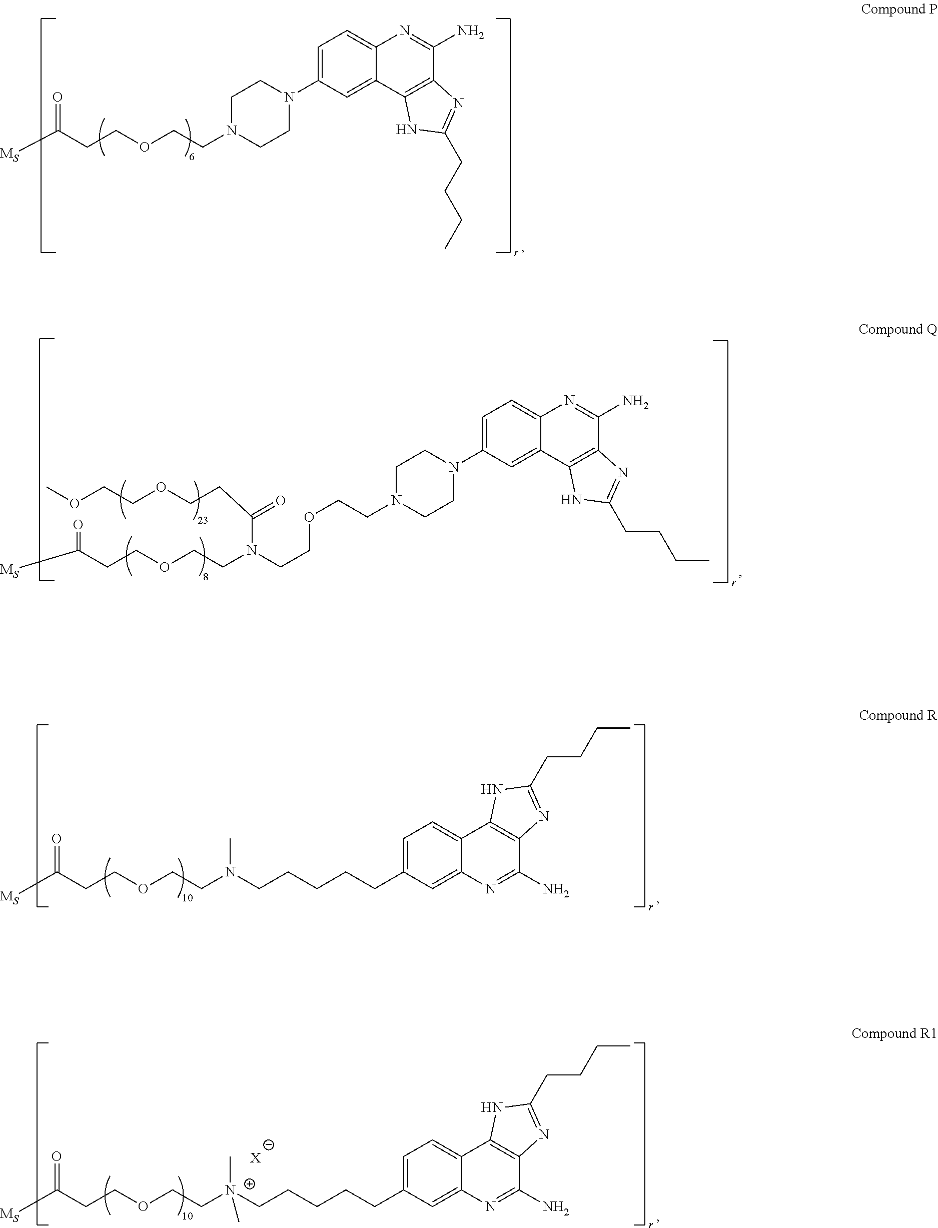 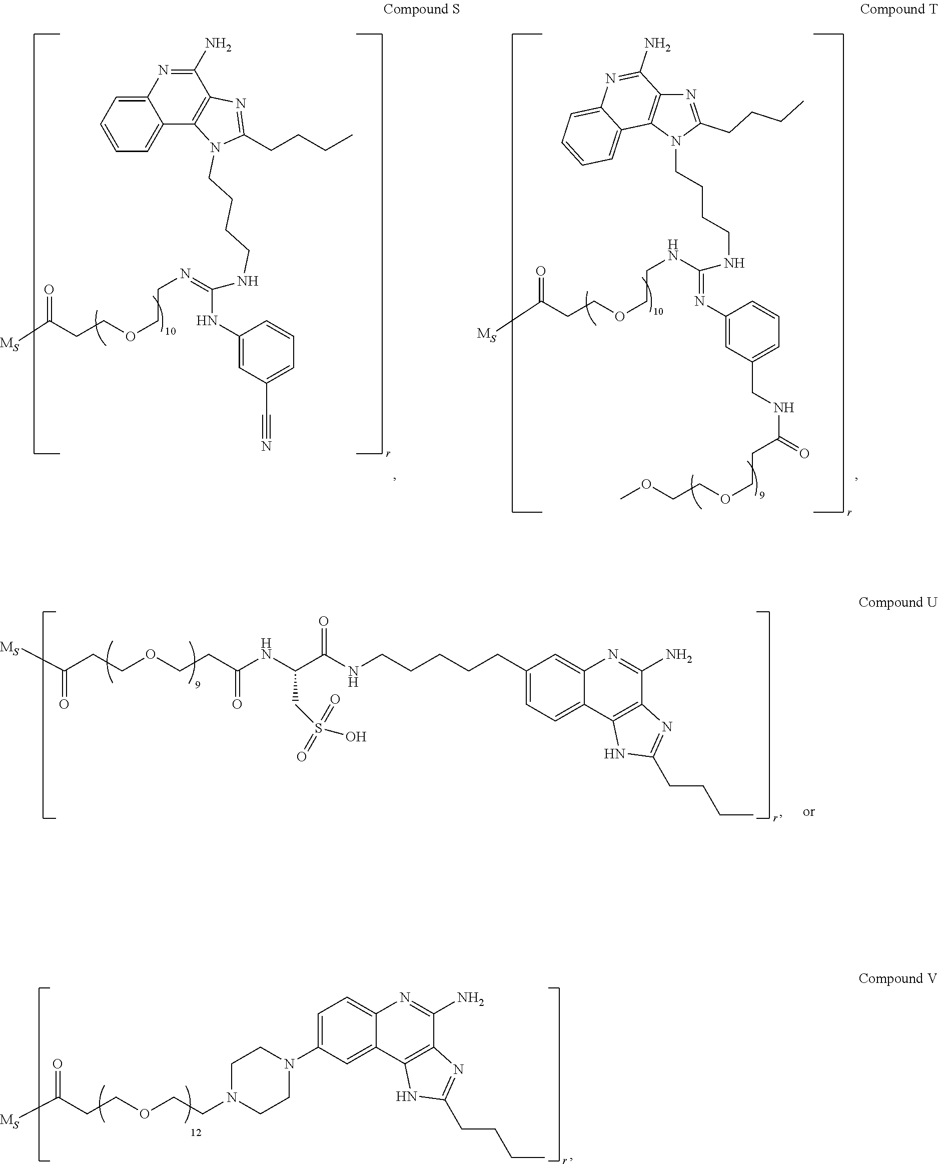
a pharmaceutically acceptable salt thereof, or a quaternary ammonium salt thereof, wherein subscript r is an integer from 1 to 50 and “M_{S}” is macromolecular support.

13. A composition comprising a plurality of macromolecule-supported compounds according to claim 1.

14. The composition of claim 13, wherein the average TLR agonist to macromolecular support ratio is from about 0.01 to about 50.

15. The composition of claim 14, wherein the average TLR agonist to macromolecular support ratio is from about 1 to about 10.

16. (canceled)

17. The composition of claim 15, wherein the average TLR agonist to macromolecular support ratio is from about 1 to about 4.

18. A method for treating cancer comprising administering a therapeutically effective amount of a macromolecule-supported compound according to claim 1 to a subject in need thereof.

19.-22. (canceled)

23. A method for treating cancer comprising administering a therapeutically effective amount of a composition according to claim 13 to a subject in need thereof.

24. A method for determining TLR engagement and/or activity comprising using a macromolecule-supported compound according to claim 1 in a chemical assay.

25. A method for determining TLR engagement and/or activity comprising using a composition according to claim 12 in a chemical assay.
